(19) United States
(12) Patent Application Publication
(10) Pub. No.: US 2010/0158927 A1
(43) Pub. Date: Jun. 24, 2010

(54) ANTIBODIES, METHODS AND KITS FOR DIAGNOSING AND TREATING MELANOMA

(75) Inventors: Yoram Reiter, Haifa (IL); Cyril Cohen, Nesher (IL); Eynav Klechevsky, Haifa (IL); Yael Michaeli, Binyamina (IL); Maya Haus-Cohen, Haifa (IL); Galit Denkberg, Nofit (IL)

Correspondence Address: MARTIN D. MOYNIHAN d/b/a PRTSI, INC., P.O. BOX 16446, ARLINGTON, VA, 22215, US

(73) Assignee: Technion Research & Development Foundation Ltd., Haifa (IL)

(21) Appl. No.: 12/450,479

(22) PCT Filed: Mar. 27, 2008
(86) PCT No.: PCT/IL2008/000436

§ 371 Date: Feb. 2, 2010

Related U.S. Application Data
(60) Provisional application No. 60/907,343, filed on Mar. 29, 2007.
(60) Provisional application No. 60/924,549, filed on May 21, 2007.

(51) Int. Cl. A61K 39/395; G01N 33/574; C12N 9/96; C12N 5/02; C07K 16/00

(52) U.S. Cl. 424/178.1; 435/7.23; 435/188; 435/375; 530/387.9

(57) ABSTRACT

A method of diagnosing melanoma and antibodies capable of same are disclosed. The method comprises contacting a cell of the subject with an antibody comprising an antigen recognition domain capable of binding to an MHC-I molecule being complexed with a tyrosinase peptide, wherein the antibody does not bind the MHC-I in the absence of the complexed peptide, and wherein the antibody does not bind the peptide in an absence of the MHC, under conditions which allow immunocomplex formation, wherein a presence of the immunocomplex or level thereof is indicative of the melanoma. Methods for treating melanoma and antibodies capable of same are also disclosed. Pharmaceutical compositions comprising antibodies are also disclosed.

### FIELD AND BACKGROUND OF THE INVENTION

The present invention relates to methods of treating and diagnosing melanoma and, more particularly, to antibodies capable of same.

A key advance in immunology in the past decade has been the elucidation of the antigenic basis of tumor-cell recognition and destruction. The ultimate effector cell that mediates the immune activity against tumors is the cytotoxic T cell (CTL). Protein antigens, recognized by CTLs through their clonotypic and specific T cell receptor, consist of peptide fragments which are bound within the antigen binding cleft of the major histocompatibility complex (MHC) class I molecules on the cell surface. Antigens are exposed to immune-system scrutiny by loading peptide fragments of newly synthesized cellular proteins onto MHC-class-I molecules, which are then transported to the cell surface.

As in normal cells, the surface of tumor cells contains MHC-peptide antigens that reflect their expressed ‘proteome’. Tumor antigens include the gene products arising de novo which are unique to individual cancer cells (e.g., an epitope from a mutated β-catenin gene), the gene products derived from an aberrant expression of non-mutated genes (e.g., NY-ESO-1), the products of which are normally expressed only in testes or fetal tissues and non-mutated, and cell-lineage-specific proteins (also termed “differentiation antigens”). A similar diversity of tumor antigens is recognized by MHC class II-restricted CD4+ T cells.

Melanomas are aggressive, frequently metastatic tumors derived from either melanocytes or melanocyte related nevus cells. Even when melanoma is apparently localized to the skin, up to 30% of patients develop systemic metastasis. Classic treatment modalities of melanoma include surgery, radiation and chemotherapy. In the past decade immunotherapy and gene therapy have emerged as new and promising methods for treating melanoma.

Shared melanoma-associated antigens (Ag) (e.g., MART-1, gp100 and Tyrosinase) expressed among a variety of melanoma patients can be recognized by cytotoxic CD8+ T lymphocytes derived from melanoma patients. T-cell lines which specifically recognize the HLA restricted antigens expressed by tumor cells were generated and used to identify the genes encoding tumor antigens and the peptide epitopes derived therefrom. The fate of a specific T cell clone is determined in a secondary lymphoid organ where it is “educated” by a professional antigen presenting cell (pAPC). Upon pAPC encounter, a constellation of events ensues, inter alia, co-stimulatory signaling, various interactions between membrane determinants in the immunological synapse, as well as cytokines secreted by the pAPC and the T cell, which ultimately determine whether a specific T cell clone will undergo an activation process, tolerance, or apoptosis. Activation of a T cell effectuates morphological, genetic, and biochemical changes that enhance its proliferation, migration, and effector functions. Activated CTLs survive only days to weeks after elimination of the antigen source; however a small proportion of these T cells transform and constitute subpopulations of memory T cells with distinctly different surface markers.

Current immunotherapy approaches are designed to induce and enhance T cell reactivity against tumor antigens. Intensive research on cancer peptides has culminated in clinical trials involving therapeutic vaccination of cancer patients with antigenic peptides or proteins. Such vaccinations often result in tumor-reactive CTLs in patients (see for example, A. Lev, et al., 2002; C. J. Cohen, et al., 2003). U.S. Pat. Appl. No. 20050158332 discloses MHC Class II restricted melanoma antigens of the tyrosinase sequence recognized by CD4+ T-lymphocytes and their therapeutic use in increasing immunogenicity by enhancing the binding of the peptide to the MHC Class II molecule. U.S. Pat. Appl. No. 20030144482 discloses MART-1 antigenic peptides capable of causing a cellular or humoral immune response in a mammal. However, vaccination alone only sporadically induces tumor regression in patients with metastatic disease. In addition, even in transgenic mice in which all T cells have been engineered to express a tumor-reactive TCR, tumors still progressively grow.

The lack of inflammatory rejection of tumors by immunized patients and TCR transgenic mice is not well understood at the cellular and molecular level. Many mechanisms could account for the failure of antigen-specific CD8+ T cells to eliminate antigen-expressing tumor cells in vivo. For instance, the tumor-antigen-specific T cells themselves could be functionally deficient, rendered anergic, or unable to fully differentiate in the tumor environment. In addition, the tumor environment could lack a ‘danger signal’ or other innate immune stimulation, preventing a general inflammatory reaction from evolving. Alternatively, active immune regulatory mechanisms such as CD4+CD25+ T cells might impede any endogenous immune reaction to cancer cells. Whatever the mechanism, without an inflammatory immune response, the CD8+ T cells of the adaptive immune system are rendered ineffective. As a tumor grows and metastasizes, additional systemic immune suppression could develop, and antigen-escape variants of the tumor could arise.

Several studies have demonstrated that the inability of a patient's immune system to elicit an effective immune response against a tumor is often due to poor antigen presentation [Koopman L A, et al J Exp Med 20: 961-976; Gamido F, et al., Immunol Today 18: 89-95; Rosenberg S A, Bennink J R. 1993. J Exp Med 177: 265-272]. In addition, the CD8+ T cell-mediated cytotoxic effect on targeted pathogenic cells requires at least a minimum density of antigen presentation. However, to date, the mechanisms of antigen presentation are not fully understood.

The advent of MHC-peptide tetramers has provided a new tool for studying antigen-specific T cell populations in health and disease, by monitoring tetramer-T cell binding via flow cytometry. However, to date there are limited tools available to detect, visualize, count, and study antigen (i.e., MHC-peptide complex) presentation. Antibodies that specifically recognize class I MHC-peptide complexes have been previously described, see for example U.S. Pat. Appl. No. 20050255101. However, the currently available antibodies could not explain the hyporesponsiveness of T cells to melanoma differentiation antigens. In addition, specific antibodies directed against MHC-I molecules complexed with additional melanoma differentiation antigens such as Mart-1 or Tyrosinase have not as yet been described.

There is thus a widely recognized need for, and it would be highly advantageous to have, TCR-like antibodies capable of diagnosing and treating melanoma devoid of the above limitations.

### SUMMARY OF THE INVENTION

According to an aspect of the present invention there is provided an antibody comprising an antigen recognition domain capable of binding to an MHC-I molecule being complexed with a tyrosinase peptide comprising an amino acid sequence as set forth in SEQ ID NO: 1, wherein the antibody does not bind the MHC-I in the absence of the complexed peptide, and wherein the antibody does not bind the peptide in an absence of the MHC.

According to an aspect of the present invention there is provided a pharmaceutical composition comprising the antibody of the present invention.

According to an aspect of the present invention there is provided a method of detecting a melanoma cell, comprising contacting the cell with an antibody comprising an antigen recognition domain capable of binding to an MHC-I molecule being complexed with a tyrosinase peptide, wherein the antibody does not bind the MHC-I in the absence of the complexed peptide, and wherein the antibody does not bind the peptide in an absence of the MHC, under conditions which allow immunocomplex formation, wherein a presence of the immunocomplex or level thereof is indicative of the melanoma cell.

According to an aspect of the present invention there is provided a method of diagnosing a melanoma in a subject in need thereof, comprising contacting a cell of the subject with an antibody comprising an antigen recognition domain capable of binding to an MHC-I molecule being complexed with a tyrosinase peptide, wherein the antibody does not bind the MHC-I in the absence of the complexed peptide, and wherein the antibody does not bind the peptide in an absence of the MHC, under conditions which allow immunocomplex formation, wherein a presence of the immunocomplex or level thereof is indicative of the melanoma.

According to an aspect of the present invention there is provided a method of identifying if a subject is suitable for TCRL-based epitope directed therapy, comprising determining a level of epitope presentation on at least one cell of the subject using an antibody comprising an antigen recognition domain capable of binding to an MHC-I molecule being complexed with a peptide fragment of the antigen, wherein the antibody does not bind the MHC-I in the absence of the complexed peptide fragment of the antigen, and wherein the antibody does not bind the peptide fragment of the antigen in an absence of the MHC, wherein a level value higher than a predetermined threshold is indicative of an individual being suitable for TCRL-based epitope directed therapy.

According to an embodiment of this aspect of the present invention, a level value below a predetermined threshold is indicative of an individual not being suitable for TCRL-based epitope directed therapy.

According to an aspect of the present invention there is provided a method of identifying if a subject is suitable for CTL-based epitope directed therapy, comprising determining a level of epitope presentation on at least one cell of the subject using an antibody comprising an antigen recognition domain capable of binding to an MHC-I molecule being complexed with a peptide fragment of the antigen, wherein the antibody does not bind the MHC-I in the absence of the complexed peptide fragment of the antigen, and wherein the antibody does not bind the peptide fragment of the antigen in an absence of the MHC, wherein a level value lower than a predetermined threshold is indicative of an individual being suitable for CTL-based epitope directed therapy.

According to an aspect of the present invention there is provided a method of treating a melanoma, comprising administering to a subject in need thereof a therapeutically effective amount of an antibody comprising an antigen recognition domain capable of binding to an MHC-I molecule being complexed with a tyrosinase peptide, wherein the antibody does not bind the MHC-I in the absence of the complexed tyrosinase peptide, and wherein the antibody does not bind the tyrosinase peptide in an absence of the MHC, thereby treating the melanoma.

According to an aspect of the present invention there is provided a method of killing or ablating a cell displaying a tyrosinase peptide on a surface MHC molecule, the method comprising contacting the target cell with an antibody comprising an antigen recognition domain capable of binding to an MHC-I molecule being complexed with a tyrosinase peptide, wherein the antibody does not bind the MHC-I in the absence of the complexed tyrosinase peptide, and wherein the antibody does not bind the tyrosinase peptide in an absence of the MHC, thereby killing or ablating the cell.

According to an aspect of the present invention there is provided an antibody comprising an antigen recognition domain capable of binding to an MHC-I molecule being complexed with a MART-1 peptide comprising an amino acid sequence as set forth in SEQ ID NO: 21, wherein the antibody does not bind the MHC-I in the absence of the complexed peptide, and wherein the antibody does not bind the peptide in an absence of the MHC.

According to some embodiments of the invention, the antibody comprises an antigen recognition domain which comprise complementarity determining region (CDR) amino acid sequences as set forth in SEQ ID NOs: 59-64.

According to some embodiments of the invention, the antibody comprises complementarity determining region (CDR) amino acid sequences as set forth in SEQ ID NOs: 59-64.

According to some embodiments of the invention, the antibody binds to an MHC-I molecule being complexed with a tyrosinase peptide with a disassociation constant less than 100 nM.

According to some embodiments of the invention, the antibody comprises an antigen recognition domain which comprises complementarity determining region (CDR) amino acid sequences as set forth in SEQ ID NOs: 886-891.

According to some embodiments of the invention, the antibody comprises an antigen recognition domain which comprises complementarity determining region (CDR) amino acid sequences as set forth in SEQ ID NOs: 894-899.

According to some embodiments of the invention, the antibody comprises complementarity determining region (CDR) amino acid sequences as set forth in SEQ ID NOs: 886-891

According to some embodiments of the invention, the antibody comprises complementarity determining region (CDR) amino acid sequences as set forth in SEQ ID NOs: 894-899.

According to some embodiments of the invention, the antibody binds to an MHC-I molecule being complexed with a tyrosinase peptide with a disassociation constant less than 10 nM.

According to some embodiments of the invention, the antibody is an IgG1 antibody.

According to some embodiments of the invention, the antibody is conjugated to a therapeutic moiety.

According to some embodiments of the invention, the therapeutic moiety is selected from the group consisting of a cytotoxic moiety, a toxic moiety, a cytokine moiety and a bi-specific antibody moiety.

According to some embodiments of the invention, the toxic moiety is PE38 KDEL.

According to some embodiments of the invention, the antibody is attached to a detectable moiety.

According to some embodiments of the invention, the detectable moiety is a fluorescent protein or an enzyme.

According to some embodiments of the invention, the antibody is an antibody fragment.

According to some embodiments of the invention, the antibody fragment is selected from the group consisting of an Fab fragment, an F(ab′)₂ fragment and a single chain Fv fragment.

According to some embodiments of the invention, the cell is a skin cell.

According to some embodiments of the invention, the tyrosinase peptide comprises an amino acid sequence as set forth in SEQ ID NO: 1.

According to some embodiments of the invention, the antibody comprises an antigen recognition domain capable of binding to an MHC-I molecule being complexed with a tyrosinase peptide with a disassociation constant less than 100 nM, wherein the antibody does not bind the MHC-I in the absence of the complexed peptide, and wherein the antibody does not bind the peptide in an absence of the MHC.

According to some embodiments of the invention, the antibody comprises an antigen recognition domain which comprises complementarity determining region (CDR) amino acid sequences as set forth in SEQ ID NOs: 47-52.

According to some embodiments of the invention, the antibody comprises an antigen recognition domain which comprises complementarity determining region (CDR) amino acid sequences as set forth in SEQ ID NOs: 53-58.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the biotechnology art.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph depicting the binding of soluble purified Fabs with TCR-like specificity. Binding ELISA assay of anti-HLA-A2/Tyrosinase-specific clones with immobilized HLA-A2-peptide complexes containing Tyrosinase D 369-377 peptide (SEQ ID NO:1), and control HLA-A2-restricted peptides. Anti-HLA mAb W6/32 (W6), was used to determine the correct folding and stability of the bound complexes during the binding assay. Note the specific binding of A2, A12, E5 and D11 soluble purified Fabs to MHC class I complexes of HLA-A2− Tyrosinase D 369-377 peptide as compared to the absence of binding to MHC-class I complexes of HLA-A2 and control antigenic peptides such as Gag (SEQ ID NO:2), Tyr N (SEQ ID NO:3), 2092M (SEQ ID NO:4), 280 (SEQ ID NO:5), 540 (SEQ ID NO:6), and TARP (SEQ ID NO:7.

FIGS. 2a-e are flow cytometry analyses depicting the binding of HLA-A2/Tyr—specific TCR-like antibody to antigen presenting cells (APCs). FIG. 2a-Flow cytometry analysis of the binding of Fab TA2 to JY APCs pulsed with Tyrosinase 369-377 peptide or the control peptides described in FIG. 1 as well as 154 (SEQ ID 20), Tax (SEQ ID NO: 26), 280 m (SEQ ID NO: 28), pol (SEQ ID NO: 65), 865 (SEQ ID NO: 29) and Mart (SEQ ID NO: 22) peptides were used); FIGS. 2b-c—Flow cytometry analyses of FabTA2 in a monomeric and tetrameric form to JY APCs pulsed with the Tyrosinase 369-377 peptide (FIG. 2b) or to control peptide (209 2M peptide SEQ ID NO. 4; FIG. 2c); FIGS. 2d-e—Flow cytometry analyses of the binding of TA2 whole IgG antibody to JY cells pulsed with Tyrosinase 369-377 peptide (FIG. 2d) or control peptide (209 2M peptide; SEQ ID NO. 4) (FIG. 2e).

FIGS. 3a-e are RT-PCR analyses of melanoma cell lines depicting the expression of melanoma antigens. RNA was extracted from the 624.38 (FIG. 3a), 501A (FIG. 3b), 2224 (FIG. 3c), 1352 (FIG. 3d) or 1938 (FIG. 3e) melanoma cell lines and RT-PCR was performed using PCR primers specific for the genes MelanA/Mart1 (SEQ ID NOs:8 and 9; lane 4), Pmel17/gp100 (SEQ ID NOs:10 and 11; lane 3), Tyrosinase (SEQ ID NOs:12 and 13; lane 2) and GAPDH (SEQ ID NOs:14 and 15; lane 1). Note that while the 624.38, 501A, 2224 and 1352 expressed all three melanoma-related genes (Tyrosinase, gp100 and Mart1, FIGS. 3a-d), the 1938 cell line expressed only the positive control GAPDH gene (FIG. 3e).

FIGS. 4a-g are flow cytometry analyses depicting the detection of HLA-A2-Tyrosinase complexes on the surface of melanoma cell by TA2 TCR-like antibody. FIGS. 4a-f—624.38 (FIG. 4a), 501A (FIG. 4b), and TC-2224 (FIG. 4c), which express both the HLA-A2 and the Tyrosinase genes, as well as HLA-AT 1352 cells (FIG. 4d), and Tyr⁻ 1938 cells (FIG. 4e), were reacted with TA2 Ab, followed by incubation with PE-labeled anti-human Ab. TC-2224 and TC-1352 were reacted with TA2 IgG, 624.38, 501A and 1938 were reacted with the TA2 Fab. Note the specific binding of the TA2 Ab with the melanoma cells expressing HLA-A2 and tyrosinase as compared to the negative binding of the TA2 Ab with the TC-1352 (HLA-A2⁻/Tyr⁺) or 1938 (HLA-A2⁺/Tyr⁻) cells which served as controls. FIGS. 4f-g are comparative flow cytometry analysis of TA2 Fab and whole IgG antibody. FIG. 4f—Staining with 5 μg Fab and 1 μg IgG on Tyrosinase-pulsed JY cells. FIG. 4g—Titration of the binding of TA2 whole IgG on melanoma 624.38 cells.

FIGS. 5a-d depict the killing of melanoma cells by Tyrosinase-specific CTLs. FIGS. 5a-b are flow cytometry analyses of melanoma 624.38 (FIG. 5a) and TC-2183 (FIG. 5b) cells using the TA2 whole IgG TCR-like antibody labeled with PE-conjugated anti-human antibody, specific to the HLA-A2/tyrosianse complexes. Note the high expression level of HLA-A2/tyrosianse complexes on the surface of melanoma 624.38 cells (FIG. 5a) as compared to the low expression in TC-2183 cells (FIG. 5b). FIGS. 5c-d are graphs depicting cytotoxicity assays of 624.38 (FIG. 5c) and TC-2183 (FIG. 5d) melanoma cells by the TyrosinaseD-369-377-specific CTLs. Melanoma cells were either peptide-pulsed (YMD=Tyrosinase D 369-377 peptide) or un-pulsed with the TyrosinaseD-369-377 peptide and the effect of the TyrosinaseD-369-377-specific CTLs on cell killing was determined. Results are presented as the specific lysis (in percentages) as a function of the effector (CTL) to target (Melanoma cells) ratio.

FIGS. 6a-d are confocal microscopy images of immuno-fluorescence analysis depicting the binding of HLA-A2/Tyrosinase-specific TCR-like antibody to melanoma cells. The 501A (FIGS. 6a-c) or 1938 (FIG. 6d) melanoma cells were subjected to double immuno-fluorescence analysis using labeled TA2 FAB (green) and anti-HLA-A2 MAb BB7 (red). Nuclei were stained with DRAQ5. FIG. 6a—TA2 labeling; FIG. 6b—BB7 labeling; FIGS. 6c-d—merged images of TA2 and BB7 labeling. Note the specific labeling of TA2 and BB7 on the cell membrane of the 501A melanoma cells and the absence of labeling of both antibodies on the cell membrane of 1938 cells. FIG. 6d—1938 reacted with TA2 and not BB7. After incubation with TA2 or BB7, anti-human alexa 488 or anti-mouse alexa 594 antibodies were used, respectively.

FIGS. 7a-e depict quantification of Tyrosinase-HLA-A2 complexes by TA2. FIGS. 7a, b, d and e—Flow cytometry analyses. FIG. 7c—a standard curve of the flow cytometry analyses. 501A (HLA-A2⁺/Tyr⁺) (FIGS. 7a-b) and TC-1352 (HLA-A2⁻/Tyr⁺) (FIG. 7d-e) melanoma cells were incubated with biotinylated TA2 Fab (FIGS. 7a and d), followed by detection with PE-labeled streptavidin. Expression of HLA-A2 complexes was monitored with PE conjugated-BB7.2 mAb (FIGS. 7b and e). For a standard curve, calibration beads with known numbers of PE molecules per bead were used (FIG. 7c). To perform the standard curve, 1 ml of PBS 0.1% BSA was added to the beads, the beads were located in the middle of the FSC SSC dot blot and were analyzed for their fluorescence intensities in various FL2 values. MFI=mean fluorescence intensities; FL=the value of FL2 that was used; The quantification results obtained from the flow cytometry analyses are summarized in Table 2.

FIGS. 8a-d depict the expression hierarchy of melanoma differentiation antigens by TCR-like antibodies. FIGS. 8a-d are comparative flow cytometry analyses of the expression of HLA-A2-Tyr/Mart-1/gp100 complexes on the surface of TC-2224 (FIG. 8a), 501A (FIG. 8b), 624.38 (FIG. 8c) and 1352 (FIG. 8d) melanoma cells. Melanoma cells were incubated with anti-HLA-A2-Tyr TA2, anti-HLA-A2-Mart-1 CLA12, anti-HLA-A2-gp100-209 1A7, and anti-HLA-A2-gp100 280 2F1, TCR-like antibodies, all in the IgG form. HLA-A2 expression was monitored with MAb BB7.2. Gene expression of the antigens is shown. Note the relatively high representation of the TA2-positive cells in TC-2224, 501A and 634.38 cells are compared to the lower representation of 1A7, 2F1 or CLA12-positive cells.

FIGS. 9a-b are flow cytometry analyses depicting the effect of Tyrosinase protein stability on the reactivity of TA2 TCR-like antibody with melanoma cells. 501A melanoma cells were treated (FIG. 9b) or not (FIG. 9a) with 1 mM DOPA for 16 hours, following which the cells were incubated with TA2 followed by incubation with PE-labeled anti-human Ab and the mean fluorescence intensities (MFI) were determined. Quantification of the effect of DOPA on MFI is summarized in Table 5 (Example 6 of the Examples section which follows).

FIGS. 10a-d are Western blot analyses (FIGS. 10a-b) and quantification thereof (FIGS. 10c-d) depicting the degradation of differentiation antigens in melanoma cells. Protein synthesis in melanoma 501A cells was arrested by cyclohexamide and the stability of Tyrosinase (FIGS. 10a and c), Mart1 (FIGS. 10b and d) and gp100. The cells were lysed after 0, 2, 4 and 6 h. Equal amounts of sample were loaded on SDS-PAGE and electroblotted onto nitrocellulose. Protein was measured over time in cell extracts that were subjected to Western blot analysis using the specific monoclonal antibodies (The blots were probed with T311 mouse anti-Tyrosinase, HMB-45 mouse anti-gp100 or A103 mouse anti-Mart-1 followed by incubation with a secondary horseradish peroxidase-conjugated antibody. Note the rapid degradation of Tyrosinase (FIG. 10c) as compared to that of Mart1 (FIG. 10d) or gp100 (−0.152) as revealed by the line slope (−0.25 in FIG. 10c vs. −0.1 in FIG. 10d). There was a hierarchy of degradation rate or protein stability with Mart-1 being the most stable with a t1/2 of ˜6.5 hrs, gp100 with moderate stability (t1/2 of ˜4.5 hrs) and Tyrosinase being the less stable protein of the three antigens with a t1/2 of ˜2.5 hrs.

FIGS. 11a-c depict the relative expression of melanoma differentiation antigens by real-time PCR. Real-time PCR analysis was performed on RNA samples of 31 melanoma cells lines derived from patients using RT-PCR primers specific to Tyrosinase, Melan-A and gp100. FIG. 11a is an example of real time PCR amplification graph; FIG. 11b—A histogram depicting the relative gene expression of tyrosinase, Melan-A and gp100 expressed as 2^{-ΔCT} units for 21 out of the 31 melanoma cell lines examined. FIG. 11c illustrates the relative gene expression of tyrosinase, Melan-A and gp100 expressed as 2^{-ΔCT} units for 21 out of the 31 melanoma cell lines examined.

FIGS. 12a-c are flow cytometry analyses depicting the quantification of the number of HLA-A2/Tyrosinase complexes on melanoma cells. Melanoma lines 624.38 (FIG. 12a), TC-2207 (FIG. 12b), TC-1760 (FIG. 12c) expressing high, moderate, and low levels of Tyrosinase, respectively, were incubated with biotinylated TA2 Fab followed by detection with PE-labeled streptavidin.

FIG. 13 is a bar graph depicting the relative gene expression of Tyrosinase, Melan-A and gp100 melanoma antigens on the 501A, 624.38, 1352 and TC-2224 melanoma cells.

FIGS. 14a-i depict cytotoxicity assays of peptide-pulsed cells demonstrating that high antigen density induces hypo-responsiveness in memory activated CTLs. FIGS. 14a-d are histograms depicting the relative cytotoxicity as a function of peptide concentration. JY B-lymphoblast APCs were pulsed with increasing concentrations of the melanoma differentiation antigens gp100_{G209-217} (SEQ ID NO:4; FIG. 14a) and MART1₂₇₋₃₅ (SEQ ID NO:22; FIG. 14b), the EBV derived peptide BMLF-1₂₈₀₋₂₈₈ (SEQ ID NO:24; FIG. 14c) and the CMV derived peptide pp 65₄₉₅₋₅₀₃ (SEQ ID NO:25; FIG. 14d). Peptide-pulsed JY APCs were subsequently exposed to the appropriate HLA-A2-restricted and specific CTL clones/line. In each assay pulsing with an irrelevant HLA-A2-peptide was used as a negative control. FIG. 14e—JY APCs cells were pulsed with varying MART1₂₇₋₃₅ peptide (SEQ ID NO:22) concentrations ranging from 500 μM down to 10 pM. The fluorescence intensity of binding of a TCR-like antibody, CLA12, which specifically recognizes HLA-A2/MART1₂₇₋₃₅ (SEQ ID NO:22) complexes was determined by flow cytometry and directly correlated with QuantiBrite-phycoerythrin (PE) calibration beads that were used to create a calibration curve of Mean Fluorescence Intensities (MFI) for 0-10000 PE molecules for each cytometer setting. The MFI resulting from the binding of the TCR-like antibody was converted into number of PE molecules which directly correlate to the number of HLA-A2-peptide complexes detected by the TCR-like antibody. Peptide titrations correlated accurately with the fluorescent signal. Calculating MFI for each peptide concentration created a range of ˜1000 to a very few HLA-peptide complexes on the surface of peptide-pulsed cells. Standard deviation of each spot is statistically significant toward its nearby spots. FIG. 14f—The cytotoxicity of MART1₂₇₋₃₅-specific CTL clone acting against JY APCs pulsed at various peptide concentrations was correlated to the number of HLA-A2/MART1₂₇₋₃₅ complexes present on the target cell surface as detected by the HLA-A2/MART1₂₇₋₃₅-specific CLA12 TCR-like antibody. An irrelevant HLA-A2-restricted peptide was used as a negative control. FIGS. 14g-h—A431 (FIG. 14g) and ATAC4 (FIG. 14h) human epithelial carcinoma HLA-A2 negative which express EGFR and CD25, respectively, were incubated with single-chain HLA-A2/scFv fusion proteins to deposit variable amounts of HLA-A2/EBV and HLA-A2/209 peptide complexes, respectively on the surface of the target cells. Targets were exposed to the appropriate EBV and gp100₂₀₉ specific CTLs and lysis was measured. The number of HLA-A2/peptide complexes deposited on the target cell surface was determined using a PE conjugated monoclonal antibody BB7.2 which specifically recognizes HLA-A2 and PE calibration curves. FIG. 14i-Human HLA-A2 positive fibroblasts were infected with CMV and cytotoxicity of CMV pp65₄₉₅₋₅₀₃-specific CTLs was determined as a function of time post infection and number of HLA-A2/pp 65 complexes as determined by the TCR-like antibody H9 as described above.

FIGS. 15a-d depict the binding of TCR-like antibodies to peptide-pulsed human pAPCs and quantitation of the number of peptide-MHC molecules on the cell surface. High affinity (˜10-20 nM) TCR-like Fab antibodies CLA12 and 1A9 specific for HLA-A2 in complex with the MART-1₂₇₋₃₅ (FIG. 15a) and gp100₂₀₉₋₂₁₇ (SEQ ID NO:4) (FIG. 15b) HLA-A2-restricted peptides, respectively, were incubated with pAPCs pulsed with peptide concentrations ranging from 500 μM to 1 pM. PE-labeled secondary monoclonal antibodies specific to the Kappa or Lambda light chains of the TCR-like antibody were used to create a 1:1 ratio between the TCR-like Fab fragment and the PE-labeled secondary MAb. Mean fluorescence intensity (MFI) was calculated by using calibration curves of PE MFI at each cytometer settings. The MFI of each peptide loading concentration was translated into the number of specific HLA-A2-MART-1 (FIG. 15c) or HLA-A2-gp100 (FIG. 15d) complexes on the surface of the pAPC. This approach allowed the detection of as low as 10-20 specific peptide-MHC complexes on the surface of the target cell with high statistical significance. Peptide-MHC complexes could be detected on the surface of the pulsed pAPC with the high affinity TCR-like antibodies at peptide pulsing concentration as low as 10-100 pM.

FIGS. 16a-b depict HLA-A2 deposition on the surface of HLA-A2 negative epidermoid carcinoma cells using single-chain HLA-A2-scFv fusion molecules. FIG. 16a—A schematic diagram of the HLA-A2-scFv construct in which a scFv antibody fragment which recognizes EGFR or CD25 (Tac/p55) is genetically fused to the C-terminus of a single-chain HLA-A2 construct in which the β-2 microglobulin and HLA-A2 heavy chain genes are linked through a flexible peptide linker [(GGGGS)₃; SEQ ID NO:23). These recombinant fusion molecules were expressed in E. coli as intracellular inclusion bodies and refolded in vitro in the presence of the appropriate HLA-A2-restricted peptide (e.g., EBV derived peptide or gp100-209 peptide). FIG. 16b—A431 (EGFR positive) cells (shown in the Figure) or ATAC4 (CD25 transfected A431) cells (not shown) were incubated with various concentrations of the purified scHLA-A2-scFv fusion molecules and were subsequently washed. Detection of HLA-A2 deposition was performed using PE-labeled anti-HLA-A2 monoclonal antibody BB7.2. Measuring the MFI of PE-labeled Mab BB7.2 binding and conversion of MFI values into the number of PE molecules using the QuantiBrite PE calibration beads (BD), as described above, allowed the specific determination of the number of peptide-HLA-A2 complexes deposited on the surface of target cells at each concentration of recombinant fusion molecule. Determination was performed with fusion molecule concentration ranging from 10 pM to 600 nM. This approach allowed the detection of as low as 10-20 specific peptide-MHC complexes on the surface of the target cell with high statistical significance.

FIGS. 17a-d depict impaired proliferation after exposure of activated CTLs to high antigen (i.e., MHC-peptide complex) density. FIG. 17a shows that the antigen density induced unresponsiveness is long lasting even 3 and 7 days after exposure to high antigen density. FIG. 17b shows proliferation of CTLs as measured by ³H-Leucine incorporation measured 4, 16, and 36 hours after exposure of MART-1₂₇₋₃₅-specific CTL clone JKF6. The CTLs were exposed to increasing antigen densities by pulsing JY target cells. This figure shows that when CTLs are exposed to high antigen densities they reduce proliferation. FIG. 17c shows a graph depicting the relative RNA amount (in percentages) as a function of MHC-peptide complex/Target cell. This figure shows the RNA content of CTLs exposed to high antigen densities is significantly low compared to low or moderate densities which indicate inhibition in gene expression and reduced metabolic function. FIG. 17d shows that cell exposed to high antigen density do not proliferate after their exposure to high antigen density and that they do not die or there is no induction of apoptosis.

FIGS. 18a-i are FACS analyses depicting impaired expression of CD8 and CD3 after exposure of activated CTLs to high antigen density. MART-1₂₇₋₃₅-specific CTL clone JKF6 was exposed for 8 (FIGS. 18a-c), 24 (FIGS. 18d-f) or 48 (FIGS. 18g-i) hours to MART-1₂₇₋₃₅ peptide pulsed JY APC cells representing various antigen densities: low (10 sites; FIGS. 18c, f and i), intermediate-optimal (100 sites; FIGS. 18b, e and h) or high (650 sites; FIGS. 18a, d and g). Shown are the percentages of each subpopulation (CD3 or CD8). Note the impaired surface expression of CD3 or CD8 after exposure of the activated CTL to high antigen density.

FIGS. 19a-i are FACS analyses depicting impaired expression of CD45R0 after exposure of activated CTLs to high antigen density. MART-1₂₇₋₃₅-specific CTL clone JKF6 was exposed for 8 (FIGS. 19a-c), 24 (FIG. 19d-f) or 48 (FIGS. 19g-i) hours to MART-1₂₇₋₃₅ peptide pulsed JY APC cells representing various antigen densities: low (10 sites; FIGS. 19c, f and i), intermediate-optimal (100 sites; FIGS. 19b, e and h) or high (650 sites; FIGS. 19a, d and g). Shown are the percentages of cells expressing CD45R0. Note the impaired surface expression of CD45R0 after exposure of the activated CTL to high antigen density.

FIGS. 20a-i are FACS analyses depicting impaired expression of CD85 after exposure of activated CTLs to high antigen density. MART-1₂₇₋₃₅₋specific CTL clone JKF6 was exposed for 8 (FIGS. 20a-c), 24 (FIGS. 20d-f) or 48 (FIGS. 20g-i) hours to MART-1₂₇₋₃₅ peptide pulsed JY APC cells representing various antigen densities: low (10 sites; FIGS. 20c, f and i), intermediate-optimal (100 sites; FIGS. 20b, e and h) or high (650 sites; FIGS. 20a, d and g). Shown are the percentages of CD85-expressing cells. Note the impaired surface expression of CD85 after exposure of the activated CTL to high antigen density.

FIGS. 21a-i are FACS analyses depicting expression of CD152 after exposure of activated CTLs to high antigen density. MART-1₂₇₋₃₅-specific CTL clone JKF6 was exposed for 8 (FIGS. 21a-c), 24 (FIGS. 21d-f) or 48 (FIGS. 21g-i) hours to MART-1₂₇₋₃₅ peptide pulsed JY APC cells representing various antigen densities: low (10 sites; FIGS. 21c, f and i), intermediate-optimal (100 sites; FIGS. 21b, e and h) or high (650 sites; FIGS. 21a, d and g). Shown are the percentages of CD152-expressing cells. Note the similar expression pattern in all cells regardless of antigen density.

FIGS. 22a-i are FACS analyses depicting expression of Annexin V after exposure of activated CTLs to high antigen density. MART-1₂₇₋₃₅-specific CTL clone JKF6 was exposed for 8 (FIGS. 22a-c), 24 (FIGS. 22d-f) or 48 (FIGS. 22g-i) hours to MART-1₂₇₋₃₅ peptide pulsed JY APC cells representing various antigen densities: low (10 sites; FIGS. 22c, f and i), intermediate-optimal (100 sites; FIGS. 22b, e and h) or high (650 sites; FIGS. 22a, d and g). Shown are the percentages of Annexin V-expressing cells. Note the similar expression pattern in all cells regardless of antigen density.

FIGS. 23a-i are histograms depicting the dynamics of the expression of key surface molecules after exposure of activated CTLs to high antigen density. CD8^{High}, CD45R0^{High}, CD85^{Dim} homogenous CTLs were exposed to JY pulsed pAPCs. Two distinct subpopulations of CD8^{High}, CD45R0^{High}, CD85^{Dim} or CD8^{Low}, CD45R0^{Low}, CD85⁻ phenotypes were created after exposure. The percentage of High and Low subpopulations is shown as a function of exposure time and antigen density on target cells for each of the surface markers: CD8 (FIGS. 23a-c), CD45R0 (FIGS. 23d-f), CD85 (FIGS. 23g-i). Results are shown following exposure of activated CTLs for 8 hours (FIGS. 23a, d and g), 24 hours (FIGS. 23b, e and h) and 48 hours (FIGS. 23c, f and i) to the JY pulsed pAPCs. High expression level population is marked with filled diamods and low expression level population is marked with empty triangles. A significant transition point marked with an arrow was observed when CTLs were exposed to peptide concentration above 1×10⁻⁷ M, corresponding to ˜100 complexes/cell, most profoundly 48 hours post exposure.

FIG. 24a-b shows the distribution of CD8 high and low subpopulation in CTLs exposed to high antigen densities 3 or 7 days post exposure. The data indicate that the phenotypic changes in these CTLs are long lasting and persist even 7 days after exposure.

FIGS. 25a-d depict distinct gene expression signatures indicative of anergy in CTLs exposed to high density of antigen. FIG. 25a—A dendogram of 5877 genes which altered at least 2 fold in at least one of the treated versus untreated CTLs. Dendogram indicates levels of similarity between samples. Green=low expression: Red=high expression. CTL samples exposed to low antigen densities were clustered together with CTLs at time 0, before exposure to antigen (right branch), indicating high similarity between the gene expression profiles of these samples. CTLs exposed to optimal (100) or high (700) antigen densities were clustered together at each time point (4, 16 or 36 hours). Dendogram distances indicate a common starting point for these samples and progressively increasing differences between CTLs exposed to optimal versus high antigen densities with a maximal effect after 36 hours. FIG. 25b—Gene expression signature of 1070 probe sets which altered at least 2 fold between CTLs exposed to high or optimal antigen density, 36 hours post exposure. The clustering image demonstrates relatively high similarity between CTLs exposed to optimal or high antigen densities 4 and 16 hours post exposure. However, a profound shift toward two distinct expression profiles is demonstrated after 36 hours. This indicates separation of gene expression profile into two distinct types of CTLs; those with a gene signature of an optimal stimulation and function achieved by exposure to optimal antigen density, versus anergic CTL gene signature observed after exposure to high antigen densities. FIG. 25c—A functional classification of the 1070 probe sets which were altered by at least 2 fold between high and optimal antigen density 36 hours post exposure. Classification was performed using GO classification at level 3. FIG. 15d—A representative list of genes from the gene array analysis of 1070 probe sets described in FIGS. 25b and c. Shown are fold changes in the expression of representative genes from CTL exposed to optimal versus high antigen density. Alterations in gene expression related to cell cycle control, cell death, immune function, membrane potential, energy metabolism and signal transduction are shown. Note the genes from this analysis which exhibit major change in expression between optimal and high antigen density after 36 hours of exposure to antigen. The controls and normalizations for these DNA arrays experiments are summarized in the general methods section.

FIG. 26 is a schematic illustration depicting a proposed model for activated CTL function as a self referential sensory organ that is highly sensitive to antigen density; the -Y model. CTLs that were properly primed can be exposed to low antigen density which is translated into a weak and transient activation state. Optimal antigen densities lead to a strong and stable activation process that result in proliferation and effective killing of target cells. However, high antigen densities on the target cell lead to cell cycle arrest and a long lasting anergic state of the CTL.

FIGS. 27a-d depict nucleic acid (FIGS. 27a, c) and amino acid (FIGS. 27b, d) sequences of TA2-VLCL (SEQ ID NOs: 16 and 17; FIGS. 27a-b) and TA2-VHCH (SEQ ID NOs:18 and 19; FIGS. 27c-d). CDR sequences are in bold and underlined.

FIG. 28 is a bar graph depicting the specific recognition of binding of the TA2 TCRL antibody IgG to the Tyrosinase/HLA-2 complex. Note the specific binding of the TA2 TCRL antibody to the MHC-TYR complex (which includes the antigenic peptide of SEQ ID NO:1) as compared to the absence of binding of the TA2 TCRL to the MHC-154 complex (which includes the antigenic peptide of SEQ ID NO:20), the absence of binding of the TA2 TRCL antibody to the Tyrosinase D (TyrD; SEQ ID NO:1) peptide alone at various concentrations (1 mg, 100 μg, 10 μg or 1 μg), the absence of binding of the TA2 TCRL antibody to the 154 peptide (GP100 154 SEQ NO. 20) at various concentrations (1 mg, 100 μg, 10 μg or 1 μg) as well as the absence of binding of anti human HRP to both peptides, the left for 154 peptide and the right for Tyr D peptide).

FIGS. 29a-c depict plasmon resonance real-time binding kinetics of anti-HLA-A2/Tyrosinase TA2 Fab and IgG. Streptavidin coated beads were loaded with biotinylated HLA-A2-Tyr D complexes. 0.2M, 0.1M, 0.05M TA2 Fab or 0.025M, 0.01M, 0.005M TA2 IgG antibody was attached to the complexes, followed by wash with PBS.

FIGS. 30a-h depict amino acid (FIGS. 30a-d) and nucleic acid (FIGS. 30e-h) sequences of CLA12-VLCL (SEQ ID NOs: 31 and 35; FIGS. 30a, e), CLA12-VHCH (SEQ ID NOs: 32 and 36; FIGS. 30b, f), CAG10-VLCL (SEQ ID NOs: 33 and 37; FIGS. 30c, g), CAG10 VHCH (SEQ ID NOs: 34 and 38; FIGS. 30d, h). CDR sequences are in bold and underlined

FIGS. 30i-p depict nucleic acid and amino acid sequences of the pRB 98 BirA tag plasmids comprising CAG10-VLCL (SEQ ID NOs: 39 and 40); CAG10-VHCH (SEQ ID NOs: 45 and 46); CLA12-VLCL (SEQ ID NO: 41 and 42) and CLA12-VHCH (SEQ ID NO: 43 and 44).

FIGS. 31a-f depict the binding of Fabs CAG10 and CLA12 to peptide-loaded antigen presenting cells. FIG. 31a depicts the fine specificity of the purified Fab clones by ELISA. TCR-like Fabs as tested by ELISA for binding refolded peptide-MHC complexes. FIGS. 31b-c—Detection of Mart-1 peptide/HLA-A2 complexes on antigen presenting cells RMA-S-HHD. RMA-S cells were loaded with specific Mart-1 (26-35 (SEQ ID NO:21) or 27L (SEQ ID NO: 27) or control TAX peptide (SEQ ID NO:26). Complexes were probed with recombinant purified Fab CAG10 (FIG. 31b) and CLA12 (FIG. 31c) and analyzed by FACS using FITC-labeled goat anti-human Fab. FIG. 31d: mAb W6/32 binding demonstrates the total peptide/HLA on the APCs cell surface. FIGS. 31d-e: Detection by FACS of Mart-1 peptide/HLA-A2 complexes with Fab CAG10 and Fab CLA12 on antigen presenting JY cells loaded with specific (FIG. 31e) or control (FIG. 31f) peptide.

FIGS. 32a-f are FACS analyses measuring binding of Fab CLA12 to melanoma cell lines. Detection of Mart-1 peptide/HLA-A2 complexes was performed on HLA-A2+Mart-1+ melanoma cell lines 501A (FIG. 32a), FM3D (FIG. 32b), 624.38 (FIG. 32c) and Stiling (FIG. 32d) and on control cell lines: the HLA-A2+Mart-1− melanoma 1938 (FIG. 32e) or the HLA-A2-Mart-1+ melanoma G-43 (FIG. 32f) by Fab CLA12. Complexes were detected by FACS using recombinant purified Fab CLA12 and FITC-labeled goat anti-human Fab. mAb BB7.2 recognizes total HLA-A2 and was used to validate HLA-A2 expression by the indicated cell lines.

FIGS. 33a-f are FACS analyses measuring binding of Fab 2F1 to melanoma cell lines. Detection of G9-280 peptide/HLA-A2 (SEQ ID NO:5) complexes on HLA-A2+gp100+ melanoma cell lines 624.38 (FIGS. 33a,d) HLA-A2+gp100− 1938 (FIGS. 33b,e) and on the HLA-A2-gp100− melanoma PC3 (FIGS. 33c, f), when cells were preloaded with G9-280 (FIGS. 33a-c) or not (d-f).

FIGS. 34a-d depict schematics and analyses of recombinant purified Fab 2F1-PE38 KDEL. FIG. 34a is a schematic representation of the Fab 2F1-PE38 fusion protein. The gene encoding PE38 that contains the translocation and ADP ribosylation domains of PE was fused to the C terminus of Fab 2F1 (Lκ) κ chain. FIG. 34b is an SDS/gel analysis on 4-20% gradient gels of recombinant Fab 2F1 Light chain-PE38 fusion (Lκ)-PE38 with the expected size of 63 KDa (Lanes 1) and Fab 2F1 heavy (H) chain—25 KDa (Lane 2) from bacterial inclusion bodies. FIG. 34c is an SDS/gel analysis on 4-20% gradient gels of Fab 2F1-PE38 KDEL reduced with expected bands at 63 KDa and 25 KDa (Lane 1); Fab 2F1-PE38 KDEL nonreduced—88 KDa (Lane 2). FIG. 34d depicts the fine specificity of the purified 2F1 Fab PE38 KDEL as analyzed by ELISA. pMHC complexes were refolded using each peptide and coated via streptavidin on an ELISA plate. An equal concentration of TCR-like Fab-toxin was incubated for 1 h at room temperature. After extensive washing, bound clones were detected with an anti-human Fab or anti-PE mAb coupled to HRP.

FIGS. 34e-f are biacore results showing affinity of CLA12 (FIG. 34e) and TA2 (FIG. 34f) to the complexed antigen.

FIGS. 35a-h are FACS analyses measuring binding of Fab-PE38 KDEL clones to peptide loaded APCs and melanoma cell lines. For FIGS. 35a-d, tap deficient A2+T2 cells were pulsed with 50 μM MART-1 (FIGS. 35a-b), 50 μM G9-280 (SEQ ID NO: 5; FIG. 35c), 50 μM G9-154 (SEQ ID NO: 20; FIG. 35d) or no peptide (control), then stained with the designated Mart-1/HLA-A2 (FIGS. 35a-b), G9-280/HLA-A2 (FIG. 35c) or G9-154/HLA-A2 (FIG. 35d)-selected Fab-toxins and analyzed by flow cytometry. For FIGS. 35e-h, detection of GP100-derived peptide/HLA-A2 complexes on HLA-A2+gp100+ melanoma cell lines Mel526 (FIG. 35e), Mel501A (FIG. 35f) and Mel624.38 (FIG. 35h) No staining was observed by Fab-toxin 2F1-PE38 KDEL or G2D12-PE38 KDEL on HLA-A2+gp100− melanoma 1938 (FIG. 35g). Complexes were detected by flow cytometry using FITC-labeled goat anti-human Fab.

FIGS. 36a-f depict the cellular binding and internalization of 2F1-PE38-FITC. JY cells were loaded with G9-280 (SEQ ID NO: 5) or G9-209 (SEQ ID NO: 4) peptide and incubated at 4° C. in the presence of 2F1-PE38-FITC. FIG. 36a—no staining was detected on JY cells loaded with G9-209. Cells were then transferred to 37° C. and monitored for the immunotoxin internalization at indicated time points 0 (FIG. 36b); 15 minutes (FIG. 36c); 30 minutes (FIG. 36d); 1 hour (FIG. 36e) and 6 hours (FIG. 36f). PI was used to detect the nucleus.

FIGS. 37a-b are graphs depicting the cytotoxic activity of 2F1-PE38-KDEL toward peptide loaded APCs. FIG. 37a: Cytotoxic activity of recombinant Fab 2F1-PE38 KDEL towards JY cells loaded with G9-280 peptide (SEQ ID NO: 5) or with other control HLA-A2 restricted peptides. Cells were incubated for 24 hr with recombinant Fab 2F1-PE38 KDEL. [³H] leucine incorporation into cellular protein was measured. The results are expressed as a percentage of control where no immunotoxin was added. FIG. 37b is the same assay as in FIG. 37a was performed on HLA-A2+ FM3D cells and HLA-A2-G43 cells loaded with the specific G9-280 (SEQ ID NO: 5), or control HLA-A2 restricted peptides.

FIGS. 38a-f are graphs depicting the cytotoxic activity of 2F1-PE38-KDEL toward melanoma cell lines. FIGS. 38a-c: Cytotoxic activity of 2F1-PE38 KDEL (FIG. 38a), G2D12-PE38 KDEL (FIG. 38b) and CLA12-PE38 KDEL (FIG. 38c) toward melanoma cell lines Mel526, Mel624.38, Mel501A, G43, 1938 after 24 h of incubation. [H³] leucine incorporation into cellular protein was measured. The results are expressed as a percentage of control where no immunotoxin was added. FIG. 39d: Cytotoxic effect of combination treatment of two immunotoxin 2F1-PE38 KDEL and CLA12-PE38 KDEL on melanoma cells Mel526, Mel624, G43. FIGS. 38e-f: Cytotoxic effect of CLA12-PE38 KDEL or 2F1-PE38 KDEL alone or CLA12-PE38 KDEL plus 2F1-PE38 KDEL on Mel526 (FIG. 38e) or Mel 624.38 (FIG. 38f). The cells were incubated with the immunotoxins for 24 h. [³H] leucine incorporation into cellular protein was measured. The results are expressed as a percentage of control where no immunotoxin was added.

FIGS. 39a-b are graphs depicting the anti-tumor activity of CLA12-PE38 KDEL on subcutaneous human melanoma tumor in SCID mice. Groups of animals were injected with 10×10⁶ 526 melanoma cells on day 0. When tumor reached 55 mm³, animals were treated i.v. on day 10, 12, 14, 16 with 0.125 mg/kg (FIG. 39a) and 0.05 mg/kg (FIG. 39b) CLA12 Fab-PE38 KDEL in PBS. Control group received diluent alone. No cytotoxicity was observed at these doses. Comparison of tumor size between treated and untreated group gave P<0.0006. Data are expressed as the mean±SD (n=4).

FIGS. 40a-b are bar graphs illustrating the monoclonal ELISA of Tyr/HLA-A2 TCR-like antibodies. FIG. 40a: Reactivity of Fab antibody clones from the conventional screening method with recombinant purified Tyr/HLA-A2 and control complex. ScHLA-A2-peptide complexes were generated by in vitro refolding as described in materials and methods. Detection was with Peroxidase-labeled anti-human Fab. FIG. 40b: Reactivity of Fab antibody clones from the Off-Rate selections with recombinant purified Tyr/HLA-A2 and control complex. ScHLA-A2-peptide complexes were generated by in vitro refolding as described in materials and methods. Detection was with Peroxidase-labeled anti-human Fab.

FIGS. 41a-g are sequence and SPR Affinity Determinations of the three different anti Tyr Fabs. FIG. 41a-d are the deduced amino acid sequence of the HLA-A2/Tyr₃₆₉₋₃₇₇ Fabs, B2, MC1 (bold letters indicating the position of the CDR's). FIG. 41e-g are SPR Binding curves of the three Fabs with 5 different concentrations. The chip was covered with Fab anti Fab to which the Fab Abs were conjugated, the ligand the analyte the HLA-A2/Tyr complex. The chip was covered with Fab anti-human Fab, the three anti HLA-A2-Tyr Fabs were bound (i.e. the ligand), and then the HLA-A2-Tyr complex was bound (i.e. the analyte).

FIGS. 42a-k are graphs illustrating the results of flow cytometry analysis of the binding of MC1 Fabs to tumor cells. Detection was with PE-labeled anti-human Fab.

FIGS. 43a-k are graphs illustrating the results of flow cytometry analysis of the binding of B2 Fabs to tumor cells. Detection was with PE-labeled anti-human Fab.

FIGS. 44a-k are graphs illustrating the results of flow cytometry analysis of the binding of TA2 Fabs to tumor cells. Detection was with PE-labeled anti-human Fab.

FIGS. 45a-d are graphs illustrating that the MC1, B2, TA2 Fabs recognized Tyrosinase positive and HLA-A2 positive cells (501A melanoma cells) with a very high intensity corresponding to their improved affinity. The detection limit of TA2 was 10 μg/ml on positive cells while B2 had detection limit of 1 μg/ml on positive cells and MC1 reacted in 0.1 μg/ml on positive cells.

FIGS. 46a-d are graphs illustrating binding of TA2 and MC1 Fab-PE38 fusion molecules to tumor cells as measured by flow cytometry analysis. Binding of TA2 and MC1 Fab-PE38 to Tyr positive and negative tumor cells was monitored by PE-labeled anti-human Fab.

FIGS. 47a-d are graphs illustrating the cytotoxic activity of TA2 Fab/scFv-PE38 fusion and MC1 Fab/scFv-PE38 KDEL fusion following analysis of inhibition of protein synthesis in tumor cells. Tyr positive and control Tyr negative tumor cells were incubated for 20 hours with increasing concentrations of TA2 Fab/scFv-PE38 (FIG. 47a-b) or MC1 Fab/scFv-PE38 KDEL (FIG. 47c-d). Protein synthesis was determined by incorporation of 3H-Leucine into cellular proteins for 4 hours.

### DESCRIPTION OF THE PREFERRED EMBODIMENTS

The present invention is of methods of diagnosing and treating melanoma and of antibodies capable of same.

The principles and operation of the diagnostic and therapeutic methods of present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Use of antibodies that specifically recognize class I MHC-peptide complexes as therapeutic and diagnostic tools for the treatment and detection of cancer in general and melanoma specifically has already been established (e.g., U.S. Pat. Appl. No. 20050255101).

The present invention uncovers which is the optimum melanoma-associated class I MHC-peptide complex for such antibodies to target and therefore paves the way for the discovery of more potent antibodies both for the treatment and diagnosis of melanoma.

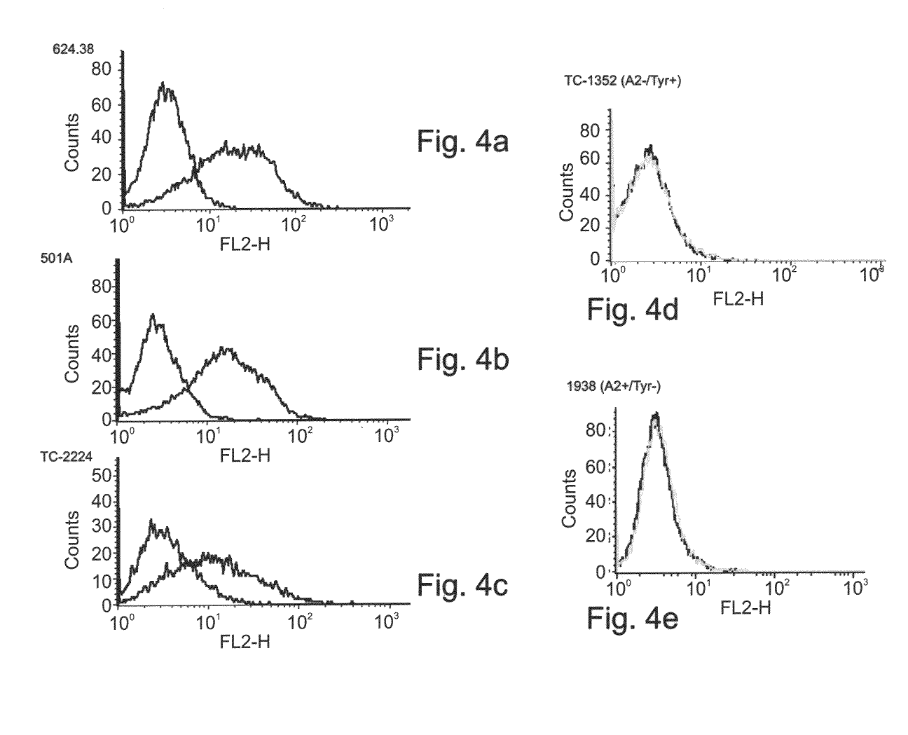

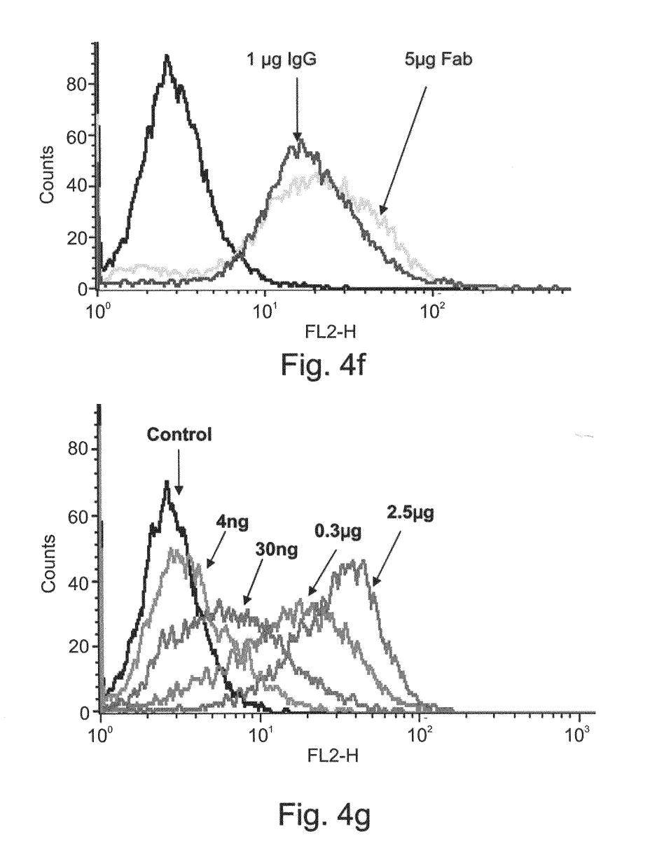

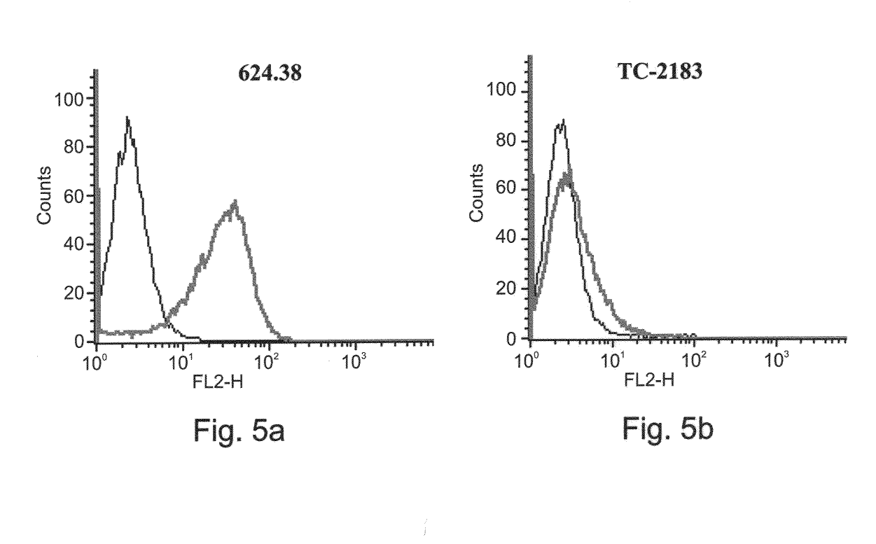

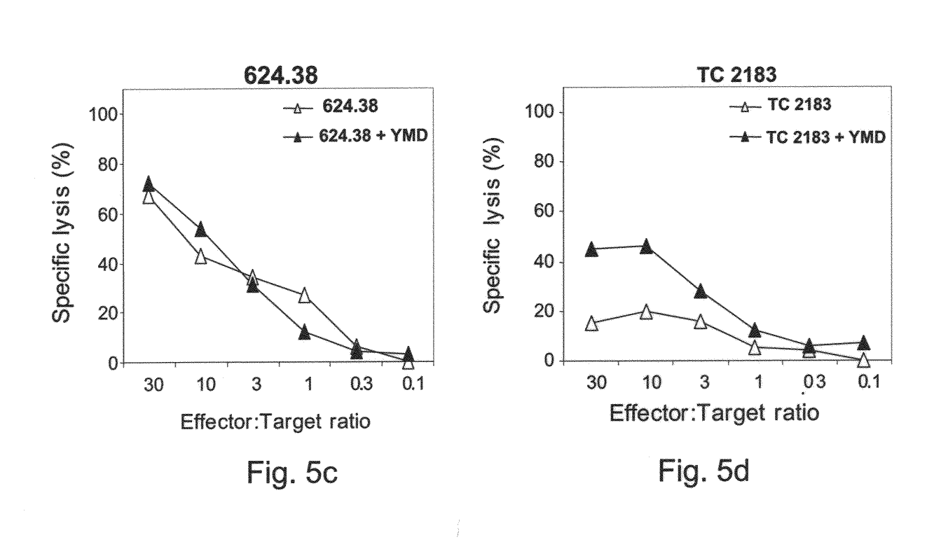

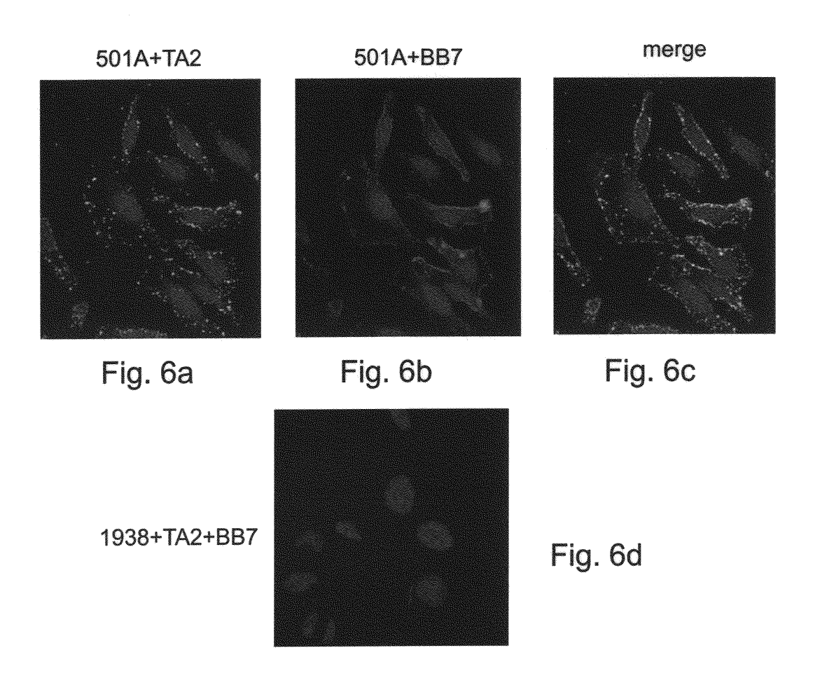

Whilst reducing the present invention to practice, the present inventors have generated phage-derived human T cell receptor (TCR)-like antibodies to the three major differentiation antigens known to be expressed on melanoma cells. The present inventors have shown, using one such antibody specific to the MHC-Tyrosinase₃₆₉₋₃₇₇ complex, that the Tyrosinase₃₆₉₋₃₇₇ epitope is highly presented on melanoma cell lines as analyzed by flow cytometry (FIGS. 4a-g) and confocal microscopy (FIGS. 6a-d). Verification of the observation that high numbers of tyrosinase peptides are presented on HLA-A2 complexes was obtained by performing cytotoxicity tests with cytotoxic T cells (CTLs) that specifically recognize the HLA-A2− Tyrosinase₃₆₉₋₃₇₇ epitope (FIGS. 5a-d).

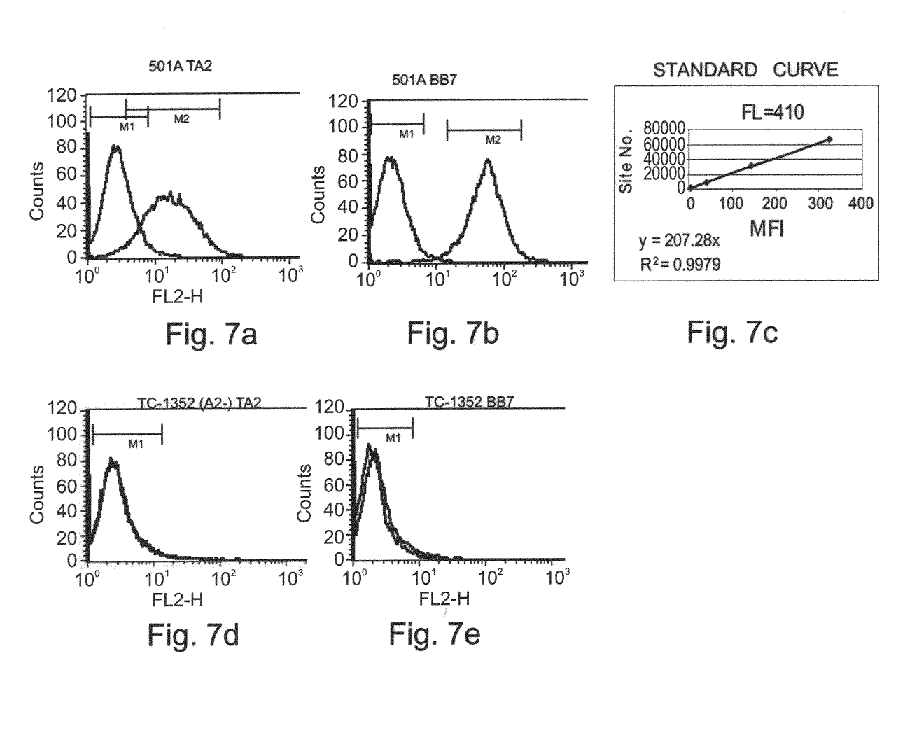

Using Fab tetramers generated around a single streptavidin-PE molecule, which are able to bind up to four different HLA-Tyr complexes, the present inventors were able to accurately determine by flow cytometry analysis the minimal number of tyrosinase complexes presented on the cell surface (FIGS. 7a-e). Thus, the percent of HLA-A2 peptide complexes presenting tyrosinase peptides was estimated to be as high as 20%. The discovery that the tyrosinase epitope is so densely presented on melanoma cells promotes this epitope as an ideal target both for antibody-mediated drug delivery and for antibody-mediated diagnosis of melanoma.

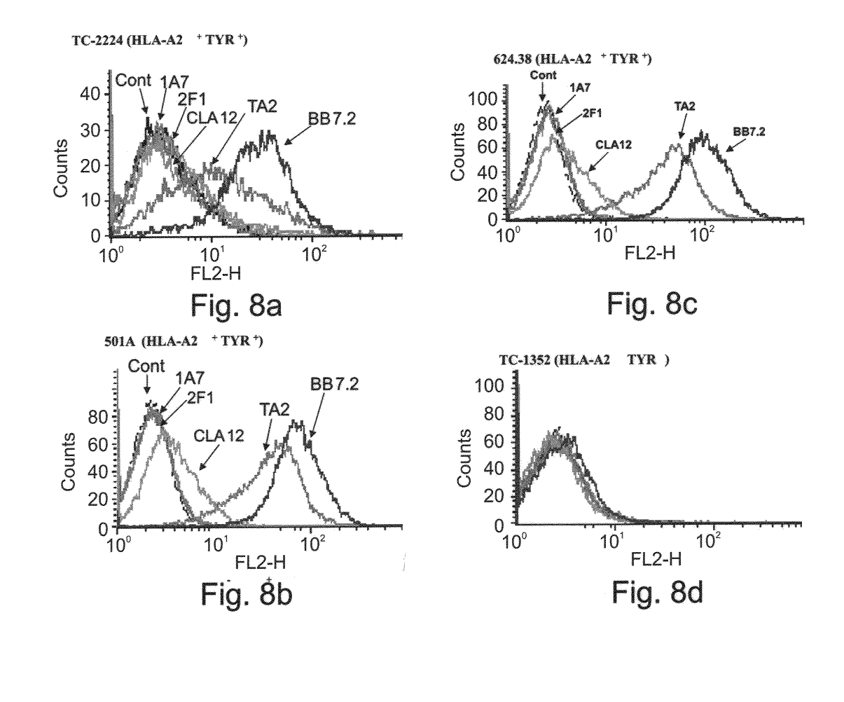

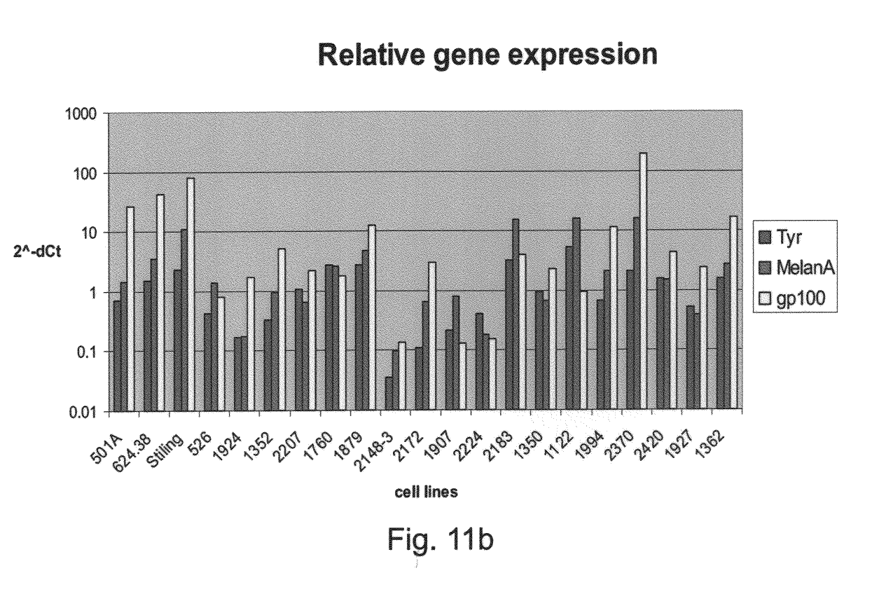

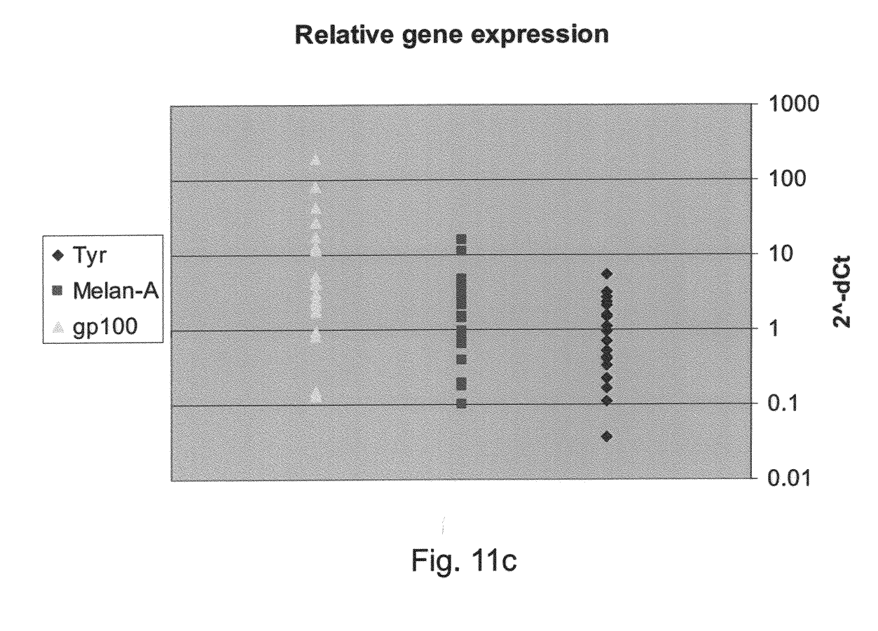

The present inventors have unexpectedly found that presentation of melanoma T cell epitopes does not correlate with their gene expression profile. Accordingly HLA-A2-peptide complexes derived from Tyrosinase were found to be expressed and presented on the surface of melanoma cells at unexpectedly high numbers as compared to Gp100 or Mart-1 epitopes, as analyzed by flow cytometry (FIGS. 8a-c) even though the relative expression level of Tyrosinase was lower than that of Gp100 and Mart-1 (see Tables 3 and 4 herein below and FIGS. 11a-c). This discovery leads to the conclusion that analysis of the presentation of melanoma epitopes is a better gauge for determining if a patient is a good candidate for epitope-mediated therapy as opposed to analysis of expression of melanoma antigens.

Without being bound to theory, the present inventors suggest that the instability of the tyrosinase protein may be the cause of this unexpectedly high presentation. Furthermore, the present inventors suggest that the high presentation of HLA-A2-Tyr complexes may explain the relatively low immune response against the Tyrosinase 369-377 epitope since it is known that continual exposure of T cells to antigen maintains an unresponsive state and result in adaptive tolerance (i.e., anergy, B. Rocha, et al., 1995; L. S. Taams, et al., 1999; R. H. Schwartz, 2003; F. Ramsdell and B. J. Fowlkes, 1992). Thus, without being bound to theory the present inventors propose that anergized T cells may be wholly or partly due to the high presentation on melanoma cells.

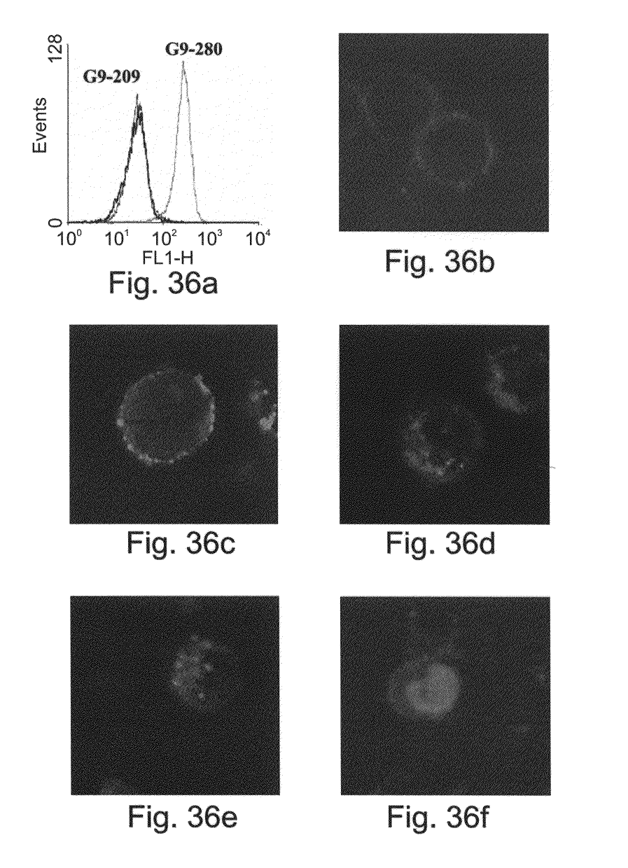

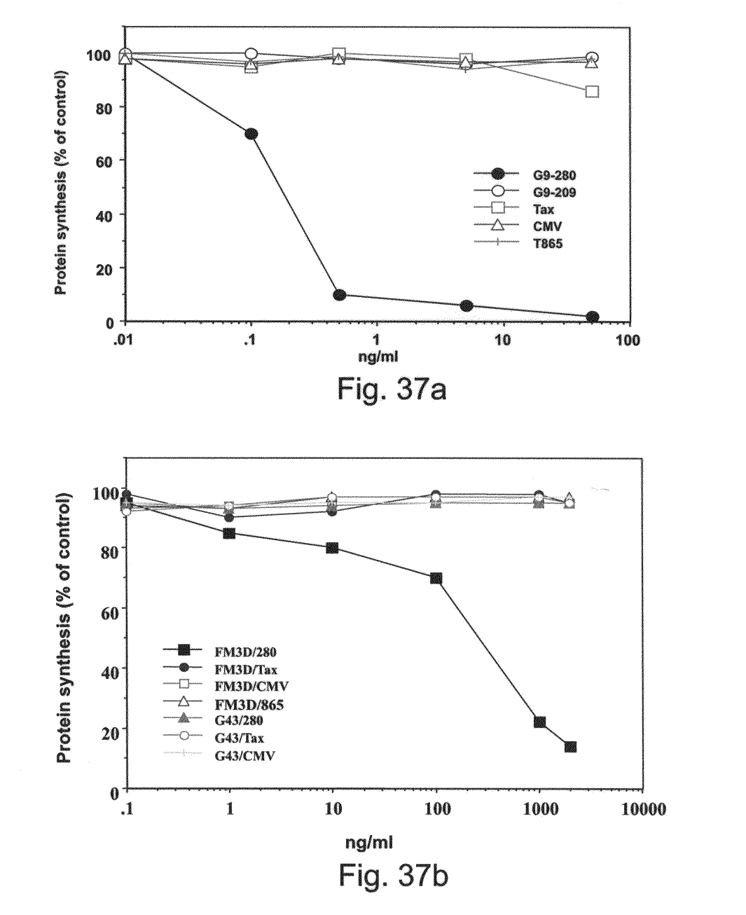

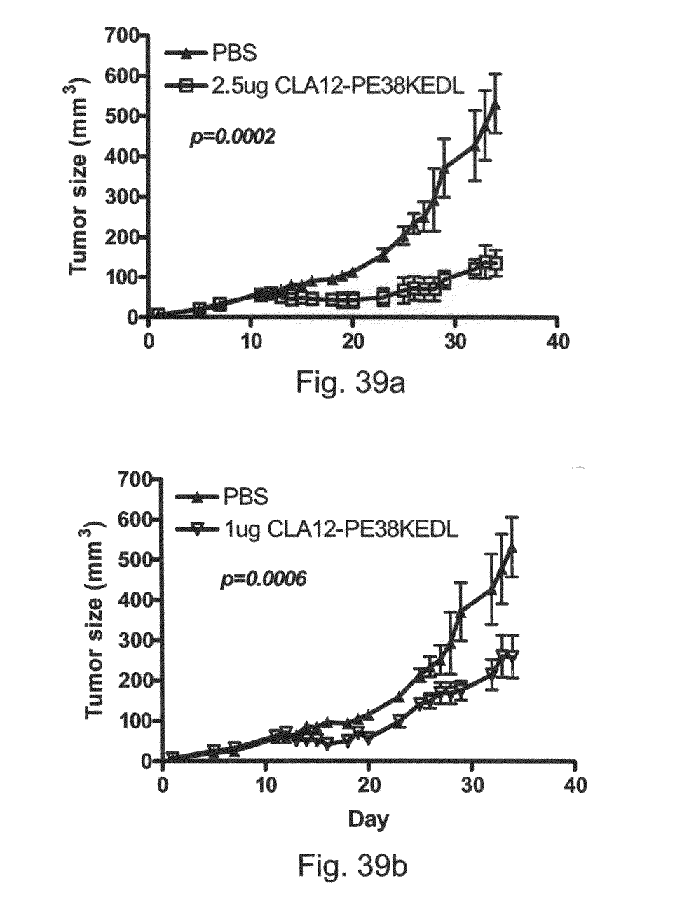

Whilst further reducing the invention to practice, the present inventors have generated TCR-like antibody-toxin fusion proteins and have shown that such fusion molecules can undergo internalization into melanoma cells (FIGS. 36a-f and 46a-d) and kill them (FIGS. 37a-b, 38a-c and 47a-d). Moreover, the present inventors have shown that the generated antibody-toxin fusion proteins were capable of inducing regression of melanoma in vivo (FIGS. 39a-b) in irradiated mice implanted with human melanoma cells. These experiments provide a proof of principle that specific MHC complexes can be used in humans as target therapy for melanoma.

Thus, according to one aspect of the present invention, there is provided a method of detecting a melanoma cell. The method comprises contacting the cell with an antibody comprising an antigen recognition domain capable of binding to an MHC-I molecule being complexed with a tyrosinase peptide, wherein the antibody does not bind the MHC-I in an absence of the complexed peptide, and wherein the antibody does not bind the peptide in an absence of the MHC. The contacting is effected under conditions which allow immunocomplex formation, wherein a presence of the immunocomplex or level thereof is indicative of the melanoma cell.

The term “detecting”, as used herein, refers to the act of detecting, perceiving, uncovering, exposing, visualizing or identifying a cell. The precise method of detecting is dependent on the detectable moiety to which the antibody is attached as further described herein below.

As used herein, the term “melanoma cell” refers to a malignant melanocyte of mammalian origin, preferably human. Typically, the melanoma cell comprises tumor associated antigens on its cell surface such as tyrosinase peptides, Mart-1 peptides and/or Gp100 peptides associated with MHC molecules.

Single cells may be used in accordance with the teachings of the present invention as well as a plurality of cells. The cells may be from any biological sample such as melanoma cell-lines, primary melanoma cultures and cellular samples, e.g. skin cell biopsies (surgical biopsies including incisional or excisional biopsy, fine needle aspirates and the like). Methods of biopsy retrieval are well known in the art.

It will be appreciated that if the skin cells are taken from a subject (i.e. biopsies), the method of the present invention may also be used to diagnose melanoma in a subject. The melanoma may be at any stage e.g. IA, IB, IIA, IIB, IIC, IIIA, IIIB, IIIC and IV.

As used herein the term “diagnosing” refers to classifying a melanoma, determining a severity of melanoma (grade or stage), monitoring melanoma progression, forecasting an outcome of the melanoma and/or prospects of recovery.

The subject may be a healthy subject (e.g., human) undergoing a routine well-being check up. Alternatively, the subject may be at risk of having melanoma (e.g., a genetically predisposed subject, a subject with medical and/or family history of cancer, a subject who has been exposed to carcinogens, occupational hazard, environmental hazard] and/or a subject who exhibits suspicious clinical signs of melanoma [e.g., a change in the appearance of a mole).

As mentioned herein above, the method comprises contacting the melanoma cell with an antibody which is able to specifically bind a tyrosinase derived peptide restricted to an MHC-I complex.

As used herein, the phrase “major histocompatibility complex (MHC)” refers to a complex of antigens encoded by a group of linked loci, which are collectively termed H-2 in the mouse and HLA in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against foreign class I glycoproteins, while helper T-cells respond mainly against foreign class II glycoproteins.

Major histocompatibility complex (MHC) class I molecules are expressed on the surface of nearly all cells. These molecules function in presenting peptides which are mainly derived from endogenously synthesized proteins to CD8+ T cells via an interaction with the αβ T-cell receptor. The class I MHC molecule is a heterodimer composed of a 46-kDa heavy chain which is non-covalently associated with the 12-kDa light chain β-2 microglobulin. In humans, there are several MHC haplotypes, such as, for example, HLA-A2, HLA-A1, HLA-A3, HLA-A24, HLA-A28, HLA-A31, HLA-A33, HLA-A34, HLA-B7, HLA-B45 and HLA-Cw8, their sequences can be found at the kabbat data base, at htexttransferprotocol://immuno.bme.nwu.edu. Further information concerning MHC haplotypes can be found in Paul, B. Fundamental Immunology Lippincott-Rven Press.

Tyrosinase peptides that bind to class I MHC molecules (also referred to herein interchangeably as HLA-restricted tyrosinase epitopes, HLA-restricted tyrosinase epitopes and MHC-restricted tyrosinase antigens) are derived from the tyrosinase enzyme (Genebank Accession No: AH003020) and are typically 8-10 amino acids long, bind to the heavy chain α1-α2 groove via two or three anchor residues that interact with corresponding binding pockets in the MHC molecule.

Tyrosinase is a membrane-associated N-linked glycoprotein and it is the key enzyme in melanin synthesis. It is expressed in all healthy melanocytes and in nearly all melanoma tumor samples (H. Takeuchi, et al., 2003; S. Reinke, et al., 2005). Peptides derived from this enzyme are presented on MHC class I molecules and are recognized by autologuos cytolytic T lymphocytes in melanoma patients (T. Wolfel, et al., 1994; Brichard, et al., 1993).

According to one embodiment, the antibody of the present invention specifically recognizes an MHC class I complexed-tyrosinase peptide as set forth in SEQ ID NO: 1, although it should be appreciated that the present invention envisages antibodies recognizing other MHC class I complexed-tyrosinase peptides. Such tyrosinase peptides known to be restricted on MHC class I complexes and expressed on melanoma cells are described by Renkvist et al, Cancer immunology immunotherapy 2001 50:3-15 and Novellino L, et al., March 2004 update. Cancer Immunol Immunother. 54:187-207, 2005. Additional tumor tyrosinase HLA-restricted peptides derived from tumor associated antigens (TAA) can be found at the website of the Istituto Nazionale per lo Studio e la Cura dei Tumori at hypertexttransferprotocol://worldwideweb.istitutotumori.mi.it.

As used herein the term “peptide” refers to native peptides (either proteolysis products or synthetically synthesized peptides) and further to peptidomimetics, such as peptoids and semipeptoids which are peptide analogs, which may have, for example, modifications rendering the peptides more stable while in a body, or more immunogenic. Such modifications include, but are not limited to, cyclization, N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptidomimetic compounds are well known in the art and are specified in Quantitative Drug Design, C. A. Ramsden Gd., Chapter 17.2, F. Choplin Pergamon Press (1992), which is incorporated by reference as if fully set forth herein. Further details in this respect are provided hereinunder.

As used herein in the specification and in the claims section below the term “amino acid” is understood to include the 20 naturally occurring amino acids; those amino acids often modified post-translationally in vivo, including for example hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Further elaboration of the possible amino acids usable according to the present invention and examples of non-natural amino acids useful in MHC-I HLA-A2 recognizable peptide antigens are given herein under.

Based on accumulated experimental data, it is nowadays possible to predict which of the peptides of a protein will bind to MHC, class I. The HLA-A2 MHC class I has been so far characterized better than other HLA haplotypes, yet predictive and/or sporadic data is available for all other haplotypes.

With respect to HLA-A2 binding peptides, assume the following positions (P1-P9) in a 9-mer peptide:
P1-P2-P3-P4-P5-P6-P7-P8-P9

The P2 and P2 positions include the anchor residues which are the main residues participating in binding to MHC molecules. Amino acid resides engaging positions P2 and P9 are hydrophilic aliphatic non-charged natural amino (examples being Ala, Val, Leu, Ile, Gln, Thr, Ser, Cys, preferably Val and Leu) or of a non-natural hydrophilic aliphatic non-charged amino acid (examples being norleucine (Nle), norvaline (Nva), α-aminobutyric acid). Positions P1 and P3 are also known to include amino acid residues which participate or assist in binding to MHC molecules, however, these positions can include any amino acids, natural or non-natural. The other positions are engaged by amino acid residues which typically do not participate in binding, rather these amino acids are presented to the immune cells. Further details relating to the binding of peptides to MHC molecules can be found in Parker, K. C., Bednarek, M. A., Coligan, J. E., Scheme for ranking potential HLA-A2 binding peptides based on independent binding of individual peptide side-chains. J Immunol. 152,163-175,1994., see Table V, in particular. Hence, scoring of HLA-A2.1 binding peptides can be performed using the HLA Peptide Binding Predictions software interface hypertexttransferprotocol://worldwideweb.bimas.dcrt.nih.gov/molbio/hla_bind/index. This software is based on accumulated data and scores every possible peptide in an analyzed protein for possible binding to MHC HLA-A2.1 according to the contribution of every amino acid in the peptide. Theoretical binding scores represent calculated half-life of the HLA-A2.1-peptide complex.

Hydrophilic aliphatic natural amino acids at P2 and P9 can be substituted by synthetic amino acids, preferably Nleu, Nval and/or α-aminobutyric acid. P9 can be also substituted by aliphatic amino acids of the general formula —HN(CH₂)ₙCOOH, wherein n=3-5, as well as by branched derivatives thereof, such as, but not limited to,

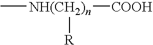

wherein R is, for example, methyl, ethyl or propyl, located at any one or more of the n carbons.

The amino terminal residue (position P1) can be substituted by positively charged aliphatic carboxylic acids, such as, but not limited to, H₂N(CH₂)ₙCOOH, wherein n=2-4 and H₂N—C(NH)—NH(CH₂)ₙCOOH, wherein n=2-3, as well as by hydroxy Lysine, N-methyl Lysine or ornithine (Orn). Additionally, the amino terminal residue can be substituted by enlarged aromatic residues, such as, but not limited to, H₂N—(C₆H₆)—CH₂—COOH, p-aminophenyl alanine, H₂N—F(NH)—NH—(C₆H₆)—CH₂—COOH, p-guanidinophenyl alanine or pyridinoalanine (Pal). These latter residues may form hydrogen bonding with the OH— moieties of the Tyrosine residues at the MHC-1 N-terminal binding pocket, as well as to create, at the same time aromatic-aromatic interactions.

Derivatization of amino acid residues at positions P4-P8, should these residues have a side-chain, such as, OH, SH or NH₂, like Ser, Tyr, Lys, Cys or Orn, can be by alkyl, aryl, alkanoyl or aroyl. In addition, OH groups at these positions may also be derivatized by phosphorylation and/or glycosylation. These derivatizations have been shown in some cases to enhance the binding to the T cell receptor.

Longer derivatives in which the second anchor amino acid is at position P10 may include at P9 most L amino acids. In some cases shorter derivatives are also applicable, in which the C terminal acid serves as the second anchor residue.

Cyclic amino acid derivatives can engage position P4-P8, preferably positions P6 and P7. Cyclization can be obtained through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COOH)—C(R)H—NH₂, wherein n=1-4, and further wherein R is any natural or non-natural side chain of an amino acid.

Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Orn, Dab or Dap.

Peptide bonds (—CO—NH—) within the peptide may be substituted by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time. Preferably, but not in all cases necessary, these modifications should exclude anchor amino acids.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

The term “antibody” as used herein includes intact molecules as well as functional fragments thereof, such as Fab, F(ab′)₂, Fv and scFv that are capable of specific binding to a human major histocompatibility complex (MHC) class I-restricted tyrosinase epitope. These functional antibody fragments are defined as follows: (i) Fab, the fragment which contains a monovalent antigen-binding fragment of an antibody molecule, can be produced by digestion of whole antibody with the enzyme papain to yield an intact light chain and a portion of one heavy chain; (ii) Fab′, the fragment of an antibody molecule that can be obtained by treating whole antibody with pepsin, followed by reduction, to yield an intact light chain and a portion of the heavy chain; two Fab′ fragments are obtained per antibody molecule; (iii) F(ab′)₂, the fragment of the antibody that can be obtained by treating whole antibody with the enzyme pepsin without subsequent reduction; F(ab′)₂ is a dimer of two Fab′ fragments held together by two disulfide bonds; (iv) Fv, defined as a genetically engineered fragment containing the variable region of the light chain and the variable region of the heavy chain expressed as two chains; and (c) scFv or “single chain antibody” (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference) and are further described herein below.

An exemplary method for generating antibodies capable of specifically binding a tyrosinase peptide restricted to an MHC-I complex is described in the Examples section herein below.

Specifically, such antibodies may be generated by (i) immunizing a genetically engineered non-human mammal having cells expressing the human major histocompatibility complex (MHC) class I, with a soluble form of a MHC class I molecule being complexed with the HLA-restricted epitope; (ii) isolating mRNA molecules from antibody producing cells, such as splenocytes, of the non-human mammal; (iii) producing a phage display library displaying protein molecules encoded by the mRNA molecules; and (iv) isolating at least one phage from the phage display library, the at least one phage displaying the antibody specifically bindable (preferably with an affinity below 10 nanomolar) to the human major histocompatibility complex (MHC) class I being complexed with the HLA-restricted epitope. The genetic material of the phage isolate is then used to prepare a single chain antibody or other forms of antibodies as is further described herein below. The genetic material of the phage isolate is then used to prepare a single chain antibody or other forms of antibodies as is further described herein below and which are conjugated to identifiable or therapeutic moieties. Preferably, the non-human mammal is devoid of self MHC class I molecules. Still preferably, the soluble form of a MHC class I molecule is a single chain MHC class I polypeptide including a functional human β-2 microglobulin amino acid sequence directly or indirectly covalently linked to a functional human MHC class I heavy chain amino acid sequence.

Recombinant MHC class I and class II complexes which are soluble and which can be produced in large quantities are described in, for example, references 24, 55 and 56-68 and further in U.S. patent application Ser. No. 09/534,966 and PCT/IL01/00260 (published as WO 01/72768), all of which are incorporated herein by reference. Soluble MHC class I molecules are available or can be produced for any of the MHC haplotypes, such as, for example, HLA-A2, HLA-A1, HLA-A3, HLA-A24, HLA-A28, HLA-A31, HLA-A33, HLA-A34, HLA-B7, HLA-B45 and HLA-Cw8, following, for example the teachings of PCT/IL01/00260, as their sequences are known and can be found at the kabbat data base, at http://immuno.bme.nwu.edu/, the contents of the site is incorporated herein by reference. Such soluble MI-IC class I molecules can be loaded with suitable HLA-restricted epitopes and used for vaccination of non-human mammal having cells expressing the human major histocompatibility complex (MHC) class I as is further detailed hereinbelow.

Non-human mammal having cells expressing a human major histocompatibility complex (MHC) class I and devoid of self major histocompatibility complex (MHC) class I can be produced using (i) the sequence information provided in the kabbat data base, at http://immuno.bme.nwu.edu/, which is incorporated herein by reference and pertaining to human MHC haplotypes, such as, for example, HLA-A2, HLA-A1, HLA-A3, HLA-A24, HLA-A28, HLA-A31, HLA-A33, HLA-A34, HLA-B7, HLA-B45 and HLA-Cw8, (ii) conventional constructs preparation techniques, as described in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); and (iii) conventional gene knock-in/knock-out techniques as set forth, for example, in U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866; in International Publications WO 94/23049, WO93/14200, WO 94/06908 and WO 94/28123; as well as in Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8): 1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261, 1993; Lamb et al., Nature Genetics, 5: 22-29, 1993; Pearson and Choi, Proc. Natl. Acad. Sci. USA, 1993. 90:10578-82; Rothstein, Methods in Enzymology, 194:281-301, 1991; Schedl et al., Nature, 362: 258-261, 1993; Strauss et al., Science, 259:1904-1907, 1993, all of which are incorporated herein by reference. Of particular interest is the paper by Pascolo et al., published in J. Exp. Med. 185: 2043-2051, 1997, which describe the preparation of mice expressing the human HLA-A2.1, H-2 Db and HHD MHC class I molecules and devoid of mice MHC class I altogether.

An exemplary antibody capable of binding to an MHC class I complexed with a tyrosinase epitope comprises complementarity determining region (CDR) amino acid sequences as set forth in SEQ ID NOs: 59-64.

According to one embodiment of this aspect of the present invention, the antibody binds to the MHC class I/tyrosine epitope complex with a dissociation constant less than 100 nM. According to another embodiment the dissociation constant is less that 10 nM.

Such antibodies have been described in Example 12 of the Examples section herein below. Thus, according to this embodiment, the antibody may comprise complementarity determining region (CDR) amino acid sequences as set forth in SEQ ID NOs: 886-891 or complementarity determining region (CDR) amino acid sequences as set forth in SEQ ID NOs: 894-899.

It will be appreciated that although the present inventors have discovered that tyrosinase epitopes are the optimal target for melanoma detection due to their very high presentation on melanoma cells, the present invention also contemplates the use of antibodies capable of recognizing other class I MHC-peptide complexes such as class I MHC-Mart-1 complexes.

Thus, according to another aspect of the present invention, there is provided an antibody comprising an antigen recognition domain which comprises complementarity determining region (CDR) amino acid sequences as set forth in SEQ ID NOs: 47-52.

According to another aspect of the present invention, there is provided an antibody comprising an antigen recognition domain which comprises complementarity determining region (CDR) amino acid sequences as set forth in SEQ ID NOs: 53-58.

According to one embodiment the antibodies of the present invention are IgG1 antibodies. Exemplary methods for generating IgG1 antibodies are described in the general materials and experimental methods of the Examples section herein below.

As mentioned herein above, the antibodies of the present invention may be antibody fragments. Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5 S fragment denoted F(ab′)₂. This fragment can be further cleaved using a thiol reducing agent, and optionally a blocking group for the sulfhydryl groups resulting from cleavage of disulfide linkages, to produce 3.5 S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence.

The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

It will be appreciated that once the CDRs of an antibody are identified, using conventional genetic engineering techniques, expressible polynucleotides encoding any of the forms or fragments of antibodies described herein can be devised and modified in one of many ways in order to produce a spectrum of related-products as further described herein below.

Thus, for example polynucleotides as set forth in SEQ ID NOs: 16 and 18 may be used in nucleic acid constructs for the expression of the TA2 antibody. Polynucleotides as set forth in SEQ ID NOs: 903-906 may be used in nucleic acid constructs for the expression of the MC1 antibody. Polynucleotides as set forth in SEQ ID NOs: 907-910 may be used in nucleic acid constructs for the expression of the B2 antibody.

Thus, polynucleotide sequences such as those described herein above are inserted into expression vectors (i.e., a nucleic acid construct) to enable expression of the recombinant antibody. The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

A variety of prokaryotic or eukaryotic cells can be used as host-expression systems to express the antibodies of the present invention. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In bacterial systems, a number of expression vectors can be advantageously selected depending upon the use intended for the antibody expressed. For example, when large quantities of antibody are desired, vectors that direct the expression of high levels of the protein product, possibly as a fusion with a hydrophobic signal sequence, which directs the expressed product into the periplasm of the bacteria or the culture medium where the protein product is readily purified may be desired. Certain fusion protein engineered with a specific cleavage site to aid in recovery of the polypeptide may also be desirable. Such vectors adaptable to such manipulation include, but are not limited to, the pET series of E. coli expression vectors [Studier et al., Methods in Enzymol. 185:60-89 (1990)].

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the antibody coding sequence can be driven by a number of promoters. For example, viral promoters such as the 35 S RNA and 19 S RNA promoters of CaMV [Brisson et al., Nature 310:511-514 (1984)], or the coat protein promoter to TMV [Takamatsu et al., EMBO J. 6:307-311 (1987)] can be used. Alternatively, plant promoters can be used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed antibody.

Various methods can be used to introduce the expression vector of the present invention into the host cell system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Transformed cells are cultured under effective conditions, which allow for the expression of high amounts of recombinant antibody. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the recombinant cell, secreted into the fermentation medium, secreted into a space between two cellular membranes, such as the periplasmic space in E. coli; or retained on the outer surface of a cell or viral membrane.

Following a predetermined time in culture, recovery of the recombinant antibody is effected.

The phrase “recovering the recombinant antibody” used herein refers to collecting the whole fermentation medium containing the antibody and need not imply additional steps of separation or purification.

Thus, antibodies of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

To facilitate recovery, the expressed coding sequence can be engineered to encode the antibody of the present invention and fused cleavable moiety. Such a fusion protein can be designed so that the antibody can be readily isolated by affinity chromatography; e.g., by immobilization on a column specific for the cleavable moiety. Where a cleavage site is engineered between the antibody and the cleavable moiety, the antibody can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

As mentioned, the antibody of the present invention is contacted with potential melanoma cells under conditions which allow immunocomplex formation, wherein a presence of the immunocomplex or level thereof is indicative of the melanoma cell.

The contacting may be effected in vitro (i.e. in a cell line), ex vivo or in vivo.

As mentioned, the method of the present invention is effected under conditions sufficient to form an immunocomplex (e.g. a complex between the antibodies of the present invention and the tyrosinase derived peptide complexed to an MHC-I); such conditions (e.g., appropriate concentrations, buffers, temperatures, reaction times) as well as methods to optimize such conditions are known to those skilled in the art, and examples are disclosed herein.

Determining a presence or level of the immunocomplex of the present invention is dependent on the detectable moiety to which the antibody is attached.

Examples of detectable moieties that can be used in the present invention include but are not limited to radioactive isotopes, phosphorescent chemicals, chemiluminescent chemicals, fluorescent chemicals, enzymes, fluorescent polypeptides and epitope tags. The detectable moiety can be a member of a binding pair, which is identifiable via its interaction with an additional member of the binding pair, and a label which is directly visualized. In one example, the member of the binding pair is an antigen which is identified by a corresponding labeled antibody. In one example, the label is a fluorescent protein or an enzyme producing a colorimetric reaction.

Further examples of detectable moieties, include those detectable by Positron Emission Tomagraphy (PET) and Magnetic Resonance Imaging (MRI), all of which are well known to those of skill in the art.

When the detectable moiety is a polypeptide, the immunolabel (i.e. the antibody conjugated to the detectable moiety) may be produced by recombinant means or may be chemically synthesized by, for example, the stepwise addition of one or more amino acid residues in defined order using solid phase peptide synthetic techniques. Examples of polypeptide detectable moieties that can be linked to the antibodies of the present invention using recombinant DNA technology include fluorescent polypeptides, phosphorescent polypeptides, enzymes and epitope tags.

Expression vectors can be designed to fuse proteins encoded by the heterologous nucleic acid insert to fluorescent polypeptides. For example, antibodies can be expressed from an expression vector fused with a green fluorescent protein (GFP)-like polypeptide. A wide variety of vectors are commercially available that fuse proteins encoded by heterologous nucleic acids to the green fluorescent protein from Aequorea victoria (“GFP”), the yellow fluorescent protein and the red fluorescent protein and their variants (e.g., Evrogen). In these systems, the fluorescent polypeptide is entirely encoded by its amino acid sequence and can fluoresce without requirement for cofactor or substrate. Expression vectors that can be employed to fuse proteins encoded by the heterologous nucleic acid insert to epitope tags are commercially available (e.g., BD Biosciences, Clontech).

Alternatively, chemical attachment of a detectable moiety to the antibodies of the present invention can be effected using any suitable chemical linkage, direct or indirect, as via a peptide bond (when the detectable moiety is a polypeptide), or via covalent bonding to an intervening linker element, such as a linker peptide or other chemical moiety, such as an organic polymer. Such chimeric peptides may be linked via bonding at the carboxy (C) or amino (N) termini of the peptides, or via bonding to internal chemical groups such as straight, branched or cyclic side chains, internal carbon or nitrogen atoms, and the like. Such modified peptides can be easily identified and prepared by one of ordinary skill in the art, using well known methods of peptide synthesis and/or covalent linkage of peptides. Description of fluorescent labeling of antibodies is provided in details in U.S. Pat. Nos. 3,940,475, 4,289,747, and 4,376,110.

Exemplary methods for conjugating two peptide moieties are described herein below:

SPDP Conjugation:

Any SPDP conjugation method known to those skilled in the art can be used. For example, in one illustrative embodiment, a modification of the method of Cumber et al. (1985, Methods of Enzymology 112: 207-224) as described below, is used.

A peptide, such as an identifiable or therapeutic moiety, (1.7 mg/ml) is mixed with a 10-fold excess of SPDP (50 mM in ethanol) and the antibody is mixed with a 25-fold excess of SPDP in 20 mM sodium phosphate, 0.10 M NaCl pH 7.2 and each of the reactions incubated, e.g., for 3 hours at room temperature. The reactions are then dialyzed against PBS.

The peptide is reduced, e.g., with 50 mM DTT for 1 hour at room temperature. The reduced peptide is desalted by equilibration on G-25 column (up to 5% sample/column volume) with 50 mM KH₂PO₄ pH 6.5. The reduced peptide is combined with the SPDP-antibody in a molar ratio of 1:10 antibody:peptide and incubated at 4° C. overnight to form a peptide-antibody conjugate.

Glutaraldehyde Conjugation:

Conjugation of a peptide (e.g., an identifiable or therapeutic moiety) with an antibody can be accomplished by methods known to those skilled in the art using glutaraldehyde. For example, in one illustrative embodiment, the method of conjugation by G. T. Hermanson (1996, “Antibody Modification and Conjugation, in Bioconjugate Techniques, Academic Press, San Diego) described below, is used.

The antibody and the peptide (1.1 mg/ml) are mixed at a 10-fold excess with 0.05% glutaraldehyde in 0.1 M phosphate, 0.15 M NaCl pH 6.8, and allowed to react for 2 hours at room temperature. 0.01 M lysine can be added to block excess sites. After-the reaction, the excess glutaraldehyde is removed using a G-25 column equilibrated with PBS (10% v/v sample/column volumes)

Carbodiimide Conjugation:

Conjugation of a peptide with an antibody can be accomplished by methods known to those skilled in the art using a dehydrating agent such as a carbodiimide. Most preferably the carbodiimide is used in the presence of 4-dimethyl aminopyridine. As is well known to those skilled in the art, carbodiimide conjugation can be used to form a covalent bond between a carboxyl group of peptide and an hydroxyl group of an antibody (resulting in the formation of an ester bond), or an amino group of an antibody (resulting in the formation of an amide bond) or a sulfhydryl group of an antibody (resulting in the formation of a thioester bond).

Likewise, carbodiimide coupling can be used to form analogous covalent bonds between a carbon group of an antibody and an hydroxyl, amino or sulfhydryl group of the peptide. See, generally, J. March, Advanced Organic Chemistry: Reaction's, Mechanism, and Structure, pp. 349-50 & 372-74 (3d ed.), 1985. By means of illustration, and not limitation, the peptide is conjugated to an antibody via a covalent bond using a carbodiimide, such as dicyclohexylcarbodiimide. See generally, the methods of conjugation by B. Neises et al. (1978, Angew Chem., Int. Ed. Engl. 17:522; A. Hassner et al. (1978, Tetrahedron Lett. 4475); E. P. Boden et al. (1986, J. Org. Chem. 50:2394) and L. J. Mathias (1979, Synthesis 561). The level of immunocomplex may be compared to a control sample from a non-diseased subject, wherein an up-regulation of immunocomplex formation is indicative of melanoma. Preferably, the subject is of the same species e.g. human, preferably matched with the same age, weight, sex etc. It will be appreciated that the control sample may also be of the same subject from a healthy tissue, prior to disease progression or following disease remission.

It will be appreciated that antibodies of the present invention may also be used to ascertain if a melanoma patient is a suitable candidate for epitope-related therapy via in vivo or ex-vivo testing. As mentioned above, the present inventors found that the expression level of a melanoma antigen (as measured by RT-PCR) did not reflect its presentation numbers on the membrane of a melanoma cell. Thus, antibodies of the present invention may be used as tools to evaluate the level (i.e. density) of a particular melanoma antigen on a cell, wherein a level higher than a predetermined threshold is indicative of an individual being suitable for TCRL epitope-directed therapy and a level lower than a predetermined threshold is indicative of an individual being suitable for CTL epitope-directed therapy. Alternatively, or additionally a level below a predetermined threshold is indicative of an individual not being suitable for TCRL epitope-directed therapy and a level above a predetermined threshold is indicative of an individual not being suitable for CTL epitope-directed therapy.

CTL epitope directed therapy aims to generate tumor-reactive CD8+ T cells in response to administration of tumor-specific peptide epitopes to bring about the destruction of the tumor cells. However, despite much effort, vaccination approaches to date can at best induce objective cancer regressions consistent with standard oncologic criteria in only a small minority of patients with solid cancers. One possible explanation of the lack of success of CTL epitope directed therapy is the phenomenon of anergy which dictates that continual exposure of T cells to antigens maintains an unresponsive state and result in adaptive tolerance (B. Rocha, et al., 1995; L. S. Taams, et al., 1999; R. H. Schwartz, 2003; F. Ramsdell and B. J. Fowlkes, 1992). The present invention seeks to overcome this obstacle by determining the level of a particular epitope by examining their levels using the antibodies of the present invention.

Further information on CTL epitope directed therapy can be obtained in the following review articles—Acres B, et al., Curr Opin Drug Discov Devel. 2007 March; 10(2):185-92. Finn O J. Immunol. Res. 2006;36(1-3):73-82. Chen W, McCluskey J. Adv Cancer Res. 2006; 95:203-47. Klebanoff C A, Gattinoni L, Restifo N P. Immunol Rev. 2006 June; 211:214-24. Slingluff C L Jr, Engelhard V H, Ferrone S. Clin Cancer Res. 2006 Apr. 1; 12(7 Pt 2):2342s-2345s. Mocellin S, Lise M, Nitti D. Tumor immunology. Adv Exp Med. Biol. 2007; 593:147-56. Gattinoni L, Powell D J Jr, Rosenberg S A, Restifo N P. Nat Rev Immunol. 2006 May; 6(5):383-93. Rosenberg S A. N Engl J. Med. 2004 Apr. 1; 350(14):1461-3. Rosenberg S A. Nature. 2001 May 17; 411(6835):380-4, each of which is incorporated herein by reference in its entirety.

The predetermined thresholds may be estimated as shown in the Example section herein below. An exemplary threshold above which it may be determined that a patient is suitable for TCRL epitope-directed therapy (or unsuitable for CTL epitope directed therapy) is above about 500, more preferably above about 800, more preferably above about 1000, more preferably above about 1200 and even more preferably above about 1500 MHC-peptide complexes per cell that present a single epitope. An exemplary threshold below which it may be determined that a patient is not suitable for TCRL epitope-directed therapy (or suitable for CTL epitope directed therapy) is below about 400, more preferably below about 300, more preferably below about 200, and even more preferably below about 100 MHC-peptide complexes per cell that present a single epitope.

It will be appreciated that antibodies of the present invention targeted against a variety of peptide antigens including, but not limited to gp100, Mart-1 and Tyrosinase may also be used to identify the optimal candidate target for epitope-directed therapy on an individual level.

Furthermore, the antibodies of the present invention can be used to monitor via in vivo or ex vivo testing, the therapeutic impact of epitope directed therapy to identify whether a tumoral process evolved and became refractory via mechanisms relying on or independent of loss of antigen expression.

It will be appreciated that the antibodies of the present invention may also be used to kill or ablate cells (e.g., a non-cancerous cell, e.g., a normal, benign or hyperplastic cell, or a cancerous cell, e.g., a malignant cell, e.g., a cell found in a solid tumor, a soft tissue tumor, or a metastatic lesion (e.g., a cell found in renal, urothelial, colonic, rectal, pulmonary, breast or hepatic, cancers and/or metastasis). Methods of the invention include the steps of contacting the cell with an anti-(MHC-peptide complex) ligand, e.g., an anti-(MHC-peptide complex) antibody described herein, in an amount sufficient to ablate or kill the cell.

This method can be used on cells in culture, e.g. in vitro or ex vivo. For example, cancerous or metastatic cells (e.g., renal, urothelial, colon, rectal, lung, breast, ovarian, prostatic, or liver cancerous or metastatic cells) can be cultured in vitro in culture medium and the contacting step can be effected by adding the anti-(MHC-peptide complex) ligand to the culture medium. The method can be performed on cells (e.g., cancerous or metastatic cells) present in a subject, as part of an in vivo (e.g., therapeutic or prophylactic) protocol as described herein below.

Since the present inventors discovered that tyrosinase peptides are highly presented on melanoma cells compared to other peptide antigens, it may be concluded that tyrosinase peptides may serve as attractive targets for TCRL epitope-directed therapy.

Thus, according to another aspect of the present invention there is provided a method of treating a melanoma. The method comprises administering to a subject in need thereof a therapeutically effective amount of the tyrosinase peptide directed antibodies of the present invention.

According to one embodiment of this aspect of the present invention, the antibody is an IgG1 antibody. Using IgG antibodies, tumor cell lysis can be achieved by antibody-dependent cell-mediated cytotoxicity (ADCC) or complement-dependant cytotoxicity (CDC) mechanisms (J. Golay, et al., 2004; H. Mellstedt, 2003; Modjtahedi, et al., 2003; N. Prang, et al., 2005).

According to another embodiment of this aspect of the present invention, the antibody is conjugated to a therapeutic moiety.

The therapeutic moiety can be, for example, a cytotoxic moiety, a toxic moiety, a cytokine moiety and a second antibody moiety comprising a different specificity to the antibodies of the present invention.

In a similar fashion to an immunolabel, an immunotoxin (i.e. a therapeutic moiety attached to an antibody of the present invention) may be generated by recombinant or non-recombinant means. Thus, the present invention envisages a first and second polynucleotide encoding the antibody of the present invention and the therapeutic moiety, respectively, ligated in frame, so as to encode an immunotoxin. The following table provides examples of sequences of therapeutic moieties.

TABLE 1

| | Amino Acid sequence | Nucleic Acid sequence |
|---|---|---|
| | (Genebank Accession | (Genebank Accession |
| Therapeutic Moiety | No.) | No.) |
| Pseudomonas exotoxin | AAB25018 | S53109 |
| Diphtheria toxin | E00489 | E00489 |
| interleukin 2 | CAA00227 | A02159 |
| CD3 | P07766 | X03884 |
| CD16 | AAK54251 | AF372455 |
| interleukin 4 | P20096 | ICRT4 |
| HLA-A2 | P01892 | K02883 |
| interleukin 10 | P22301 | M57627 |
| Ricin A toxin | 225988 | A23903 |

Exemplary methods of conjugating the antibodies of the present invention to peptide therapeutic agents are described herein above.

The antibodies of the present invention may be provided per se or may be administered as a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the antibodies of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (antibodies) effective to prevent, alleviate or ameliorate symptoms of a disorder (e.g., melanom) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods for Examples 1-9

Production of biotinylated single-chain MHC/peptide complexes—Single-chain MHC (scMHC)/peptide complexes were produced by in vitro refolding of inclusion bodies produced in Escherichia coli upon IPTG induction, as described (Denkberg, et al., 2000). Briefly, a scMHC, which contains the β₂-microglobulin and the extracellular domains of the HLA-A2 gene connected to each other by a flexible linker, was engineered to contain the BirA recognition sequence for site-specific biotinylation at the C terminus (scMHC-BirA). In vitro refolding was performed in the presence of peptides, as described (Denkberg, et al., 2000). Correctly folded MHC/peptide complexes were isolated and purified by anion exchange Q-Sepharose chromatography (Pharmacia, Peapack, N.J.), followed by site-specific biotinylation using the BirA enzyme (Avidity, Denver, Colo.).

Selection of phage Abs on biotinylated complexes—Selection of phage Abs on biotinylated complexes was performed, as described [Denkberg G, et al., Proc Natl Acad Sci USA. 2002 Jul. 9; 99(14):9421-6. Epub 2002 Jul. 1]. Briefly, a large human Fab library containing 3.7×10¹⁰ different Fab clones was used for the selection. Phages were first preincubated with streptavidin-coated paramagnetic beads (200 μl; Dynal, Oslo, Norway) to deplete the streptavidin binders. The remaining phages were subsequently used for panning with decreasing amounts of biotinylated scMHC-peptide complexes. The streptavidin-depleted library was incubated in solution with soluble biotinylated scHLA-A2/Tyrosinase complexes (500 nM for the first round, and 100 nM for the following rounds) for 30 minutes at room temperature (RT). Streptavidin-coated magnetic beads (200 μl for the first round of selection, and 100 μl for the following rounds) were added to the mixture and incubated for 10-15 minutes at RT. The beads were washed extensively 12 times with PBS/Tween 0.1%, and additional two washes were with PBS. Bound phages were eluted with triethylamine (100 mM, 5 minutes at RT), followed by neutralization with Tris-HCl (1 M, pH 7.4), and used to infect E. coli TG1 cells (OD=0.5) for 30 minutes at 37° C. The diversity of the selected Abs was determined by DNA fingerprinting using a restriction endonuclease (BstNI), which is a frequent cutter of Ab V gene sequences.

Expression and purification of soluble recombinant Fab Abs—Fab Abs were expressed and purified, as described recently [Denkberg G, et al., Proc Natl Acad Sci U S A. 2002 Jul. 9; 99(14):9421-6. Epub 2002 Jul. 1]. TG1 or BL21 cells were grown to OD₆₀₀=0.8-1.0 and induced to express the recombinant Fab Ab by the addition of IPTG for 3-4 hours at 30° C. Periplasmic content was released using the B-PER solution (Pierce, Rockford, Ill.), which was applied onto a prewashed TALON column (Clontech, Palo Alto, Calif.). Bound Fabs were eluted using 0.5 ml of 100 mM imidazole in PBS. The eluted Fabs were dialyzed twice against PBS (overnight, 4° C.) to remove residual imidazole. Specificity of the produced Fabs was verified by ELISA analysis, as described [Denkberg G, et al., Proc Natl Acad Sci USA. 2002 Jul. 9; 99(14):9421-6. Epub 2002 Jul. 1].

Flow cytometry—EBV-transformed B-lymphoblast JY cells were washed with serum-free RPMI medium and incubated overnight with medium containing 10 μM Tyrosinase 369-377 peptide [YMDGTMSQV (SEQ ID NO:1) or control peptides: Gag (SEQ ID NO:2), Tyr N (SEQ ID NO:3), 2092M (SEQ ID NO:4), 280 (SEQ ID NO:5), 540 (SEQ ID NO:6), TARP (SEQ ID NO:7), 154 (SEQ ID NO: 20), Tax (SEQ ID NO: 26), 280 m (SEQ ID NO: 28), pol (SEQ ID NO: 65) 865 (SEQ ID NO: 29) and Mart (SEQ ID NO: 22)). Cells (10⁶) were incubated 1 hour at 4° C. with 1-2 μg specific antibody, followed by incubation for 45 minutes at 4° C. with PE-labeled anti-human antibody. Cells were finally washed and analyzed by a FACStar flow cytometer (BD Biosciences, San Jose, Calif.). Melanoma cells were examined for endogenous antigen expression by staining with 2-5 μg of the specific antibodies.

cDNA production and quantitative real-time PCR analysis—mRNA was isolated from melanoma cell lines with oligo (dT) magnetic beads using Dynabeads mRNA DIRECT Kit (Dynal) according to manufacture's instructions. mRNA was converted into cDNA using cloned-AMV reverse transcriptase (invitrogen) according to manufacture's instructions. Real time PCR was performed using Assays-on Demand Gene Expression Assays (Applied Biosystems). Assay IDs Hs00165976_ml, Hs00173854_ml, Hs00194133_ml were used for tyrosinase, gp100 and Mart-1 expression assays, respectively. For all real time PCR reactions, total volume of 20 μl contained 15 ng of mRNA converted to cDNA and 10 μl TaqMan Universal PCR Mastermix (Applied Biosystems). Primers and FAM-labeled probes were added to each reaction at the final concentration of 0.9 μM and 0.25 μM, respectively. A “no template” control that contained all the above reagents was also included to detect the presence of contaminating DNA. 5 ng of Glyceraldehyde-3-phosphate dehydrogenase (GAPDH) mRNA converted to cDNA was used as an internal control gene for mRNA expression and for analyzing relative expression (Assay ID Hs99999905_ml). Amplification and fluorescence detection was conducted in an ABI/Prism 7700 sequence detector (Applied Biosystems, USA) with a program of 50° C. for 2 minutes, 95° C. for 10 minutes, 40 cycles of 95° C. for 15 seconds and 60° C. for one minute.

The amount of target genes was determined from the comparative C_{T} method. The target genes were normalized to GAPDH and were expressed as ΔC_{T}(C_{T}-threshold cycle of target gene minus C_{T} of GAPDH).

Production of Fab tetramers—The light and heavy chains of the Fab were PCR amplified and cloned separately into pRB vector. The C terminus of the Fab light chain was fused to the BirA tag for site specific biotinylation. Each of the vectors was transformed into E. coli BL21 cells and expressed upon IPTG induction as inclusion bodies. The inclusion bodies, containing light or heavy chains of the Fab were isolated, solubilized, refolded with each other and purified by ion exchange chromatography. The recombinant Fab was biotinylated and tetramers were generated by adding fluorescently-labeled streptavidin (Reinke, et al., 2005).

Construction of whole IgG antibody—The heavy and light Fab genes were cloned for expression as human IgG1 kappa antibody into mammalian backbone of the eukaryotic expression vector pCMV/myc/ER. For the heavy chain, the multiple cloning site, the myc epitope tag, and the ER retention signal of pCMV/myc/ER were replaced by a cloning site containing recognition sites for BssHI and NheI followed by the human IgG1 constant heavy chain region cDNA isolated by RT-PCR from human lymphocyte total RNA. A similar construct was generated for the light chain. Each shuttle expression vector carries a different antibiotics resistance gene. Expression was facilitated by co-transfection of the two constructs into human embryonic kidney HEK293 cell, by using FuGENE 6 Transfection Reagent (Roche). After co-transfection, cells were grown on selective media. Antibody-producing cells were adapted to growth in 0.5% serum followed by purification using proteinA affinity chromatography.

Immunofluorescence: Cells were fixed for 10 minutes at RT with 0.1% formaldehyde, rinsed with 0.1% BSA-PBS, incubated for 1 hour at 4° C. with primary antibody, followed by incubation with the fluorescence-labeled secondary antibody goat anti-human Alexa Fluor 488 or goat anti-mouse Alexa Fluor 594 (Molecular Probes). For staining of nuclear DNA DRAQ5 (Alexis biochemicals) was used. A BioRad MRC1024 confocal microscope was employed for analysis.

Determination of protein stability: 501A melanoma cells were incubated with Cyclohexamide (100 μg/ml, Sigma) to inhibit protein synthesis. The cells were lysed after 0, 2, 4 and 6 hours in PBS containing 10% NP-40, 1% DOC, 2 mM EDTA, 10 mM Tris pH=7, 1 mM PMSF and protease inhibitor cocktail. The lysate was passed through needle and was incubated 10 minutes on ice. Cells were centrifuged at 14 000 r.p.m. for 5 minutes at 4° C. and the supernatant was collected. Equal amounts of sample were loaded on SDS-PAGE and electroblotted onto nitrocellulose. The blots were probed with T311 mouse anti-Tyrosinase, HMB-45 mouse anti-gp100 or A103 mouse anti-Mart-1 followed by incubation with a secondary horseradish peroxidase-conjugated antibody and detection by chemiluminescence. For negative control, Panc-1 cells, which do not express tyrosinase, gp100 and mart-1, were used. The resulting bands were quantified with multi gauge v2.2 software. The degradation rate is expressed as half-life (t_{1/2}), the time for degradation of 50% of the sample. The degradation rate of each protein was evaluated by three to eight independent determinations of t_{1/2}. The data are expressed as mean±SD.

DNA microarray: The gene expression profile of JKF6 memory T cell clone as a function of time post exposure to various antigen densities on target cells was analyzed using Affymetrix human gene DNA array chip. Each Time-Antigen spot was composed of 3 independent biological assays. CTLs were incubated with pulsed JY APCs loaded with a peptide concentration that corresponds to antigen density of 10,100 and 800 sites per cell. CTLs were incubated for 4, 16 or 36 hours with peptide-pulsed JY target cells which subsequently after incubation were depleted using CD19 easySep© (Stem cell®) magnetic beads. JY Depleted cultures contained 97.3% or more CD8+ T cells with no detectable contamination of CD 19 positive JY target cells.

All experiments were performed using Affymetrix Hu133A 2.0 oligonucleotide arrays, as described at (url1). Total RNA from each sample was used to prepare biotinylated target RNA (url2). Briefly, 5 μg of mRNA pooled from 3 independent biological assays was used to generate first-strand cDNA by using a T7-linked oligo(dT) primer. After second-strand synthesis, in vitro transcription was performed with biotinylated UTP and CTP (Affymetrix), resulting in approximately 300-fold amplification of mRNA. The target cDNA generated from each sample was processed as per manufacturer's recommendation using an Affymetrix GeneChip Instrument System (url2). Briefly, spike controls were added to 15 μg fragmented cRNA before overnight hybridisation. Arrays were then washed and stained with streptavidin-phycoerythrin, before being scanned on an Affymetrix GeneChip scanner. A complete description of these procedures is available at (url2). Additionally, quality and amount of starting RNA was confirmed using an agarose gel. After scanning, array images were assessed by eye to confirm scanner alignment and the absence of significant bubbles or scratches on the chip surface. 3′/5′ ratios for GAPDH and beta-actin were confirmed to be within acceptable limits (0.97-0.96 and 1.14-1.2), and BioB spike controls were found to be present on all chips, with BioC, BioD and CreX also present in increasing intensity. When scaled to a target intensity of 150 (using Affymetrix MAS 5.0 array analysis software), scaling factors for all arrays were within acceptable limits (1.355-1.655), as were background, Q values and mean intensities. Details of quality control measures can be found at www.ncbi.nlm.nih.gov/geo/ or at http://eng.sheba.co.il/genomics.

Data analysis: The probe sets contained in the Affymetrix Human Hu133A2 oligonucleotide array or oligonucleotide arrays were filtered using Mas 5 algorithm. Treated and control samples were compared. The comparison generated a list of “active genes” representing probe sets changed by at least 2 fold (as calculated from the MAS 5 Log Ratio values)(LR>=1 or LR<=−1) and detected as “Increased” or as “Decrease” (I or D, p-value 0.0025) or Marginal Increased” or as Marginal Decrease (MI or MD, p-value 0.003) in all treated sample as compared to the control samples (CTLs at time 0) in at least one time point. This list excluded up-regulated genes in all treated samples with signals lower than 20 or detected as absent, and down-regulated gene with base line signals lower than 20 and detected as absent in the control samples. For further filtering we used the probe sets changed by at least 2 fold (between signals) between the 2 treated samples at 36 hrs: 700 sites per target −36 hrs (P22) and 100 sites per target −36 hrs (P21). Hierarchical clustering was performed using Spotfire DecisionSite for Functional Genomics (Somerville, Mass.).

Genes were classified into functional groups using the G0 annotation tool (G. Dennis, Jr., et al., 2003). Over-representation calculations were done using Ease (D. A. Hosack, et al., 2003) Functional classifications with an “Ease score” lower than 0.05 were marked as over represented.

### Example 1

### Isolation of Fab TCRL Antibodies Capable of Specific Binding to MHC-TYRD₃₆₉₋₃₇₇ Complex

Experimental Results

Generation of MHC-Tyrosinase₃₆₉₋₃₇₇ complex—Previous studies performed by the present inventors have shown the generation of recombinant antibodies with peptide-specific, HLA-A2-restricted specificity to tumor and viral T cell epitopes using large antibody phage libraries. These molecules are termed TCR-like antibodies. To generate such antibodies with a specificity to the HLA-A2/Tyrosinase₃₆₉₋₃₇₇ complex, recombinant peptide-HLA-A2 complexes were generated that present the Tyrosinase peptide (SEQ ID NO: 1) using a single chain MHC construct. In this construct, the extracellular domains of HLA-A2 were connected into a single chain molecule with β₂ microglobulin using a 15-amino acid flexible linker. The complexes were bacterially produced in E. Coli BL21 cells as intracellular inclusion bodies and refolded with Tyrosinase 369-377 peptide by redox-shuffling buffering system. Correctly folded complexes were purified by ion exchange chromatography on Q-Sepharose column, followed by biotinylation of the complexes by BirA ligase.

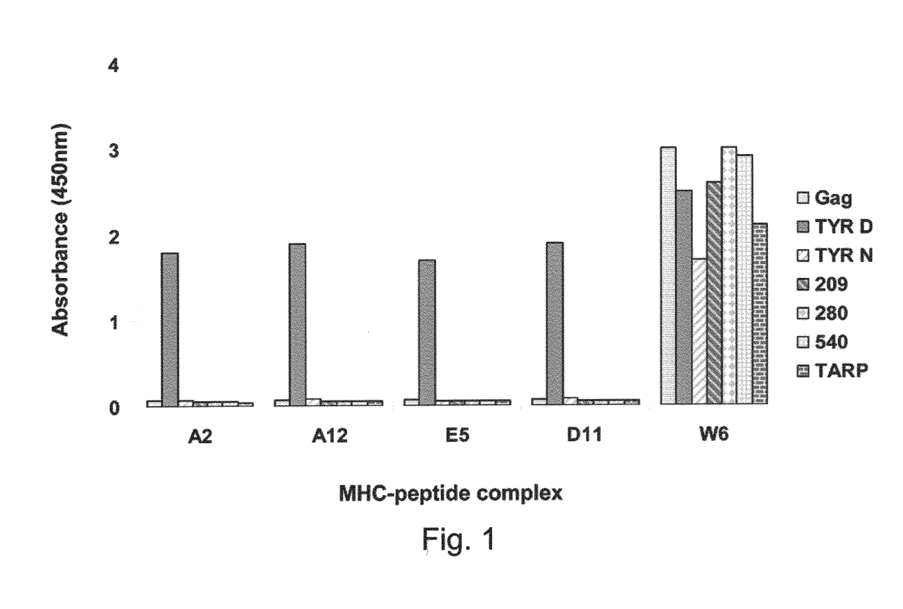

Isolation of TCR-like antibodies specific to the MHC'-Tyrosinase₃₆₉₋₃₇₇ complex—To isolate TCR-like antibodies, a large naïve Fab antibody phage display library [de Haard H J, J Biol. Chem. 1999 Jun. 25; 274(26):18218-30] containing 3.7×10¹⁰ different Fab clones was screened with the produced complexes. Following four rounds of selection, a two-fold enrichment in phage output was observed. Specific clones were detected by an ELISA assay in which binding was tested with specific and non-specific complexes. Soluble Fab fragments were produced from the phage clones, using E. Coli BL21 cells for expression and metal affinity chromatography for purification. The specificity of the soluble Fab was determined by ELISA on biotinylated MHC-peptide complexes that were immobilized to BSA-biotin-streptavidin-coated wells (FIG. 1). To determine the correct folding of the bound complexes and their stability during the binding assay, anti-HLA mAb W6/32 was used [Barnstable C J, Cell. 1978 May; 14(1):9-20]. This Ab recognizes HLA complexes only when folded correctly and when containing a peptide. FIG. 1 shows the reactivity of several clones (A2, A12, E5, D11) in an ELISA assay with purified HLA-A2-Tyr complexes as well as with control HLA-A2 complexes displaying other HLA-A2-restricted peptides. The soluble Fabs reacted specifically with the MHC class I complex containing the Tyr 369-377 peptide (SEQ ID NO:1) but not with the other 6 control peptides (SEQ ID NOs:2-7).

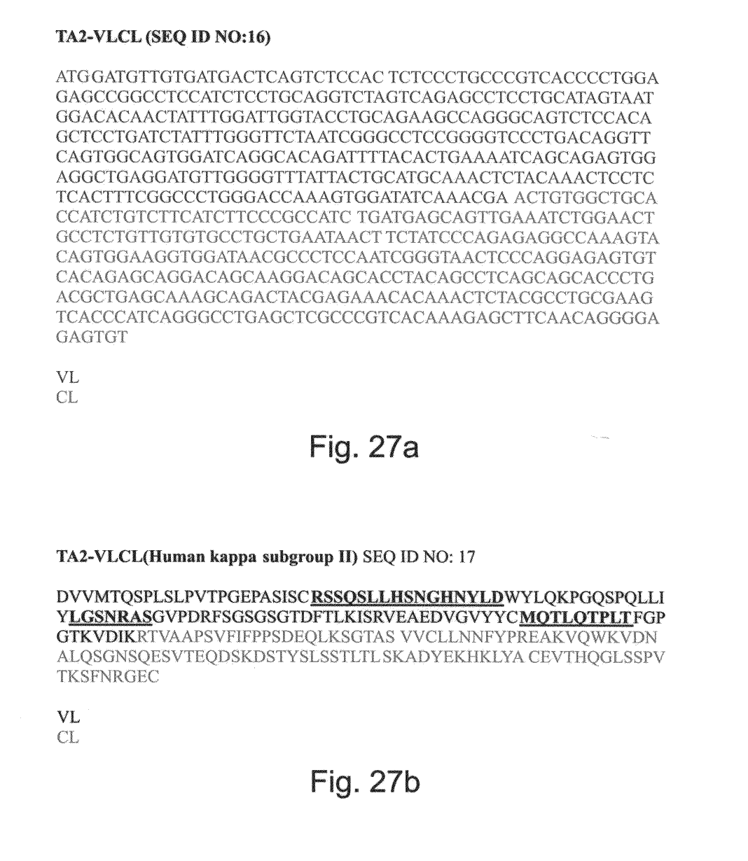

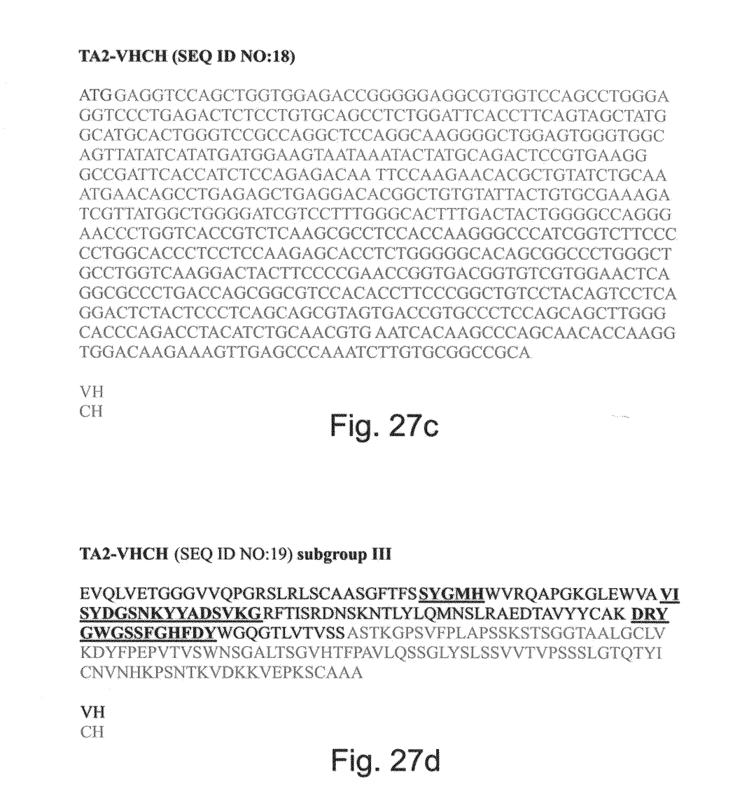

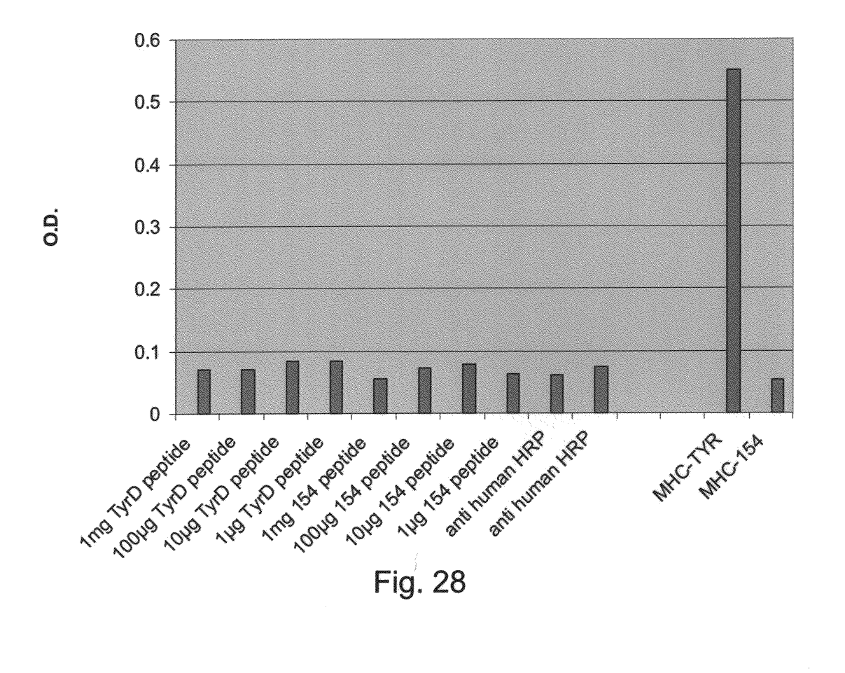

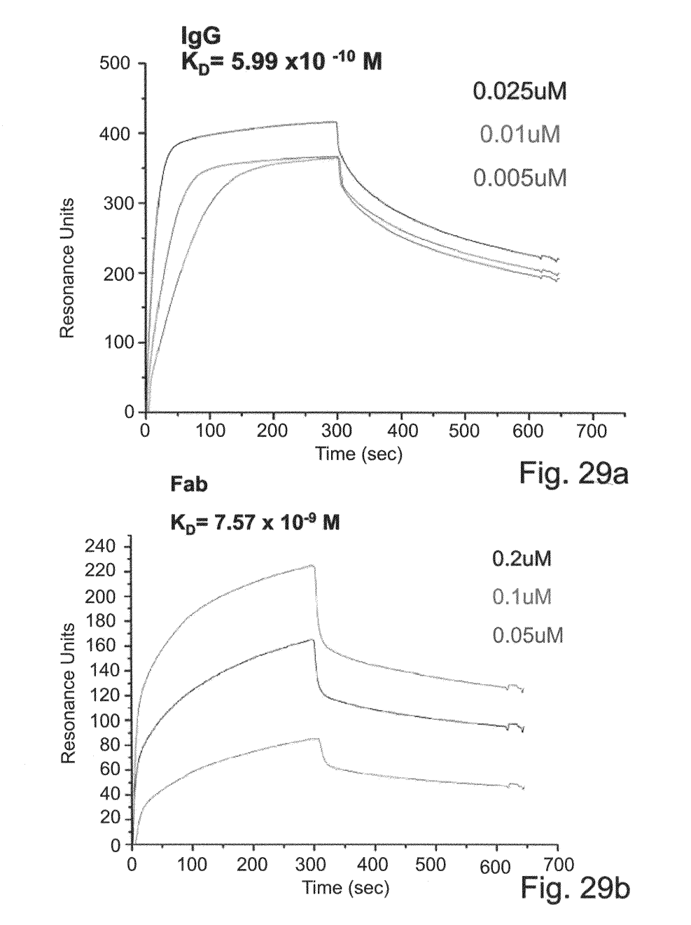

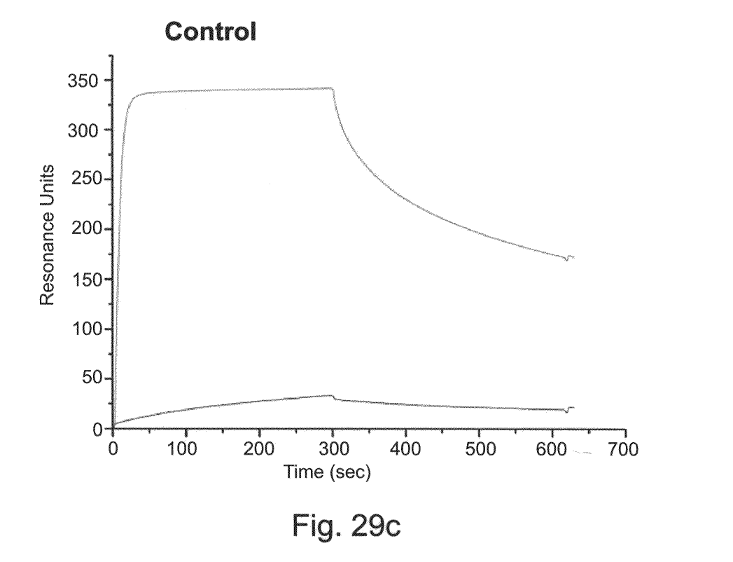

Determination of the CDR sequences of the Fab TCRL capable of binding to MHC-Tyrosinase D369-377 complex—To determine a DNA pattern of the selected clones, a DNA fingerprint assay was performed, by PCR amplification followed by BstNI restriction reaction. The results showed only one pattern indicating one positive anti-Tyrosinase 369-377 clone isolated from the library (data not shown). The final clone used further in this study was designated TA2 (from phage Fab A2 of FIG. 1). The DNA and deduced amino acid sequences of TA2 VH+CH1 and VL+CL (heavy chain+light chain) are presented in FIGS. 27a-d and are set forth by SEQ ID NOs: 16-19. The CDR sequences of the VL are set forth by amino acids 24-39, 55-61 and 94-102 of SEQ ID NO: 17. The CDR sequences of the VH are set forth by amino acids 94-102, 50-66 and 99-112 of SEQ ID NO: 19. The nucleic acid sequences encoding the VL CDR sequences are set forth by nucleic acids 70-117, 152-183 and 280-306 of SEQ ID NO: 16; the nucleic acid sequences encoding the VH CDR sequences are set forth by nucleic acids 91-105, 147-198 and 295-336 of SEQ ID NO:18.

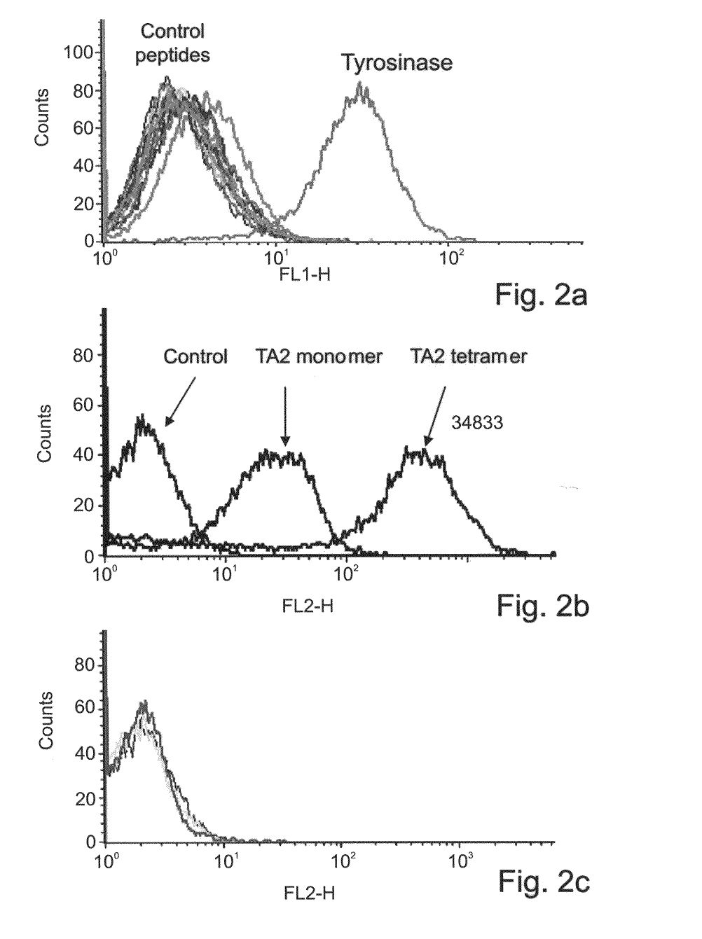

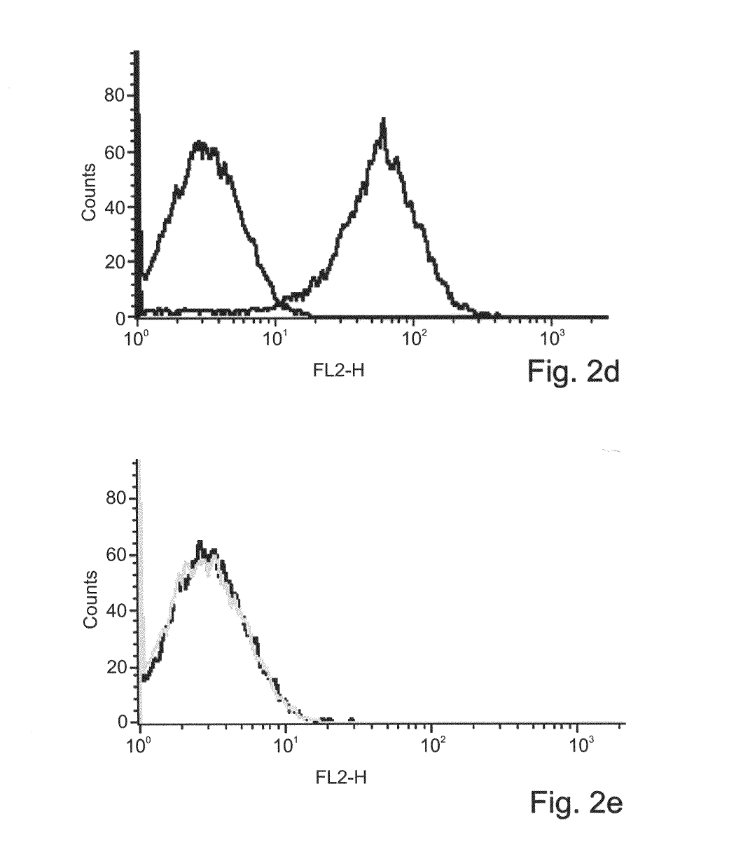

The soluble TA2 Fab antibody is capable of binding to MHC-Tyrosinase complexes expressed on antigen presenting cells (APCs)—To test the ability of the anti-HLA-A2/Tyr TA2 Fab to bind the target in its native form as expressed on antigen presenting cells, EBV-transformed B-lymphoblasts HLA-A2⁺ JY cells were loaded with Tyrosinase 369-377 YMDGTMSQV peptide (SEQ ID NO: 1) or control peptides (SEQ ID NOs: 2-7). Peptide-loaded cells were incubated with the soluble purified Fab, followed by incubation with FITC labeled anti-human antibody. FIG. 2a shows specific binding of Fab TA2 to cells loaded with the Tyrosinase peptide (SEQ ID NO:1) but not to cells loaded with the control peptides (SEQ ID NOs: 2-7, 20, 22, 26, 28, 29 and 65).

The soluble TA2 Fab antibody is sensitive to a point mutation in the Tyrosinase 369-377 peptide—One of the peptides used in this assay was the unmodified Tyrosinase 369-377 peptide containing Asparagine (N) instead of Aspartic acid (D) at position 371 (YMNGTMSQV; SEQ ID NO:3). As shown in FIG. 2a, low level reactivity of the antibody with this peptide was observed, compared with the native peptide (SEQ ID NO:1) presented on the surface of cells, which contains the amino-acid Asp at position 371. This emphasizes the fine specificity of the TA2 Fab to HLA-A2-Tyrosinase 369-377 complex.

Altogether, these results demonstrate the specific binding of the TA2 Fab antibody to the HLA-A2-Tyrosinase 369-377 complex.

### Example 2

### Generation of TA2 IgG Antibody

Since Fab fragments isolated from phage libraries are monovalent, the reactivity and sensitivity of the Fab can be improved by increasing its avidity. This was achieved by using two strategies: (i) generating Fab tetramers as was shown previously for other TCR-like antibodies (Cohen, et al., 2003) and (ii) transforming a TCR-like Fab fragment into a whole bi-valent IgG molecule.

Experimental Results

Generation of TA2 Fab tetramers—To generate Fab tetramers, the light and heavy chain encoding sequences of the TA2 Fab were PCR amplified and cloned separately into an pET-based expression vector. The C terminus of the TA2 Fab light chain was fused to the BirA tag (SEQ ID NO: 66)) for site specific biotinylation. Each of the vectors were transformed into E. coli BL21 cells and expressed as inclusion bodies which were further refolded together and purified by ion exchange chromatography. The purified recombinant Fab was biotinylated and tetramers were generated by adding fluorescently-labeled streptavidin. Binding of the TA2 Fab tetramer was examined by Flow cytometry with JY-pulsed cells. As shown in FIG. 2b, the staining intensity was significantly improved when cells were incubated with the TA2 Fab tetramer compared to the staining with TA2 Fab monomer. The Fab tetramers maintained their binding specificity (FIG. 2c).

Generation of TA2 IgG—For the second approach, the antibody Fab domains were implanted onto an IgG1 antibody scaffold. The heavy and light genes encoding the human TA2 Fab were cloned into a human IgG1 kappa antibody shuffling vector based on the backbone of the eukaryotic expression vector pCMV/myc/ER, as described in the General Materials and Experimental Methods section. Each shuttle expression vector carries a different selective gene and thus expression of TCR-like whole IgG molecules was facilitated by co-transfection of the two constructs into HEK293 cells. After co-transfection of HEK293 cells with the two heavy and light chain gene-containing plasmids, cells were grown on selective media. To determine the presence of specific human IgG in culture supernatants, flow cytometry analysis were performed using JY APCs which were loaded with Tyrosinase 369-377 peptide or control peptide and incubated with culture supernatants originating from a single colony. 12 of 14 clones tested were peptide specific and bound only MHC-Tyrosinase 369-377 complexes, and not the control complexes (data not shown). The Tyrosinase-HLA-A2-specific TA2-IgG antibody was purified from HEK293 cells and purified using protein A affinity chromatography. SDS-PAGE analysis of the purified protein revealed homogenous, pure IgG with the expected M.W. of ˜200 kDa (data not shown).

The TA2 IgG maintains the specificity of the TA2 Fab antibody—In order to examine the specificity of the purified TA2-IgG, JY cells loaded with specific or control peptide, were incubated with the Ab, followed by incubation with PE-labeled anti-human Ab. Flow cytometry analysis demonstrated specific binding to peptide-loaded cells (FIG. 2d) as well as maintenance of specificity as shown by lack of binding of the IgG antibody to cells loaded with control peptides (FIG. 2e).

Overall these data demonstrate the fine unique specificity of the HLA-A2-Tyrosinase-specific Fab or whole IgG Abs. These results demonstrate the feasibility of transforming phage-derived Fab antibodies into whole IgGs without loss of specificity but with improved avidity and binding reactivity.

The sensitivity of ligand recognition of the TA2 IgG is improved—To demonstrate that ligand recognition sensitivity is improved by using a whole IgG Ab, titration experiments were performed in which peptide-loaded JY cells were incubated with a broad range of Fab or whole Ab concentrations. Specific detection of Tyrosinase-MHC complexes was achieved with 60-fold lower IgG Ab concentration, compared with the detection achieved with the TA2 Fab (data not shown).

Generation of TCR-like antibodies to gp100 (209, 280 and 154) and MelanA (Mart1)—TCR-like antibodies to the other 2 major differentiation antigens gp100 and MelanA (Mart1) were also generated using the same strategy described above. For gp100 TCR-like antibodies directed to the 209, 280, and 154 T cells epitopes were characterized and reported previously [Galit Denkberg et al., Proc Natl Acad Sci USA. 2002 Jul. 9; 99(14): 9421-9426]. Mart1 TCR-like antibodies to the T cell epitope 26-35 (SEQ ID NO:21) were generated and characterized as described above (data not shown). All TCR-like Fab antibodies to gp100, Mart1, and Tyrosinase were transformed into Fab tetramers or whole IgG molecules as described above.

Altogether, these results demonstrate the generation of IgG antibodies from Fabs directed against MHC-class I complexes with melanoma specific epitopic peptides derived from gp100, Mart-1 or Tyrosinase.

### Example 3

### Presentation of MHC Class I-Tyrosinase Complexes on Melanoma Cells

Experimental Results

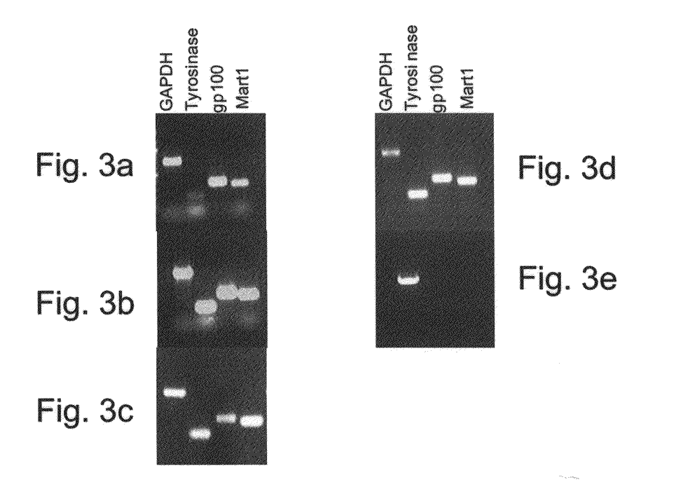

The melanoma lines 624.38, 501A, TC-2224 and TC-1352, but not 1938 express all three melanoma differentiation antigens—To study expression of melanoma differentiation-derived HLA-A2-peptide complexes, 5 lines derived from melanoma patients were used. To determine gene expression of the differentiation antigens, mRNA was isolated from the melanoma cell lines and RT-PCR analysis was performed using specific PCR primers for MelanA/Mart1 (SEQ ID NOs:8 and 9), Pmel17/gp100 (SEQ ID NOs:10 and 11), Tyrosinase (SEQ ID NOs:12 and 13) and GAPDH (control, SEQ ID NOs:14 and 15). As show in FIGS. 3a-e, the amplification results show that the melanoma cell lines 624.38, 501A, TC-2224 and TC-1352 express all three melanoma differentiation antigens [i.e., MelanA (Mart-1), gp100 and Tyrosinase]. No expression of the three differentiation antigens was detected in the cell line 1938.

Large numbers of HLA-A2/Tyrosinase₃₆₉₋₃₇₇ complexes are present on the surface of melanoma cells—To explore whether the HLA-A2/Tyrosinase TCR-like TA2 Ab is capable of binding endogenously derived MHC-Tyrosinase complexes on the surface of tumor cells, flow cytometry analysis was performed on cell lines derived from melanoma patients. Cells were incubated with TA2 anti-HLA-A2-Tyrosinase 369-377 Ab followed by incubation with PE-labeled anti-human antibody. HLA expression on these cell lines was determined by flow cytometry using monoclonal antibodies W6/32, for total HLA expression, and BB7.2 for HLA-A2 expression (Data not shown). As shown in FIG. 4a-c, the TA2 antibody recognized Tyrosinase and HLA-A2 positive (624.38, 501A with TA2 Fab and TC-2224 with TA2 IgG) cells with a very high intensity, which implies that large numbers of HLA-A2-Tyrosinase complexes are presented on the surface of the melanoma cells. No reactivity was detected with Tyrosinase-negative (1938 with TA2 Fab) or HLA-A2-negative (TC-1352 with TA2 IgG) cells (FIGS. 4d and e). Further evidence for the high reactivity of TA2 with melanoma cells can be seen in experiments in which the reactivity of the TA2 Fab and whole IgG were compared in titration experiments. As is shown in FIG. 4g, the whole TA2 IgG molecule could be titrated down to 4 ng yet still demonstrate a clear reactivity with the melanoma cells. As shown in FIG. 4f, the amount of whole IgG required to achieve a comparable intensity to that observed with the Fab fragment was 5-fold lower (1 μg of IgG vs. 5 μg of Fab). To achieve such a degree of binding it is know that a density of several thousands of sites is required based on experimental data and titrations further presented herein below. It will be appreciated that such low amount of antibody which can specifically stain cells can be achieved only if sufficient complexes are present on the surface of the cells. In addition, it should be mentioned that such an intense reactivity for TCR-like antibodies was not observed before in studies performed by the present inventors or by other groups (Cohen, et al. 2003; Denkberg, et al., 2003; Lev, et al., 2002).

Immunofluorescence detection of HLA-A2/Tyrosinase complexes on the surface of melanoma cells—To further study the high HLA-A2-Tyrosinase presentation on melanoma cells, the present inventors attempted to visualize these complexes on the surface of melanoma cells by confocal microscopy. The 501A and 1938 melanoma cells were reacted with the TA2 anti-HLA-A2-Tyr antibody (FAB) as well as with anti-HLA-A2 BB7.2 mAb, and examined by confocal microscopy. As shown in FIGS. 6a-d, the 501A melanoma cells were stained very intensely for HLA-A2 expression using the anti-HLA-A2 BB7.2 mAb (FIG. 6b) as well as for Tyr⁺/HLA-A2⁺ using the TA2 Ab (FIG. 6a) indicating the large number of Tyrosinase-derived complexes expressed on the surface of these cells. Interestingly, the specific HLA-A2-Tyr complexes are organized in unique clusters on the surface of the melanoma cells (FIGS. 6a and c). In comparison, no significant staining was shown on 1938 melanoma cells.

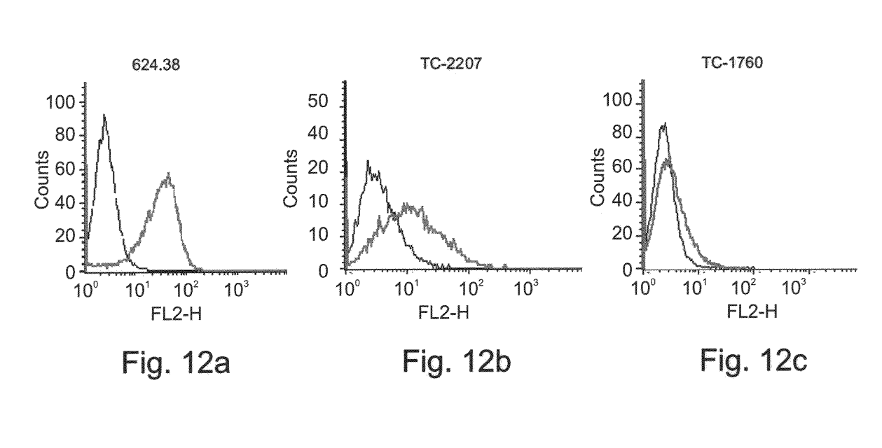

Direct quantization of the number of HLA-A2/Tyrosinase₃₆₉₋₃₇₇ complexes on the surface of melanoma cells—The specific detection of Tyrosinase-HLA-A2 complexes with the TA2 Ab enabled determination of the number of HLA-A2-Tyrosinase complexes displayed on the surface of melanoma cells. To this end, Fab tetramers (C. J. Cohen, et al. 2003) which are generated around a single streptavidin-PE molecule, and which are able to bind up to four different HLA-Tyr complexes were used. Using this strategy, the minimal number of complexes presented on the cell surface was determined. The level of fluorescence intensity on melanoma cells stained with TA2 Fab tetramers was compared with the fluorescence intensities of calibration beads with known numbers of PE molecules per bead (QuantiBRITE PE beads), thus providing a means of quantifying PE-stained cells using a flow cytometer. The number of HLA-A2 complexes expressed on the surface of the melanoma lines tested was measured by staining with PE-labeled anti-HLA-A2.1 mAb BB7.2 and was determined to be in the range of 10,000-20,000 molecules/cell. As shown in FIGS. 7a-e, the minimal number of HLA-A2-Tyrosinase complexes or HLA-A2 molecules displayed on the Tyr⁺/HLA-A2⁺ 501A melanoma cells could be calculated from the measured MFI obtained using TA2 (18.33) and BB7 (56.37) antibodies, respectively. Thus, the number of HLA-A2-Tyrosinase complexes on 501A cells was found to be approximately 3,780 complexes/cell, which is about 20% of the total HLA-A2-Tyrosinase complexes (11,668) on the surface of 501A cells (Table 2, hereinbelow). In addition, as is further shown in FIGS. 12a-c and Table 2 hereinbelow, in other melanoma cells the number of HLA-A2 complexes estimated with mAb BB7.2-PE conjugated was about ˜12,000-20,000 (in the melanoma cells that exhibit high HLA-A2 tyrosinase presentation, the number of HLA-A2 is 12000-20000. Cells that exhibit low HLA-A2-Tyr complexes express ˜5000 HLA-A2 complexes) while the number of HLA-A2/Tyr complexes displayed on high expressing cells such as 624.38 Tyr⁺/HLA-A2⁺ melanoma cell line was determined as ˜3500 (average number of ˜10 experiments), which is ˜20% of the total number of HLA-A2 complexes. The cell lines 501A and TC-2224, which were also stained with high reactivity, revealed similar results (Table 2 and data not shown). The cell line TC-2207, which was stained at a moderate level, displayed a minimal number of 800-1100 HLA-A2-Tyr complexes (Table 2). Other cell lines, such as TC-1760, which exhibit low reactivity with the TA2 Ab, displayed 100-300 HLA-A2-Tyr complexes (FIG. 12c and Table 2, hereinbelow).

TABLE 2
Determination of the number of HLA-A2-
Tyrosinase complexes on melanoma cells

| | 501A | 624.38 | TC-2207 | TC-1760 |
|---|---|---|---|---|
| HLA-A2 (BB7.2) | 11,668 | 10,900 | 11,680 | 5,480 |
| HLA-A2/Tyr | 3,780 | 4,100 | 1,120 | 120 |
| (TA2) | | | | |

Table 2: Quantization of the number of HLA-A2-Tyrosinase complexes on melanoma cells. 501A, 624.38, TC-2207, TC-1760 melanoma cells were interacted with TA2 Fab tetramers generated around a single streptavidin-PE molecule and the number of HLA-A2-Tyrosinase complexes was determined using a PE calibration curve as shown for example in FIGS. 7a-e.

Altogether, these results demonstrate, for the first time the determination of the minimal number of MHC-peptide complexes on cells using TCR-L antibodies. In addition, these results demonstrate the high representation of MHC-tyrosinase complexes on melanoma cells (up to 40% of all HLA-A2-peptide complexes).

### Example 4

### MHC-I/Tyrosinase Mediated Cytotoxic Effect of Melanoma Cells

To further verify the observation made by the TA2 TCR-like antibody that high numbers of HLA-A2-Tyr complexes are expressed on the surface of melanoma cells, cytotoxicity experiments were performed with CTLs that specifically recognize the HLA-A2-Tyr₃₆₉₋₃₇₇ epitope. To this end, two melanoma cell lines were used: 624.38, which express high levels of the HLA-A2-Tyr complexes on the surface, as shown by flow cytometry via the reactivity of the TA2 antibody (FIG. 5a), and TC-2183, which express low levels of these complexes (FIG. 5b). For cytotoxicity assays, chromium-labeled target melanoma cells were incubated overnight in the presence or absence of 10 μM Tyrosinase peptide 369-377 and subsequently exposed to increasing Effector (anti-HLA-A2-Tyr CTLs) to Target (tyrosinase-MHC complex presented on melanoma cell) (E:T) ratios. As shown in FIGS. 5c-d, the addition of the Tyrosinase peptide significantly increased by 2.5-fold specific lysis of TC-2183 melanoma target cells, which express low numbers of HLA-A2/Tyrosinase complexes according to the FACS analysis (as shown in FIG. 5b), compared to target cells exposed to CTL lysis without prior pulsing with Tyrosinase peptide. Thus, the pulsing with peptide increased the number of complexes on the surface and resulted in enhanced killing. In contrast, the addition of the tyrosinase peptide to 624.38 melanoma target cells, which express high levels of HLA-A2-Tyr complexes (as shown in FIG. 5a), did not significantly affect the degree of lysis accomplished with the anti-HLA-A2-Tyr CTLs. These results suggest that the 624.38 target cells express endogenously-derived high levels of the HLA-A2-Tyr complexes and in contrast to TC-2183 cells, which express low levels, additional pulsed peptide does not contribute to increased killing. This data further suggests that the native endogenously-derived expression of the HLA-A2/Tyr epitope on the surface of melanoma target cells is high.

### Example 5

### Presentation of MHC Class I-Melanoma Antigen Complexes on Melanoma Cells

Experimental Results

Expression hierarchy of T cell epitopes derived from melanoma differentiation antigens as revealed by reactivity of TCR-like antibodies—In order to examine the expression level of HLA-A2 in complex with the peptides Tyrosinase 369-377, Mart-1 27-35, gp100 209-217 and gp100 280-288, FACS analysis was performed on melanoma cell lines. For this purpose, the present inventors used whole IgG antibodies of the CLA12 antibody which recognizes the Mart1 derived epitope 27-35 (SEQ ID NO:22), the 1A7 and 2F1 antibodies which recognize the gp100-derived epitopes 209 (SEQ ID NO:4) and 280 (SEQ ID NO:5), respectively, and anti-Tyrosinase Ab TA2, all in context of HLA-A2 (A. Lev, et al., 2002). For the construction of the whole Ab, the heavy and light Fab genes were cloned for expression as human IgG1 antibody into mammalian backbone of eukaryotic expression vectors, and expression was facilitated by co-transfection of the two vectors, as described under the General Materials and Experimental Methods section, hereinabove. As shown in FIGS. 8a-d, flow cytometry analyses of the staining of three melanoma cells lines (TC-2224, 624.38 and 501A) with the various TCR-like whole IgGs revealed a clear distinctive expression hierarchy. All three lines were intensely stained with BB7.2, indicating high level of expression of the HLA-A2 molecules on the surface (FIGS. 8a-c). The reactivity with the anti HLA-A2/Tyrosianse TA2 antibody was significantly high confirming the results shown in FIGS. 4a-h. The expression of HLA-A2/Mart1 complexes (as detected using the CLA12 Ab) was low to modest depending on the cell line used and the expression of HLA-A2/gp100 complexes (as detected using the 1A7 and 2F1) was very low (FIGS. 8a-c). No detection of complexes by these HLA-A2-restricted TCR-like antibodies was observed on control melanoma cells which were HLA-A2 negative (TC-1352; FIG. 8d). These results suggest a clear expression hierarchy of the three major antigens with the number of HLA-A2-Tyrosinase complexes presented on melanoma cells being significantly higher than the number of HLA-A2-Mart1 or HLA-A2-gp100 complexes. Thus, a large number of HLA-A2/Tyrosinase complexes are presented on the surface of melanoma cells with no direct correlation to the expression of other major antigens such as gp100.

The expression level of Tyrosinase mRNA in melanoma cells is significantly lower than that of Gp100 or Mart-1—To further investigate the high level of presentation of the HLA-A2/Tyrosinase complexes on the surface of melanoma cells the relative expression of the three major melanoma differentiation antigens, i.e., Tyrosinase, gp100 and Mart-1, was examined. This was performed to exclude the possibility that the high numbers of HLA-A2-Tyr complexes are due to a significant over-expression of the Tyrosinase mRNA in melanoma cells compared to the other antigens.

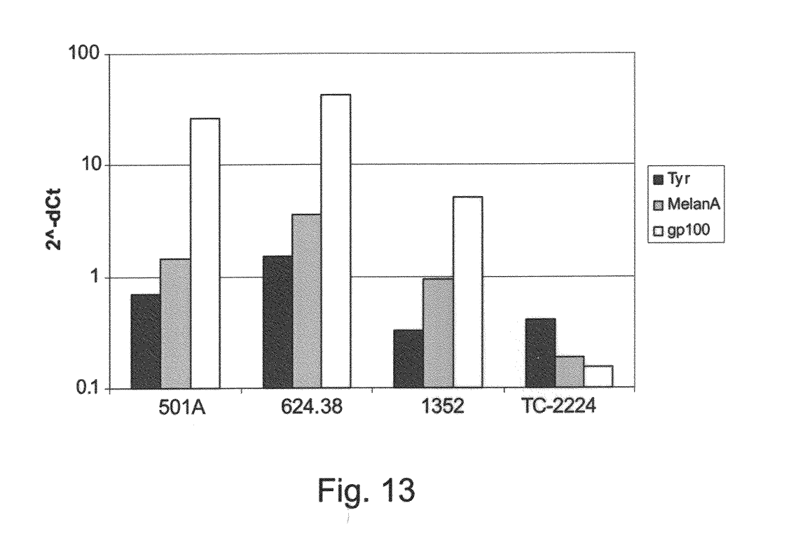

To this end, cDNA was produced from 5 melanoma cell lines, of them the 501A, 624.38, TC-2224, TC-1352 cell lines which express tyrosinase mRNA (as shown in FIGS. 3a-e), the 1938 cell line which expresses HLA-A2 but not the tyrosinase mRNA and the TC-1352 melanoma cell line expresses tyrosinase mRNA but not HLA-A2. The cDNA was subjected to real-time PCR analysis using the glyceraldehyde-3-phosphate-dehydrogenase (GAPDH) as a control gene for verification of cDNA production and for normalizing the relative expression of the three target genes. The relative expression of the target genes was determined as 2_{-ΔCT}. ΔC_{T} is defined as the threshold cycle of the target gene minus threshold cycle of the control gene, GAPDH. As shown in FIG. 13 and Table 3, hereinbelow, in the 501A, 624.38 and TC-1452 melanoma cell lines the relative expression level of Mart-1 was about 2.5 higher than that of tyrosinase. Moreover, in these cells, the relative expression level of gp100 was about 15-37 higher than that of tyrosinase. Thus, the expression level of Tyrosinase mRNA is the lowest in the expression hierarchy of the three genes with gp100 being the highest in 3 out of 4 lines. In TC-2224 cells Tyrosinase was somewhat higher in expression levels compared to the two other genes.

TABLE 3
Relative gene expression by real-time PCR

| | | | | Relative | Relative |
|---|---|---|---|---|---|
| | | | | expression of | expression of |
| | ΔCt | ΔCt | ΔCt | Mart-1 vs. | gp100 vs. |
| Cell line | Tyr | Mart-1 | gp100 | Tyrosinase | Tyrosinase |
| 501A | 0.504 | −0.524 | −4.731 | 2.03 | 37.66 |
| 624.38 | −0.596 | −1.843 | −5.405 | 2.37 | 28.03 |
| TC-2224 | 1.302 | 2.419 | 2.717 | 0.46 | 0.37 |
| TC-1352 | 1.596 | 0.092 | −2.357 | 2.83 | 15.48 |
| 1938 | — | — | — | — | — |

The expression level of tyrosinase mRNA is significantly lower than other melanoma antigens in multiple melanoma cell lines—To further substantiate the results obtained for the three melanoma cell lines (501A, 624.38 and TC-2224), the relative expression level of the melanoma antigens was determined in 31 melanoma cell lines. Briefly, cDNA was produced from 31 melanoma cell lines, followed by real-time PCR analysis. 21 of 31 (67%) melanoma cells were found to express all three melanoma differentiation antigens. As is shown in FIGS. 11a-c, there is a strict correlation between Melan-A (Mart-1) and Tyrosinase expression level (r-Pearson Correlation Coefficients (rP)=0.8 with confidence level of 95%) in all 21 melanoma cell lines with an average of 2.6-fold higher transcript number for Melan-A compared to Tyrosinase. These results are in agreement with published data [Giese, T., et al., J. Invest Dermatol. 124, 633-637 (2005)]. As is further shown in FIGS. 11a-c, the expression level of gp100 was higher than Mart-1 or Tyrosinase, but no correlation was observed.

No correlation between the high representation of tyrosinase/HLA-A2 complexes on melanoma cells and tyrosinase mRNA expression—Table 4, hereinbelow, summarizes the results of FACS analysis performed on 21 melanoma cell lines, which express the three differentiation antigens, as determined by real-time PCR. 18 of 21 (85%) melanoma cells express HLA-A2. 12 of 18 (66%) melanoma cells, which express Tyrosinase and HLA-A2, were recognized by the TA2 Ab. 50% of these cell lines were recognized with very high reactivity, which implies the high presentation number of HLA-A2-Tyr complexes on the surface of these cells. In contrast, only 6 of 18 (33%) were recognized by the anti-Mart-1 or anti-gp100 antibodies. The low reactivity of these antibodies with melanoma cells implies that low numbers of Mart-1 and gp100 complexes are presented on these cells. The relative expression level of melanoma antigens by real-time PCR is presented in Table 4 herein below. Presentation levels were revealed by reactivity with TCRLs.

All TCRL antibodies for gp100, Mart1 and Tyrosinase possess a similar binding affinity as demonstrated by real-time SPR (Surface Plasmon Resonance) binding studies (See Tables 7 and 8 herein below).

TABLE 4
Expression and presentation of melanoma antigens on melanoma cell lines

| | | mRNA | | | Presentation | | | |
|---|---|---|---|---|---|---|---|---|
| | Cell line | Tyrosinase | Mart1 | gp100 | Tyrosinase | Mart1 | gp100 | A2 |
| 1 | 501A | + | ++ | +++ | +++ | ++ | + | +++ |
| 2 | 624.38 | ++ | ++ | +++ | +++ | ++ | + | +++ |
| 3 | 1938 | − | − | − | − | − | − | +++ |
| 4 | stiling | ++ | +++ | +++ | +++ | ++ | + | +++ |
| 5 | 526 | + | ++ | + | − | − | + | +++ |
| 6 | SW | − | − | + | − | − | − | +++ |
| 7 | 1924 | + | + | ++ | +++ | + | + | +++ |
| 8 | 1352 | + | + | +++ | − | − | − | − |
| 9 | 2207 | ++ | + | ++ | +++ | + | + | +++ |
| 10 | 1760 | ++ | ++ | ++ | +++ | − | − | +++ |
| 11 | 1879 | ++ | +++ | +++ | ++ | − | − | +++ |
| 12 | 2081 | − | − | + | − | ++ | − | +++ |
| 13 | 2148-3 | + | + | + | + | − | − | +++ |
| 14 | 2119 | − | − | + | − | − | − | +++ |
| 15 | 2436 | + | + | + | + | − | − | +++ |
| 16 | 1913 | − | − | + | − | − | − | ++ |
| 17 | 2172 | + | + | ++ | + | − | − | + |
| 18 | 1961 | + | + | + | − | − | − | +++ |
| 19 | 2028-1 | − | − | − | − | − | − | +++ |
| 20 | 1907 | + | + | + | + | − | − | ++ |
| 21 | 2224 | + | + | + | +++ | − | − | +++ |
| 22 | 2183 | + | + | + | + | − | − | +++ |
| 23 | 2319 | + | − | + | − | − | − | +++ |
| 24 | 1350 | + | + | ++ | − | − | − | − |
| 25 | 1122 | +++ | +++ | + | − | − | − | − |
| 26 | 1994 | + | ++ | +++ | + | − | − | + |
| 27 | 2370 | ++ | ++ | +++ | − | − | − | − |
| 28 | 2420 | ++ | ++ | +++ | +++ | + | − | +++ |
| 29 | 1927 | + | + | ++ | + | − | − | +++ |
| 30 | 1362 | ++ | ++ | +++ | − | − | − | − |
| 31 | 1851 | − | + | − | − | + | − | +++ |

The results of the real-time PCR analysis negate the possibility that the high amount of HLA-A2-Tyr complexes is the result of overexpression of the Tyrosinase protein in melanoma cell lines. Tyrosinase was expressed at a lower level than Melan-A and gp100 in most of the cell lines examined, thus there is no correlation between the relative gene expression and the presentation of the specific HLA-A2 complexes.

Altogether, the presentation of tumor epitopes with such a high magnitude has not been described until now. This information is particularly important when targets for immunotherapy are considered.

### Example 6

### Stabilization of Tyrosinase Protein Induces a Decrease in the Tyrosinase-MHC-I Complexes Presented on Melanoma Cells

Following verification that the high presentation of HLA-A2-Tyr complexes on the surface of melanoma cells is not due to overexpression of the Tyrosinase gene, the effect of Tyrosinase protein stability on complex presentation was examined. Since the melanoma cells exhibit an amelanotic phenotype, the present inventors have hypothesized that the Tyrosinase protein is inactive, thus large amounts of the protein are being degraded by the proteasome, leading to high presentation of MHC-tyrosinase complexes on the cell surface. It was shown elsewhere [R. Halaban, et al., J. Biol. Chem. 276(15), 11933 (2001)] that DOPA can induce conformational changes favorable for the exit of Tyrosinase from the endoplasmic reticulum (ER) to the Golgi apparatus and restore melanin synthesis thus inducing activity of inactive Tyrosinase. To test the effect of DOPA on presentation of HLA-A2/Tyrosinase complexes, in order to eliminate endogenously derived complexes prior to treatment with DOPA, the peptides presented on MHC were eluted using citrate-phosphate buffer as previously described (Storkus W J, et al., J. Immunother. 1993 August; 14(2):94-103]. The cells were then incubated for 20 hours with 1 mM DOPA, and the presentation of the Tyrosinase/MHC complexes was determined by flow cytometry using the TA2 antibody.

Experimental Results

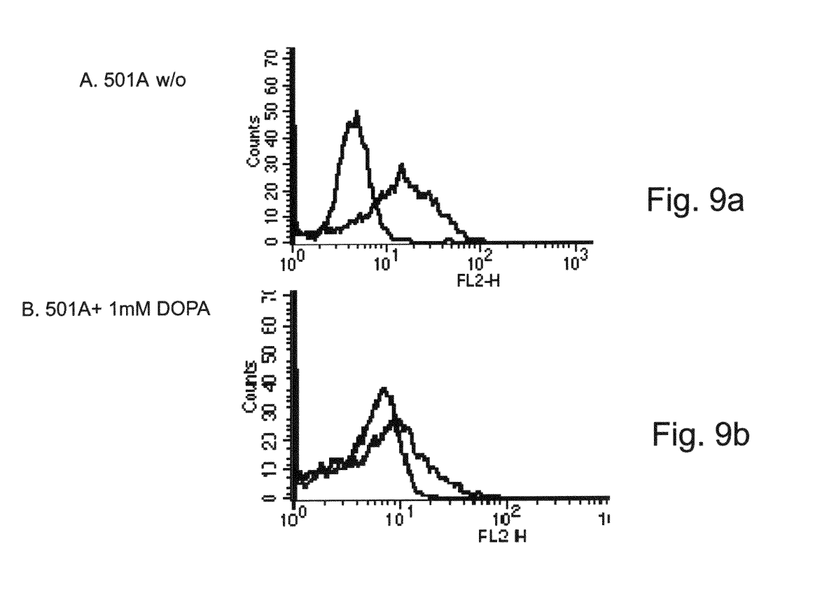

Stabilization of Tyrosinase protein induces decrease in the number of HLA-A2/Tyrosinase₃₆₉₋₃₇₇ complexes on the surface of melanoma cells—Flow cytometry analysis of DOPA-treated melanoma cells was performed by incubating these cells with the TA2 Ab followed by incubation with PE-labeled anti-human Ab. As shown in FIGS. 9a-b for a representative experiment, the presentation of HLA-A2-Tyr complexes was significantly decreased following incubation with DOPA. Quantization of the results is presented in Table 5, hereinbelow.

TABLE 5
Quantization of HLA-A2-Tyr complexes on the surface
of melanoma cells after DOPA treatment

| | MFI signal | MFI background |
|---|---|---|
| 501A | 17.61 | 4.54 |
| 501A + 1 mM DOPA | 9.81 | 6.11 |

As shown in Table 5, hereinabove, there was a 50-75% decrease in the MFI of DOPA treated vs. untreated cells suggesting a significant decrease in the number of HLA-A2/Tyrosinase complexes following DOPA treatment. Thus, without being bound by any theory, these results demonstrate that the high presentation of HLA-A2-Tyr complexes results from the instability of the Tyrosinase protein in melanoma cells. Thus, stabilization of the Tyrosinase protein results in a significant decrease in the number of HLA-A2/Tyrosinase complexes on the surface of the cell.

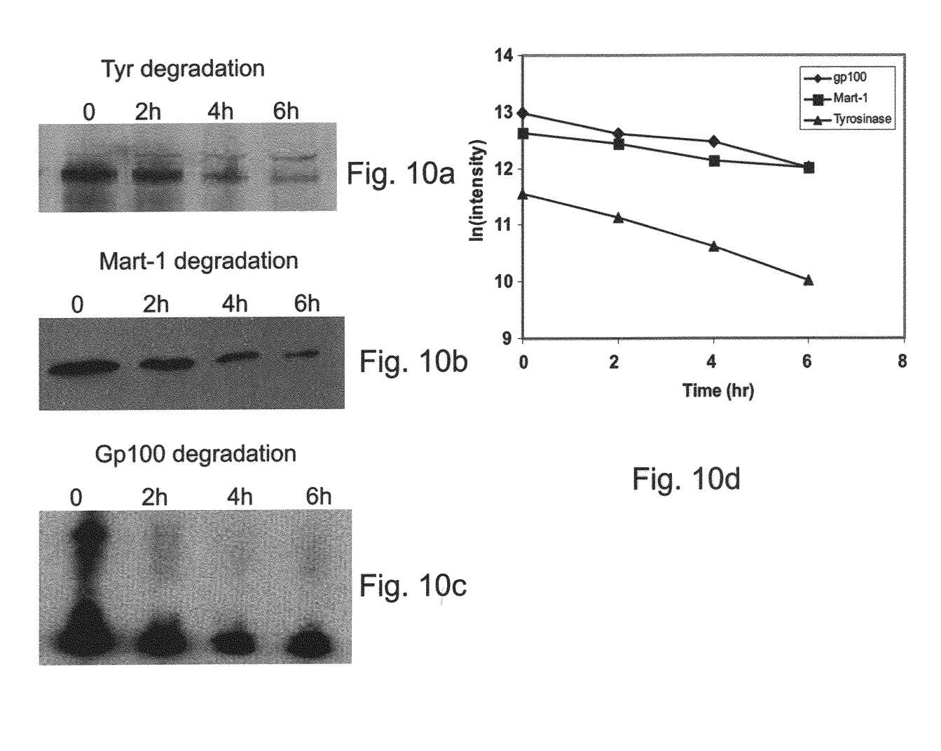

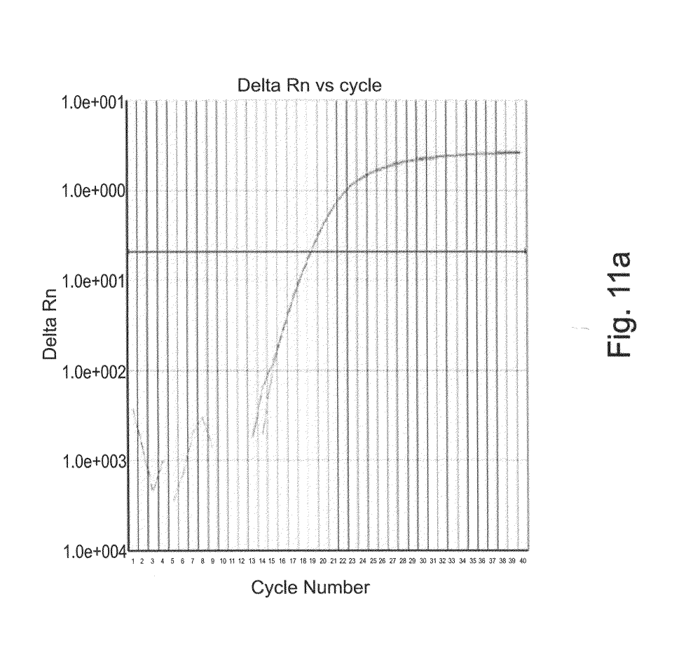

Determination of protein stability and its correlation to presentation of melanoma-derived differentiation antigens—To further investigate the relationship between protein stability and antigen presentation, the rate of protein degradation was determined in melanoma cells. To this end, the 501A melanoma cells which express HLA-A2 and the three melanoma antigens as determined by real-time PCR (Table 4, hereinabove) and reactivity with the TCR-like antibodies (FIG. 8b) were used. To determine the half-life of the proteins (gp100, tyrosinase and Mart-1), protein degradation was arrested by treating the cells with Cyclohexamide followed by cell harvesting at various time points. Protein degradation was monitored by running SDS-PAGE of cell extracts at fixed protein amount followed by Western blotting and detection of the amount of each antigen by using Peroxidase-labeled antibodies specific for gp100, Mart 1, and Tyrosinase whole proteins. Antibody reactivity on blots was quantified by scanning densitometry using a densitometry computer program. As shown in FIGS. 10a-d, there was a hierarchy of degradation rate or protein stability with Mart-1 being the most stable with a t1/2 of ˜6.5 hours, gp100 with moderate stability (t1/2 of ˜4.5 hours) and Tyrosinase being the less stable protein of the three antigens with a t1/2 of ˜2.5 hours (the half life was calculated as ln(2)/k, —k is the slope of the graph). These results indicate the possible relationships between protein stability and the presentation hierarchy of the three melanoma antigen as observed with the TCR-like antibodies. It is possible that rapid degradation of the antigen results in a more efficient intracellular processing and presentation as observed for Tyrosinase which exhibits a relative short half-life compared to the other antigens but extremely high level presentation on the cell surface.

### Example 7

### High Antigen Density Induces Hypo-Responsiveness in Memory Activated CTLS

To study the role of antigen density on the response of activated memory CTLs, the present inventors used activated human HLA-A2-restricted CD8+CTL clones and lines with viral or tumor antigen specificity, which originated from tumor infiltrating lymphocytes (TILs) (J. Zhou, et al., 2004) or peripheral blood. These CTLs were established as effector memory CD8+ T cells by virtue of their characteristic phenotype, i.e., potent cytotoxic activity, cytokine secretions—IL-2, IFNγ, TNFα—but lack of IL-10 or IL-4. They exhibit surface expression of the memory phenotype; i.e., CD3⁺, CD8⁺, TCRαβ⁺, CD45RO⁺, CD45RA⁻, CD62L⁻, CCR7⁻, CD56⁻, CD85⁻, CD69^{low}, and CD25^{low}.

The memory CTLs were exposed to increasing antigen (MHC-peptide complexes) densities using three experimental systems: (i) peptide-loaded pAPCs; (ii) deposition of HLA-A2-peptide complexes on cells lacking endogenous expression of HLA-A2 through the use of an antibody-HLA-A2 fusion molecule (A. Lev, et al. 2004) and (iii) the use of viral-infected HLA-A2 target cells.

Experimental Results

(i) Peptide-Loaded pAPCs

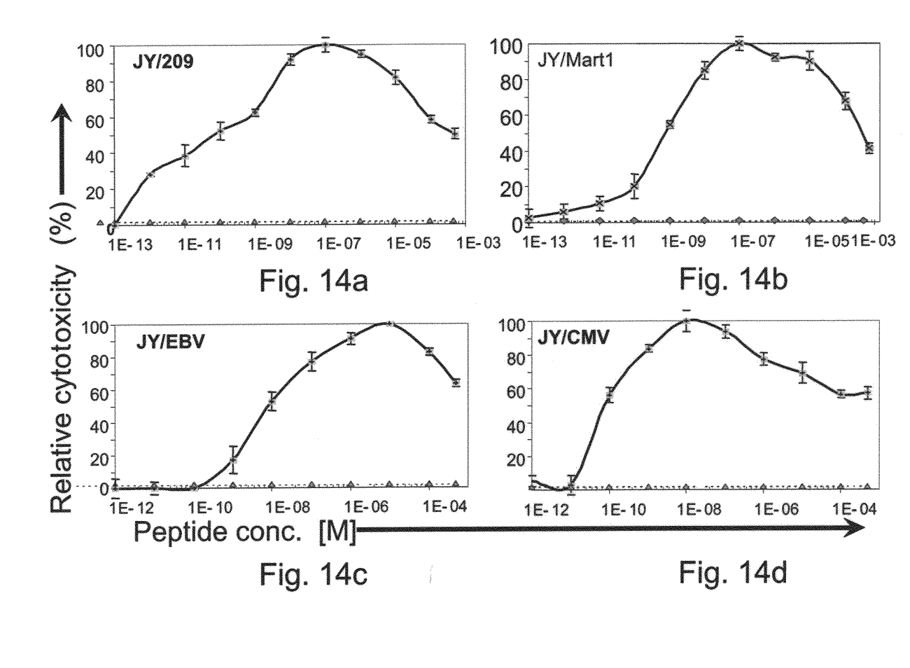

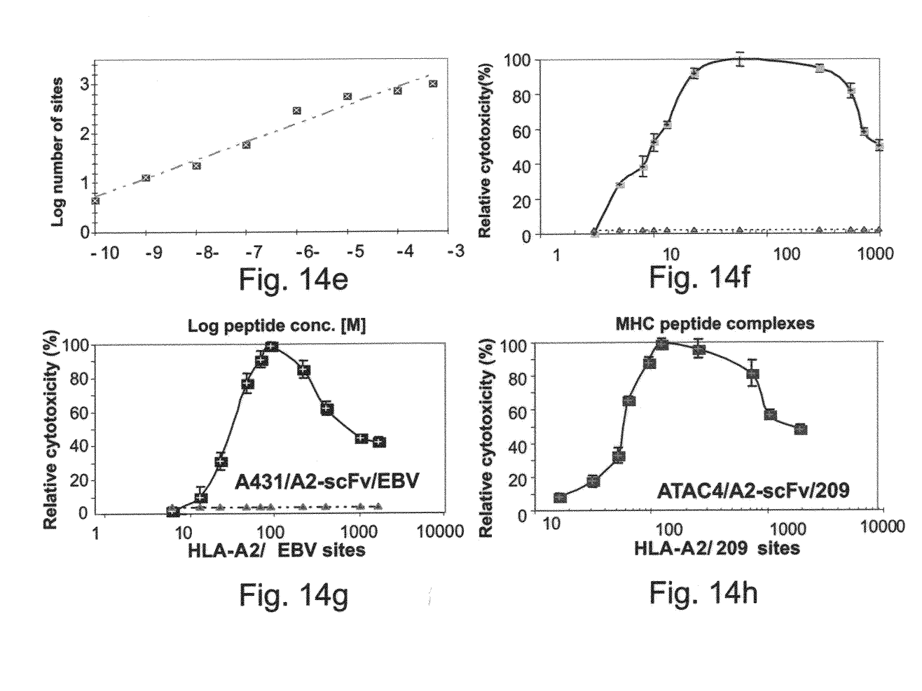

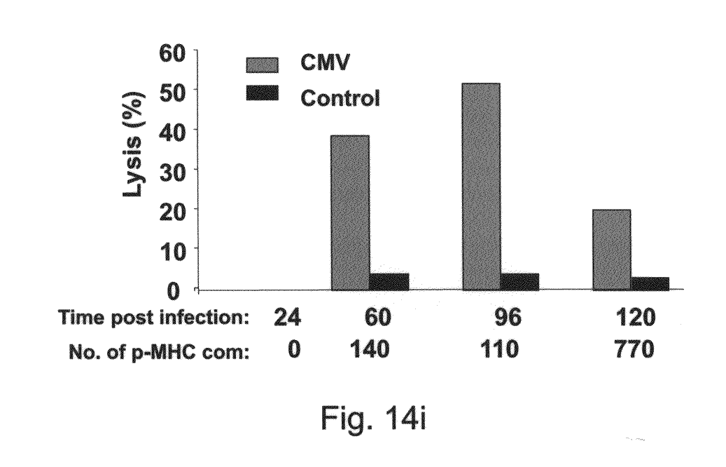

High MHC-peptide complexes presented on cells induce hypo-responsiveness of CTLs—As shown in FIGS. 14a-d, the lytic activity induced by the CTL clones increased in a dose dependent manner from low to intermediate concentrations of epitopic peptide. However, further increase in peptide concentration significantly impaired the lytic activity of the CTLs, up to 60% decrease from optimal lytic activity (FIGS. 14a-d). These results were observed with 4 different human HLA-A2-restricted activated CD8+ T cell clones which recognize the melanoma differentiation antigens gp100-derived T cell epitope 209-217 (FIG. 14a) or 280-288 (data not shown) and MART-1 derived epitope 27-35 (FIG. 14b). Moreover, lytic activity was also tested in CTL lines which recognize viral-derived T cell epitopes. FIGS. 14c and d depict the results with two CTL lines originated from peripheral blood, which recognize the EBV BMLF-1 derived peptide 280-288 (FIG. 14c) and the CMV pp 65 derived peptide 495-503 (FIG. 14d). The killing assays presented depict 5-hour cytotoxicity experiments but similar results were observed with 2 and 16-hours assays indicating that CTL function is stably impaired. Overall these results show that the optimal peptide concentration, which is required to induce maximum killing, is approximately in the range of 1×10⁻⁷ M. The binding affinities of these peptides to HLA-A2 were similar and in the high range of the HLA Peptide Binding Predictions algorithm (hypertexttransferprotocol://bimas.dcrt.nih.gov/molbio/hla_bind/). Furthermore, according to peptide titration data, the affinities of the CTL for the two antigens are similar (data not shown).

These results suggest that high antigen densities induce hypo-responsiveness in properly activated T cell lines and clones.

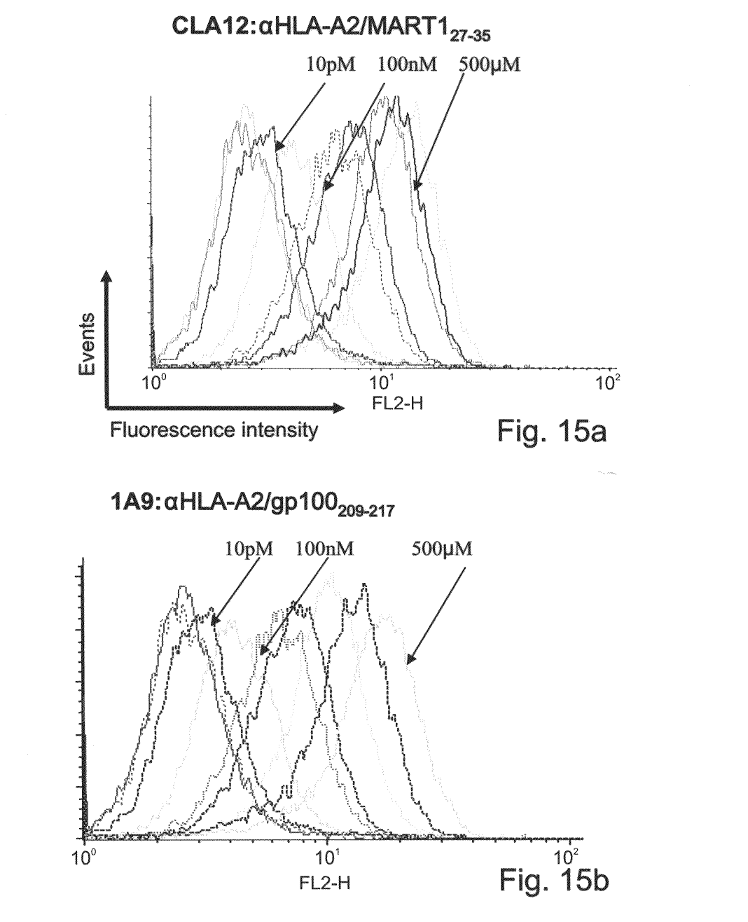

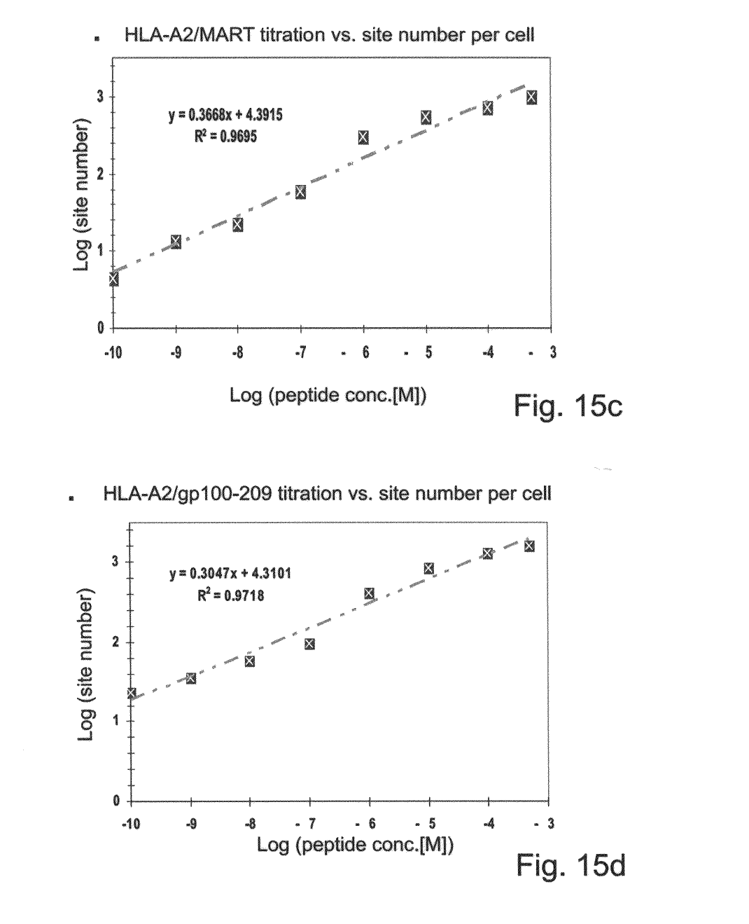

Quantification of the number of HLA-A2-peptide complexes required to achieve optimal lysis by CTLs—In order to directly quantify the specific antigen densities which mediate optimal cytotoxic activity or induce hypo-responsiveness of CTL activity, aliquots of TCR-like antibodies which were previously developed by the present inventors (C. J. Cohen, et al., 2003; G. Denkberg, et al., 2003) were utilized. TCR-like antibodies bind to specific HLA-A2/peptide complexes with a peptide-specific, MHC-restricted manner but in contrast to the low affinity of TCRs these antibodies exhibit a high affinity binding in the nM range (C. J. Cohen, et al., 2003; G. Denkberg, et al., 2003). Titration of binding of such a TCR-like antibody is shown in FIGS. 15a-d. Fluorescently (PE)-labeled secondary monoclonal antibody and PE-calibration curves were used to enumerate the number of peptide-MHC complexes for each peptide pulsing concentration as detected by the TCR-like antibodies (FIG. 14e). As shown in FIG. 14f, for MART1₂₇₋₃₅ HLA-A2-restricted CTL clone JKF6, these calibration curves enabled quantification of the number of HLA-A2-peptide complexes required to achieve initiation, optimal, and inhibition of lysis by the appropriate CTL. Similar results were observed with other CTL specificities shown in FIGS. 14a, c and d. Such direct quantification strategy reveals in all cases that very few complexes are required to initiate killing (10 complexes yielded>20-50% of cytotoxicity). An average of 80-120 specific HLA-peptide complexes on the surface of each target cell, termed as the optimal antigen density, induced maximal CTL mediated killing, however higher levels of antigen densities in the range of 500-700 HLA-A2-peptide complexes significantly reduced the CTLs lytic activity.

(ii) Deposition of HLA-A2-Peptide Complexes on Cells Lacking Endogenous Expression of HLA A2 (e.g., Non-Lymphoid Cancerous Epidermal Cells) Through the Use of an Antibody-HLA-A2 Fusion Molecule

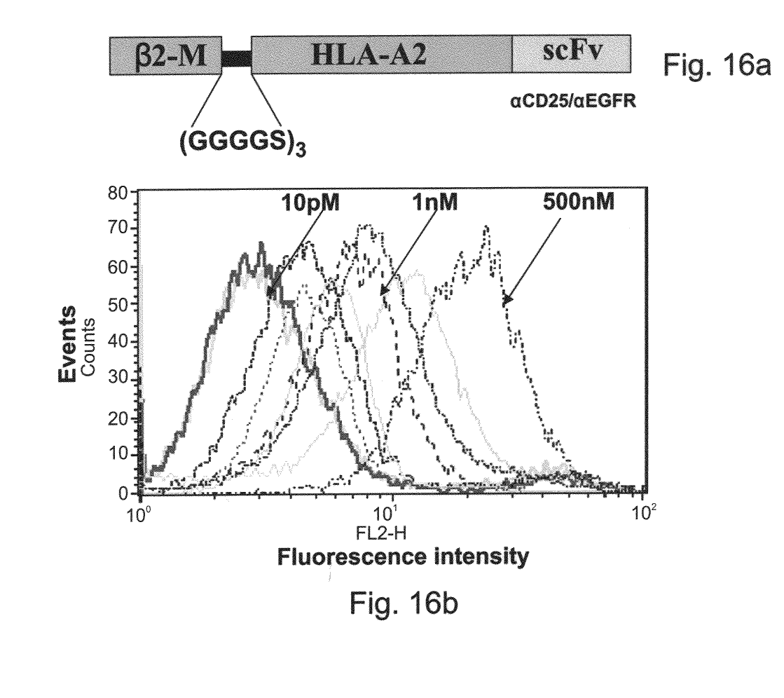

In this approach, targeted deposition of HLA-A2-peptide complexes on target cells devoid of HLA-A2 expression is facilitated through a recently developed genetic fusion that was generated by the present inventors (A. Lev, et al., 2004). In this fusion molecule, a cell targeting scFv antibody fragment is genetically fused to a single-chain HLA-A2 molecule (FIG. 16a). These fusion proteins enabled the present inventors to coat HLA-A2 negative cell lines with different densities of HLA-A2 molecules bearing a specific peptide (FIG. 16b). HLA-A2 negative A431 epidermoid carcinoma cells expressing the Epidermal growth factor receptor (EGFR) were coated with an αEGFR scFv HLA-A2 fusion which carries the HLA-A2-restricted EBV BMLF-1 derived peptide 280-288 (SEQ ID NO:24) (FIG. 14g) or ATAC4 cells (A431 stably transfected with CD25/Tac) were coated with an αTac scFv HLA-A2 fusion which carries the melanoma antigen gp100-derived epitope 209-217 (SEQ ID NO:4) (FIG. 14h). These fusion molecules were effectively used to titrate and segregate low, optimal and high antigen density cell colonies. Subsequently, HLA-A2 restricted lysis of these cohorts by the appropriate activated CD8+ CTLs was observed as shown in FIGS. 14g and 14h. Quantification of the number of αTac scFv HLA-A2 209 and αEGFR scFv HLA-A2-EBV fusion molecules bound to the target HLA-A2 negative carcinoma cells at various concentrations was measured with PE-labeled HLA-A2-specific MAb BB7.2 and PE calibration curves.

Lytic activity for deposited HLA-A2-peptide complexes was similar to that of peptide-loaded pAPCs (FIGS. 14a-d), namely the CTLs killing was initiated at 10-20 complexes, exhibited a peak of optimal lytic activity at intermediate HLA-peptide densities of ˜100 complexes/cell, and was significantly inhibited by 40-60% at high antigen densities of >500 complexes/cell. These results were also observed with 3 other T cell clones and lines (data not shown). The pattern of inhibition of CTL lytic activity at high antigen densities was similar whether peptide-loaded pAPCs or HLA-A2 deposition on target cells have been used.

(iii) Use of viral-infected HLA A2 target cells—In this experimental approach normal human HLA-A2+ fibroblasts were infected with CMV and the cytotoxic activity of CMV-pp 65-derived epitope 495-503 (SEQ ID NO:25)—specific CTL line was determined. The number of pp 65-derived HLA-A2/pp65₄₉₅₋₅₀₃ complexes on the surface of the CMV-infected cells was determined using the TCR-like antibody H9 as described above. As shown in FIG. 14i, optimal CTL cytotoxic activity towards virus-infected cells was observed 96 hours post infection when the number of HLA-A2/pp65₄₉₅₋₅₀₃ complexes was ˜100 sites per cell. When the density of pp 65-derived HLA-A2-peptide complex was ˜700 sites, CTL hypo-responsiveness was observed in a commensurate manner to the two other experimental systems. In comparison with peptide pulsing or MHC deposition, this experimental model reflects the innate physiological processes in which intracellular viral derived peptides are translocated in the MHC complex to the cell surface and the density of such endogenously-derived viral peptide-MHC complexes is increased as a function of time after infection.

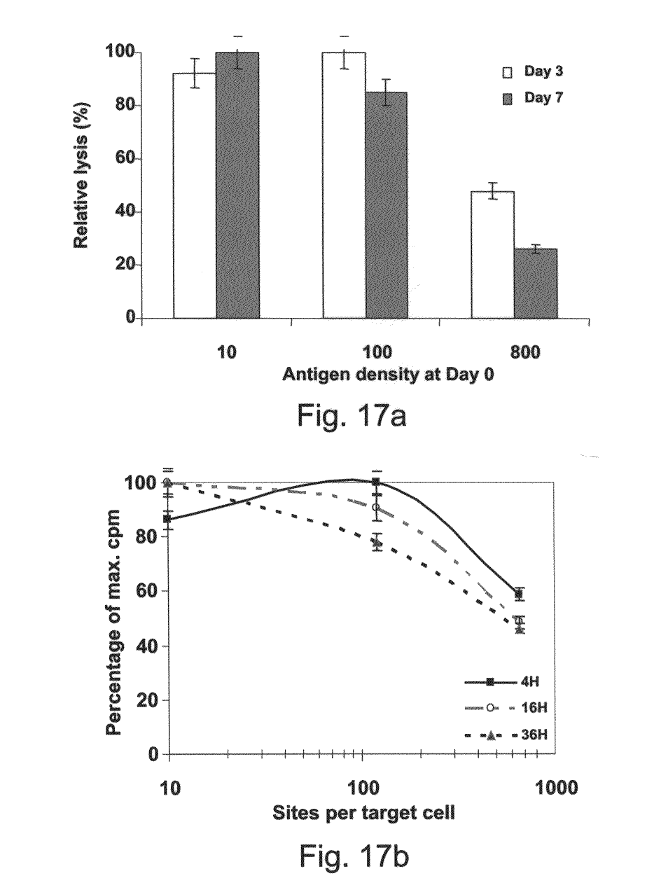

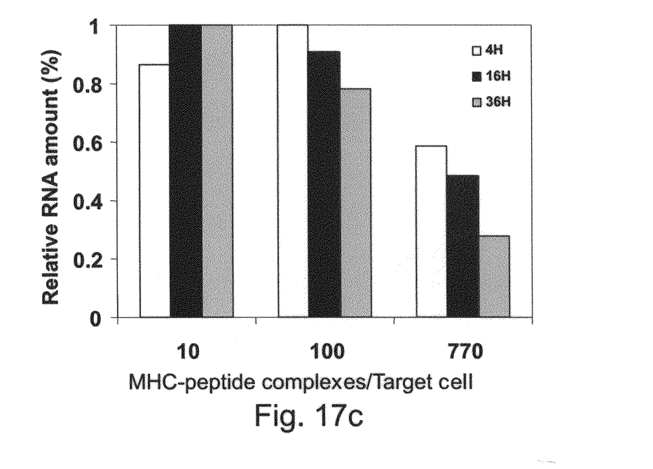

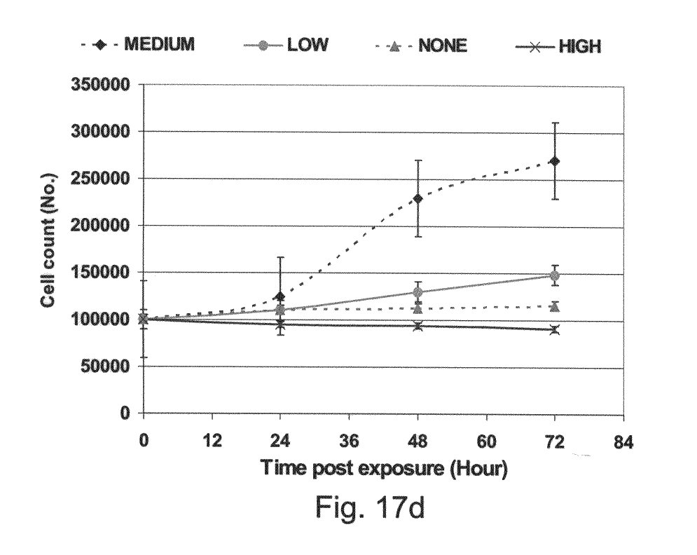

Interestingly the phenomenon of antigen density-induced non-responsiveness is persistent. As shown in FIG. 17a, CTLs exposed initially to low or intermediate antigen density maintained their proper cytotoxic activity when they re-encounter antigens at optimal density after a week. However, the CTLs that were exposed initially to high antigen densities remained anergenic. They exhibited 75% reduction in lytic activity compared to CTLs exposed to low or intermediate antigen densities. In addition, as shown in FIGS. 17b and 17c significant inhibition in CTL proliferation and reduction in total RNA content was observed when exposed to high density (500-700 complexes) but not to low or intermediate densities of antigen.

### Example 8

### Impaired Proliferation and Cytokine Secretion After Exposure of Activated CTLS to High Antigen Density

To further investigate the molecular mechanisms that control antigen-induced CTL hypo-responsiveness two major parameters were examined in T cell biology; secretion of cytokines and expression of surface molecules associated with CTL function.

### Experimental Results

As shown in Table 6, hereinbelow, significant changes were observed over time in the secretion pattern of the Th1 cytokines IL-5, IL-2, and IFNγ but not TNFα.

TABLE 6
Effect of antigen density on Th1 cytokines

| Condition | IL-2 | IL-5 | IFNγ | TNFα |
|---|---|---|---|---|
| 4 h - High Ag Density | 3856 | 79 | 748 | 179.3 |
| 4 h - Optimal Ag Density | 5100 | 122 | 696 | 215 |
| 4 h - Low Ag Density | 431 | 58 | 195 | 24 |
| 16 h - High Ag Density | 2032 | 202 | 1182 | 523 |
| 16 h - Optimal Ag Density | 3593 | 355 | 2037 | 473.9 |
| 16 h - Low Ag Density | 211 | 158 | 338 | 52 |
| 36 h - High Ag Density | 826 | 393 | 1152 | 175 |
| 36 h - Optimal Ag Density | 1386 | 642 | 1492 | 156 |
| 36 h - Low Ag Density | 91 | 171 | 270 | 6 |

Table 6: The effect of antigen (MHC-peptide complexes) presented on cells on the secretion of Th1 cytokines. TNFα, IL-5, IL-2, and IFNγ secretion from MART-1₂₇₋₃₅-specific CTL clone JKF6 exposed to various antigen densities as measured by cytometric bead assay (BD). Ag—antigen; h—hours after exposure; high - 700 complexes; optimal - 100 complexes; low - 10 complexes

As shown in Table 6, hereinabove, the profile of cytokine release correlated with the killing pattern observed as a function of antigen density. Thus, CTLs exposed to optimal densities of peptide-MHC (˜100 complexes) secret a certain level of cytokines. However, once the threshold of peptide-MHC density has reached its upper limit, cytokine secretion decreased significantly, corresponding to a reduction in T cell function, to wit, anergy.

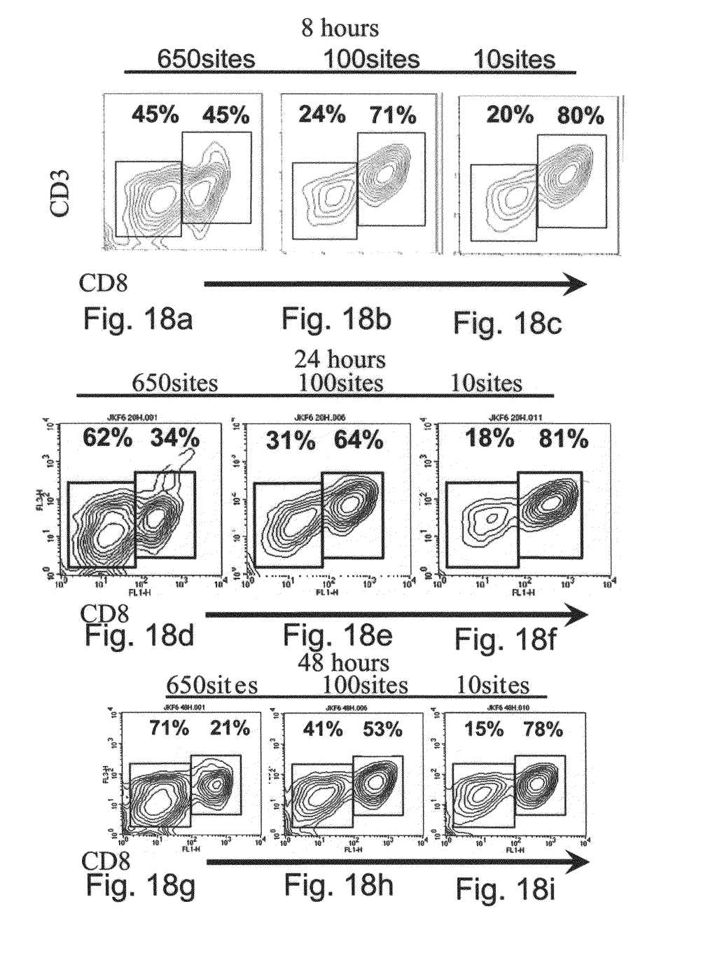

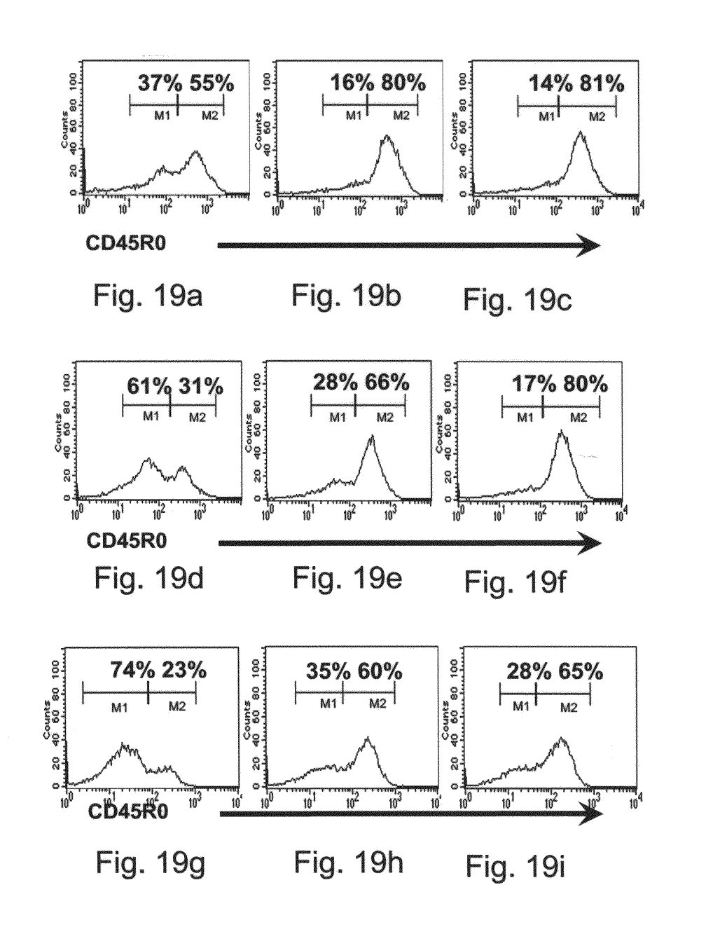

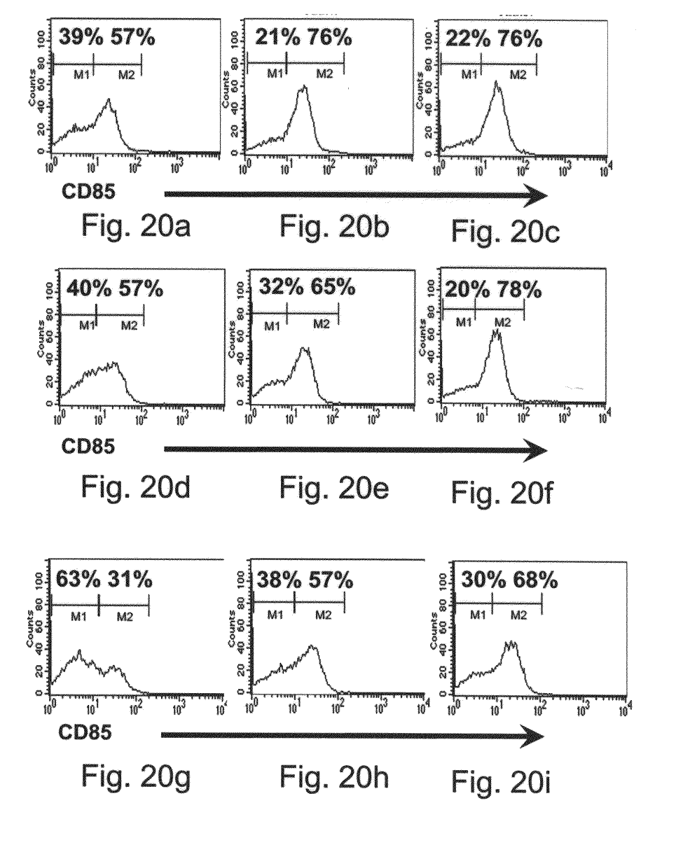

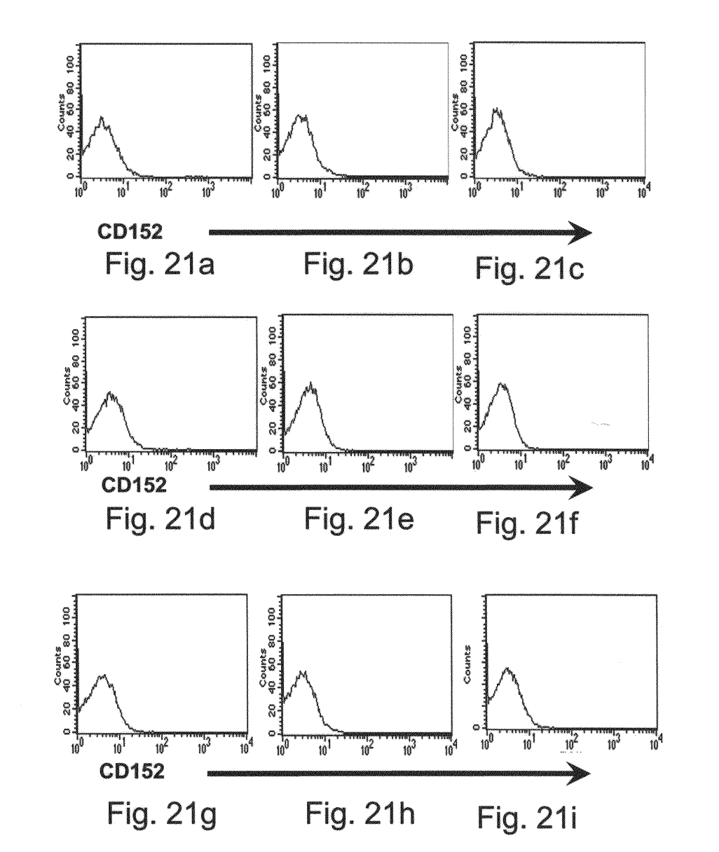

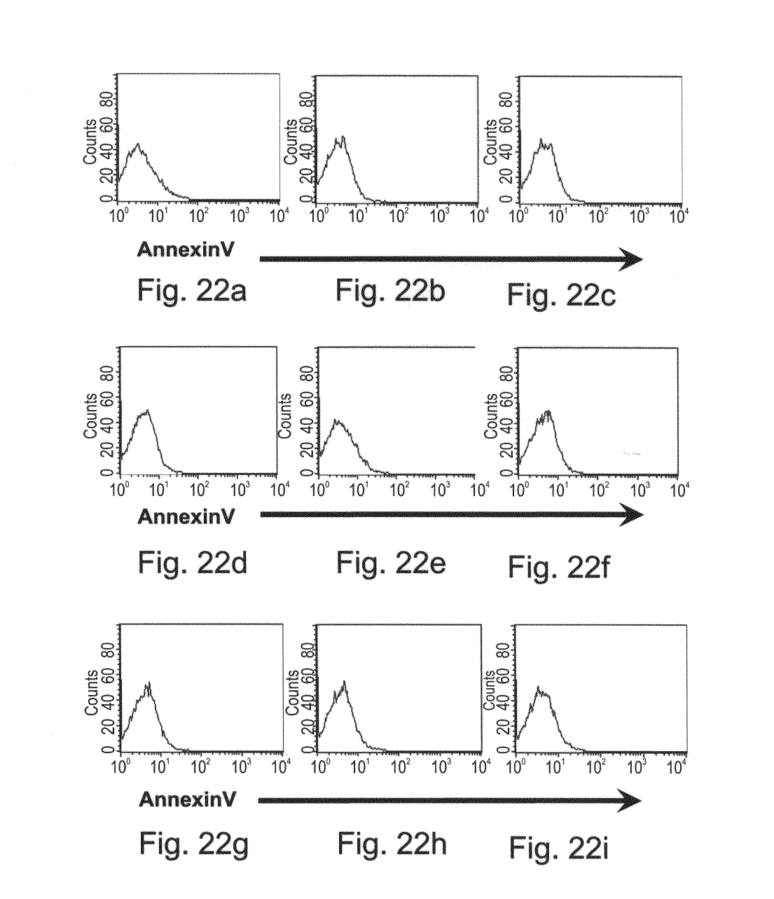

Impaired expression of key functional molecules, after exposure of activated CTLs to high antigen density—The expression of key surface molecules that are associated with effector CTL function and immunological synapse formation as a function of time and antigen density were studies. Exposure to increasing densities of antigen reveals significant and correlative alterations in surface molecules expression (FIGS. 18-22). Major differences were observed in the expression patterns of CD8 and CD3 (FIGS. 18a-i), CD45RO (FIGS. 19a-i), and CD85 (FIGS. 20a-i) as well as down regulation of 70% in TCRαβ. Moreover, as shown in FIGS. 18-20, expression of CD8+ CD45RO and CD85 was palpably altered, CD8 and CD45RO surface expression decreased concordance with increasing antigen density on the target cell and time of exposure. Expression of CD85 (LIR1) was also altered upon CTLs exposure to increasing densities of antigen. In contrast CD152 (FIGS. 21a-i) and staining with AnnexinV (FIGS. 22a-i) indicative of apoptosis was not altered.

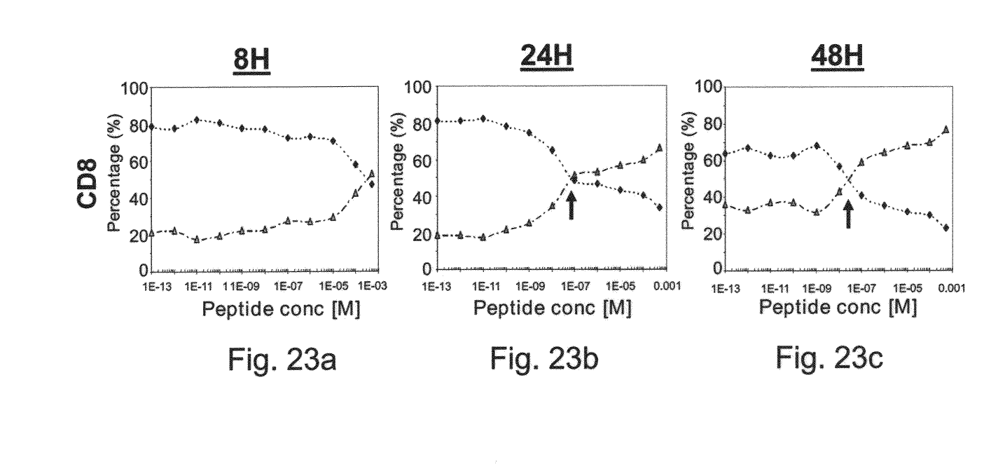

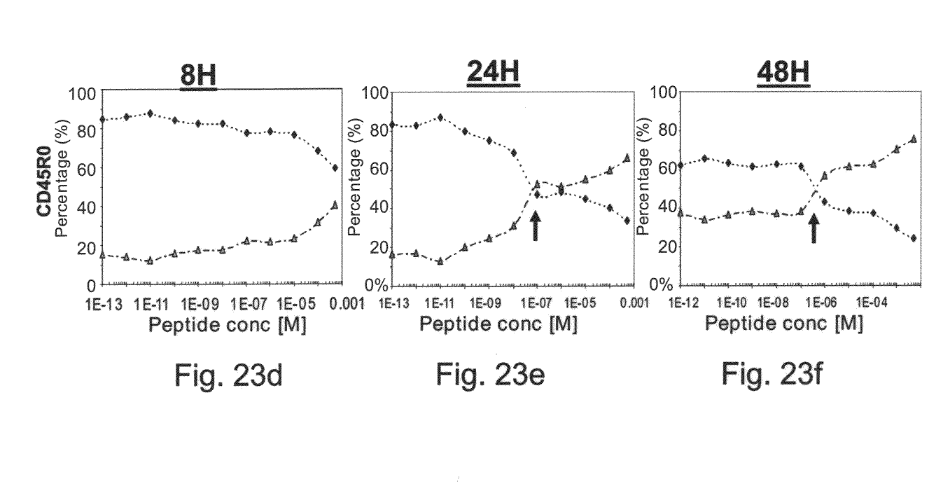

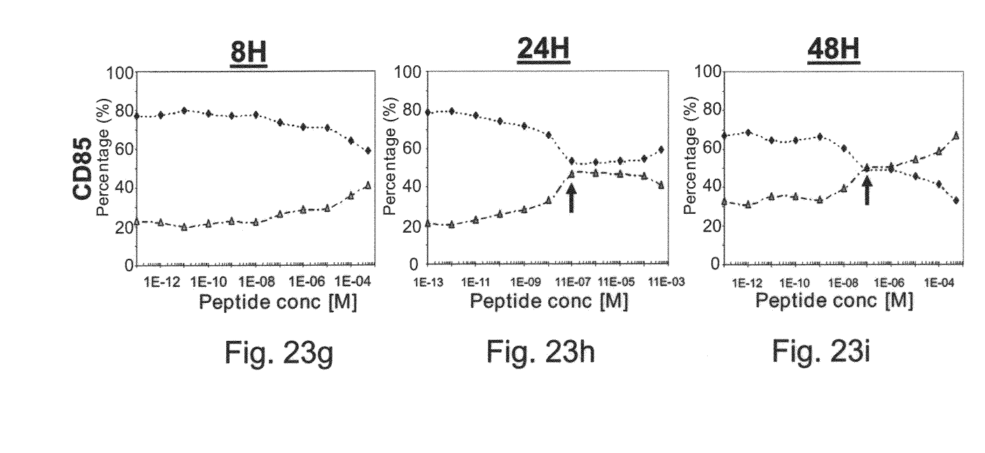

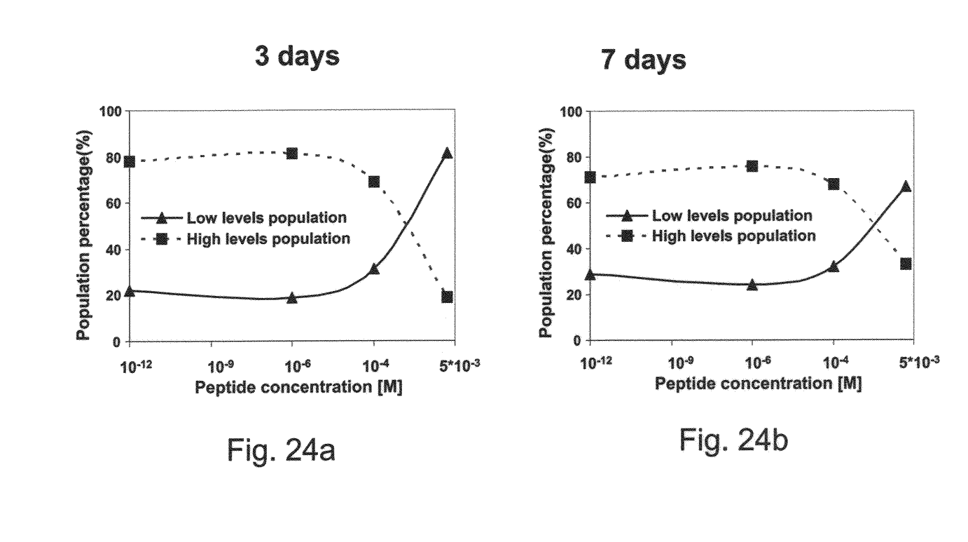

FIGS. 23a-i summarize the alterations in numerous experiments for the expression pattern of the three key surface markers as a function of time after exposure to increasing concentrations of peptide (antigen density). The percentage of CD8^{high}/CD8^{low}, CD45RO^{high}/CD45RO^{low}, and CD85^{Dim}/CD85⁻ CTLs subpopulations is shown. Most pronounced are the results observed 48 hours after encounter with high antigen densities in which there is a clear shift or conversion point between the “high” and “low” phenotype that occurs at peptide concentration of ˜1×10⁻⁷ M which correlates to antigen density of 100 peptide-MHC complexes per cell. The results obtained demonstrate that high antigen densities engender changes in the expression pattern of key molecules that are required for proper T cell function and synaptic formation/interaction. These results demonstrate that three critically important parameters in CTL function namely, proper cytotoxic activity, secretion of Th1 cytokines, and expression of key surface molecules intercept at an optimal antigen density of 100 complexes per target cell and that above a threshold of ˜250 complexes per target they become hyporesponsive with concurrent down regulation of respective parameters.

### Example 9

### Distinct Gene Expression Signature Indicative of Anergy in CTLS Exposed to High Density of Antigen

The fact that maximal alterations in expression of surface markers and cytokine secretion occurred after a relative long period of time (maximal effect after 48 hours) suggested alteration in gene expression. Therefore, the present inventors have performed a comparative genome-wide microarray analysis (Affymetrix) of genes expressed by CTLs exposed to low, optimal, and high antigen density as a function of time.

Experimental Results

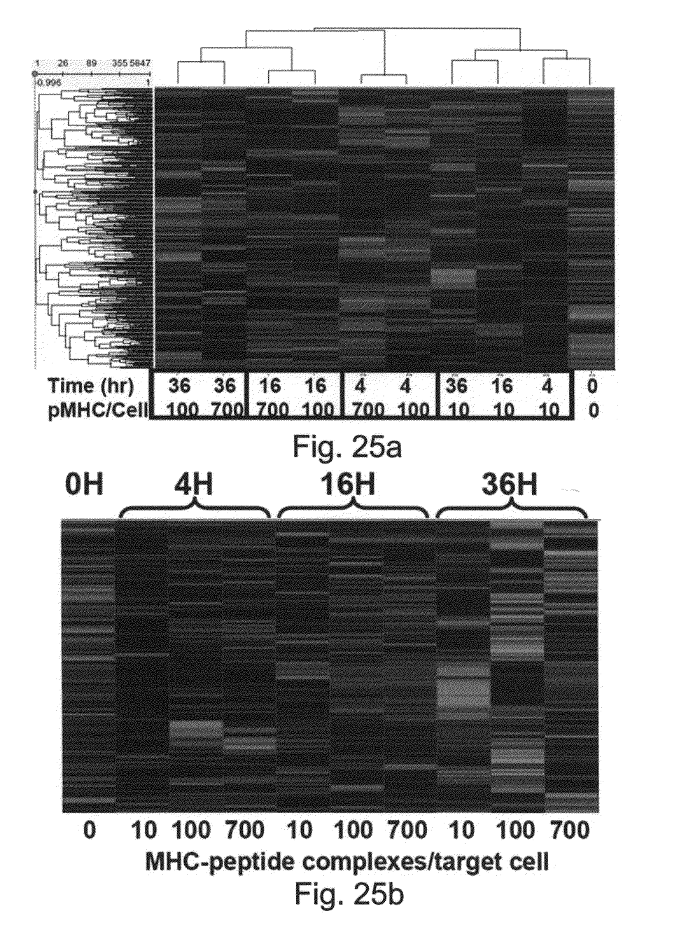

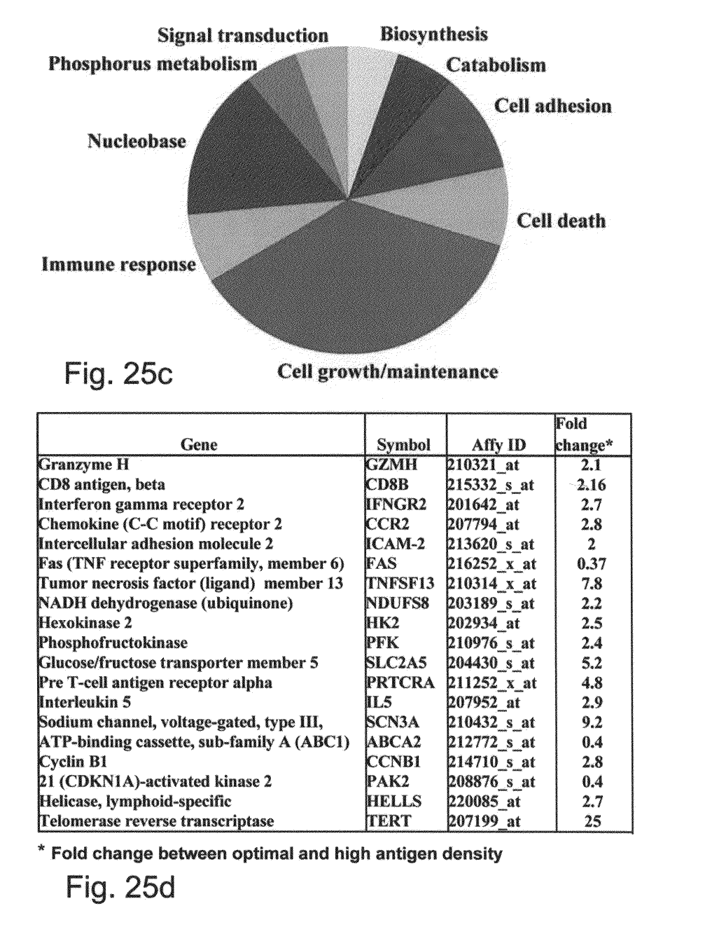

Comparison between treated and non treated samples identified a list of 5877 probe sets changed by at least 2 fold in one or more of the samples compared with time 0 (FIG. 25a). Comparing CTL samples exposed to high versus optimal antigen densities for 36 hours identified 1070 probe sets that are differentially expressed by at least two folds between these two samples (FIG. 25b). Classification of these 1070 genes revealed four major categories of genes for which significant alternations in gene expression were observed between optimal and high density: energy metabolism, membrane potential, cell signaling and apoptosis versus cell cycle control (FIG. 25c). A representative list of genes from the gene array analysis of 1070 probe sets described in FIGS. 25b and c is shown along with the fold changes in the expression of representative genes from CTL exposed to optimal versus high antigen density (FIG. 25d).

The gene analysis results of CTLs exposed to high antigen density revealed that energy metabolism was marked by inhibition of glycolysis indicative by reduction of key glycolysis enzymes such as hexokinase and phosphofructokinase. Changes in membrane potential were reflected by alteration in ion channels expression. Severe depolarization of membrane potential and decreased calcium compliance might result from these alterations. Further impairments were observed in T cell signaling in general including alterations genes of the RAS, MAPK, and IP3 pathways as well as alterations in TCR complex signaling. Gene expression profiles related to cell cycle control and apoptosis indicate a signature of cell cycle arrest with no marked shift toward apoptosis.

Overall, the gene chip analysis presents a signature of an anergic T cell with ablated energy metabolism, altered membrane potential, impaired signaling, and cell cycle arrest. Without being bound by any theory, these data explains the severely impaired response of memory activated CTLs to high antigen density.

Analysis and Discussion

In previous studies, the present inventors have demonstrated the production of recombinant TCR-like antibodies, which can specifically recognize HLA-A2 in complex with peptides derived from MAA (melanoma associated antigens) or other tumor and viral proteins. The importance of TCR-like antibodies for research purposes was demonstrated by obtaining precise information about the antigen presentation of MHC-class I complexes on cancer cells, as well as viral-infected cells (C. J. Cohen, et al., 2003; G. Denkberg, et al., 2003; A. Lev, et al., 2002). These molecules also enable the quantification of the specific MHC class I complexes on the surface of cells (C. J. Cohen, et al., 2003). In addition, the TCR-like antibodies are important for purposes of research since they provide valuable information about potential targets for immunotherapy. These antibodies can also actively participate in immunotherapy as targeting molecules, considering their high affinity and specificity. By generating a whole IgG antibody, tumor cell lysis can be achieved by antibody-dependent cell-mediated cytotoxicity (ADCC) or complement-dependant cytotoxicity (CDC) mechanisms (J. Golay, et al., 2004; H. Mellstedt, 2003; Modjtahedi, et al., 2003; N. Prang, et al., 2005).

In the present study, a TCR-like antibody against the Tyrosinase epitope 369-377 was isolated. Tyrosinase is a membrane-associated N-linked glycoprotein and it is the key enzyme in melanin synthesis. It is expressed in all healthy melanocytes and in nearly all melanoma tumor samples (H. Takeuchi, et al., 2003; S. Reinke, et al., 2005). Peptides derived from this enzyme are presented on MHC class I molecules and are recognized by autologuos cytolytic T lymphocytes in melanoma patients (T. Wolfel, et al., 1994;. Brichard, et al., 1993). The Tyrosinase HLA-A2-associated epitope 369-377, YMDGTMSQV, is generated by posttranslational conversion of the sequence YMNGTMSQV. Only YMDGTMSQV and not YMNGTMSQV is presented by HLA-A*0201 on cells expressing full-length tyrosinase (J. C. Skipper, et al., 1996; C. A. Mosse, et al., 1998). Following protein synthesis, the Tyrosinase is folded in the ER. Correctly folded Tyrosinase is transported via the trans Golgi network to melanosomes (K. Jimbow, et al., 2000). The proposed model for Tyrosinase epitope presentation process includes glycosylation on the N₃₇₃ residue during synthesis of the full-length protein in the ER, followed by reverse translocation of the enzyme to the cytosol, deglycosylation accompanied by deamination (thus conversion of N₃₇₃ to D), degradation by the proteasome, and TAP mediated transport of the resulting peptide fragments into the ER for HLA-A2 binding (V. H. Engelhard., 2002). Loss of pigmentation is frequently observed in human melanoma cells. In these amelanotic melanoma cell lines, tyrosinase failed to reach the melanosomes, and retained in the ER. The aberrant accumulation of Tyrosinase in the ER of melanoma cells results from tumor induced metabolic changes. The acidification of the ER-Golgi boundary of melanoma cells which is hostile to Tyrosinase maturation is the cause of the amelanotic phenotype (R. Halaban, et al., 2002). It has been shown that the substrates DOPA and tyrosine can induce conformational change favorable for the exit of Tyrosinase from the ER to the Golgi and restore melanin synthesis (R. Halaban, et al., 2001). Here the present inventors show that the TCR-like antibody, specific to the Tyrosinase epitope 369-377 presented on melanoma cells, is capable of recognizing melanoma cell lines with a high reactivity, which implies that very high amounts of the Tyrosinase epitope are presented on the surface of melanoma cells. In addition, and without being bound by any theory, the present inventors propose that the inactive Tyrosinase, which results in the abundant amelnotic phenotype, is the cause of the very high presentation of tyrosinase-HLA-A2 complexes on the surface of melanoma cells. In addition, the use of DOPA, which causes active Tyrosinase and melanin synthesis, prevents the high presentation of tyrosinase-HLA-A2 complexes. Moreover, the present inventors have found that among the three antigens analyzed, Tyrosinase which is highly presented on the cell surface is the fastest in degradation thus its relative low stability may contribute to the high level of presentation that was observed. However, protein stability may be only one reason for this finding. Other possibilities may include efficiency of processing that relates to the particular composition of the antigen.

Mart-1 27-35 is a very common immunogenic epitope for HLA-A2-restricted melanoma-specific TIL. It is known as an immunodominant epitope and CD8⁺ CTLs specific for this epitope are frequently found in melanoma patients (Y. Kawakami, et al., 1994). In contrast, the generation of a Tyrosinase-specific response in melanoma patients is a relatively infrequent event. In several studies, Tyrosinase was hardly detectable by the TILs used (Y. Kawakami, et al., 2000). In addition, flow cytometric analysis of PBMCs stained with tetramers showed that Tyrosinase peptide 369-377-specific CD8+ T cells were hardly detectable in peripheral blood of melanoma patients. However, significant numbers of such cells were detected after short-term stimulation of CD8+ lymphocytes with Tyrosinase peptide (D. Valmori, et al., 1999). The results presented here can explain the low immunogenicity of the Tyrosinase epitope. Continual exposure of T cells to antigen maintains an unresponsive state and result in adaptive tolerance (B. Rocha, et al., 1995; L. S. Taams, et al., 1999; R. H. Schwartz, 2003; F. Ramsdell and B. J. Fowlkes, 1992). Without being bound by any theory, the relatively low immune response against Tyrosinase 369-377 epitope can be a result of the high presentation of HLA-A2-Tyr complexes. It may be assumed that HLA-A2-Tyr 369-377 complexes are not presented on healthy melanocytes membrane, because in these cells the Tyrosinase protein is stable and melanin synthesis is accomplished. Thus, the tolerated T cells are a result of the high presentation on melanoma cells and not a result of self presentation on healthy melanocytes.

Since the level of epitope presentation impacts the effectiveness of different immunotherapies differently this information can be used diagnostically to select which type (antibody- or CTL-based) of immunotherapy can or should be used against a particular antigen or for a particular patient. For example a CTL-based immunotherapy could be contraindicated for a patient whose tumor expressed the targeted epitope at the high levels observed here for the tyrosinase 369-377 epitope in many cell lines. Conversely a low level of expression of the targeted epitope can indicate that a CTL-based immunotherapy would be more advantageous than an antibody based one.

The information presented here describes the unique presentation hierarchy of melanoma differentiation tumor antigens. According to these results, Tyrosinase 369-377 is presented with thousands of copies on melanoma cell lines. Presentation of a tumor antigen with such magnitude was not described until now. This information is particularly important when targets for immunotherapy are considered.

The most important question with respect to immunotherapeutic and diagnostic applications of TCR-like antibodies relates to the low density and turnover of the specific epitope on the target cell surface. With regard to the density and targeted killing of cells the present inventors have previously showed in a murine model, that to achieve efficient killing with a TCR-like immunotoxin molecule a density of several hundreds to a thousand MHC-peptide complexes is required for selective elimination of APCs (W. J. Storkus, et al., 1993). The present data suggest that the concept that T cell epitopes are expressed at low numbers on target tumor cells is not universal and there are probably many exceptions represented by antigens such as Tyrosianse. These targets may be ideal for antibody-mediated drug delivery, as well as tumor cell lysis achieved by antibody-dependent cell-mediated cytotoxicity (ADCC) or complement-dependant cytotoxicity (CDC) mechanisms. Exploring these highly expressed T cell epitopes may shed new light on the biology of antigen processing and presentation as well as the process of tolerance. For the clinical aspects such epitopes that are tumor associated and are expressed at high levels open new opportunities for therapeutic interventions particularly in cases where T cell responses fail because of high level expression and tolerance or anergy of antigen-specific CD8+ T cells. In such cases TCR-like antibodies can replace cellular immunity and constitute an attractive therapeutic modality.

Current literature describes two major mechanisms for vetting T cells, thymus selection and priming by pAPCs in lymph nodes (T. R. Mempel, et al., 2004; S. C. Jameson, et al., 1994). The data presented here suggests a third new control point that occurs at the peripheral in situ tissue level. Activated memory CD8+ cytotoxic T cells possess properties of a self-referential sensory organ that is highly sensitive to antigen density. Killing is initiated at very low antigen density and reaches optimal killing activity at around 100 MHC-peptide complexes, however, once a certain upper threshold of about 250 MHC-peptide complexes is being detected on the surface of the target cell significant inhibition of CTL activity was observed which induces hyporesponsiveness or anergy of activated CD8+ T cell lines or clones. This hyporesponsive response is characterized by a significant long term decrease in cytotoxic activity, inhibition of proliferation, and a substantially remodeled anergic T cell.

This work uncovers control mechanism that functions as a negative regulator of CTL cytotoxic activity. Although conservation of this phenomena is yet to be studied in vivo, it is however, well appreciated that self antigens are in general richly expressed, thus one may hypothesize that this novel modality may serve to protect localized tissue from an errant auto reactive attack, by essentially revoking its license to kill.

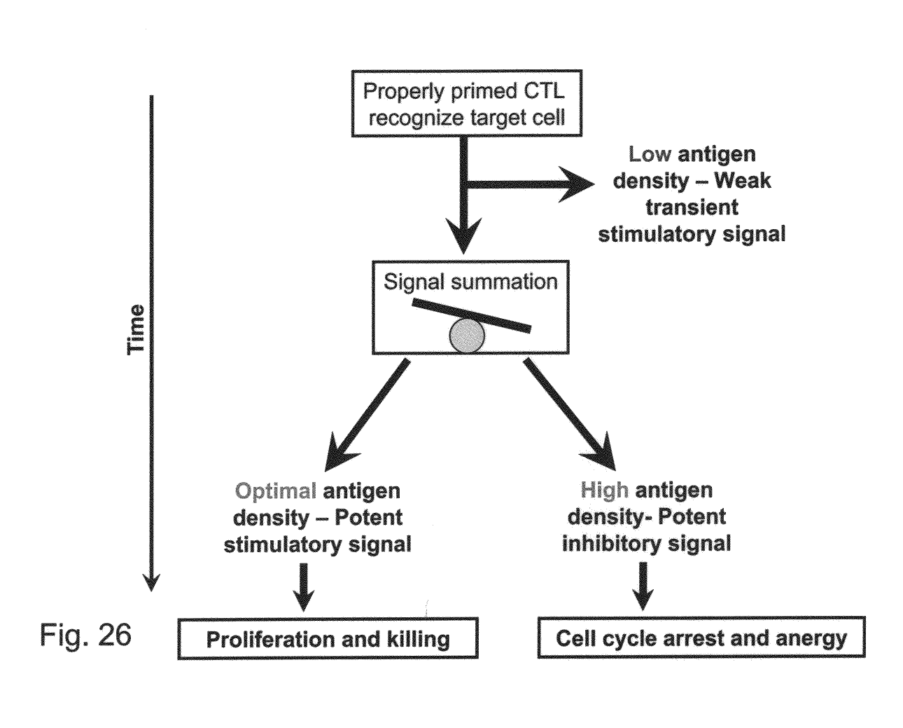

This induced anergy might explain as least in part—peripheral tolerance, a safe guard mechanism that prevents auto reactivity when T cells sense high antigens densities which signal a dangerous auto reactive circumstance. The results presented here may correlate with the recent finding that agonist/endogenous peptide-MHC heterodimers can regulate T cell activation and sensitivity in CD4+ lymphocytes (M. Krogsgaard, et al., 2005). According to these findings which predict that also CD8+ T cells use self-peptide-MHC complexes in their response, initial killing at very low antigen densities (1-20 complexes) involves agonist/endogenous heterodimer complexes. Optimal killing is achieved at ˜100 complexes when most of agonist/endogenous heterodimers are shifted into agonist homodimers when the number of agonist peptide-MHC complexes on the surface increase. However, the new safe guard control mechanism on CTL function, described herein, starts to operate when the number of agonist homodimers or larger MHC-peptide structures are generated due to high antigen density of >250 complexes (see schematic model in FIG. 26).

Most profound in this machinery are anergic state gene expression signatures at high antigen densities compared with a full activation signature at the optimal density. Impairment in T cell function is due to major alterations in TCR signaling and function, membrane permeability, and alterations in energy metabolism and cell cycle control. Glycolysis was largely ablated which indicate that energy metabolism may play a critical role in the control of function of T cells. It was recently suggested that T cell metabolic needs are governed by eternal signals (transcriptional and translations responses as well as co-stimulation) (C. J. Fox, et al., 2005). Once T cells do not receive these signals they fail to increase their energy metabolism to meet the hyperbioenergetic demands of cell growth and are either deleted or rendered unresponsive to mitogenic signals. T cells exposed to high antigen densities are not subject to enhanced apoptosis but rather cell cycle arrest. Thus, these results further strengthen the anergic model for effector memory CTLs rather than deletion through antigen-induced cell death or apoptosis.

Taken in the context of the multitude of signaling and gene alteration events that occur in the high antigen density induced anergenic T cells, it is hoped that this work will further elucidate on the underlying mechanism of T cell biology, with ramifications to extending into understanding more about the control of immune function and regulation such as peripheral tolerance, control of viral infections, anti tumor immune responses, hypersensitivity, and autoimmunity.

In light of the unexpectedly high density of Tyrosinase 369-377 MHC class I (A2+) complexes in Tyrosinase antigen+ cells, TCRL specific for this epitope-MHC combination can be used to diagnose melanoma.

Taking into account the lack of correlation between antigen expression or gene expression and the density of MHC-peptide complexes, TCRL can be used as a more reliable reagent/procedure (in vivo or ex vivo) to measure target epitope presentation by tumor cells, in support of identifying patients that are candidates for epitope directed therapy. Alternatively, excluding patients that fail to express epitope-MHC (as detected by TCRL) with or without whole protein or gene expression, from being treated by epitope directed therapies.

### Example 10

### Cytotoxicity of Toxin-Complexed Antibodies of the Present Invention

Materials and Methods

Peptides and cell lines: The HLA-A2-restricted peptides used for specificity studies are gp100₁₅₄₋₁₆₂: KTWGQYWQV (SEQ ID NO: 20); gp100₂₀₉₋₂₁₇: IMDQVPFSV (G9-209—SEQ ID NO: 4); gp100₂₈₀₋₂₈₈: LLLTVLTVL (G9-280—SEQ ID NO: 5); HTLV-1_{TAX11-19}: LLFGYPVYV (TAX—SEQ ID NO: 26); CMV P65₄₉₅₋₅₀₃: NLVPMVATV (SEQ ID NO: 25); TARP₂₉₋₃₇: FLRNFSLML (SEQ ID NO: 7); XAGE-1 (SEQ ID NO: 30); Mart-1₂₆₋₃₅ EAAGIGILTV (Mart-1 26-35 SEQ ID NO: 21); Mart-1_{27L} ELAGIGILTV (Mart-1 27L—SEQ ID NO: 27); hTERT₈₆₅₋₈₇₃: RLVDDFLLV (SEQ ID NO:29).

Cell lines used in this study: B cell line RMAS-HHD, which is transfected with a single-chain β₂m-HLA-A2 gene, the EBV-transformed HLA-A2⁺ JY cells, melanoma cell lines: HLA-A2+gp100+Mart-1+: Mel624.38, Mel526, Mel501A, FM3D, Stiling. HLA-A2+gp100-Mart-1-: Mel1938 HLA-A2-gp100+Mart-1+: HA24, G-43; HLA-A2-gp100-Mart-1-: PC3.

Selection and characterization of recombinant Fabs with specificity for Mart-1/HLA-A2: The generation and characterization of a panel of Fabs specific for peptide/HLA-A2 were previously described in detail [Lev et al., Cancer Res 62, 3184-94 (2002)]. Phage Abs were selected for binding to single-chain MHC-peptide complexes [Denkberg G et al., Eur J Immunol 30, 3522-32 (2000)] using a large human Fab library containing 3.7×10¹⁰ different Fab clones [Lev et al., Cancer Res 62, 3184-94 (2002)]. The binding specificity of the phage clones selected were tested against soluble Mart-1/HLA-A2 complexes in ELISA assays. Mart-1/HLA-A2-specific Fab Abs were expressed and purified as previously described [Lev et al., Cancer Res 62, 3184-94 (2002)]. The eluted Fabs were dialyzed twice against PBS (overnight, 4° C.) to remove residual imidazole.

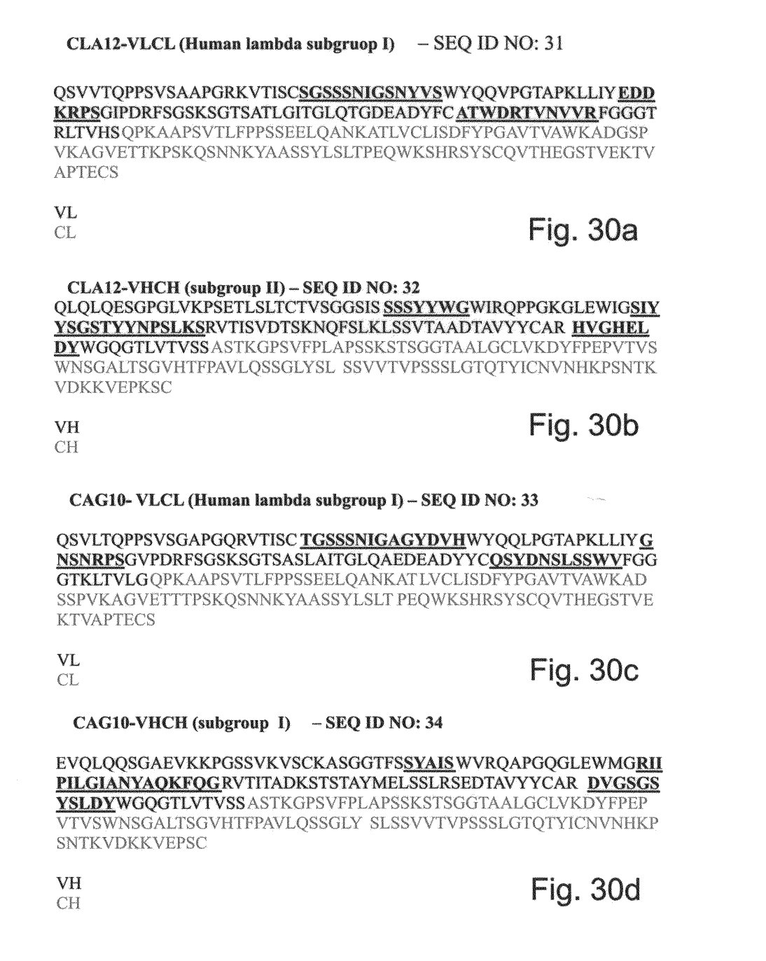

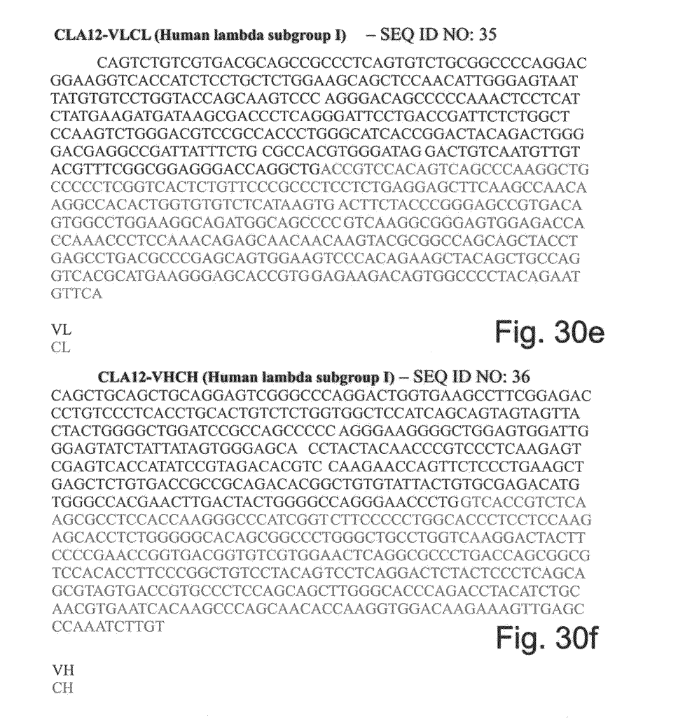

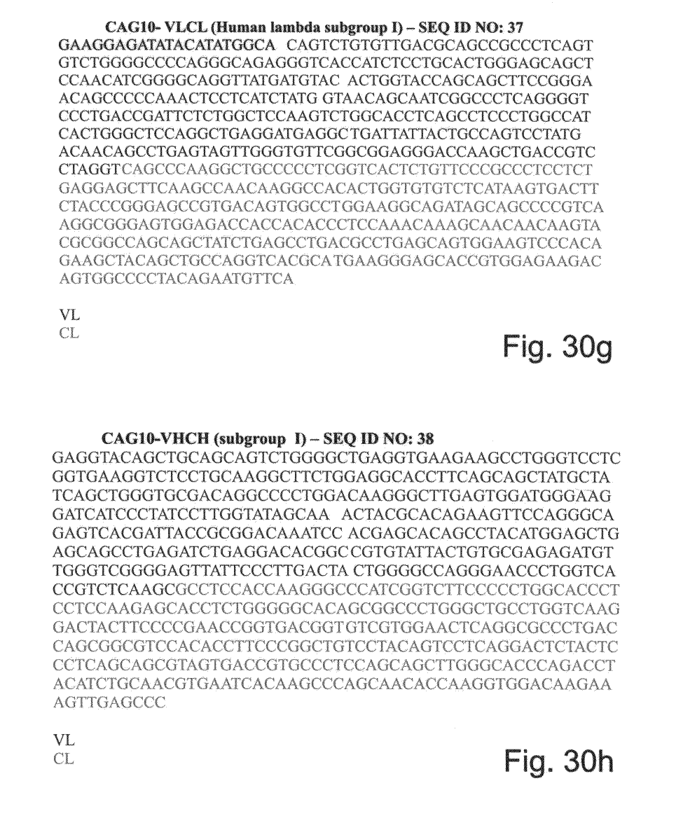

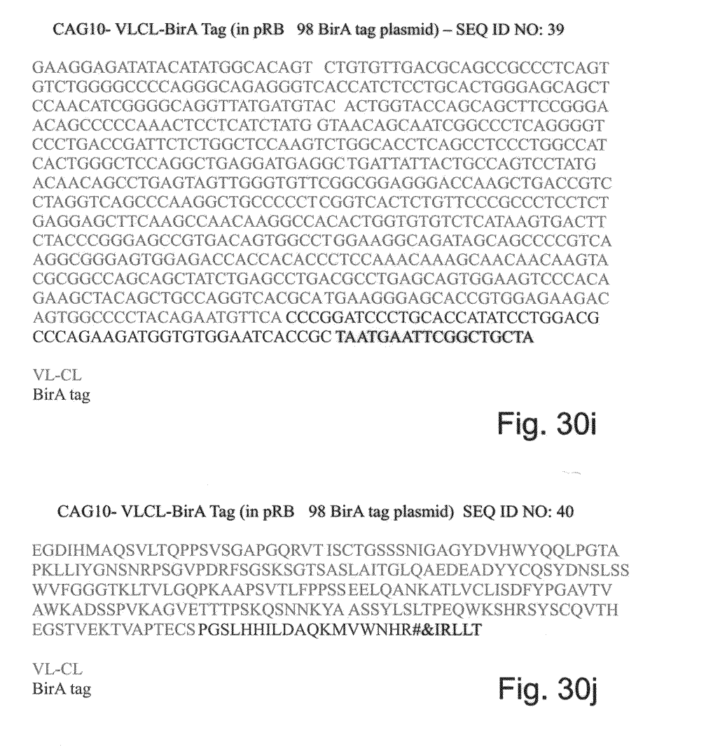

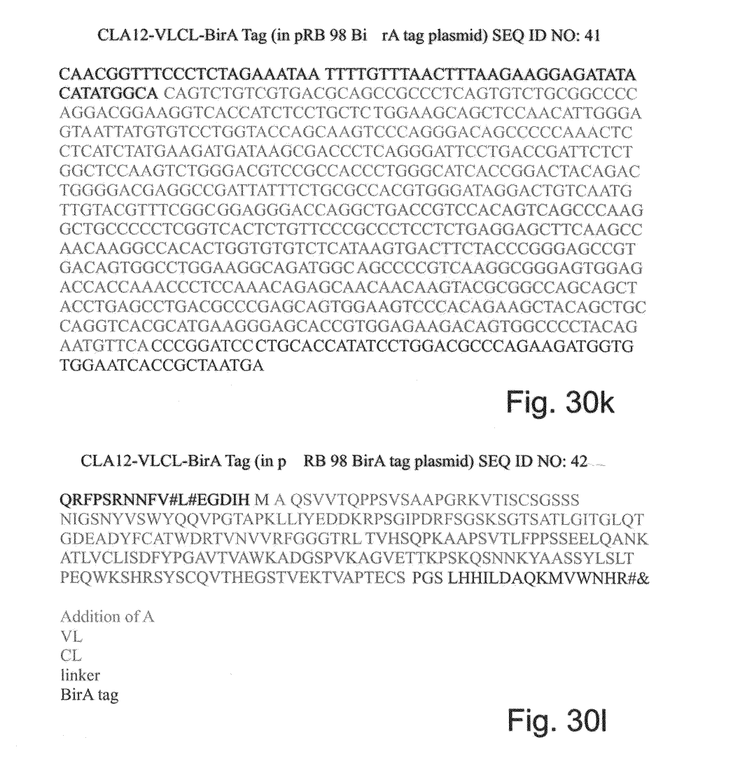

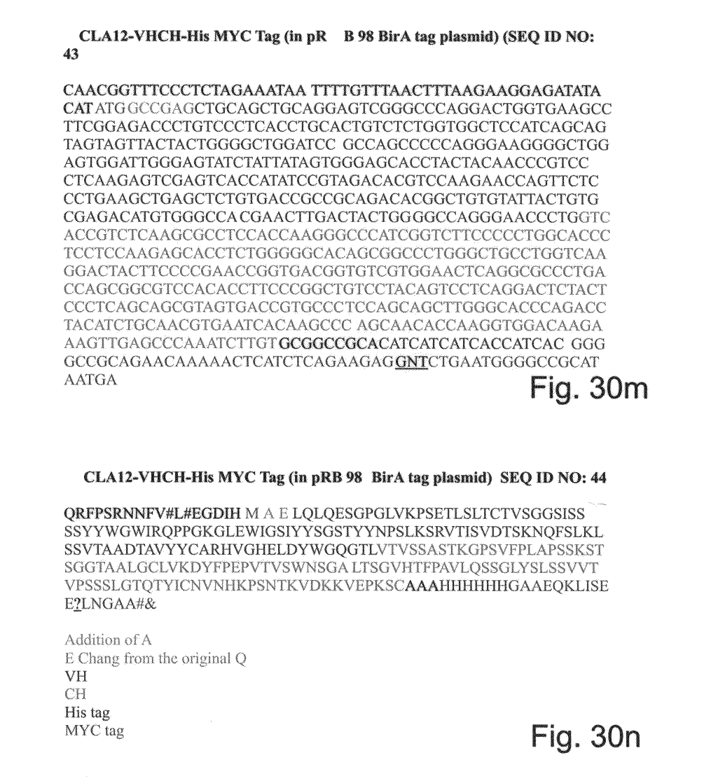

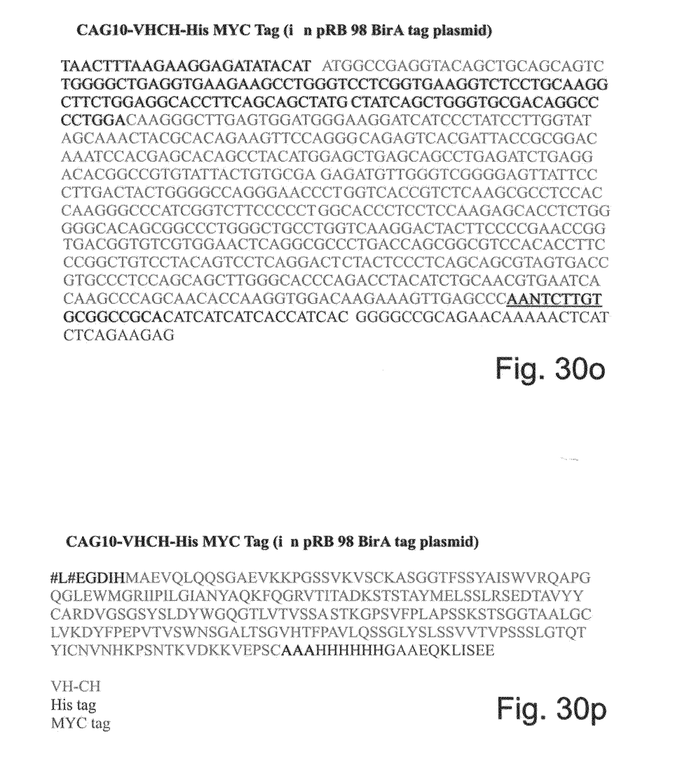

The DNA and deduced amino acid sequences of CLA12 VH+CH1 and VL+CL (heavy chain+light chain) are presented in FIGS. 30a-b and FIGS. 30e-f and are set forth by SEQ ID NOs:31-32 and 35-36. The CDR sequences of the VL are set forth by amino acids 23-35, 51-57 and 90-100 of SEQ ID NO:31; The CDR sequences of the VH are set forth by amino acids 31-37, 52-67 and 100-107 of SEQ ID NO:32. The nucleic acid sequences encoding the VL CDR sequences are set forth by nucleic acids 67-105, 161-171 and 268-300 of SEQ ID NO:35; the nucleic acid sequences encoding the VH CDR sequences are set forth by nucleic acids 91-111, 154-201 and 298-321 of SEQ ID NO:36.

The DNA and deduced amino acid sequences of CAG10 VH+CH1 and VL+CL (heavy chain+light chain) are presented in FIGS. 30c-d and FIGS. 30g-h and are set forth by SEQ ID NOs:33-34 and 37-38. The CDR sequences of the VL are set forth by amino acids 23-36, 52-58 and 91-101 of SEQ ID NO:33; The CDR sequences of the VH are set forth by amino acids 31-35, 50-66 and 99-109 of SEQ ID NO:34. The nucleic acid sequences encoding the VL CDR sequences are set forth by nucleic acids 67-108, 154-174 and 271-303 of SEQ ID NO:37; the nucleic acid sequences encoding the VH CDR sequences are set forth by nucleic acids 91-105, 148-198 and 295-328 of SEQ ID NO:38.

Construction, expression, and production of melanoma specific Fab-PE38 KDEL and Fab-PE38 KDEL: The light chains and the heavy chain containing the variable and constant region 1 (V_{L}C_{L} or V_{H}C_{H}) of Fabs 2F1, G2D12, CAG10 and CLA12, were cloned separately by PCR The V_{L}C_{L} PCR fragments were cloned into the expression vector pULI9 for the construction of Fab-PE38KED [Brinkmann et al, J Immunol 150, 2774-82 (1993)]. The resulting plasmids encode the Fab V_{L}C_{L} fused to a gene encoding PE38 KDEL. The V_{H}C_{H} of the Fabs were cloned into the same expression vector after the toxin gene was removed. The expression vectors for the Fab-PE38 KDEL fusion proteins are driven by the T7 promoter. These constructs were expressed in Escherichia coli BL21 λDE3 and accumulated in insoluble intracellular inclusion bodies. The recombinant Fab-PE38 KDEL and Fab Fab-PE38 KDEL were produced from inclusion bodies by established protocols of solubilization and refolding [Lev et al., Cancer Res 62, 3184-94 (2002)]. Fab-PE38 KDEL fusions were purified by ion-exchange chromatography on Q-Sepharose and Mono-Q.

ELISA with purified Fab Abs or Fab-PE38 KDEL immunotoxin: The binding specificities of individual soluble Fabs and recombinant Fab-PE38 KDEL immunotoxin were determined by ELISA using biotinylated scMHC-peptide complexes. pMHC complexes were refolded using each peptide and coated via streptavidin on an ELISA plate (Falcon). After extensive washing, plates were blocked with PBS/2% skim milk and incubated with various concentrations of soluble purified Fab or Fab-PE38 KDEL for 1 h at room temperature. Bound clones were detected with an anti-human Fab mAb coupled to HRP or HRP-conjugated anti-PE (for Fab-immunotoxin). Detection was performed using tetramethylbenzidine reagent (Sigma-Aldrich, St. Louis, Mo.).

Measurement of melanoma-specific p/HLA-A2-specific Fabs or Fabs-PE38 KDEL immunotoxin binding to cell surface peptide/MHC complexes: RMA-S•HHD or JY cells (10⁶) were pulsed overnight with 50 μM peptide at 26° C. or 37° C., respectively. RMAS-HHD cells were subsequently incubated at 37° C. for 2-3 h to stabilize cell surface expression of MHC-peptide complexes. The cells were then washed in FACS assay medium (PBS, 2% BSA, and 0.09% sodium azide), and incubated for 1 h at 4° C. with 20 μg/ml Fabs or Fab-toxin and FITC-labeled goat anti-human IgG (Fab-specific; The Jackson Laboratory, Bar Harbor, Me.). Cells were washed 3 times with PBS and analyzed by FACSCalibur (BD Biosciences, Mountain View, Calif.). Melanoma cells were trypsinized and stained with the Fab or Fab-toxin as described above. The level of total HLA-A2 expression was detected using the mouse anti-human HLA-A2 (clone BB7.2).

Fab-toxin affinity measurement: Fab-toxin was labeled with [¹²⁵I] using Bolton-Hunter reagent. ¹²⁵I-labeled Fab-toxin (3-5×10⁵ cpm/10⁶ cells) was incubated with JY cells that were loaded with specific or irrelevant peptide and with increasing concentrations of unlabeled Fab-toxin for 1 h at RT. The cells were washed extensively with PBS, and the bound radioactivity was measured by a gamma counter. The apparent binding affinity of the recombinant immunotoxin was determined as the concentration of competitor (soluble purified Fab-toxin) required for 50% inhibition of ¹²⁵I-labeled Fab-toxin binding to the cells. Nonspecific binding was determined by adding a 20- to 40-fold excess of unlabeled Fab-toxin, and by measuring radioactive on cells loaded with irrelevant peptide.

Internalization assay: JY cells were loaded with specific or control peptide, washed and incubated with 20-30 μg/ml FITC labeled Fab-toxin for 1 h on ice. Cells were then washed and resuspended in RPMI containing 10% FCS. Half of the cells were kept on ice while the other half was incubated at 37° C. At indicated time points, a sample was removed, washed, and fixed in Tris/glycerol/polyvinyl alcohol mounting solution. Specimens were examined with a Zeiss confocal laser fluorescence inverted microscope (LSM 410, Carl Zeiss, Oberkochen, Germany) using simultaneous lasers with excitation wavelength 488 nm.

Cytotoxicity assays on JY APCs and melanoma cell lines: JY cells were incubated overnight with 0.1 mM specific peptide or control peptides at 37° C. Peptide-loaded cells were then washed twice with medium and incubated for 24 h with increasing concentrations of recombinant Fab-PE38 KDEL. For melanoma killing assay, 5*10⁴ cells were plated in each well of a flat bottom 96 well plate for 36 h. Graduate amounts of Fab-toxin were then added for an additional 24 h. Protein synthesis inhibition is measured by incorporation of [³H] leucine into cell proteins. IC₅₀ is the concentration of immunotoxin which causes 50% inhibition of protein synthesis.

Antitumor activity (in vivo antitumor assay): The antitumor activity of Fab-PE38 KDEL fusion was determined in SCID mice bearing human cancer cells. Mel526 cells (10×10⁶) were injected s.c. into irradiated NOD-SCID 132M deficient mice on day 0. Tumors (about 0.05 cm³ in size) developed in animals by day 10 after tumor implantation. Starting on day 10, animals were treated with i.v. injections of CLA12 Fab-PE38 KDEL diluted in 0.2 ml of PBS. Therapy was given once every other day on days 10, 12, and 14; treatment groups consisted of 4 animals. Tumors were measured with a caliper every other day, and the volume of the tumor was calculated by using the following formula: tumor volume (cm³)=length×(width)²×0.4.

Statistical Analysis: Tumor sizes in animal experiments are expressed as mean±SD. For comparison between the two experimental groups, Mann-Whitney test was used. P<0.05 is considered statistically significant.

Experimental Results

Isolation and characterization of recombinant antibodies with T-cell receptor-like specificity to melanoma differentiation antigens gp100 and MelanA/Mart-1: Mart1 TCR-like antibodies to the T cell epitope 26-35 (SEQ ID NO:21) were generated and characterized as described in Example 2 herein above and transformed into Fab tetramers and whole IgG molecules as described above.

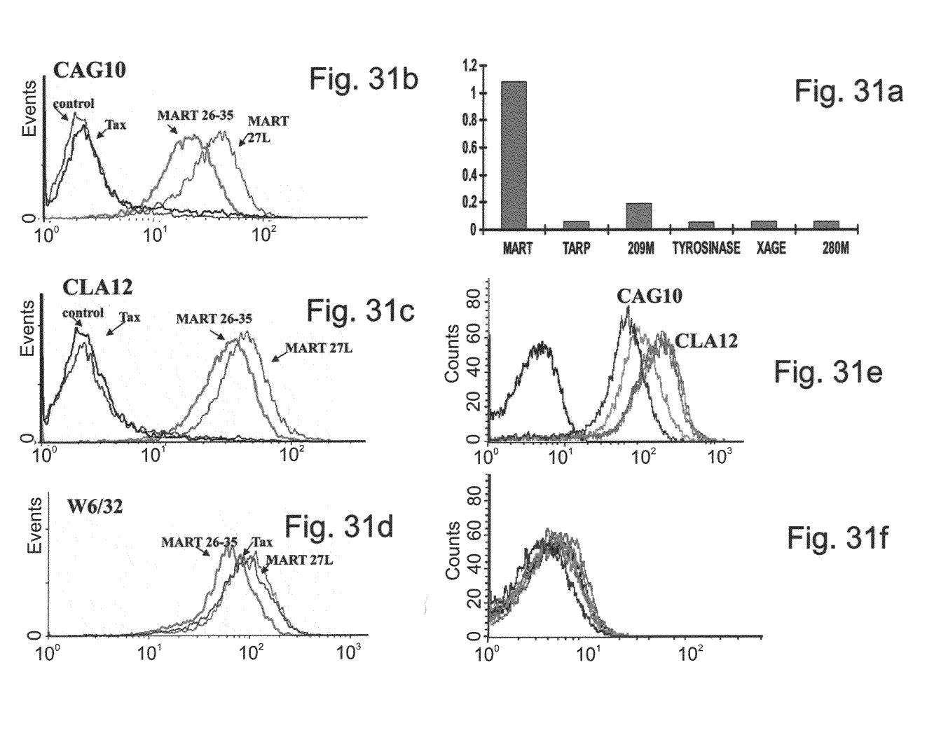

Specifically, the HLA-A2/Mart-1 complexes were exposed to a large naive repertoire of 3.7×10¹⁰ human recombinant Fab fragments displayed on the surface of phage. A 1000 to-2500-fold enrichment in phage titer was observed after three rounds of panning on the Mart-1-derived peptide-HLA-A2 complex (data not shown). After an initial screen for specificity, two Fab phage clones, CAG 10 and CLA 12 Abs were selected and produced in a soluble form in E. coli BL21 cells, then purified by IMAC as described [Lev A. et al., Cancer Res 62, 3184-94, 2002]. SDS-PAGE analysis revealed a homogenous and pure population of Fabs with the expected molecular size (data not shown). The binding specificity of these purified Fab fragments was determined by ELISA with biotinylated MHC-peptide complexes immobilized to wells through BSA-biotin-streptavidin. The correct folding and stability of the bound complexes was determined by their reactivity with the conformational-specific monoclonal antibody W6/32, which only binds correctly folded peptide-containing HLA complexes (data not shown). As shown in FIG. 31A, these soluble Fabs show a very specific recognition pattern in ELISA. For example, Fab CLA12 binds only to the Mart-1/HLA-A2 complex but not to complexes displaying other HLA-A2-restricted MHC-peptides (FIG. 31A). Further specificity studies were performed on antigen-presenting cells (APCs) that display HLA-A2/Mart-1 (FIGS. 31B-F). Two APC systems were utilized. The first consists of murine TAP2-deficient RMA-S cells transfected with the human HLA-A2 gene in a single-chain form (HLA-A2.1/Db-β32m single chain) (RMA-S-HHD cells). Mart-1-derived or control peptides were loaded on the RMA-S-HHD cells and the ability of the selected Fab antibodies to bind to peptide-loaded cells was monitored by FACS. Peptide-induced MHC stabilization of the TAP2 mutant RMA-S-HHD cells was determined by analyzing the reactivity of anti-HLA-A2 MAb W6/32. As shown in FIGS. 31B and 31C, Fabs CAG10 and CLA12 bound in a peptide-specific manner to RMAS-HHD cells that were loaded with the Mart-1 26-35 peptide (as set forth in SEQ ID NO: 21) but not control HTLV-1-derived HLA-A2-restricted peptide (Tax 11-19 peptide) as set forth in SEQ ID NO: 26. The Fabs bound RMAS-HHD cells that were loaded with the anchor modified Mart-1-derived peptide 27L (SEQ ID NO: 27), where the alanine in position 2 is replaced by a leucine residue, giving stronger binding to HLA-A2 [Rivoltini, L. et al. Cancer Res 59, 301-6 (1999); Valmori, D. et al. J Immunol 160, 1750-8 (1998)]. The Mart-1 26-35, 27L-35, and the Tax peptide were all presented at the same level on the surface of the pulsed RMAS-HHD as demonstrated by the binding of MAb W6/32 to these cells (FIG. 31D).

The second type of APCs tested were EBV-transformed B lymphoblast JY cells, which express HLA-A2, and were incubated with the Mart-1-derived or control peptides. These cells are TAP+, and consequently, displaying the exogenous peptide is facilitated by peptide exchange. Using this strategy, a mixture of exogenously and endogenously-derived peptides presented on HLA-A2 are displayed on the cell surface. As shown in FIGS. 31E and 31F, CAG10 and CLA12 bound JY cells pulsed with the Mart-1 26-35 and modified 27L peptides (FIG. 31E), but not JY cells pulsed with 5 control HLA-A2 restricted peptides as indicated (FIG. 31F). These results show that Fab CAG10 and CLA12 antibodies exhibit TCR-like fine specificity and can recognize only native HLA-A2 complexes bearing appropriate peptide in situ on the surface of cells. Similar studies were performed previously with Fab-2F1 and Fab-G2D12 which, respectively, target the gp100-derived HLA-A2-restricted epitopes G9-280 (SEQ ID NO: 5) and G9-154 (SEQ ID NO: 6) [Denkberg G et al., Proc Natl Acad Sci USA 99, 9421-6 (2002)].

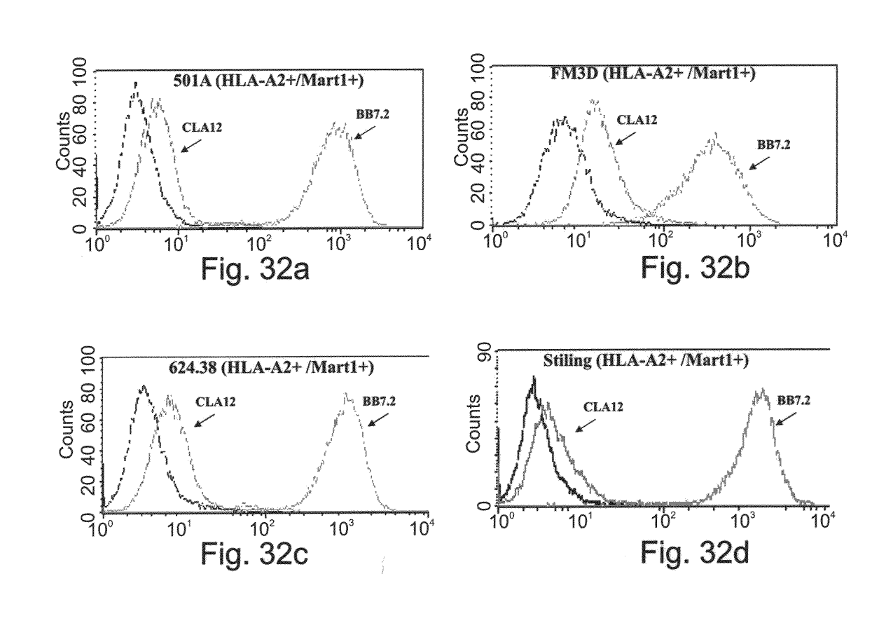

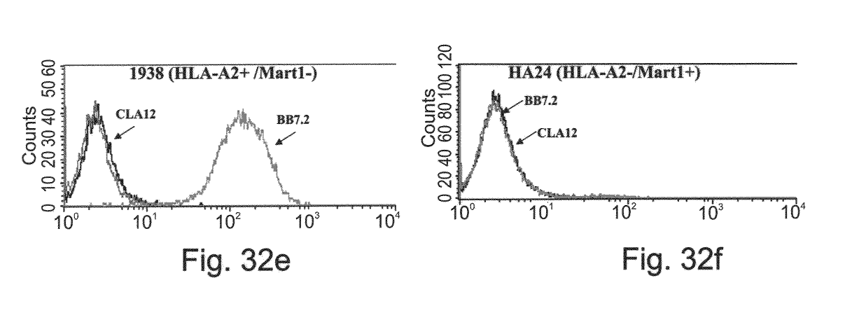

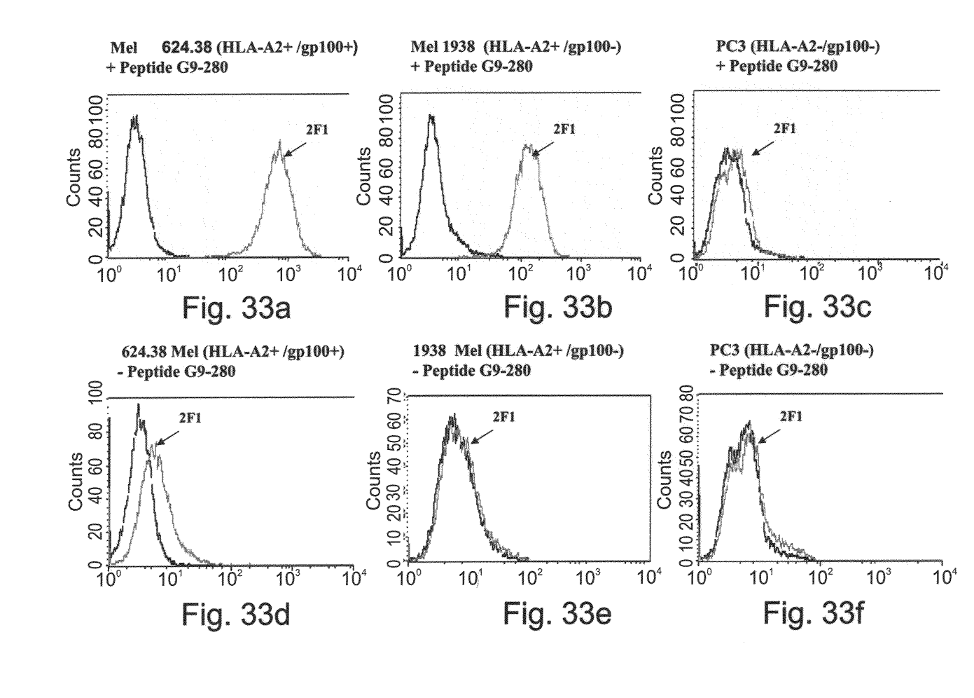

Binding of Mart-1 and gp100-specific TCR-like Fab antibodies to melanoma cells: To test whether the melanoma-specific TCR-like Fab antibodies can bind naturally processed HLA-A2-peptide complexes on the surface of tumor cells, the present inventors performed flow cytometry studies on HLA-A2+ melanoma tumor cell lines (FIGS. 32A-F and FIGS. 33A-F). In these cells, the specific peptide/HLA-A2 complex is only represented as a minor fraction out of the total surface HLA-A2 complexes. Mart-1− specific Fab antibody CLA12 reacted with the HLA-A2+/Mart-1+ melanoma lines 501A, 624.38, FM3D, and Stilling (FIGS. 32A-D), but not with HLA-A2+/Mart-1− melanoma 1938 or with melanoma HA24 cells which is HLA-A2−/Mart-1+ (FIGS. 32E-F). The expression level of HLA-A2 was monitored by MAb BB7.2. These results indicate that the TCR-like antibody CLA 12 can detect the native HLA-A2/Mart-1 epitope on the surface of melanoma cells. Similar studies were performed with TCR-like Fab antibodies which are specific for the gp100-derived epitopes in complex with HLA-A2 [Denkberg G et al., Proc Natl Acad Sci USA 99, 9421-6 (2002)]. For example, Fab 2F1 directed to the HLA-A2/gp100-G9-280 epitope binds HLA-A2 positive melanoma 624.38 (gp100+) and melanoma 1938 (gp100−) cells pulsed with the G9-280 peptide (SEQ ID NO: 5), but not to G9-280-pulsed PC3 HLA-A2 negative cells (FIGS. 33A-C). When binding to these cells was tested without pulsing, Fab 2F1 binds to HLA-A2+/gp100+624.38 melanoma cells but not to HLA-A2+/gp100− 1938 melanoma nor to HLA-A2−/gp100− PC3 cells (FIGS. 33D-F). These results further indicate the capabilities of these unique antibodies to bind in a peptide-dependent, MHC-restricted, manner to target cells which express naturally processed endogenously-derived peptide-MHC complexes.

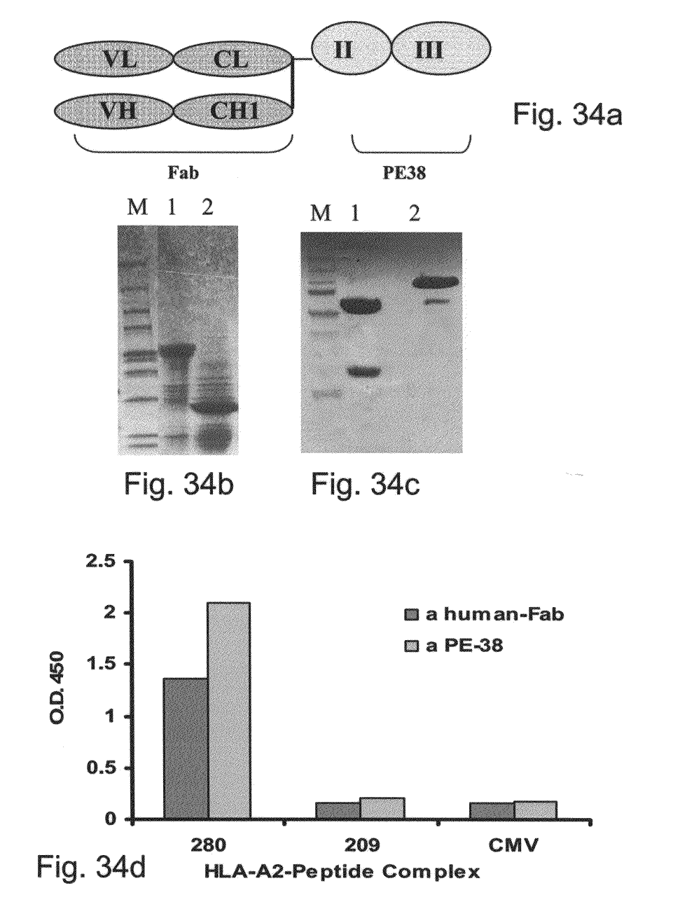

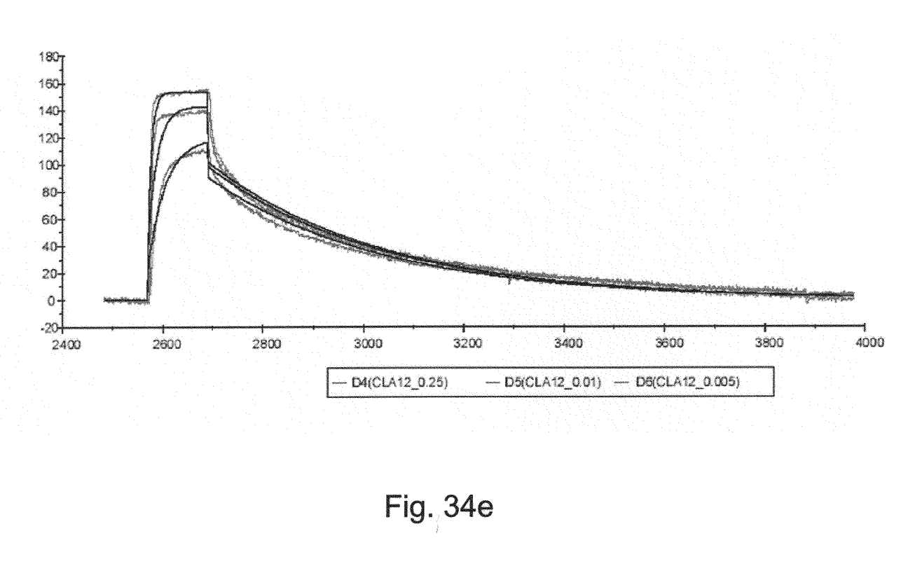

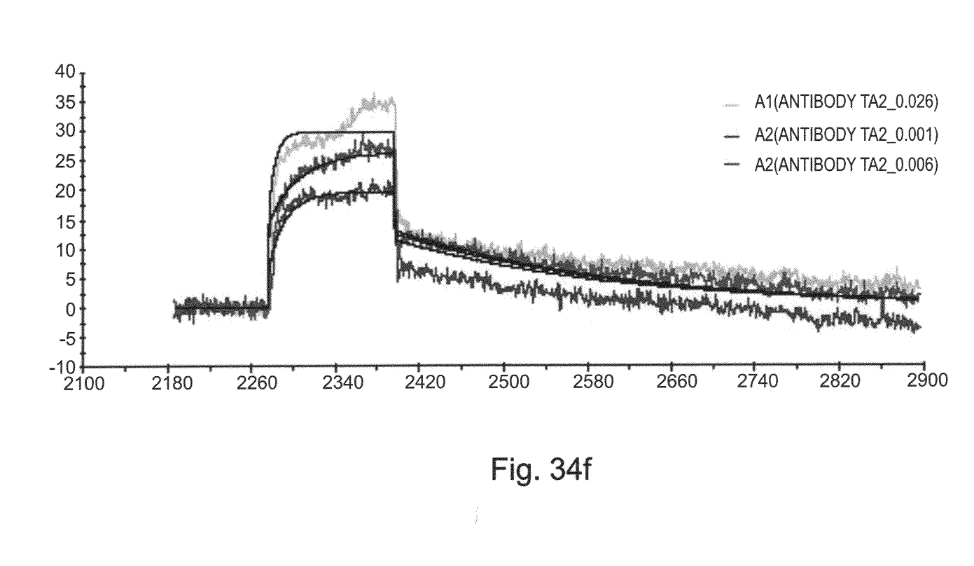

Construction and purification of TCR-like antibody-toxin fusion proteins: To explore the tumor targeting capabilities of the gp100 and MelanA/Mart-1 TCR-like antibodies, fusion molecules were generated in which the Fabs were fused to a form of Pseudomonas Exotoxin A (PE38 KDEL) [Allured, V. S., et al., Proc Natl Acad Sci USA 83, 1320-4 (1986)]. This truncated form of PE contains the translocation and ADP-ribosylation domains of whole PE but lacks the cell-binding domain, which is replaced by the Fab fragment fused at the N-terminus of the truncated toxin. In addition, the 5 C-terminal amino acids REDLK of the native PE were replaced with KDEL, which increases cytotoxicity of PE [Seetharam et al., J Biol Chem 266, 17376-81 (1991)] due to efficient binding to the ER retention receptor [Kreitman et al, Biochem J 307 (Pt 1), 29-37 (1995)] where it is translocated to the cytosol and inhibits protein synthesis. The truncated PE38 KDEL gene was fused at its N-terminus to the C-terminus of each Fab light chain (CL) as shown schematically in FIG. 34A. The Fab-PE38 KDEL fusions were expressed in E. coli BL21 (λDE3) cells and upon induction with IPTG, large amounts of recombinant protein accumulated as intracellular inclusion bodies. The inclusion bodies were analyzed by SDS-PAGE and those corresponding to the Light chain fused to PE and the Heavy chain each contained more than 90% recombinant protein (FIG. 34B). Using established renaturation protocols Fab-PE38 KDEL was refolded from solubilised inclusion bodies in a redox-shuffling refolding buffer and purified by anion-exchange chromatography on Q-Sepharose and Mono-Q columns. Highly purified Fab-PE38 KDEL fusion protein with the expected size was obtained as analyzed by SDS-PAGE under reducing and non reducing conditions (FIG. 34C lanes 1 and 2, respectively). The yield of the refolded Fab-PE38 KDEL fusions was ˜4%, thus, 4 mg of highly pure protein could be routinely obtained from the refolding of 100 mg of protein derived from inclusion bodies containing 80-90% of recombinant protein. This yield is similar to previously reported scFv-immunotoxins that were well-expressed and were produced using a similar expression and refolding system [Denkberg et al., J Immunol 171, 2197-207 (2003)].

The binding specificity of the soluble purified Fab-PE38 KDEL fusion proteins was determined by ELISA on biotinylated MHC-peptide complexes immobilized to wells through BSA-biotin-streptavidin to ensure correct folding of the complexes.

As shown in FIG. 34D, Fab 2F1-PE38 KDEL reacts specifically with the immobilized HLA-A2/gp100-G-280 complexes and not with control HLA-A2-peptide complexes. Detection of binding, as shown, was with anti-human Fab as well with anti-PE38 antibodies to detect the toxin portion of the fusion molecule. Similar results were observed with the other TCR-like fusions recognizing specifically the gp100 G9-154 epitope and the Mart-1 26-35 HLA-A2-peptide complex (not shown).

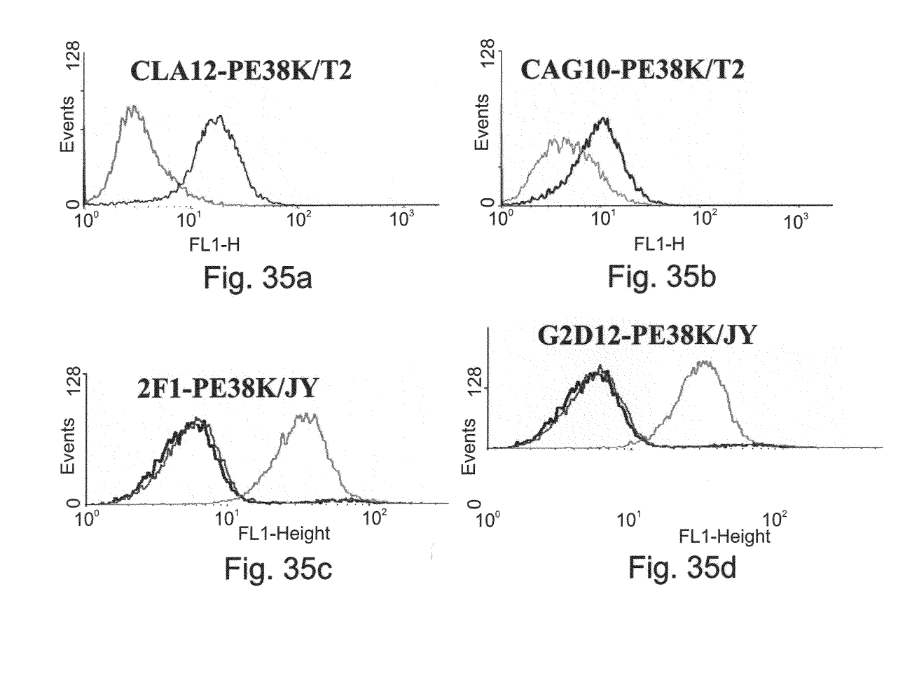

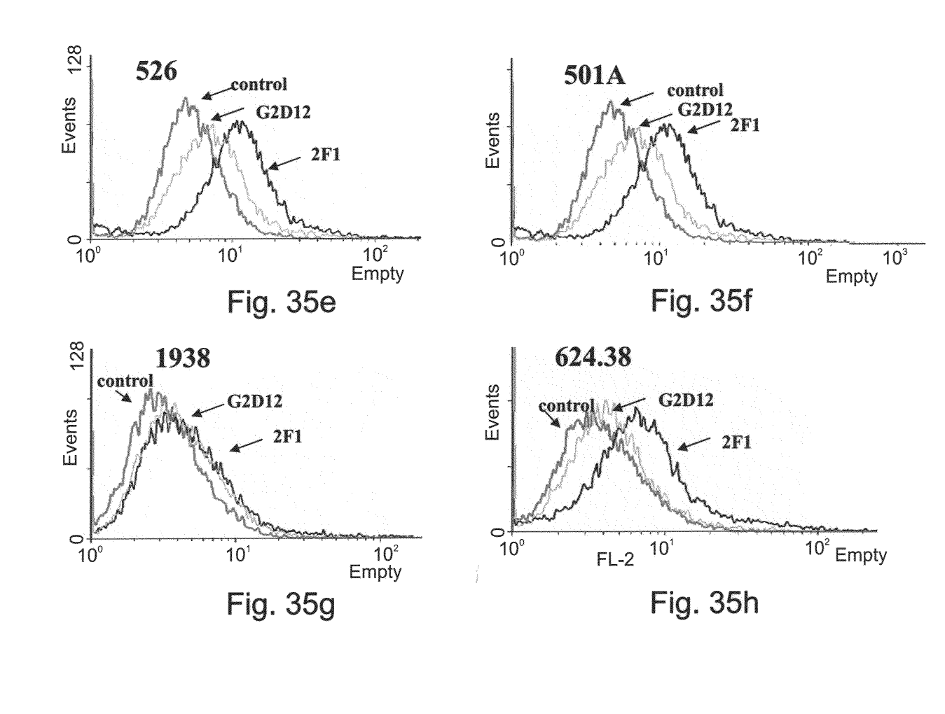

Binding of the Fab-PE38 KDEL fusions to APCs and melanoma cells displaying the gp100 and Mart-1-derived epitopes. As shown in FIGS. 32A-F and 33A-F, TCR-like Fabs can bind in an MHC-peptide-dependent manner to peptide-loaded APCs as well as to endogenous peptide/MHC complexes on the surface of melanoma tumor cells. To demonstrate that similar binding can occur with the Fab-PE38 KDEL fusion proteins, flow cytometry studies were performed on APCs and melanoma cells. As shown in FIGS. 35A-D, the Fab-PE38 KDEL fusions bound to APCs (T2 or JY cells) pulsed with the appropriate peptide, but did not bind to control pulsed cells. Binding was also tested by flow cytometry with melanoma cells. As shown in FIGS. 35E-H, the Fab-PE38 KDEL fusion proteins bound to HLA-A2+ and gp100/Mart-1+ melanoma cells 526, 501A, and 624.38, but not to 1938 melanoma cells which are HLA-A2+ but gp100/Mart-1−. These results demonstrate the ability of the TCR-like Fab-PE38 KDEL fusion molecules to bind the authentic endogenously-derived MHC-peptide complex when at a limited density on the surface of the tumor cells.

Affinities of CLA12 and TA2 to the complexed target was determined by Biacore analysis as shown in FIGS. 34 E-F (for CLA12 and TA2, respectively) and Tables 7 and 8, herein below (for CLA12 and TA2, respectively).

TABLE 7
Biacore analysis for CLA12

| | | | | | Conc | | | | | |
|---|---|---|---|---|---|---|---|---|---|---|
| | Ka | | Rmax | | of | | | Req | Kobs | |
| | (l/Ms) | Kd(l/s) | (RU) | R1(RU) | analyte | KA(l/M) | KD(M) | (RU) | (l/s) | Chi2 |
| | 5.18e6 | 2.86e⁻³ | 104 | | | 1.81e⁹ | 5.53e⁻¹⁰ | | | 16.6 |
| D4(1A7- | | | | 51.7 | 25 n | | | 102 | 0.132 | |
| 0.025) | | | | | | | | | | |
| D5(1A7- | | | | 44.1 | 10 n | | | 98.6 | 0.0546 | |
| 0.01) | | | | | | | | | | |
| D6(1A7- | | | | 25.9 | 5 n | | | 93.6 | 0.0287 | |
| 0.005) | | | | | | | | | | |

TABLE 8
Biacore analysis for TA2

| | | | | | Conc | | | | | |
|---|---|---|---|---|---|---|---|---|---|---|
| | Ka | | Rmax | | of | | | Req | Kobs | |
| | (l/Ms) | Kd(l/s) | (RU) | R1(RU) | analyte | KA(l/M) | KD(M) | (RU) | (l/s) | Chi2 |
| | 5.79e6 | 4.73e⁻³ | 13.6 | | | 1.22e⁹ | 8.18e⁻¹⁰ | | | 8.39 |
| A1(Antibody | | | | 16.6 | 25 n | | | 13.2 | 0.149 | |
| TA2-0.025) | | | | | | | | | | |
| A2(Antibody | | | | 6.98 | 10 n | | | 12.6 | 0.0626 | |
| TA2-0.01) | | | | | | | | | | |
| A3(Antibody | | | | 14.6 | 5 n | | | 11.7 | 0.0337 | |
| TA2-0.005) | | | | | | | | | | |

Internalization of TCR-like antibodies: An open question regarding the targeting and drug delivery capabilities of TCR-like antibodies is whether these molecules can induce internalization of the MHC-peptide complex. To determine if the TCR-like antibodies internalize, the Fab 2F1-PE38 KDEL fusion protein was labeled with FITC and its binding and internalization was tested on JY APCs pulsed with the appropriate gp100-derived G9-280 peptide (SEQ ID NO: 5). As shown in FIG. 36A, the FITC-labeled Fab 2F1-PE38 KDEL molecule binds specifically to G9-280 peptide-pulsed JY cells but not to cells pulsed with a control peptide G9-209. Internalization was monitored by confocal microscopy (FIGS. 36B-F). When cells were incubated with FITC-labeled Fab 2F1-PE38 KDEL at 4° C. membranous binding was observed. Similar membrane binding was observed at 37° C. at time 0 immediately after incubation with labeled fusion molecule (FIG. 36B, 0 min), and no fluorescence was observed on the negative control (JY cells+peptide G9-209, not shown). At 37° C., internalization of 2F1-PE38 KDEL-FITC became visible in JY cells loaded with peptide G9-280 (SEQ ID NO: 5) (FIGS. 36C-F). After 15 minutes the majority of stain intensity was mainly on the surface of the cell (FIG. 36C) and dense areas of fluorescence were detected, which may indicate processes of aggregation or capping of MHC-peptide complexes. After 30 minutes, cells displayed internalizing molecules in small vesicles (FIG. 36D). After 1 hour of incubation, the cells internalized more than 50% of the FITC-labeled antibody, which was detected in larger vesicles (FIG. 36E). After 6 hours, intensive staining was observed around the nucleus in the ER-Golgi compartment (FIG. 36F). Cells on ice demonstrated mainly or only membrane staining even after 3 hours of incubation (not shown).

These results show that active internalization is induced in cells after binding of TCR-like antibodies to the cell surface and therefore such molecules can deliver toxic agents or drugs into target cells.

Cytotoxic activity of TCR-like Fab-PE38 KDEL fusion toward APCs: To determine the ability of the Fab-PE38 KDEL to deliver toxin and eliminate cells that express the appropriate peptide/MHC complex, peptide-loaded APCs were used. RMAS-HHD or JY cells were loaded with the gp100-derived epitope G9-280 as well as with other control HLA-A2-restricted peptides. FACS analysis with anti-HLA-A2 antibody revealed similar expression patterns of HLA-A2 molecules with G9-280 (SEQ ID NO: 5), and other control peptide-loaded cells (not shown). The ability of the Fab-PE38 KDEL to inhibit protein synthesis was used as a measure of its cytotoxic effect.

As shown in FIG. 37A, cytotoxicity by 2F1 Fab-PE38 KDEL was observed only on JY cells loaded with the G9-280 peptide (SEQ ID NO: 5) with an IC₅₀ of ˜0.5 ng/ml. No cytotoxic activity was observed on JY cells that were loaded with other control HLA-A2-restricted peptides or cells that were not loaded with peptide. Similar results were observed when RMAS-HHD cells were loaded with the G9-280 (SEQ ID NO: 5) and control peptides. The sensitivity of JY cells to immunotoxin is mainly due to the high number of HLA-A2 molecules on the surface of the cells, measured in previous studies of the present inventors to be 1.5-2×10⁵ molecules/cell [Cohen et al., J Immunol 170, 4349-61 (2003)] and the high efficiency of peptide loading. To demonstrate cytotoxic activity towards other cells with limited HLA-A2 expression, melanoma cells FM3D were used, which express 20-fold fewer sites on their surface (1×10⁴ molecules/cell). As controls melanoma G-43 cells were used which are HLA-A2 negative. Both melanomas were pulsed with the G9-280 (SEQ ID NO: 5) and control peptides and exposed to the 2F1 Fab-PE38 KDEL fusion molecule. As shown in FIG. 37B, 2F1 Fab-PE38 KDEL induced killing in FM3D cells pulsed with the G9-280 peptide (SEQ ID NO: 5), but not with other HLA-A2-restricted control peptides nor with G-43 cells pulsed with G9-280 or control peptides. The IC₅₀ for G9-280-pulsed FM3D was ˜200 ng/ml reflecting the lower number of HLA-A2/G9-280 sites compared to G9-280-pulsed JY cells. Thus, a direct correlation between Fab-PE38 KDEL cytotoxic activity and the number of target sites per cell was observed. Similar results were observed with the Mart-1-specific CLA12 Fab-PE38 KDEL fusion proteins (data not shown). These results demonstrate that Fab-PE38 KDEL fusion proteins are very specific agents that, like a T cell receptor, can recognize particular peptide/MHC complexes. Such specificity characteristics are necessary for each new therapeutic candidate antibody that can target drugs or toxins to a defined population of cells that express a particular peptide/MHC class I complexes.

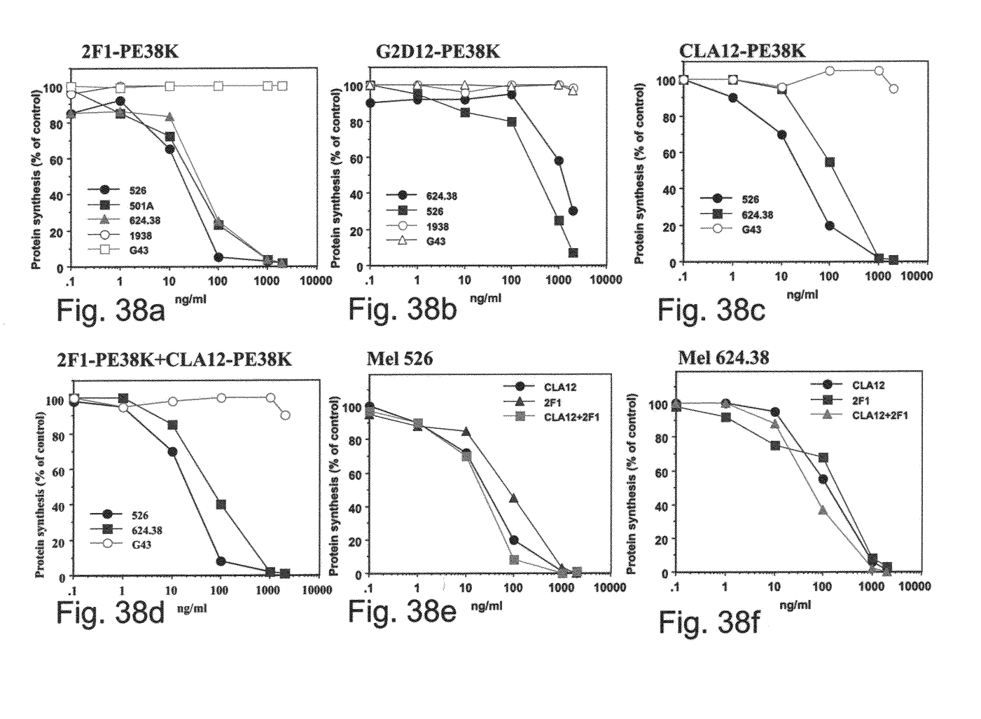

Cytotoxic activity of TCR-like Fab-PE38 KDEL fusion molecules toward melanoma cells expressing endogenous gp100 and Mart-1. To evaluate the activity of TCR-like Fab-PE38 KDEL fusion molecules to cells which express the natural endogenous target antigen melanoma cells were used that express gp100 and Mart-1 and are HLA-A2 positive. These cells and controls were exposed to increasing concentrations of TCR-like-Fab-toxin. As shown in FIGS. 38A-C, TCR-like Fab-PE38 KDEL fusion proteins exhibited cytotoxic activity on antigen+, HLA-A2+ melanoma cells (526, 501A, and 624.38) but not on HLA-A2−, antigen+ G-43 cells or on HLA-A2+, antigen-1938 cells. The IC₅₀ of the immunotoxin molecules to antigen and HLA-A2+ cells was 20-100 ng/ml depending on the cell type and the target antigen. 2F1 Fab-PE38 KDEL and CLA12 Fab-PE38 KDEL were most potent in inducing specific cell killing with an IC₅₀ of 20-30 ng/ml on 526 melanoma cells (FIGS. 38A and 38C). The present inventors further investigated whether the combined use of two immunotoxins against different epitopes results in increased cell cytotoxicity. Therefore, target melanoma cells were incubated with a mixture of 2F1 Fab-PE38 KDEL and CLA12 Fab-PE38 KDEL at increasing concentrations as indicated (FIG. 38D). As shown, the specificity was not altered and the cytotoxic activity was slightly improved. Detailed analysis on individual target cells, melanomas 526 (FIG. 38E) and 624.38 (FIG. 38F), revealed that the IC₅₀ of 2F1 Fab-PE38 KDEL and CLA12 Fab-PE38 KDEL on 526 melanoma cells was 70 and 30 ng/ml, respectively, however their combined effect yielded an IC₅₀ of 20 ng/ml. A more significant combined effect was observed on 634.38 target cells in which 2F1 and CLA12 Fab-PE38 KDEL fusion proteins exhibited an IC₅₀ of 200 and 150 ng/ml, respectively, however the combination of them yielded cytotoxic activity with an IC₅₀ of 50 ng/ml. These results indicate the capability of TCR-like antibodies fused to PE38 KDEL to induce efficient and specific cytotoxic activity on melanoma target cells that express natural endogenous differentiation antigens gp100 and Mart-1. The use of cocktails of fusion molecules targeting more than one antigen may have a slight beneficial effect on the overall cytotoxic activity as compared to the use of a single agent and likely reflects increased peptide-MHC target density for toxin delivery.

In vivo anti-tumor activity of TCR-like Fab-PE38 KDEL-KDEL fusion proteins: Based on the in vitro cytotoxic activity of CLA12 Fab-PE38 KDEL, the present inventors sought to evaluate its in vivo activity. Therefore, the ability of TCR-like Fab-PE38 KDEL was assessed to induce regression or inhibition of human melanoma in irradiated NOD SCID β2M-deficient mice. Mice had been inoculated with 10⁷ Mel-526 tumor cells and were randomly assigned to treatment groups, with 4 mice in each group. Mice were administrated with four i.v. doses each of CLA12-PE38 KDEL (0.05-0.125 mg/kg), or PBS diluent control. Treatment began on day 11 post inoculation when the tumor size reached approximately 55 mm² and was administrated at 48 h intervals for 8 days. Tumor volumes were recorded for 34 days. By day 34, tumors in mice receiving PBS diluent grew to a size averaging 530 mm³. Treatment with 0.05 mg/kg CLA12-PE38 KDEL delayed tumor development compared with control and by day 34 tumor size reached 259 mm³ (FIGS. 39A-B). The effect on tumor was dose-dependent; since treatment with 0.125 mg/kg CLA12-PE38 KDEL delayed tumor development to a greater extent, reaching 25% (135 mm³) of the average size of the tumors in the control group on day 34. Inhibition of tumor growth was statistically significant for any dose of toxin used with P value of less than 0.0006. It can therefore be concluded that CLA12-PE38 KDEL is effective in slowing the growth of HLA-A2+Mart-1+ tumors at non-toxic doses.

Discussion

This example teaches the isolation of 2 novel human recombinant Fab antibodies Fabs-CLA12 and Fab-CAG10. These Fabs recognize peptide derived from the melanoma differentiation antigen Mart-1 (Mart-1₂₆₋₃₇) in the context of HLA-A2.

Studies in mice [Reiter et al, Proc Natl Acad Sci USA 94, 4631-6 (1997)] demonstrated that a recombinant antibody which recognizes mouse H2-K^{k} class I molecules complexed with the K^{k}-restricted influenza virus-derived peptide of hemagglutinin (peptide Ha₂₅₅₋₂₆₂) can be used very efficiently to deliver a cytotoxic drug or toxin to APCs that express the suitable peptide/MHC complex. The present inventors sought to apply this immunotherapy approach to human diseases such as cancer.

An extended panel of melanoma-specific antibodies with T cell receptor-like specificity were used for targeting toxin to cells that express specific peptide/MHC complex in vitro and in vivo. 4 different human antibody-toxin hybrid molecules (immunotoxins) were generated, in which a human Fab antibody is fused to the PE derivative, PE38KDEL.

All 4 recombinant immunotoxins that were analyzed had high binding specificity to APCs and melanoma cells expressing the specific peptide/HLA-A2 complex. The immunotoxin 2F1-PE38KEDL has a high rate of internalization (within 15 min) and accumulation in the cytosol of the cell (at 6 h) where it exerts its function. When tested in vitro, Fab-PE38 KDEL immunotoxins kill APCs and melanoma cells in a peptide-dependent MHC-restricted manner. Immunotoxin G2D12-PE38 KDEL was not as potent in killing melanoma cell lines compared to 2F1-PE38 KDEL, CLA12-PE38 KDEL and CAG10-PE38KDEL, and this order correlates with lower expression of p154/HLA-A2 complexes on the surface of the melanoma cell line. Furthermore, CLA12-PE38 KDEL has specific antitumor activity in a mouse xenograft model for human melanoma. It is therefore able to penetrate solid tumor and substantially delay tumor growth in mice at doses that do not produce animal toxicity.

Having constructed immunotoxin against several melanoma epitopes, the present inventors aimed at increasing the probability of eradicating tumor cell variants. As tumor cells tend to mutate, they may lack the target antigen entirely or express it at levels too low for effective immunotoxin-mediated killing. Such mutant cells could be eradicated with cocktails of two or more immunotoxins recognizing different target antigens and this has been demonstrated by targeting lymphoma with a cocktail of anti-CD19 and anti-CD22 ricin-conjugated immunotoxins [Ghetie et al., Blood 80, 2315-20 (1992)]. In the present in vitro study, a significantly higher cytotoxic effect was not seen when using more than one immunotoxin. This may probably be due to relatively homogenous expression of the target antigen in melanoma cell lines. However, in patients carrying a large tumor mass, mutations are abundant and using a cocktail of two or more immunotoxins may show a beneficial effect.

The only immunotoxin currently being evaluated for treatment of metastatic melanoma is comprised of ricin A chain conjugated to a murine monoclonal antibody directed against high molecular weight melanoma antigens. This immunotoxin induces toxicities such as myalgia, arthralgia, hypoalbuminemia, fatigue, elevations in liver function tests; and increased peripheral edema. In addition, the use of murine antibody may induce the development of anti-immunotoxin antibodies which will result in decreased efficacy and limit repetitive dosing with an immunotoxin [Gonzalez, R. et al. Mol Biother 3, 192-6 (1991); Oratz, R. et al. J Biol Response Mod 9, 345-54 (1990); Selvaggi, K. et al. J Immunother 13, 201-7 (1993)].

Thus the present inventors propose the evaluation of a different class of toxin target for melanoma, the use of a human antibody which is less immunogenic and of smaller size which should penetrate tumor more easily and be processed more efficiently.

The data presented here are a proof of principal that specific MHC complexes can be used in humans as target therapy for melanoma. Moreover, the delivery of TCR-L-PE38 KDEL toxin could serve as a first line therapy, to debulk tumor mass and prevent further rigorous growth and is a general approach that can be readily extended to known immunodominant peptides for other HLA [Krogsgaard, M. et al. J Exp Med 191, 1395-412 (2000); Chames, P., Proc Natl Acad Sci USA 97, 7969-74 (2000)] and different cancer types.

### Example 11

### Identification of Tyrosinase Peptides

The present inventors ranked potential 8-mer, 9-mer, or 10-mer tyrosinase peptides based on a predicted half-time of dissociation to HLA class I molecules. using bioinformatics and molecular tools found on www-bimas.cit.nih.gov/molbio/hla_bind/. Tyrosinase peptides were selected from both the unmodified protein (SEQ ID NO: 67) and unmodified protein (SEQ ID NO: 68) as set forth in Table 139, all of which can be used according to the teachings of the present invention.

HLA Peptide Motif Search Results

Lengthy table referenced here
US20100158927A1-20100624-T00001
Please refer to the end of the specification for access instructions.

Lengthy table referenced here
US20100158927A1-20100624-T00002
Please refer to the end of the specification for access instructions.

Lengthy table referenced here
US20100158927A1-20100624-T00003
Please refer to the end of the specification for access instructions.

Lengthy table referenced here
US20100158927A1-20100624-T00004
Please refer to the end of the specification for access instructions.

Lengthy table referenced here
US20100158927A1-20100624-T00005
Please refer to the end of the specification for access instructions.

Lengthy table referenced here
US20100158927A1-20100624-T00006
Please refer to the end of the specification for access instructions.

Lengthy table referenced here
US20100158927A1-20100624-T00007
Please refer to the end of the specification for access instructions.

Lengthy table referenced here
US20100158927A1-20100624-T00008
Please refer to the end of the specification for access instructions.

Lengthy table referenced here
US20100158927A1-20100624-T00009
Please refer to the end of the specification for access instructions.

Lengthy table referenced here
US20100158927A1-20100624-T00010
Please refer to the end of the specification for access instructions.

Lengthy table referenced here
US20100158927A1-20100624-T00011
Please refer to the end of the specification for access instructions.

Lengthy table referenced here
US20100158927A1-20100624-T00012
Please refer to the end of the specification for access instructions.

Lengthy table referenced here
US20100158927A1-20100624-T00013
Please refer to the end of the specification for access instructions.

Lengthy table referenced here
US20100158927A1-20100624-T00014
Please refer to the end of the specification for access instructions.

Lengthy table referenced here
US20100158927A1-20100624-T00015
Please refer to the end of the specification for access instructions.

Lengthy table referenced here
US20100158927A1-20100624-T00016
Please refer to the end of the specification for access instructions.

Lengthy table referenced here
US20100158927A1-20100624-T00017
Please refer to the end of the specification for access instructions.

Lengthy table referenced here
US20100158927A1-20100624-T00018
Please refer to the end of the specification for access instructions.

Lengthy table referenced here
US20100158927A1-20100624-T00019
Please refer to the end of the specification for access instructions.

Lengthy table referenced here
US20100158927A1-20100624-T00020
Please refer to the end of the specification for access instructions.

Lengthy table referenced here
US20100158927A1-20100624-T00021
Please refer to the end of the specification for access instructions.

Lengthy table referenced here
US20100158927A1-20100624-T00022
Please refer to the end of the specification for access instructions.

Lengthy table referenced here
US20100158927A1-20100624-T00023
Please refer to the end of the specification for access instructions.

Lengthy table referenced here
US20100158927A1-20100624-T00024
Please refer to the end of the specification for access instructions.

Lengthy table referenced here
US20100158927A1-20100624-T00025
Please refer to the end of the specification for access instructions.

Lengthy table referenced here
US20100158927A1-20100624-T00026
Please refer to the end of the specification for access instructions.

Lengthy table referenced here
US20100158927A1-20100624-T00027
Please refer to the end of the specification for access instructions.

Lengthy table referenced here
US20100158927A1-20100624-T00028
Please refer to the end of the specification for access instructions.

Lengthy table referenced here
US20100158927A1-20100624-T00029
Please refer to the end of the specification for access instructions.

Lengthy table referenced here
US20100158927A1-20100624-T00030
Please refer to the end of the specification for access instructions.

Lengthy table referenced here
US20100158927A1-20100624-T00031
Please refer to the end of the specification for access instructions.

Lengthy table referenced here
US20100158927A1-20100624-T00032
Please refer to the end of the specification for access instructions.

Lengthy table referenced here
US20100158927A1-20100624-T00033
Please refer to the end of the specification for access instructions.

Lengthy table referenced here
US20100158927A1-20100624-T00034
Please refer to the end of the specification for access instructions.

Lengthy table referenced here
US20100158927A1-20100624-T00035
Please refer to the end of the specification for access instructions.

Lengthy table referenced here
US20100158927A1-20100624-T00036
Please refer to the end of the specification for access instructions.

Lengthy table referenced here
US20100158927A1-20100624-T00037
Please refer to the end of the specification for access instructions.

Lengthy table referenced here
US20100158927A1-20100624-T00038
Please refer to the end of the specification for access instructions.

Lengthy table referenced here
US20100158927A1-20100624-T00039
Please refer to the end of the specification for access instructions.

Lengthy table referenced here
US20100158927A1-20100624-T00040
Please refer to the end of the specification for access instructions.

Lengthy table referenced here
US20100158927A1-20100624-T00041
Please refer to the end of the specification for access instructions.

Lengthy table referenced here
US20100158927A1-20100624-T00042
Please refer to the end of the specification for access instructions.

Lengthy table referenced here
US20100158927A1-20100624-T00043
Please refer to the end of the specification for access instructions.

Lengthy table referenced here
US20100158927A1-20100624-T00044
Please refer to the end of the specification for access instructions.

Lengthy table referenced here
US20100158927A1-20100624-T00045
Please refer to the end of the specification for access instructions.

Lengthy table referenced here
US20100158927A1-20100624-T00046
Please refer to the end of the specification for access instructions.

Lengthy table referenced here
US20100158927A1-20100624-T00047
Please refer to the end of the specification for access instructions.

Lengthy table referenced here
US20100158927A1-20100624-T00048
Please refer to the end of the specification for access instructions.

Lengthy table referenced here
US20100158927A1-20100624-T00049
Please refer to the end of the specification for access instructions.

Lengthy table referenced here
US20100158927A1-20100624-T00050
Please refer to the end of the specification for access instructions.

Lengthy table referenced here
US20100158927A1-20100624-T00051
Please refer to the end of the specification for access instructions.

Lengthy table referenced here
US20100158927A1-20100624-T00052
Please refer to the end of the specification for access instructions.

Lengthy table referenced here
US20100158927A1-20100624-T00053
Please refer to the end of the specification for access instructions.

Lengthy table referenced here
US20100158927A1-20100624-T00054
Please refer to the end of the specification for access instructions.

Lengthy table referenced here
US20100158927A1-20100624-T00055
Please refer to the end of the specification for access instructions.

Lengthy table referenced here
US20100158927A1-20100624-T00056
Please refer to the end of the specification for access instructions.

Lengthy table referenced here
US20100158927A1-20100624-T00057
Please refer to the end of the specification for access instructions.

Lengthy table referenced here
US20100158927A1-20100624-T00058
Please refer to the end of the specification for access instructions.

Lengthy table referenced here
US20100158927A1-20100624-T00059
Please refer to the end of the specification for access instructions.

Lengthy table referenced here
US20100158927A1-20100624-T00060
Please refer to the end of the specification for access instructions.

Lengthy table referenced here
US20100158927A1-20100624-T00061
Please refer to the end of the specification for access instructions.

Lengthy table referenced here
US20100158927A1-20100624-T00062
Please refer to the end of the specification for access instructions.

Lengthy table referenced here
US20100158927A1-20100624-T00063
Please refer to the end of the specification for access instructions.

Lengthy table referenced here
US20100158927A1-20100624-T00064
Please refer to the end of the specification for access instructions.

Lengthy table referenced here
US20100158927A1-20100624-T00065
Please refer to the end of the specification for access instructions.

Lengthy table referenced here
US20100158927A1-20100624-T00066
Please refer to the end of the specification for access instructions.

Lengthy table referenced here
US20100158927A1-20100624-T00067
Please refer to the end of the specification for access instructions.

Lengthy table referenced here
US20100158927A1-20100624-T00068
Please refer to the end of the specification for access instructions.

Lengthy table referenced here
US20100158927A1-20100624-T00069
Please refer to the end of the specification for access instructions.

Lengthy table referenced here
US20100158927A1-20100624-T00070
Please refer to the end of the specification for access instructions.

Lengthy table referenced here
US20100158927A1-20100624-T00071
Please refer to the end of the specification for access instructions.

Lengthy table referenced here
US20100158927A1-20100624-T00072
Please refer to the end of the specification for access instructions.

Lengthy table referenced here
US20100158927A1-20100624-T00073
Please refer to the end of the specification for access instructions.

Lengthy table referenced here
US20100158927A1-20100624-T00074
Please refer to the end of the specification for access instructions.

Lengthy table referenced here
US20100158927A1-20100624-T00075
Please refer to the end of the specification for access instructions.

Lengthy table referenced here
US20100158927A1-20100624-T00076
Please refer to the end of the specification for access instructions.

Lengthy table referenced here
US20100158927A1-20100624-T00077
Please refer to the end of the specification for access instructions.

Lengthy table referenced here
US20100158927A1-20100624-T00078
Please refer to the end of the specification for access instructions.

Lengthy table referenced here
US20100158927A1-20100624-T00079
Please refer to the end of the specification for access instructions.

Lengthy table referenced here
US20100158927A1-20100624-T00080
Please refer to the end of the specification for access instructions.

Lengthy table referenced here
US20100158927A1-20100624-T00081
Please refer to the end of the specification for access instructions.

Lengthy table referenced here
US20100158927A1-20100624-T00082
Please refer to the end of the specification for access instructions.

Lengthy table referenced here
US20100158927A1-20100624-T00083
Please refer to the end of the specification for access instructions.

Lengthy table referenced here
US20100158927A1-20100624-T00084
Please refer to the end of the specification for access instructions.

Lengthy table referenced here
US20100158927A1-20100624-T00085
Please refer to the end of the specification for access instructions.

Lengthy table referenced here
US20100158927A1-20100624-T00086
Please refer to the end of the specification for access instructions.

Lengthy table referenced here
US20100158927A1-20100624-T00087
Please refer to the end of the specification for access instructions.

Lengthy table referenced here
US20100158927A1-20100624-T00088
Please refer to the end of the specification for access instructions.

Lengthy table referenced here
US20100158927A1-20100624-T00089
Please refer to the end of the specification for access instructions.

Lengthy table referenced here
US20100158927A1-20100624-T00090
Please refer to the end of the specification for access instructions.

Lengthy table referenced here
US20100158927A1-20100624-T00091
Please refer to the end of the specification for access instructions.

Lengthy table referenced here
US20100158927A1-20100624-T00092
Please refer to the end of the specification for access instructions.

Lengthy table referenced here
US20100158927A1-20100624-T00093
Please refer to the end of the specification for access instructions.

Lengthy table referenced here
US20100158927A1-20100624-T00094
Please refer to the end of the specification for access instructions.

Lengthy table referenced here
US20100158927A1-20100624-T00095
Please refer to the end of the specification for access instructions.

Lengthy table referenced here
US20100158927A1-20100624-T00096
Please refer to the end of the specification for access instructions.

Lengthy table referenced here
US20100158927A1-20100624-T00097
Please refer to the end of the specification for access instructions.

Lengthy table referenced here
US20100158927A1-20100624-T00098
Please refer to the end of the specification for access instructions.

Lengthy table referenced here
US20100158927A1-20100624-T00099
Please refer to the end of the specification for access instructions.

Lengthy table referenced here
US20100158927A1-20100624-T00100
Please refer to the end of the specification for access instructions.

Lengthy table referenced here
US20100158927A1-20100624-T00101
Please refer to the end of the specification for access instructions.

Lengthy table referenced here
US20100158927A1-20100624-T00102
Please refer to the end of the specification for access instructions.

Lengthy table referenced here
US20100158927A1-20100624-T00103
Please refer to the end of the specification for access instructions.

Lengthy table referenced here
US20100158927A1-20100624-T00104
Please refer to the end of the specification for access instructions.

Lengthy table referenced here
US20100158927A1-20100624-T00105
Please refer to the end of the specification for access instructions.

Lengthy table referenced here
US20100158927A1-20100624-T00106
Please refer to the end of the specification for access instructions.

Lengthy table referenced here
US20100158927A1-20100624-T00107
Please refer to the end of the specification for access instructions.

Lengthy table referenced here
US20100158927A1-20100624-T00108
Please refer to the end of the specification for access instructions.

Lengthy table referenced here
US20100158927A1-20100624-T00109
Please refer to the end of the specification for access instructions.

Lengthy table referenced here
US20100158927A1-20100624-T00110
Please refer to the end of the specification for access instructions.

Lengthy table referenced here
US20100158927A1-20100624-T00111
Please refer to the end of the specification for access instructions.

Lengthy table referenced here
US20100158927A1-20100624-T00112
Please refer to the end of the specification for access instructions.

Lengthy table referenced here
US20100158927A1-20100624-T00113
Please refer to the end of the specification for access instructions.

Lengthy table referenced here
US20100158927A1-20100624-T00114
Please refer to the end of the specification for access instructions.

Lengthy table referenced here
US20100158927A1-20100624-T00115
Please refer to the end of the specification for access instructions.

Lengthy table referenced here
US20100158927A1-20100624-T00116
Please refer to the end of the specification for access instructions.

Lengthy table referenced here
US20100158927A1-20100624-T00117
Please refer to the end of the specification for access instructions.

Lengthy table referenced here
US20100158927A1-20100624-T00118
Please refer to the end of the specification for access instructions.

Lengthy table referenced here
US20100158927A1-20100624-T00119
Please refer to the end of the specification for access instructions.

Lengthy table referenced here
US20100158927A1-20100624-T00120
Please refer to the end of the specification for access instructions.

Lengthy table referenced here
US20100158927A1-20100624-T00121
Please refer to the end of the specification for access instructions.

Lengthy table referenced here
US20100158927A1-20100624-T00122
Please refer to the end of the specification for access instructions.

Lengthy table referenced here
US20100158927A1-20100624-T00123
Please refer to the end of the specification for access instructions.

Lengthy table referenced here
US20100158927A1-20100624-T00124
Please refer to the end of the specification for access instructions.

Lengthy table referenced here
US20100158927A1-20100624-T00125
Please refer to the end of the specification for access instructions.

Lengthy table referenced here
US20100158927A1-20100624-T00126
Please refer to the end of the specification for access instructions.

Lengthy table referenced here
US20100158927A1-20100624-T00127
Please refer to the end of the specification for access instructions.

Lengthy table referenced here
US20100158927A1-20100624-T00128
Please refer to the end of the specification for access instructions.

Lengthy table referenced here
US20100158927A1-20100624-T00129
Please refer to the end of the specification for access instructions.

Lengthy table referenced here
US20100158927A1-20100624-T00130
Please refer to the end of the specification for access instructions.

Lengthy table referenced here
US20100158927A1-20100624-T00131
Please refer to the end of the specification for access instructions.

### Example 12

### Generation of Additional Antibodies with a Specificity to the HLA-A2/Tyrosinase₃₆₉₋₃₇₇ Complex

Materials and Methods

Production of Biotinylated scMHC/peptide Complexes: To construct scMHC-BirA plasmid, a peptide sequence for site specific biotinylation (LHHILDAQKMVWNHR, (SEQ ID NO: 900) the lysine residue undergoing biotinylation to by the BirA biotin ligase enzyme is marked) was fused at the C-terminus of the HLA-A2. These construct was subcloned into a pET-based expression vector for efficient expression in E. Coli.

Folding and Purification of recombinant MHC/peptide complexes, and recombinant fusion molecule: Dithioerithriol was added to a final concentration of 65 mM (10 mg/ml) to the solubilized inclusion bodies of scMHC, or fusion molecule which were incubated for >2 hours. The reduced inclusion bodies were diluted ×100 with refolding buffer (0.1M Tris-HCl pH=8, 0.5M Arginine, 0.09 mM Oxidized Glutathione, 2 mM EDTA, 0.2 mM PMSF). 5 or 10 fold molar excess of peptide (usually 1 mg/100 ml refolding buffer) was added to scMHC and the molecule containing HLA-A2, previously diluted in H₂O or DMSO, and incubated at 4-10° C. for 48 hours. After refolding, the protein was dialyzed against 100 mM Urea, 20 mM Tris-HCl pH=8, and concentrated by a Minisette system using a 10K cutoff cassette to a final volume of 200 ml. The protein was loaded on Q Sepharose anion exchange column. The column was washed with buffer A containing 5 mM NaCl, 20 mM Tris HCl pH=8, 1 mM EDTA. Relevant fractions corresponding to correctly folded MHC/peptide or fusion molecule monomers were poured on to a centricon device (30 kDa cut off) (Amicon, Beverly Mass.) and concentrated to volume 0.3-1.0 ml (usually no more than 2 mg/ml to avoid protein aggregation).

The clean fractions were frozen at −70° C. at this step, until further use.

Biotinylation of MHC/peptide complexes: The buffer was exchanged (using the centricon) with 10 mM Tris-HCl, pH=8, 50 mM NaCl. The final protein concentration was brought to 1-2 mg/ml (25-50 μM). Enzymatic biotinylation was performed at a specific lysine residue in the heavy chain C-terminal tag using biotin protein ligase—Bir A enzyme (AVIDITY, Denver, Colo.) for 16 hours at 25° C., in presence of protease inhibitors cocktail (0.1 mM PMSF, 1 μg/ml Leupeptin, 1 μg/ml Pepstatin). The buffer was exchanged and the excess biotin was removed from the biotinylated complexes using centricon 30 ultrafiltration or G-25. The MHC/peptide biotinylated monomers were frozen at −70° C.

Selection of Phage-Antibodies on Biotinylated Complexes: A large human Fab library containing 3.7×10¹⁰ different Fab clones was used for the selection [de Haard, H. J., et al., (1999) J. Biol. Chem. 274, 18218-18230]. Phage (10¹³) was first preincubated for 1 hour at room temperature in PBS containing 2% nonfat dry milk with streptavidin-coated paramagnetic beads (200 μl; Dynal, Oslo) to deplete streptavidin binders. Streptavidin-coated paramagnetic beads (200 μl; Dynal, Oslo) were also incubated in PBS+2% milk for 1 hour at room temperature. The remaining phages were subsequently incubated for 1 hour with decreasing amounts of biotinylated scMHC-peptide complexes. Streptavidin magnetic beads were added, and the mixture was incubated for 15 minutes with continuous rotation. A magnetic force was applied to pull down phages bound to biotinylated complexes. After 10 washes of the streptavidin-bound complexes with MPBS 0.1% Tween and 2 washes with PBS, bound phages were eluted by incubation for 7 minutes with 1 ml of Triethylamine (TEA) (100 mM). In this procedure, 3-4 rounds of selection were performed, the instant paragraph describing only one round of selection. In the first round 20 μg biotinylated scMHC-peptide complexes were used, and in the following rounds 5 μg.

A second protocol of Off-Rate selection was assessed and included 42 washes of the streptavidin-bound complexes with MPBS 0.1% Tween and 2 washes with PBS each time for 15 minutes. The elution mixture was neutralized by the addition of 100 μl of Tris-HCl (1M, pH 7.4) and used to infect E. coli TG1 cells (OD600=0.5) for 30 minutes at 37° C. Selected phages were rescued using M13KO7 helper phage (5×10¹¹ cfu).

For both selection protocols, the diversity of the selected antibodies was determined by DNA fingerprinting. The Fab DNA of different clones was PCR-amplified using the primers pUC-reverse (5′-AGCGGATAACAATTTCACACAGG-3′—SEQ ID NO: 901) and fd-tet-seq24 (5′-TTTGTCGTCTTTCCAGACGTTAGT-3′—SEQ ID NO: 902). The resulting PCR fragments were digested with BstNI (NEB) (2 hours, 37° C.) and analyzed by agarose gel electrophoresis.

Cell lines: JY (EBV-transformed B-lymphoblast), were maintained in RPMI-1640 supplemented with 10% FCS, 2 mM glutamine, Penicillin (100 units/ml) and Streptomycin (100 μg/ml) at 37° C. with 5% CO₂. The melanoma, leukemia, and glioblastoma cell lines were maintained in RPMI-1640 supplemented with 10% FCS, 2 mM glutamine, Penicillin (100 units/ml) and Streptomycin (100 μg/ml) at 37° C. with 5% CO₂.

Expression and purification of soluble recombinant Fab antibodies: The Fab antibody was expressed in BL21 λDE3 cells as previously described (1) and purified from the periplasmic fraction by metal-ion affinity chromatography using the hexahistidine tag: 4 μl of miniprep DNA was transformed to 100 μl 21 E. coli competent cells and plated the bacteria on 2YT/A/G agar plates and incubated at 37° C., over night. Inoculated plates into Superbroth supplemented with 12 ml/liter 40 gr/liter MgSO₄, 5 ml/liter 20% Glucose, and 100 μg/ml Ampicillin. For each liter of Superbroth, 5 plates were used (filled with colonies) which were grown to OD600 nm=0.8-1.0 and induced to express the recombinant Fab antibody by the addition of 1 mM IPTG for 3 hours at 30° C. The cells were centrifuged and the pellet was resuspended in 5 ml of a B-PER solution (Pierce) to release periplasmatic content. After 30 minutes of rotated incubation at RT, the solution was centrifuged (15000 rpm, 15 minutes) and the supernatant was incubated with 0.5 ml of pre-washed TALON beads suspension (Clontech) for 45 minutes at RT. The solution was applied onto a BioRad disposable column (Cat No. 731/1550), and after sedimentation the beads were washed three times with 10 ml of PBS/0.1% Tween20 (pH 8.0). The bound Fabs were eluted using 0.5 ml of 100 mM Imidazole in PBS. The eluted Fabs were dialyzed twice against PBS (overnight, 4° C.) to remove residual imidazole. The homogeneity and purity of the purified Fabs was determined by analysis on non-reduced and reduced SDS-PAGE.

ELISA with purified Fab antibodies: The binding specificity of individual soluble Fab fragments was determined by ELISA using biotinylated scMHC-peptide complexes. ELISA plates (Falcon) were coated overnight with BSA-biotin (1 μg/well). After having been washed, the plates were incubated (1 hour, RT) with streptavidin (1 μg/well), washed extensively and further incubated (1 hour, RT) with 0.5 μg of MHC/peptide complexes. Plates were blocked for 30 minutes at RT with PBS 2% BSA and subsequently were incubated for 1 hour at RT with various concentrations of soluble purified Fab, and after washing, with 1:1000 HRP-conjugated/anti-human antibody. Detection was performed using TMB reagent (Sigma). The HLA-A2-restricted peptides used for specificity studies of the purified Fab antibodies were: the Human Tyrosinase peptide 369-377 (YMDGTMSQV—SEQ ID NO: 1) and non-specific peptides MART1 (ELAGIGILTV—SEQ ID NO: 27).

Flow Cytometry: The EBV-transformed B-lymphoblast JY cells or virus infected cells as indicated were used to determine the reactivity of the recombinant TCRs with cell surface-expressed HLA-A2/peptide complexes About 10⁶ JY cells were washed with serum-free RPMI and incubated overnight at 37° C. in medium containing 100 μM of the peptide. The cells were incubated for 60 minutes at 4° C. with recombinant Fab antibodies or Fab-PE (10-100 μg/ml) in 100 μl. After three washes the cells were incubated with rabbit anti-PE polyclonal antibody or followed by washing twice with PBS and incubated for 60 minutes with FITC labeled anti-rabbit IgG(for Fab-PE) or with FITC-labeled anti-human Fab or with PE-labeled anti-human Fab (Jackson) (for Fab Abs). After a final wash, the cells were resuspended in ice-cold PBS.

The melanoma, leukemia, and glioblastoma HLA-A2⁺/hla-a2⁻ Tyr⁺/tyr⁻ positive cell lines were incubated for 60 minutes at 4° C. with recombinant Fab antibodies or Fab-PE (10-100 μg/ml) in 100 μl. After three washes the cells were incubated with rabbit anti-PE polyclonal antibody followed by washing twice with PBS and incubated for 60 minutes with FITC labeled anti rabbit IgG (for Fab-PE) or with FITC-labeled anti-human Fab or with PE-labeled anti-human Fab (Jackson) (for Fab Abs). After a final wash, the cells were resuspended in ice-cold PBS.

Binding Affinity determination: A SPR real-time kinetic interaction analysis system (BioRad Israel) was used to determine association (kon) and dissociation (koff) constants of the TA2, B2 (SEQ ID NOs; 892 and 893) and MC1 (SEQ ID NOs: 884 and 885) Fab antibodies. A biosensor chip (BioRad, Inc.) was activated according to the manufacturer's instructions and coupled with 86-300 RUs (response units) of Fab anti Fab in 10 mM sodium acetate (pH 4.5). Unreacted groups were blocked with 1 M ethanolamine. The kinetics of the Fabs binding to complex were measured with serial dilutions beginning with 1000 nM to 62.5 nM complex in running buffer (PBS, 0.05% (v/v) Tween, 0.01%). Binding measurements were recorded at 25° C. Data were fit to a 1:1 Langmuir binding model using Biorad evaluation software, which calculated kon koff rates. An equilibrium constant, KD, was calculated from koff/kon.

Expression and purification of Fab-PE38 fusion protein: The genes encoding the light and heavy chain of Fab TA2, MC1 were cloned separately into a T7-promotor pET-based expression vector Puli9 (KDEL version). The heavy chain gene was engineered to contain the PE38 recognition sequence at the COOH terminus (heavy-PE38). The genes encoding the variable heavy and light chain of TA2, MC1 Fab were cloned as scFv-PE-38 into a T7-promotor pET-based expression vector Puli9. (V_{H}-V_{L}-PE38).

These constructs were expressed separately in the Fab version or as one construct in the scFv version in E. coli BL21 cells and upon induction with IPTG, intracellular inclusion bodies which contain large amounts of the recombinant protein accumulated. Inclusion bodies of both chains of the Fab and the scFv construct were purified, reduced, and subsequently refolded, using a heavy PE38:light ratio of 2.5:1 for the Fab version, in a redox-shuffling buffer system containing 0.1 M Tris, 0.5 M Arginine, 0.09 mM Oxidized Glutathione (pH 7.4). Correctly folded Fab and scFv were then isolated and purified by ion-exchange chromatography Sepharose and MonoQ (Pharmacia).

Cytotoxicity assays: Melanoma cells were incubated with increasing concentrations of immunotoxin and the inhibition of protein synthesis was determined by measuring the uptake of ³H-Leucine into cellular proteins, as previously described [Reiter Y, et al., (1994) Int. J. Cancer 58, 142-149]. IC₅₀ was determined as the concentration of immunotoxin required to inhibit protein synthesis by 50%.

Results

Selection of TCR-like recombinant antibodies recognizing HLA-A2/Tyr₃₆₉₋₃₇₇: Recombinant peptide-HLA-A2 complexes that present the Tyr₃₆₉₋₃₇₇ Tyr derived peptide were generated using a scMHC construct that was described previously [Denkberg, G., Cohen, C. J., Segal, D., Kirkin, A. F., and Reiter, Y. (2000) Eur. J. Immunol. 30, 3522-3532]. In this construct, the extracellular domains of HLA-A2 are connected into a single chain molecule with B2m using a 15-aa flexible linker. The sc-MHC-peptide complexes were produced by in vitro refolding of inclusion bodies in the presence of the Tyr-derived Tyr₃₆₉₋₃₇₇ peptide. The refolded scHLA-A2/Tyr complexes were found to be very pure, homogenous, and monomeric by SDS-PAGE and size exclusion chromatography analyses (data not shown). Recombinant scMHC-peptide complexes generated by this strategy had been previously characterized in detail for their biochemical, biophysical, and biological properties, and were found to be correctly folded and functional [Denkberg, G., Cohen, C. J., Segal, D., Kirkin, A. F., and Reiter, Y. (2000) Eur. J. Immunol. 30, 3522-3532].

For selection of TCR-like Abs, a large Ab phage library was used, consisting of a repertoire of 3.7×10¹⁰ independent, human recombinant Fab clones. Initially, the library was depleted of streptavidin binders and subsequently subjected to selection in solution using soluble recombinant biotinylated scHLA-A2-peptide complexes containing the Tyr₃₆₉₋₃₇₇ peptide. In the conventional selection method, a 133-fold enrichment in phage titer was observed after three rounds of selection. In the off-rate selection the clones were picked after 2-rounds of selection where a 10-fold decrease in overall phage titer was observed between the second round and the first.

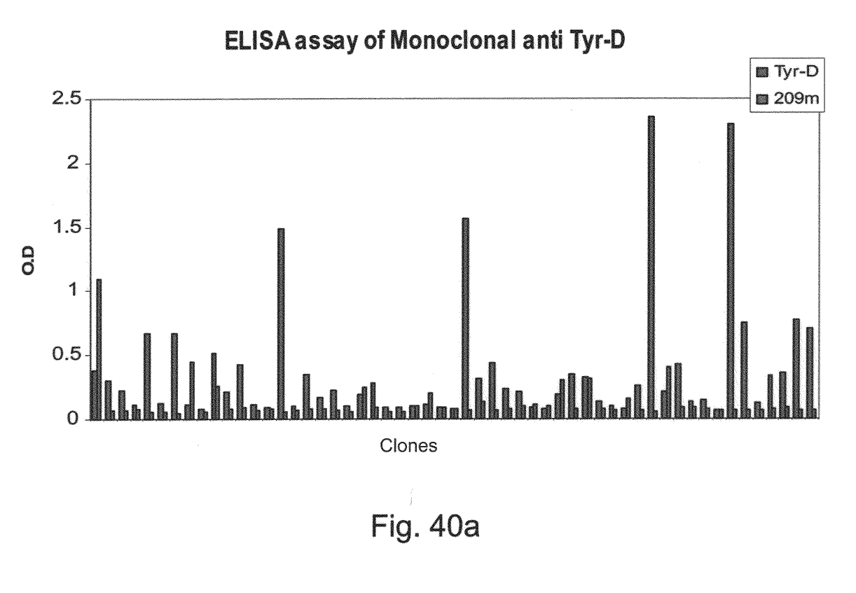

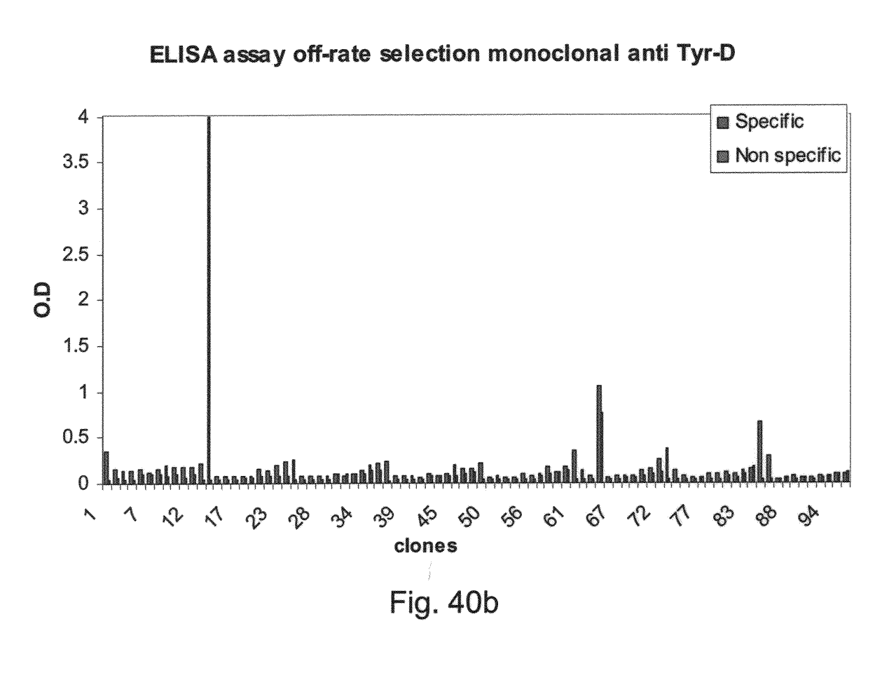

The specificity of the selected phage Abs was determined by a differential ELISA analysis on streptavidin-coated wells incubated with biotinylated scMHC HLA-A2 complexes containing either the Tyr₃₆₉₋₃₇₇ peptide or control complexes containing other HLA-A2-restricted peptides. Phage clones analyzed after the third round of selection in the conventional screening method exhibited two types of binding patterns toward the scHLA-A2-peptide complex. One class of Abs consisted of pan-MHC binders that could not differentiate between the various MHC-peptide complexes; the second type consisted of Abs that bound the MHC peptide complex in a peptide-specific manner. The ELISA screen revealed that 27 of 94 randomly selected clones screened (28%) from the third round of panning appeared to be fully peptide dependent and specific for the peptide/MHC used in the selection (i.e., the scHLA-A2/Tyr complex). A representative monoclonal ELISA analysis of TCR-like Fab clones is shown in FIG. 40a. The diversity within the selected TCR-like Fabs was assessed by DNA fingerprint analysis; 5 different antibodies with TCR-like specificity were revealed, indicating the selection of several different Abs with TCR-like specificity.

The specificity of the selected phage Abs isolated from the Off-Rate selection was determined by a differential ELISA analysis after the second round of selection on streptavidin-coated wells incubated with biotinylated scMHC HLA-A2 complexes containing either the Tyr₃₆₉₋₃₇₇ peptide or control complexes containing other HLA-A2-restricted peptides. Phage clones analyzed after the second round of selection exhibited the same two types of binding patterns toward the scHLA-A2− peptide complex. One class of Abs consisted of pan-MHC binders that could not differentiate between the various MHC-peptide complexes; the second type consisted of Abs that bound the MHC-peptide complex in a peptide-specific manner. The ELISA screen revealed that 1 of 94 randomly selected clones screened (1%) from the second round of panning appeared to be fully peptide dependent and specific for the peptide/MHC used in the selection (i.e., the scHLA-A2/Tyr complex).

A representative monoclonal ELISA analysis of TCR-like Fab clones isolated from the off-rate selection is shown in FIG. 40b. The diversity within the selected TCR-like Fab was assessed by DNA fingerprint analysis; 1 additional antibody with TCR-like specificity was revealed.

Characterization of Recombinant Soluble Fab Antibodies with TCR-Like Specificity. Of the five Fab clones recognizing the HLA-A2-Tyr₃₆₉₋₃₇₇ complex obtained by the conventional selection method, the one that exhibited the most specific peptide-dependent and TCR-like binding pattern as analyzed by the phage ELISAs, and the one positive clone from the off-rate selection, were subjected to further analysis.

The Fab clone-MC1 (from the conventional selection method) and B2 (from the off-rate selection) were sequenced and produced in a soluble form in E. coli TG1 or BL21 cells and were purified by immobilized metal ion affinity chromatography (IMAC). Yields were 2-4 mg of pure material from 1 liter of bacterial culture. SDS_PAGE analysis revealed a homogenous and pure population of Fabs with the expected molecular size (data not shown).

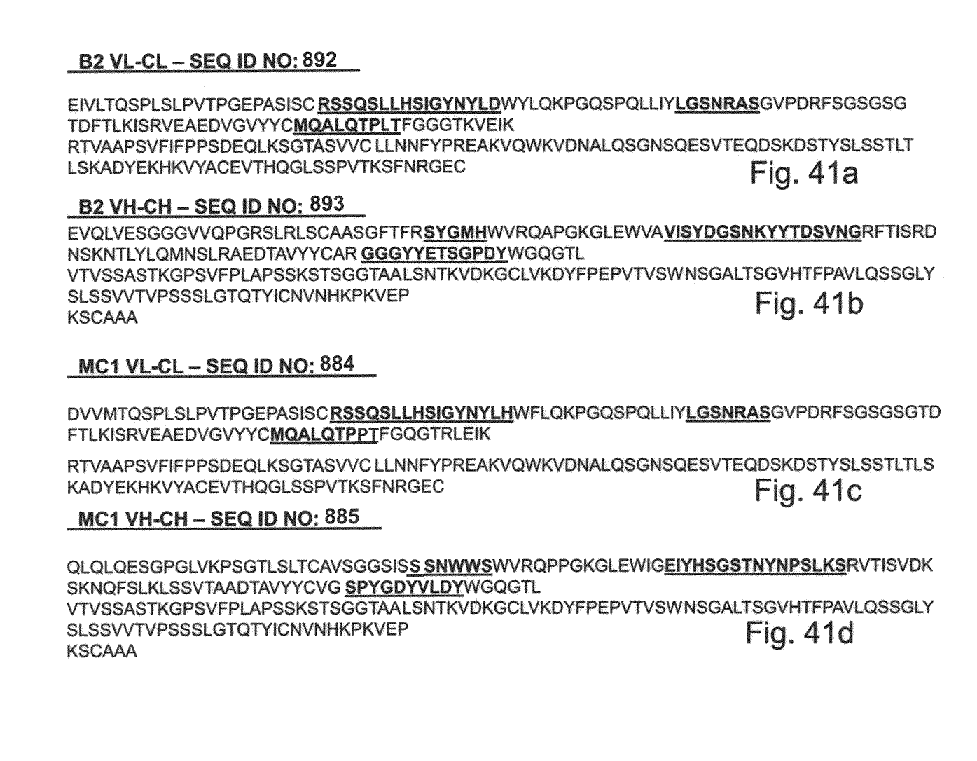

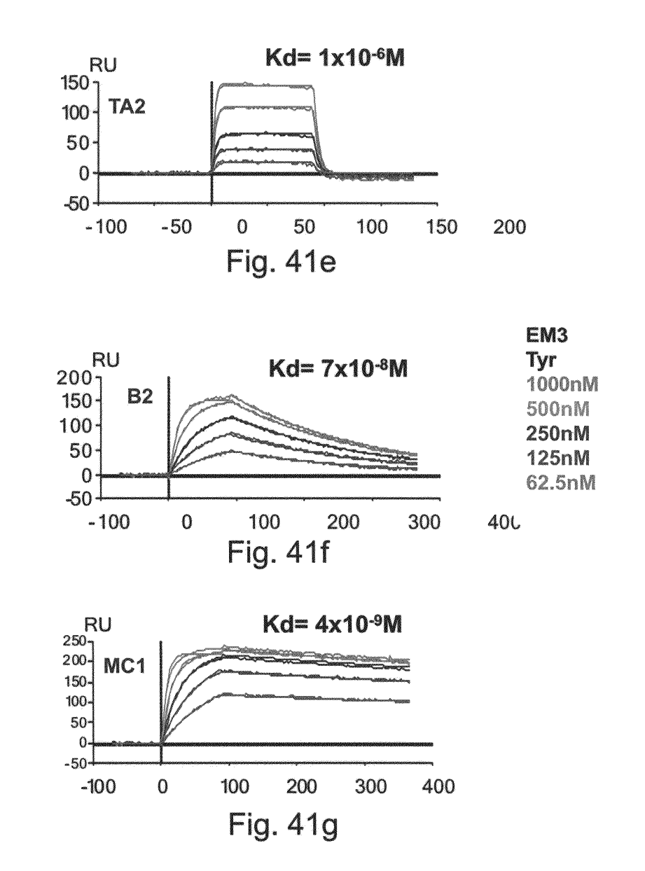

MC1 and B2 Fabs were sequenced. The B2 heavy chain belongs to subgroup III of VH and the light chain belongs to the human kappa subgroup II family. The MC1 heavy chain belongs to subgroup II of VH and the light chain belongs to the human kappa subgroup II family (FIGS. 41a-d).

The binding affinity of the purified Fab fragments MC1, B2, and TA2 that was previously isolated was determined by SPR analysis. Surface plasmon resonance (SPR) was used to measure the binding kinetics of Fabs to HLA-A2/Tyr complex on a Biorad instrument (as described in material and methods). Kinetics data for Fabs binding to HLA-A2/Tyr complex at 25° C. temperature are shown in FIGS. 41e-g. The Fab variants generally had very similar association constants but different dissociation rate constants, kₒₙ and k_{off} respectively. As a result, the mutants have different KD values of 1000 nM (TA2), 70 nM (B2) and 4 nM (MC1). The new screening methods revealed two Fab clones with improved affinity as compared to the TA2. B2 Fab had improved KD with an improvement over TA2 of about 10 fold. This improvement resulted primarily from a slower dissociation rate constant, k_{off} (Table 140, herein below, representing a mean of five experiments). MC1 Fab had improved KD with an improvement over TA2 of about 100 fold. This improvement resulted primarily from a slower dissociation rate constant, k_{off} (Table 140 herein below).

TABLE 140

| Rmax | KD | kd | ka | |
|---|---|---|---|---|
| RU | M | l/s | l/Ms | Antibody |
| 317.63 | 1.02E−06 | 0.24 | 2.37E+05 | TA2 |
| | (1000 nM) | | | |
| 174.65 | 6.69E−08 | 4.98E−03 | 7.45E+04 | B2 |
| | (70 nM) | | | |
| 231.22 | 4.15E−09 | 5.47E−04 | 1.32E+05 | MC1 |
| | (4 nM) | | | |

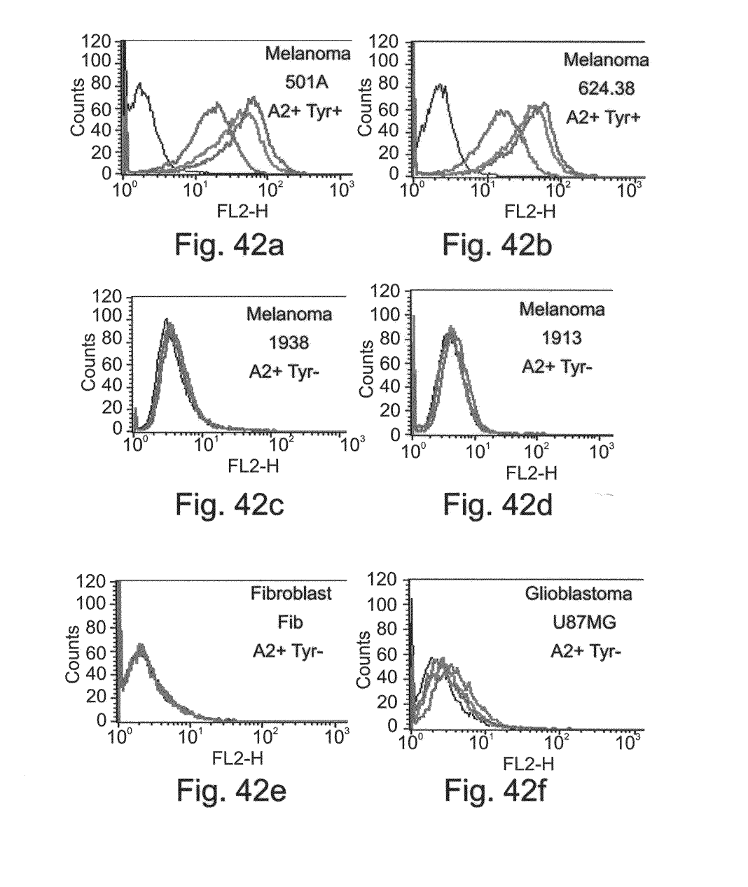

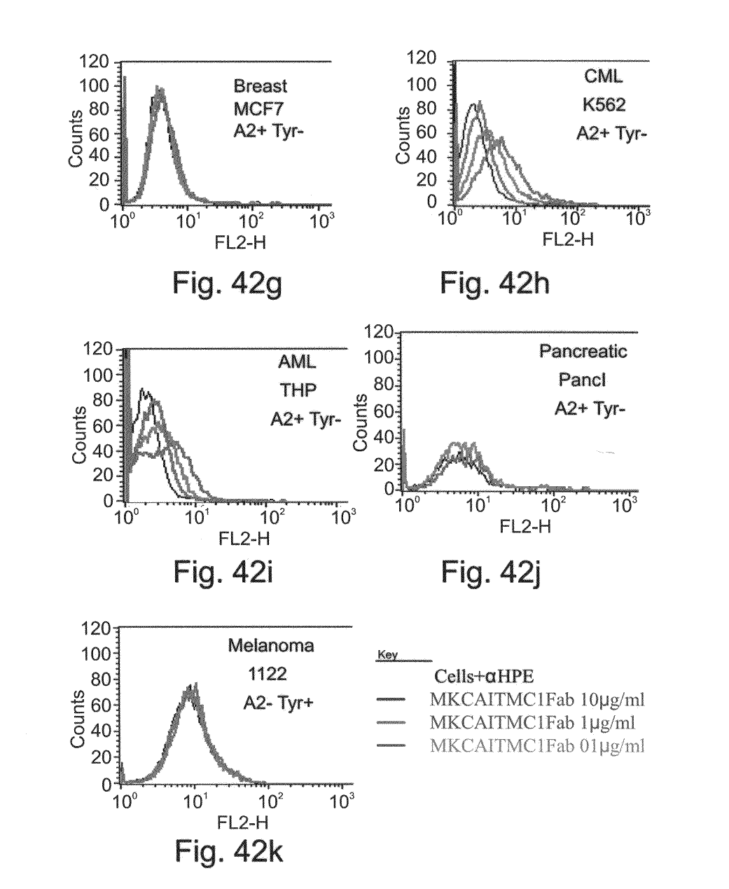

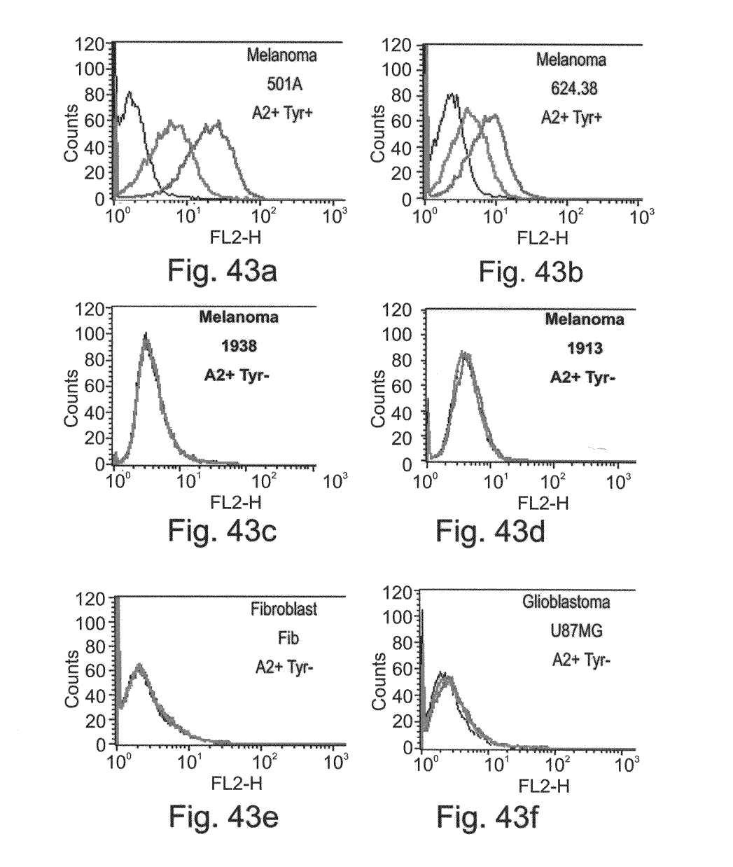

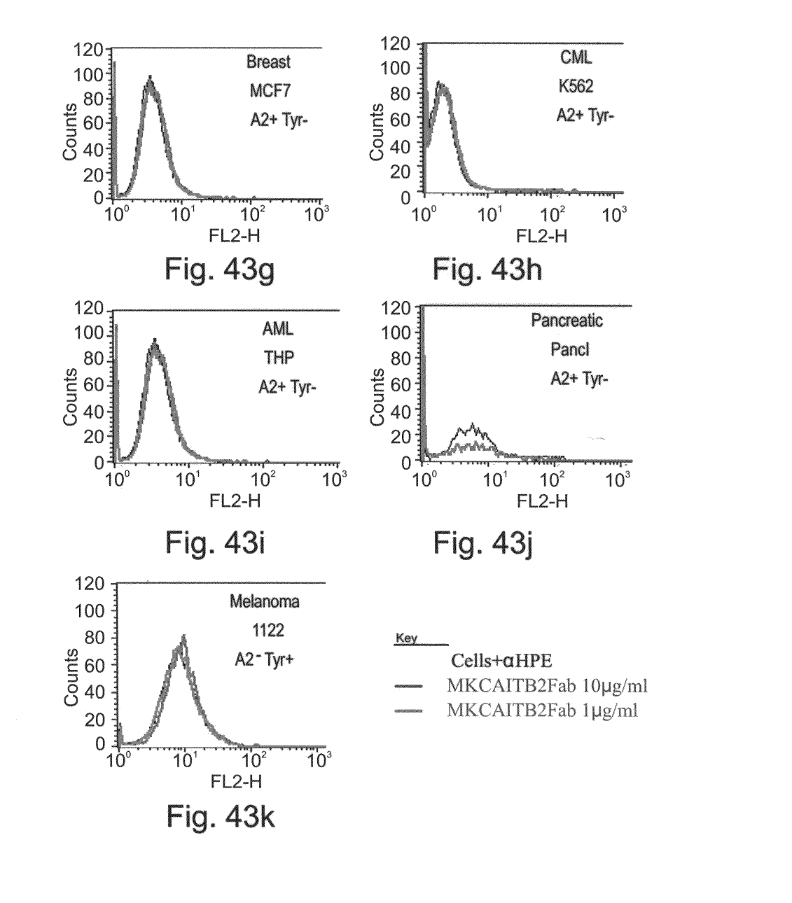

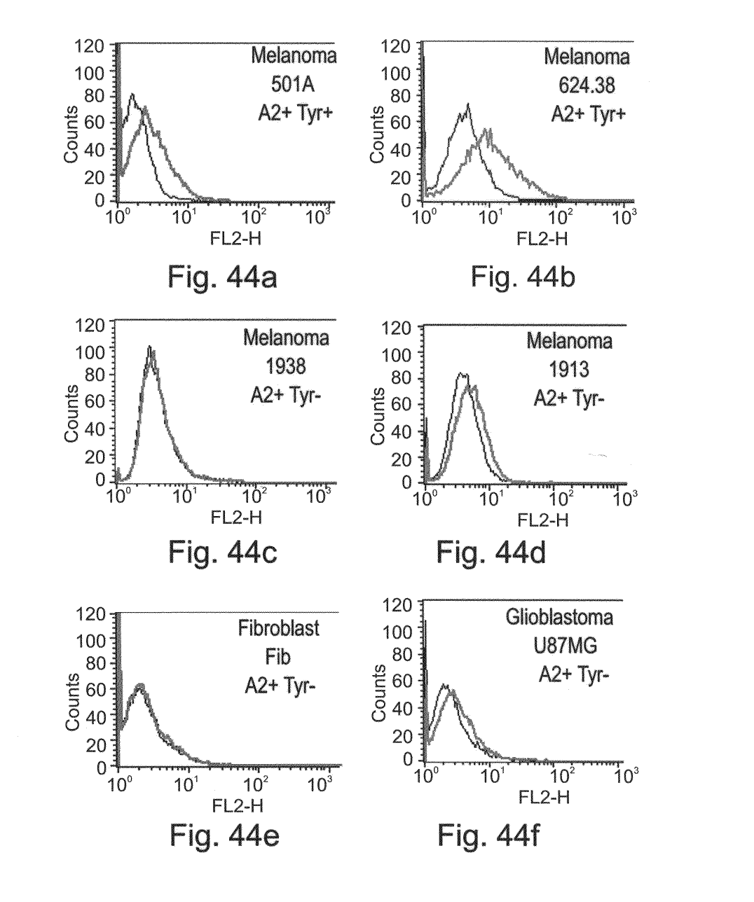

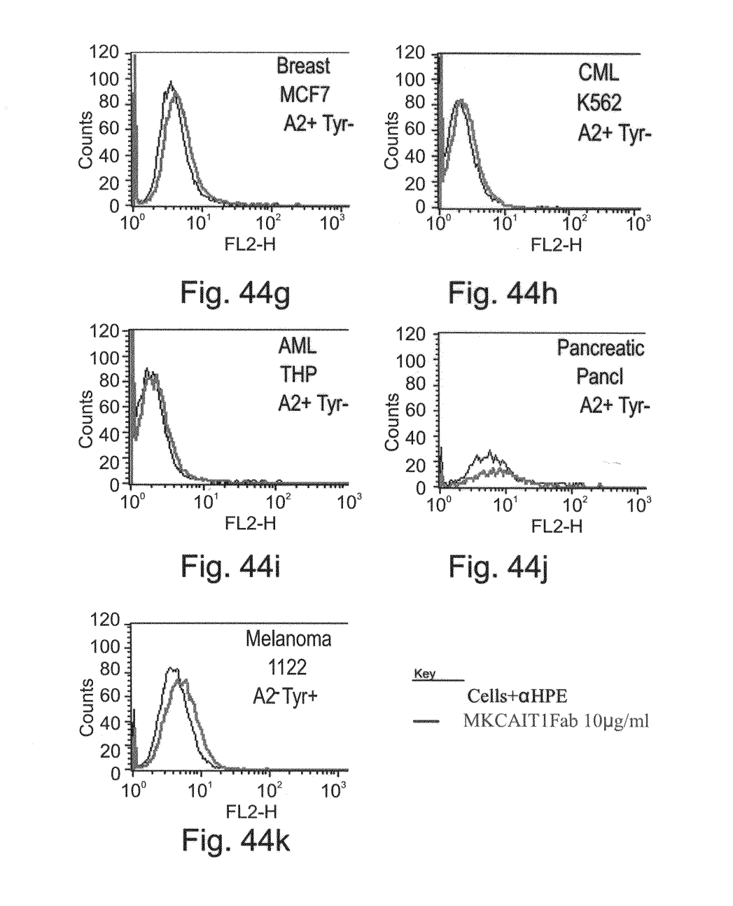

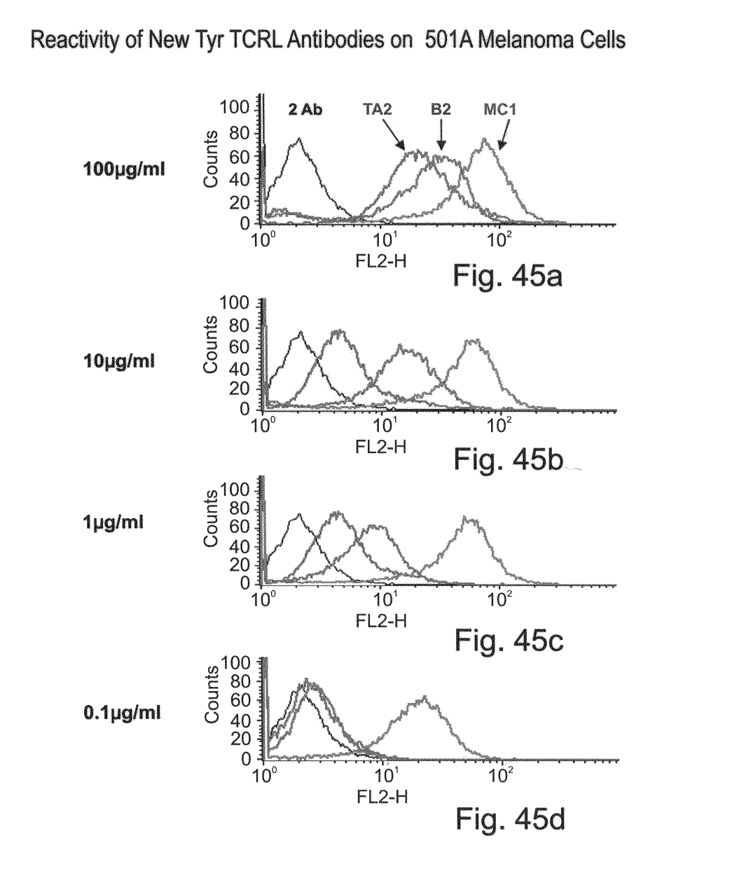

Binding of TA2, B2, MC1 Fab Antibodies to the surface of melanoma cells: To explore whether the HLA-A2/Tyrosinase TCR-like Fab Abs have different binding pattern to endogenously derived MHC-Tyrosinase complexes on the surface of tumor cells due to their different affinities, flow cytometry analysis was performed on lines derived from melanoma, leukemia and glioblastoma patients. Cells were incubated with TA2, B2, MC1 anti-Tyrosinase 369-377 Ab in different concentrations followed by incubation with PE-labeled anti-human antibody. As shown in FIGS. 42a-k, 43a-k and 44a-k, the MC1, B2, TA2 Fab respectively recognized Tyrosinase positive and HLA-A2 positive cells with a very high intensity corresponding to their improved affinity. The detection limit of TA2 was 10 μg/ml on positive cells while B2 had detection limit of 1 μg/ml on positive cells and MC1 reacted in 0.1 μg/ml on positive cells (FIG. 45a-d).

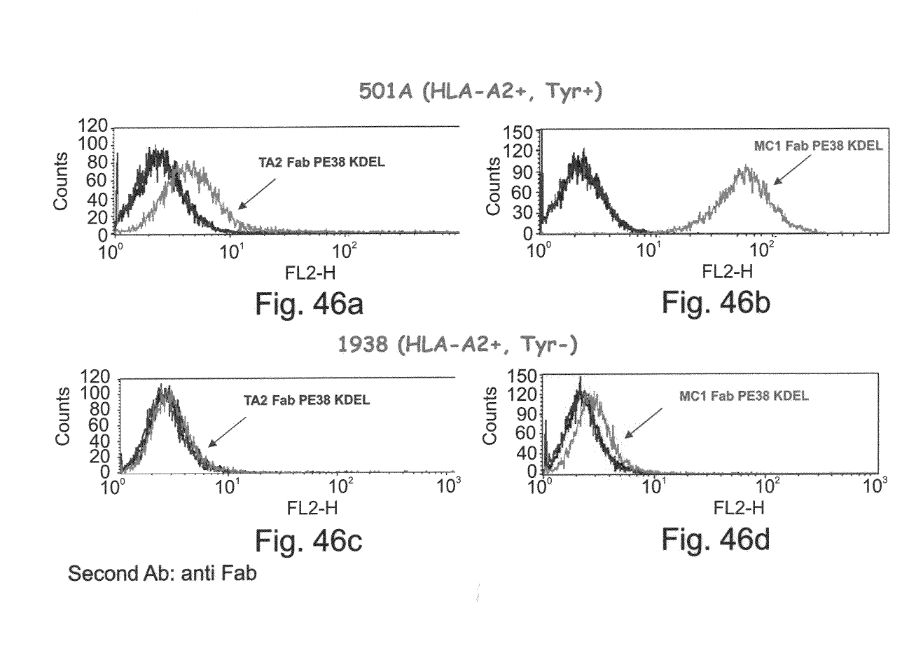

Binding of TA2, MC1 Fab-PE38 melanoma cells: To demonstrate that purified TA2 and MC1 Fab-PE38 can bind the specific MHC-peptide complex as expressed on the cell surface of Tyr-expressing tumor cells, these cells were incubated with the fusion proteins and were tested by monitoring the reactivity of anti-PE38 antibodies. As shown in FIG. 46a-d, the TA2 and MC1 Fab-toxin fusion proteins reacted specifically with HLA-A2 positive and Tyr positive 501A melanoma cells, but not with 1938 melanoma cells which express HLA-A2 but are Tyr negative.

These results demonstrate that the TA2 and MC1 Fab/scFv-PE38 retains their specificity to Tyr/HLA-A2 peptide complexes expressed on the surface of cells.

Cytotoxicity of TA2 and MC1 Fab- or scFv-PE38 towards tumor cells displaying the Tyr derived epitopes: The ability of the TA2 and MC1 Fab- or scFv-PE38 (KDEL version) to inhibit protein synthesis was used as a measure to test the specificity and biological activity of the TCR-like TA2 and MC1 Fab or scFv fusion molecules. Because the cell binding domain in the toxin was deleted, cytotoxicity induced by internalization of the Fab or scFv toxin fusion molecules reflects antigen-specific binding. To test this activity, HLA-A2 and Tyr positive 501A and 624.38 melanoma cells were incubated with increasing concentrations of the TA2 and MC1 Fab- or scFv-fusion proteins, and protein synthesis was tested by measuring incorporation of [3H]-leucine into cellular proteins. As controls HLA-A2 positive and Tyr negative 1938 melanoma cells were used.

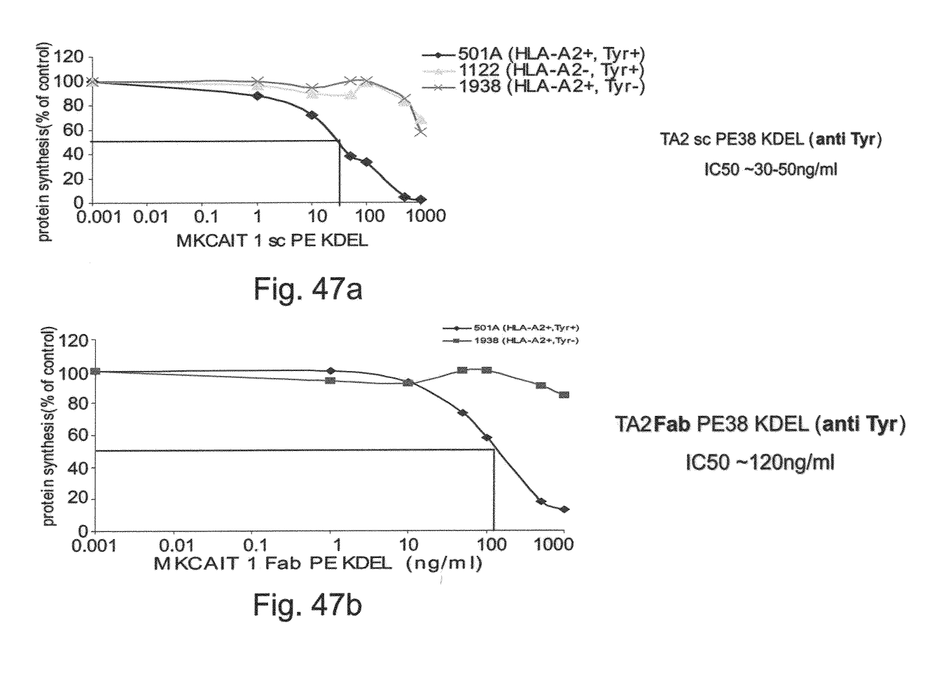

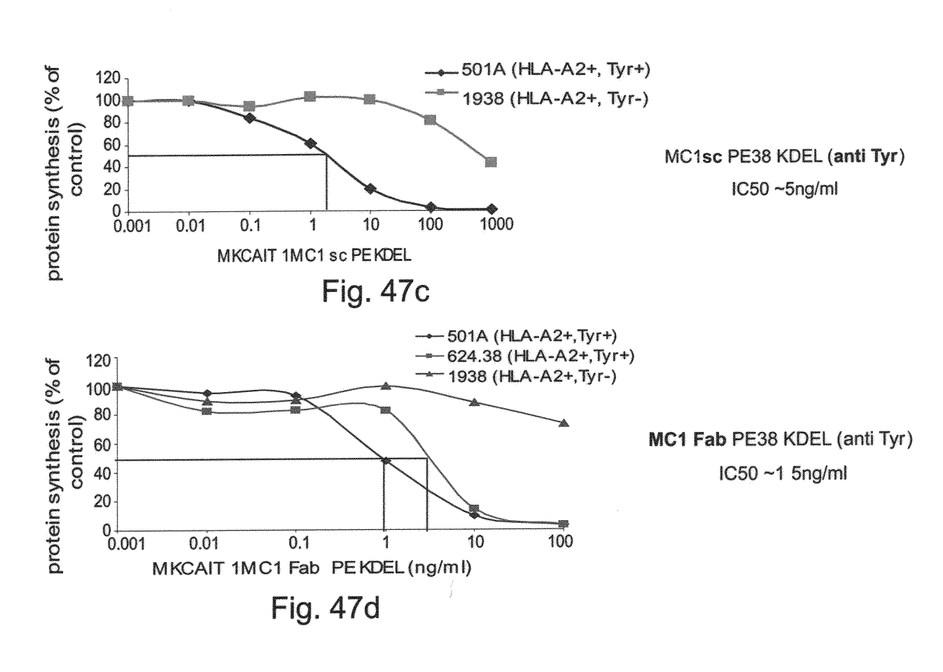

As shown in FIGS. 47a-d, the TA2 and MC1 Fab- or scFv-PE38 fusions inhibited (in a dose dependent manner) protein synthesis and were cytotoxic to 501A and 624.38 cells but not to control 1938 cells. The cytotoxic activity correlated the reactivity of the TA2 and MC1 TCR-like Fab; 501A cells reacted well with the antibodies and were killed more efficiently by the MC1 as compared to TA2.

A 100-fold increase in the IC₅₀ of the MC1 Fab-PE38 and a 10-fold increase in the IC₅₀ of the MC1 scFv-PE was observed compared to TA2 Fab/scFv-PE38 resulting from their higher affinity.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

### REFERENCES

### Additional References are Cited in Text

1. I. Komenaka, H. Hoerig, and H. L. Kaufman, “Immunotherapy for melanoma,” Clin. Dermatol. 22(3), 251 (2004).
2. G. Parmiani, et al., “Immunotherapy of melanoma,” Semin. Cancer Biol. 13(6), 391 (2003).
3. P. Romero, et al., “Antigenicity and immunogenicity of Melan-A/MART-1 derived peptides as targets for tumor reactive CTL in human melanoma,” Immunol. Rev. 188, 81 (2002).
4. V. H. Engelhard, et al., “Antigens derived from melanocyte differentiation proteins: self-tolerance, autoimmunity, and use for cancer immunotherapy,” Immunol. Rev. 188, 136 (2002).
5. T. Wolfel, et al., “Two tyrosinase nonapeptides recognized on HLA-A2 melanomas by autologous cytolytic T lymphocytes,” Eur. J. Immunol. 24(3), 759 (1994).
6. M. E. Dudley and S. A. Rosenberg, “Adoptive-cell-transfer therapy for the treatment of patients with cancer,” Nat. Rev. Cancer 3(9), 666 (2003).
7. C. J. Cohen, et al., “Recombinant antibodies with MHC-restricted, peptide-specific, T-cell receptor-like specificity: new tools to study antigen presentation and TCR-peptide-MHC interactions,” J. Mol. Recognit. 16(5), 324 (2003).
8. G. Denkberg, et al., “Selective targeting of melanoma and APCs using a recombinant antibody with TCR-like specificity directed toward a melanoma differentiation antigen,” J. Immunol. 171(5), 2197 (2003).
9. A. Lev, et al., “Isolation and characterization of human recombinant antibodies endowed with the antigen-specific, major histocompatibility complex-restricted specificity of T cells directed toward the widely expressed tumor T-cell epitopes of the telomerase catalytic subunit,” Cancer Res. 62(11), 3184 (2002).
10. C. J. Cohen, et al., “Direct phenotypic analysis of human MHC class I antigen presentation: visualization, quantitation, and in situ detection of human viral epitopes using peptide-specific, MHC-restricted human recombinant antibodies,” J. Immunol. 170(8), 4349 (2003).
11. J. Golay, et al., “Effect of alemtuzumab on neoplastic B cells,” Haematologica 89(12), 1476 (2004).
12. H. Mellstedt, “Monoclonal antibodies in human cancer,” Drugs Today (Barc.) 39 Suppl C, 1 (2003).
13. H. Modjtahedi, et al., “Targeting of cells expressing wild-type EGFR and type-III mutant EGFR (EGFRvIII) by anti-EGFR MAb ICR62: a two-pronged attack for tumour therapy,” Int. J. Cancer 105(2), 273 (2003).
14. N. Prang, et al., “Cellular and complement-dependent cytotoxicity of Ep-CAM-specific monoclonal antibody MT201 against breast cancer cell lines,” Br. J. Cancer 92(2), 342 (2005).
15. H. Takeuchi, et al., “Expression of differentiation melanoma-associated antigen genes is associated with favorable disease outcome in advanced-stage melanomas,” Cancer Res. 63(2), 441 (2003).
16. S. Reinke, et al., “Differential expression of MART-1, tyrosinase, and SM5-1 in primary and metastatic melanoma,” Am. J. Dermatopathol. 27(5), 401 (2005).
17. V. Brichard, et al., “The tyrosinase gene codes for an antigen recognized by autologous cytolytic T lymphocytes on HLA-A2 melanomas,” J. Exp. Med. 178(2), 489 (1993).
18. J. C. Skipper, et al., “An HLA-A2-restricted tyrosinase antigen on melanoma cells results from posttranslational modification and suggests a novel pathway for processing of membrane proteins,” J. Exp. Med. 183(2), 527 (1996).
19. C. A. Mosse, et al., “The class I antigen-processing pathway for the membrane protein tyrosinase involves translation in the endoplasmic reticulum and processing in the cytosol,” J. Exp. Med. 187(1), 37 (1998).
20. K. Jimbow, et al., “Assembly, target-signaling and intracellular transport of tyrosinase gene family proteins in the initial stage of melanosome biogenesis,” Pigment Cell Res. 13(4), 222 (2000).
21. V. H. Engelhard, A. G. Brickner, and A. L. Zarling, “Insights into antigen processing gained by direct analysis of the naturally processed class I MHC associated peptide repertoire,” Mol. Immunol. 39(3-4), 127 (2002).
22. R. Halaban, et al., “Abnormal acidification of melanoma cells induces tyrosinase retention in the early secretory pathway,” J. Biol. Chem. 277(17), 14821 (2002).
23. R. Halaban, et al., “Proper folding and endoplasmic reticulum to golgi transport of tyrosinase are induced by its substrates, DOPA and tyrosine,” J. Biol. Chem. 276(15), 11933 (2001).
24. G. Denkberg, et al., “Recombinant human single-chain MHC-peptide complexes made from E. coli By in vitro refolding: functional single-chain MHC-peptide complexes and tetramers with tumor associated antigens,” Eur. J. Immunol. 30(12), 3522 (2000).
25. W. J. Storkus, et al., “Identification of T-cell epitopes: rapid isolation of class I-presented peptides from viable cells by mild acid elution,” J. Immunother. 14(2), 94 (1993).
26. J. C. Skipper, et al., “Mass-spectrometric evaluation of HLA-A*0201-associated peptides identifies dominant naturally processed forms of CTL epitopes from MART-1 and gp100,” Int. J. Cancer 82(5), 669 (1999).
27. R. Halaban, et al., “Aberrant retention of tyrosinase in the endoplasmic reticulum mediates accelerated degradation of the enzyme and contributes to the dedifferentiated phenotype of amelanotic melanoma cells,” Proc. Natl. Acad. Sci. U.S. A 94(12), 6210 (1997).
28. D. Rimoldi, et al., “Subcellular localization of the melanoma-associated protein Melan-AMART-1 influences the processing of its HLA-A2-restricted epitope,” J. Biol. Chem. 276(46), 43189 (2001).
29. Y. Kawakami, et al., “Identification of the immunodominant peptides of the MART-1 human melanoma antigen recognized by the majority of HLA-A2-restricted tumor infiltrating lymphocytes,” J. Exp. Med. 180(1), 347 (1994).
30. Y. Kawakami, et al., “Recognition of shared melanoma antigens in association with major HLA-A alleles by tumor infiltrating T lymphocytes from 123 patients with melanoma,” J. Immunother. 23(1), 17 (2000).
31. D. Valmori, et al., “Analysis of the cytolytic T lymphocyte response of melanoma patients to the naturally HLA-A*0201-associated tyrosinase peptide 368-376,” Cancer Res. 59(16), 4050 (1999).
32. B. Rocha, A. Grandien, and A. A. Freitas, “Anergy and exhaustion are independent mechanisms of peripheral T cell tolerance,” J. Exp. Med. 181(3), 993 (1995).
33. L. S. Taams, Eden W. van, and M. H. Wauben, “Dose-dependent induction of distinct anergic phenotypes: multiple levels of T cell anergy,” J. Immunol. 162(4), 1974 (1999).
34. R. H. Schwartz, “T cell anergy,” Annu. Rev. Immunol. 21, 305 (2003);
35. W. R. Heath, et al., “Cross-presentation, dendritic cell subsets, and the generation of immunity to cellular antigens,” Immunol. Rev. 199, 9 (2004).
36. W. L. Redmond, B. C. Marincek, and L. A. Sherman, “Distinct requirements for deletion versus anergy during CD8 T cell peripheral tolerance in vivo,” J. Immunol. 174(4), 2046 (2005).
37. J. F. Miller, et al., “Induction of peripheral CD8+ T-cell tolerance by cross-presentation of self antigens,” Immunol. Rev. 165, 267 (1998).
38. R. H. Schwartz, “T cell anergy,” Annu. Rev. Immunol. 21, 305 (2003).
39. T. R. Mosmann and S. Sad, “The expanding universe of T-cell subsets: Th1, Th2 and more,” Immunol. Today 17(3), 138 (1996).
40. F. Sallusto, et al., “Two subsets of memory T lymphocytes with distinct homing potentials and effector functions,” Nature 401(6754), 708 (1999).
41. D. J. Irvine, et al., “Direct observation of ligand recognition by T cells,” Nature 419(6909), 845 (2002).
42. A. Grakoui, et al., “The immunological synapse: a molecular machine controlling T cell activation,” Science 285(5425), 221 (1999).
43. Baarle D. van, et al., “Significance of senescence for virus-specific memory T cell responses: rapid ageing during chronic stimulation of the immune system,” Immunol. Lett. 97(1), 19 (2005).
44. J. Zhou, et al., “Selective growth, in vitro and in vivo, of individual T cell clones from tumor-infiltrating lymphocytes obtained from patients with melanoma,” J. Immunol. 173(12), 7622 (2004).
45. A. Lev, et al., “Tumor-specific Ab-mediated targeting of MHC-peptide complexes induces regression of human tumor xenografts in vivo,” Proc. Natl. Acad. Sci. U.S.A 101(24), 9051 (2004).
46. C. J. Cohen, et al., “Recombinant antibodies with MHC-restricted, peptide-specific, T-cell receptor-like specificity: new tools to study antigen presentation and TCR-peptide-MHC interactions,” J. Mol. Recognit. 16(5), 324 (2003).
47. G. Denkberg, et al., “Selective targeting of melanoma and APCs using a recombinant antibody with TCR-like specificity directed toward a melanoma differentiation antigen,” J. Immunol. 171(5), 2197 (2003).
48. T. R. Mempel, S. E. Henrickson, and U. H. Von Andrian, “T-cell priming by dendritic cells in lymph nodes occurs in three distinct phases,” Nature 427(6970), 154 (2004).
49. S. C. Jameson, K. A. Hogquist, and M. J. Bevan, “Specificity and flexibility in thymic selection,” Nature 369(6483), 750 (1994).
50. M. Krogsgaard, et al., “Agonist/endogenous peptide-MHC heterodimers drive T cell activation and sensitivity,” Nature 434(7030), 238 (2005).
51. C. J. Fox, P. S. Hammerman, and C. B. Thompson, “Fuel feeds function: energy metabolism and the T-cell response,” Nat. Rev. Immunol. 5(11), 844 (2005).
52. G. Dennis, Jr., et al., “DAVID: Database for Annotation, Visualization, and Integrated Discovery,” Genome Biol. 4(5), 3 (2003).
53. D. A. Hosack, et al., “Identifying biological themes within lists of genes with EASE,” Genome Biol. 4(10), R70 (2003).
54. F. Ramsdell and B. J. Fowlkes, “Maintenance of in vivo tolerance by persistence of antigen,” Science 257(5073), 1130 (1992).

LENGTHY TABLES
The patent application contains a lengthy table section. A copy of the table is available in electronic form from the USPTO web site (http://seqdata.uspto.gov/?pageRequest=docDetail&DocID=US20100158927A1). An electronic copy of the table will also be available from the USPTO upon request and payment of the fee set forth in 37 CFR 1.19(b)(3).

1. An antibody or an antibody fragment comprising an antigen recognition domain capable of binding to an MHC-I molecule being complexed with a tyrosinase peptide comprising an amino acid sequence as set forth in SEQ ID NO: 1, wherein the antibody does not bind said MHC-I in the absence of said complexed peptide, and wherein the antibody does not bind said peptide in an absence of said MHC.

2. The antibody or the antibody fragment of claim 1, comprising an antigen recognition domain which comprise complementarity determining region (CDR) amino acid sequences as set forth in SEQ ID NOs: 59-64.

3. (canceled)

4. The antibody or the antibody fragment of claim 1, comprising an antigen recognition domain which comprises complementarity determining region (CDR) amino acid sequences as set forth in SEQ ID NOs: 886-891.

5. The antibody or the antibody fragment of claim 1, comprising an antigen recognition domain which comprises complementarity determining region (CDR) amino acid sequences as set forth in SEQ ID NOs: 894-899.

6. (canceled)

7. The antibody of claim 1, being an IgG1 antibody.

8. The antibody of claim 1, being conjugated to a therapeutic moiety, a toxic moiety or a detectable moiety.

9. (canceled)

10. The antibody of claim 8, wherein said toxic moiety is PE38 KDEL.

11-13. (canceled)

14. The antibody of claims 1, wherein said antibody fragment is selected from the group consisting of an Fab fragment, an F(ab′)₂ fragment and a single chain Fv fragment.

15. A pharmaceutical composition comprising the antibody of claim 1.

16. A method of detecting a melanoma cell, comprising contacting the cell with an antibody comprising an antigen recognition domain capable of binding to an MHC-I molecule being complexed with a tyrosinase peptide, wherein the antibody does not bind said MHC-I in the absence of said complexed peptide, and wherein the antibody does not bind said peptide in an absence of said MHC, under conditions which allow immunocomplex formation, wherein a presence of said immunocomplex or level thereof is indicative of the melanoma cell.

17. A method of diagnosing a melanoma in a subject in need thereof, comprising contacting a cell of the subject with an antibody comprising an antigen recognition domain capable of binding to an MHC-I molecule being complexed with a tyrosinase peptide, wherein the antibody does not bind said MHC-I in the absence of said complexed peptide, and wherein the antibody does not bind said peptide in an absence of said MHC, under conditions which allow immunocomplex formation, wherein a presence of said immunocomplex or level thereof is indicative of the melanoma.

18. (canceled)

19. The method of claim 16, wherein said tyrosinase peptide comprises an amino acid sequence as set forth in SEQ ID NO: 1.

20-23. (canceled)

24. The method of claim 16, wherein said antibody is attached to a detectable moiety.

25. (canceled)

26. A method of identifying if a subject is suitable for TCRL-based epitope directed therapy, comprising determining a level of epitope presentation on at least one cell of the subject using an antibody comprising an antigen recognition domain capable of binding to an MHC-I molecule being complexed with a peptide fragment of said antigen, wherein the antibody does not bind said MHC-I in the absence of said complexed peptide fragment of said antigen, and wherein the antibody does not bind said peptide fragment of said antigen in an absence of said MHC, wherein a level value higher than a predetermined threshold is indicative of an individual being suitable for TCRL-based epitope directed therapy.

27. The method of claim 26, wherein a level value below a predetermined threshold is indicative of an individual not being suitable for TCRL-based epitope directed therapy.

28. A method of identifying if a subject is suitable for CTL-based epitope directed therapy, comprising determining a level of epitope presentation on at least one cell of the subject using an antibody comprising an antigen recognition domain capable of binding to an MHC-I molecule being complexed with a peptide fragment of said antigen, wherein the antibody does not bind said MHC-I in the absence of said complexed peptide fragment of said antigen, and wherein the antibody does not bind said peptide fragment of said antigen in an absence of said MHC, wherein a level value lower than a predetermined threshold is indicative of an individual being suitable for CTL-based epitope directed therapy.

29. A method of treating a melanoma, comprising administering to a subject in need thereof a therapeutically effective amount of an antibody comprising an antigen recognition domain capable of binding to an MHC-I molecule being complexed with a tyrosinase peptide, wherein said antibody does not bind said MHC-I in the absence of said complexed tyrosinase peptide, and wherein said antibody does not bind said tyrosinase peptide in an absence of said MHC, thereby treating the melanoma.

30. The method of claim 29, wherein said tyrosinase peptide comprises an amino acid sequence as set forth in SEQ ID NO: 1.

31-37. (canceled)

38. A method of killing or ablating a cell displaying a tyrosinase peptide on a surface MHC molecule, the method comprising contacting the target cell with an antibody comprising an antigen recognition domain capable of binding to an MHC-I molecule being complexed with a tyrosinase peptide, wherein said antibody does not bind said MHC-I in the absence of said complexed tyrosinase peptide, and wherein said antibody does not bind said tyrosinase peptide in an absence of said MHC, thereby killing or ablating the cell.

39. The method of claim 38, wherein said tyrosinase peptide comprises an amino acid sequence as set forth in SEQ ID NO: 1.

40-46. (canceled)

47. An antibody comprising an antigen recognition domain capable of binding to an MHC-I molecule being complexed with a MART-1 peptide comprising an amino acid sequence as set forth in SEQ ID NO: 21, wherein the antibody does not bind said MHC-I in the absence of said complexed peptide, and wherein the antibody does not bind said peptide in an absence of said MHC.

48. The antibody of claim 47, comprising an antigen recognition domain which comprise complementarity determining region (CDR) amino acid sequences as set forth in SEQ ID NOs: 47-52.

49. The antibody of claim 47, comprising an antigen recognition domain which comprises complementarity determining region (CDR) amino acid sequences as set forth in SEQ ID NOs: 53-58.


SEQUENCE LISTING (910 sequences)

SEQ ID NO: 1 - protein, 9 aa, Artificial sequence
  Tyrosinase D 369-377 derived peptide
YMDGTMSQV

SEQ ID NO: 2 - protein, 9 aa, Artificial sequence
  Gag derived peptide
SLYNTVATL

SEQ ID NO: 3 - protein, 9 aa, Artificial sequence
  Tyr N derived peptide
YMNGTMSQV

SEQ ID NO: 4 - protein, 9 aa, Artificial sequence
  gp100 209-217 derived peptide (G9-209 2M)
IMDQVPFSV

SEQ ID NO: 5 - protein, 9 aa, Artificial sequence
  gp100 280-288 derived peptide (G9-280)
YLEPGPVTA

SEQ ID NO: 6 - protein, 9 aa, Artificial sequence
  hTERT 540-548 derived peptide
ILAKFLHWL

SEQ ID NO: 7 - protein, 9 aa, Artificial sequence
  TARP 29-37 peptide
FLRNFSLML

SEQ ID NO: 8 - DNA, 21 bp, Artificial sequence
  Single strand DNA oligonucleotide
gtgccctgaccctacaagatg

SEQ ID NO: 9 - DNA, 21 bp, Artificial sequence
  Single strand DNA oligonucleotide
cataagcaggtggagcattgg

SEQ ID NO: 10 - DNA, 20 bp, Artificial sequence
  Single strand DNA oligonucleotide
tattgaaagtgccgagatcc

SEQ ID NO: 11 - DNA, 19 bp, Artificial sequence
  Single strand DNA oligonucleotide
tgcaaggaccacagccatc

SEQ ID NO: 12 - DNA, 24 bp, Artificial sequence
  Single strand DNA oligonucleotide
atggaacgcccgagggacctttac

SEQ ID NO: 13 - DNA, 24 bp, Artificial sequence
  Single strand DNA oligonucleotide
tgagaggcatccgctatcccagta

SEQ ID NO: 14 - DNA, 20 bp, Artificial sequence
  Single strand DNA oligonucleotide
tattgggcgcctggtcacca

SEQ ID NO: 15 - DNA, 20 bp, Artificial sequence
  Single strand DNA oligonucleotide
ccaccttcttgatgtcatca

SEQ ID NO: 16 - DNA, 660 bp, Artificial sequence
  TA2-VLCL coding sequence
atggatgttgtgatgactcagtctccactctccctgcccgtcacccctggagagccggcctccatctcctgcaggtctagtcagagcctcctgcatagtaatggacacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaactctacaaactcctctcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 17 - protein, 219 aa, Artificial sequence
  TA2-VLCL(Human kappa subgroup II)
DVVMTQSPLSLPVTPGEPASISCRSSQSLLHSNGHNYLDWYLQKPGQSPQLLIYLGSNRASGVPDRFSGSGSGTDFTLKISRVEAEDVGVYYCMQTLQTPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 18 - DNA, 690 bp, Artificial sequence
  TA2-VH CH coding sequence
atggaggtccagctggtggagaccgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatcgttatggctggggatcgtcctttgggcactttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgca

SEQ ID NO: 19 - protein, 229 aa, Artificial sequence
  TA2-VH CH
EVQLVETGGGVVQPGRSLRLSCAASGFTFSSYGMHWVRQAPGKGLEWVAVISYDGSNKYYADSVKGRFTISRDNSKNTLYLQMNSLRAEDTAVYYCAKDRYGWGSSFGHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 20 - protein, 9 aa, Artificial sequence
  gp100 154-162 peptide
KTWGQYWQV

SEQ ID NO: 21 - protein, 10 aa, Artificial sequence
  MART1 26-35  peptide
EAAGIGILTV

SEQ ID NO: 22 - protein, 9 aa, Artificial sequence
  MART1 27-35  peptide
AAGIGILTV

SEQ ID NO: 23 - protein, 15 aa, Artificial sequence
  Flexible peptide linker
GGGGSGGGGSGGGGS

SEQ ID NO: 24 - protein, 9 aa, Artificial sequence
  EBV BMLF-1 280-288 derived peptide
GLCTLVAML

SEQ ID NO: 25 - protein, 9 aa, Artificial sequence
  CMV P65 495-503 derived peptide
NLVPMVATV

SEQ ID NO: 26 - protein, 9 aa, Artificial sequence
  HTLV-1 TAX1 1-19 derived peptide
LLFGYPVYV

SEQ ID NO: 27 - protein, 10 aa, Artificial sequence
  Mart-1 27L peptide
ELAGIGILTV

SEQ ID NO: 28 - protein, 9 aa, Artificial sequence
  gp100 280M peptide
YLEPGPVTV

SEQ ID NO: 29 - protein, 9 aa, Artificial sequence
  hTERT865-873 derived peptide
RLVDDFLLV

SEQ ID NO: 30 - protein, 10 aa, Artificial sequence
  XAGE-1 peptide
GVFPSAPSPV

SEQ ID NO: 31 - protein, 216 aa, Artificial sequence
  CLA12-VLCL (Human lambda subgruop I)
QSVVTQPPSVSAAPGRKVTISCSGSSSNIGSNYVSWYQQVPGTAPKLLIYEDDKRPSGIPDRFSGSKSGTSATLGITGLQTGDEADYFCATWDRTVNVVRFGGGTRLTVHSQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 32 - protein, 221 aa, Artificial sequence
  CLA12-VHCH (subgroup II)
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHVGHELDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 217 aa, Artificial sequence
  CAG10- VLCL (Human lambda subgroup I)
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDNSLSSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 222 aa, Artificial sequence
  CAG10-VHCH (subgroup  I)
EVQLQQSGAEVKKPGSSVKVSCKASGGTFSSYAISWVRQAPGQGLEWMGRIIPILGIANYAQKFQGRVTITADKSTSTAYMELSSLRSEDTAVYYCARDVGSGSYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPSC

SEQ ID NO: 35 - DNA, 648 bp, Artificial sequence
  CLA12-VLCL (Human lambda subgroup I)  coding sequence
cagtctgtcgtgacgcagccgccctcagtgtctgcggccccaggacggaaggtcaccatctcctgctctggaagcagctccaacattgggagtaattatgtgtcctggtaccagcaagtcccagggacagcccccaaactcctcatctatgaagatgataagcgaccctcagggattcctgaccgattctctggctccaagtctgggacgtccgccaccctgggcatcaccggactacagactggggacgaggccgattatttctgcgccacgtgggataggactgtcaatgttgtacgtttcggcggagggaccaggctgaccgtccacagtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 36 - DNA, 663 bp, Artificial sequence
  CLA12-VHCH (Human lambda subgroup I)  coding sequence
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacatgtgggccacgaacttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 37 - DNA, 672 bp, Artificial sequence
  CAG10- VLCL (Human lambda subgroup I) coding sequence
gaaggagatatacatatggcacagtctgtgttgacgcagccgccctcagtgtctggggccccagggcagagggtcaccatctcctgcactgggagcagctccaacatcggggcaggttatgatgtacactggtaccagcagcttccgggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacaacagcctgagtagttgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 38 - DNA, 660 bp, Artificial sequence
  CAG10-VHCH (subgroup  I) coding sequence
gaggtacagctgcagcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagatgttgggtcggggagttattcccttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagccc

SEQ ID NO: 39 - DNA, 744 bp, Artificial sequence
  CAG10- VLCL-BirA Tag coding sequence
gaaggagatatacatatggcacagtctgtgttgacgcagccgccctcagtgtctggggccccagggcagagggtcaccatctcctgcactgggagcagctccaacatcggggcaggttatgatgtacactggtaccagcagcttccgggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacaacagcctgagtagttgggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcacccggatccctgcaccatatcctggacgcccagaagatggtgtggaatcaccgctaatgaattcggctgcta

SEQ ID NO: 40 - protein, 242 aa, Artificial sequence
  CAG10- VLCL-BirA Tag
EGDIHMAQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDNSLSSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSPGSLHHILDAQKMVWNHR

SEQ ID NO: 41 - DNA, 768 bp, Artificial sequence
  CLA12-VLCL-BirA Tag
caacggtttccctctagaaataattttgtttaactttaagaaggagatatacatatggcacagtctgtcgtgacgcagccgccctcagtgtctgcggccccaggacggaaggtcaccatctcctgctctggaagcagctccaacattgggagtaattatgtgtcctggtaccagcaagtcccagggacagcccccaaactcctcatctatgaagatgataagcgaccctcagggattcctgaccgattctctggctccaagtctgggacgtccgccaccctgggcatcaccggactacagactggggacgaggccgattatttctgcgccacgtgggataggactgtcaatgttgtacgtttcggcggagggaccaggctgaccgtccacagtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcacccggatccctgcaccatatcctggacgcccagaagatggtgtggaatcaccgctaatga

SEQ ID NO: 42 - protein, 236 aa, Artificial sequence
  CLA12-VLCL-BirA Tag
MAQSVVTQPPSVSAAPGRKVTISCSGSSSNIGSNYVSWYQQVPGTAPKLLIYEDDKRPSGIPDRFSGSKSGTSATLGITGLQTGDEADYFCATWDRTVNVVRFGGGTRLTVHSQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECSPGSLHHILDAQKMVWNHR

SEQ ID NO: 43 - DNA, 807 bp, Artificial sequence
  CLA12-VHCH-His MYC Tag coding sequence
caacggtttccctctagaaataattttgtttaactttaagaaggagatatacatatggccgagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacatgtgggccacgaacttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcataatga

SEQ ID NO: 44 - protein, 249 aa, Artificial sequence
  -VHCH-His MYC Tag
MAELQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHVGHELDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 45 - DNA, 759 bp, Artificial sequence
  CAG10-VHCH-His MYC Tag coding sequence
taactttaagaaggagatatacatatggccgaggtacagctgcagcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagagatgttgggtcggggagttattcccttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaantcttgtgcggccgcacatcatcatcaccatcacggggccgcagaacaaaaactcatctcagaagag

SEQ ID NO: 46 - protein, 244 aa, Artificial sequence
  CAG10-VHCH-His MYC Tag
MAEVQLQQSGAEVKKPGSSVKVSCKASGGTFSSYAISWVRQAPGQGLEWMGRIIPILGIANYAQKFQGRVTITADKSTSTAYMELSSLRSEDTAVYYCARDVGSGSYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPSCAAAHHHHHHGAAEQKLISEE

SEQ ID NO: 47 - protein, 13 aa, Artificial sequence
  CLA12-VLCL CDR
SGSSSNIGSNYVS

SEQ ID NO: 48 - protein, 7 aa, Artificial sequence
  CLA12-VLCL CDR
EDDKRPS

SEQ ID NO: 49 - protein, 11 aa, Artificial sequence
  CLA12-VLCL CDR
ATWDRTVNVVR

SEQ ID NO: 50 - protein, 7 aa, Artificial sequence
  CLA12-VHCH CDR
SSSYYWG

SEQ ID NO: 51 - protein, 16 aa, Artificial sequence
  CLA12-VHCH CDR
SIYYSGSTYYNPSLKS

SEQ ID NO: 52 - protein, 8 aa, Artificial sequence
  CLA12-VHCH CDR
HVGHELDY

SEQ ID NO: 53 - protein, 14 aa, Artificial sequence
  CAG10- VLCL CDR
TGSSSNIGAGYDVH

SEQ ID NO: 54 - protein, 7 aa, Artificial sequence
  CAG10- VLCL CDR
GNSNRPS

SEQ ID NO: 55 - protein, 11 aa, Artificial sequence
  CAG10- VLCL CDR
QSYDNSLSSWV

SEQ ID NO: 56 - protein, 5 aa, Artificial sequence
  CAG10-VHCH CDR
SYAIS

SEQ ID NO: 57 - protein, 17 aa, Artificial sequence
  CAG10-VHCH CDR
RIIPILGIANYAQKFQG

SEQ ID NO: 58 - protein, 11 aa, Artificial sequence
  CAG10-VHCH CDR
DVGSGSYSLDY

SEQ ID NO: 59 - protein, 16 aa, Artificial sequence
  TA2-VLCL CDR
RSSQSLLHSNGHNYLD

SEQ ID NO: 60 - protein, 7 aa, Artificial sequence
  TA2-VLCL CDR
LGSNRAS

SEQ ID NO: 61 - protein, 9 aa, Artificial sequence
  TA2-VLCL CDR
MQTLQTPLT

SEQ ID NO: 62 - protein, 5 aa, Artificial sequence
  TA2-VHCH CDR
SYGMH

SEQ ID NO: 63 - protein, 17 aa, Artificial sequence
  TA2-VHCH CDR
VISYDGSNKYYADSVKG

SEQ ID NO: 64 - protein, 14 aa, Artificial sequence
  TA2-VHCH CDR
DRYGWGSSFGHFDY

SEQ ID NO: 65 - protein, 8 aa, Artificial sequence
  Pol derived peptide
ILEPVHGV

SEQ ID NO: 66 - DNA, 54 bp, Artificial sequence
  Bir A tag expressing sequence
cccggatccctgcaccatatcctggacgcccagaagatggtgtggaatcaccgc

SEQ ID NO: 67 - protein, 529 aa, Artificial sequence
  Single strand DNA oligonucleotide
MLLAVLYCLLWSFQTSAGHFPRACVSSKNLMEKECCPPWSGDRSPCGQLSGRGSCQNILLSNAPLGPQFPFTGVDDRESWPSVFYNRTCQCSGNFMGFNCGNCKFGFWGPNCTERRLLVRRNIFDLSAPEKDKFFAYLTLAKHTISSDYVIPIGTYGQMKNGSTPMFNDINIYDLFVWIHYYVSMDALLGGYEIWRDIDFAHEAPAFLPWHRLFLLRWEQEIQKLTGDENFTIPYWDWRDAEKCDICTDEYMGGQHPTNPNLLSPASFFSSWQIVCSRLEEYNSHQSLCNGTPEGPLRRNPGNHDKSRTPRLPSSADVEFCLSLTQYESGSMDKAANFSFRNTLEGFASPLTGIADASQSSMHNALHIYMNGTMSQVQGSANDPIFLLHHAFVDSIFEQWLRRHRPLQEVYPEANAPIGHNRESYMVPFIPLYRNGDFFISSKDLGYDYSYLQDSDPDSFQDYIKSYLEQASRIWSWLLGAAMVGAVLTALLAGLVSLLCRHKRKQLPEEKQPLLMEKEDYHSLYQSHL

SEQ ID NO: 68 - protein, 529 aa, Artificial sequence
  Single strand DNA oligonucleotide
MLLAVLYCLLWSFQTSAGHFPRACVSSKNLMEKECCPPWSGDRSPCGQLSGRGSCQNILLSNAPLGPQFPFTGVDDRESWPSVFYNRTCQCSGNFMGFNCGNCKFGFWGPNCTERRLLVRRNIFDLSAPEKDKFFAYLTLAKHTISSDYVIPIGTYGQMKNGSTPMFNDINIYDLFVWIHYYVSMDALLGGYEIWRDIDFAHEAPAFLPWHRLFLLRWEQEIQKLTGDENFTIPYWDWRDAEKCDICTDEYMGGQHPTNPNLLSPASFFSSWQIVCSRLEEYNSHQSLCNGTPEGPLRRNPGNHDKSRTPRLPSSADVEFCLSLTQYESGSMDKAANFSFRNTLEGFASPLTGIADASQSSMHNALHIYMDGTMSQVQGSANDPIFLLHHAFVDSIFEQWLRRHRPLQEVYPEANAPIGHNRESYMVPFIPLYRNGDFFISSKDLGYDYSYLQDSDPDSFQDYIKSYLEQASRIWSWLLGAAMVGAVLTALLAGLVSLLCRHKRKQLPEEKQPLLMEKEDYHSLYQSHL

SEQ ID NO: 69 - protein, 9 aa, Artificial sequence
  HLA restricted peptide
AAMVGAVLT

SEQ ID NO: 70 - protein, 10 aa, Artificial sequence
  HLA restricted peptide
AAMVGAVLTA

SEQ ID NO: 71 - protein, 9 aa, Artificial sequence
  HLA restricted peptide
AANFSFRNT

SEQ ID NO: 72 - protein, 10 aa, Artificial sequence
  HLA restricted peptide
AANFSFRNTL

SEQ ID NO: 73 - protein, 9 aa, Artificial sequence
  HLA restricted peptide
ACVSSKNLM

SEQ ID NO: 74 - protein, 9 aa, Artificial sequence
  HLA restricted peptide
ADASQSSMH

SEQ ID NO: 75 - protein, 10 aa, Artificial sequence
  HLA restricted peptide
ADASQSSMHN

SEQ ID NO: 76 - protein, 9 aa, Artificial sequence
  HLA restricted peptide
ADVEFCLSL

SEQ ID NO: 77 - protein, 10 aa, Artificial sequence
  HLA restricted peptide
ADVEFCLSLT

SEQ ID NO: 78 - protein, 10 aa, Artificial sequence
  HLA restricted peptide
AEKCDICTDE

SEQ ID NO: 79 - protein, 9 aa, Artificial sequence
  HLA restricted peptide
AFLPWHRLF

SEQ ID NO: 80 - protein, 10 aa, Artificial sequence
  HLA restricted peptide
AFLPWHRLFL

SEQ ID NO: 81 - protein, 9 aa, Artificial sequence
  HLA restricted peptide
AFVDSIFEQ

SEQ ID NO: 82 - protein, 10 aa, Artificial sequence
  HLA restricted peptide
AFVDSIFEQW

SEQ ID NO: 83 - protein, 9 aa, Artificial sequence
  HLA restricted peptide
AGHFPRACV

SEQ ID NO: 84 - protein, 10 aa, Artificial sequence
  HLA restricted peptide
AGHFPRACVS

SEQ ID NO: 85 - protein, 9 aa, Artificial sequence
  HLA restricted peptide
AGLVSLLCR

SEQ ID NO: 86 - protein, 10 aa, Artificial sequence
  HLA restricted peptide
AGLVSLLCRH

SEQ ID NO: 87 - protein, 10 aa, Artificial sequence
  HLA restricted peptide
AHEAPAFLPW

SEQ ID NO: 88 - protein, 9 aa, Artificial sequence
  HLA restricted peptide
AKHTISSDY

SEQ ID NO: 89 - protein, 10 aa, Artificial sequence
  HLA restricted peptide
AKHTISSDYV

SEQ ID NO: 90 - protein, 9 aa, Artificial sequence
  HLA restricted peptide
ALHIYMDGT

SEQ ID NO: 91 - protein, 10 aa, Artificial sequence
  HLA restricted peptide
ALHIYMDGTM

SEQ ID NO: 92 - protein, 9 aa, Artificial sequence
  HLA restricted peptide
ALHIYMNGT

SEQ ID NO: 93 - protein, 10 aa, Artificial sequence
  HLA restricted peptide
ALHIYMNGTM

SEQ ID NO: 94 - protein, 9 aa, Artificial sequence
  HLA restricted peptide
ALLAGLVSL

SEQ ID NO: 95 - protein, 10 aa, Artificial sequence
  HLA restricted peptide
ALLAGLVSLL

SEQ ID NO: 96 - protein, 9 aa, Artificial sequence
  HLA restricted peptide
ALLGGYEIW

SEQ ID NO: 97 - protein, 10 aa, Artificial sequence
  HLA restricted peptide
ALLGGYEIWR

SEQ ID NO: 98 - protein, 9 aa, Artificial sequence
  HLA restricted peptide
AMVGAVLTA

SEQ ID NO: 99 - protein, 10 aa, Artificial sequence
  HLA restricted peptide
AMVGAVLTAL

SEQ ID NO: 100 - protein, 9 aa, Artificial sequence
  HLA restricted peptide
ANAPIGHNR

SEQ ID NO: 101 - protein, 9 aa, Artificial sequence
  HLA restricted peptide
ANDPIFLLH

SEQ ID NO: 102 - protein, 10 aa, Artificial sequence
  HLA restricted peptide
ANDPIFLLHH

SEQ ID NO: 103 - protein, 9 aa, Artificial sequence
  HLA restricted peptide
ANFSFRNTL

SEQ ID NO: 104 - protein, 10 aa, Artificial sequence
  HLA restricted peptide
ANFSFRNTLE

SEQ ID NO: 105 - protein, 9 aa, Artificial sequence
  HLA restricted peptide
APAFLPWHR

SEQ ID NO: 106 - protein, 10 aa, Artificial sequence
  HLA restricted peptide
APAFLPWHRL

SEQ ID NO: 107 - protein, 9 aa, Artificial sequence
  HLA restricted peptide
APEKDKFFA

SEQ ID NO: 108 - protein, 10 aa, Artificial sequence
  HLA restricted peptide
APEKDKFFAY

SEQ ID NO: 109 - protein, 9 aa, Artificial sequence
  HLA restricted peptide
APIGHNRES

SEQ ID NO: 110 - protein, 10 aa, Artificial sequence
  HLA restricted peptide
APIGHNRESY

SEQ ID NO: 111 - protein, 9 aa, Artificial sequence
  HLA restricted peptide
APLGPQFPF

SEQ ID NO: 112 - protein, 10 aa, Artificial sequence
  HLA restricted peptide
APLGPQFPFT

SEQ ID NO: 113 - protein, 9 aa, Artificial sequence
  HLA restricted peptide
ASFFSSWQI

SEQ ID NO: 114 - protein, 10 aa, Artificial sequence
  HLA restricted peptide
ASFFSSWQIV

SEQ ID NO: 115 - protein, 10 aa, Artificial sequence
  HLA restricted peptide
ASPLTGIADA

SEQ ID NO: 116 - protein, 9 aa, Artificial sequence
  HLA restricted peptide
ASQSSMHNA

SEQ ID NO: 117 - protein, 10 aa, Artificial sequence
  HLA restricted peptide
ASQSSMHNAL

SEQ ID NO: 118 - protein, 9 aa, Artificial sequence
  HLA restricted peptide
ASRIWSWLL

SEQ ID NO: 119 - protein, 10 aa, Artificial sequence
  HLA restricted peptide
AVLTALLAGL

SEQ ID NO: 120 - protein, 9 aa, Artificial sequence
  HLA restricted peptide
AVLYCLLWS

SEQ ID NO: 121 - protein, 10 aa, Artificial sequence
  HLA restricted peptide
AVLYCLLWSF

SEQ ID NO: 122 - protein, 9 aa, Artificial sequence
  HLA restricted peptide
AYLTLAKHT

SEQ ID NO: 123 - protein, 10 aa, Artificial sequence
  HLA restricted peptide
AYLTLAKHTI

SEQ ID NO: 124 - protein, 9 aa, Artificial sequence
  HLA restricted peptide
CCPPWSGDR

SEQ ID NO: 125 - protein, 10 aa, Artificial sequence
  HLA restricted peptide
CCPPWSGDRS

SEQ ID NO: 126 - protein, 9 aa, Artificial sequence
  HLA restricted peptide
CDICTDEYM

SEQ ID NO: 127 - protein, 10 aa, Artificial sequence
  HLA restricted peptide
CDICTDEYMG

SEQ ID NO: 128 - protein, 9 aa, Artificial sequence
  HLA restricted peptide
CGNCKFGFW

SEQ ID NO: 129 - protein, 9 aa, Artificial sequence
  HLA restricted peptide
CGQLSGRGS

SEQ ID NO: 130 - protein, 10 aa, Artificial sequence
  HLA restricted peptide
CGQLSGRGSC

SEQ ID NO: 131 - protein, 9 aa, Artificial sequence
  HLA restricted peptide
CKFGFWGPN

SEQ ID NO: 132 - protein, 10 aa, Artificial sequence
  HLA restricted peptide
CKFGFWGPNC

SEQ ID NO: 133 - protein, 9 aa, Artificial sequence
  HLA restricted peptide
CLLWSFQTS

SEQ ID NO: 134 - protein, 10 aa, Artificial sequence
  HLA restricted peptide
CLLWSFQTSA

SEQ ID NO: 135 - protein, 9 aa, Artificial sequence
  HLA restricted peptide
CLSLTQYES

SEQ ID NO: 136 - protein, 9 aa, Artificial sequence
  HLA restricted peptide
CNGTPEGPL

SEQ ID NO: 137 - protein, 10 aa, Artificial sequence
  HLA restricted peptide
CNGTPEGPLR

SEQ ID NO: 138 - protein, 9 aa, Artificial sequence
  HLA restricted peptide
CPPWSGDRS

SEQ ID NO: 139 - protein, 10 aa, Artificial sequence
  HLA restricted peptide
CPPWSGDRSP

SEQ ID NO: 140 - protein, 9 aa, Artificial sequence
  HLA restricted peptide
CQCSGNFMG

SEQ ID NO: 141 - protein, 10 aa, Artificial sequence
  HLA restricted peptide
CQCSGNFMGF

SEQ ID NO: 142 - protein, 9 aa, Artificial sequence
  HLA restricted peptide
CQNILLSNA

SEQ ID NO: 143 - protein, 10 aa, Artificial sequence
  HLA restricted peptide
CQNILLSNAP

SEQ ID NO: 144 - protein, 9 aa, Artificial sequence
  HLA restricted peptide
CRHKRKQLP

SEQ ID NO: 145 - protein, 10 aa, Artificial sequence
  HLA restricted peptide
CRHKRKQLPE

SEQ ID NO: 146 - protein, 9 aa, Artificial sequence
  HLA restricted peptide
CSGNFMGFN

SEQ ID NO: 147 - protein, 10 aa, Artificial sequence
  HLA restricted peptide
CSGNFMGFNC

SEQ ID NO: 148 - protein, 9 aa, Artificial sequence
  HLA restricted peptide
CSRLEEYNS

SEQ ID NO: 149 - protein, 10 aa, Artificial sequence
  HLA restricted peptide
CSRLEEYNSH

SEQ ID NO: 150 - protein, 9 aa, Artificial sequence
  HLA restricted peptide
CTDEYMGGQ

SEQ ID NO: 151 - protein, 10 aa, Artificial sequence
  HLA restricted peptide
CTDEYMGGQH

SEQ ID NO: 152 - protein, 9 aa, Artificial sequence
  HLA restricted peptide
CTERRLLVR

SEQ ID NO: 153 - protein, 10 aa, Artificial sequence
  HLA restricted peptide
CTERRLLVRR

SEQ ID NO: 154 - protein, 10 aa, Artificial sequence
  HLA restricted peptide
CVSSKNLMEK

SEQ ID NO: 155 - protein, 9 aa, Artificial sequence
  HLA restricted peptide
DAEKCDICT

SEQ ID NO: 156 - protein, 10 aa, Artificial sequence
  HLA restricted peptide
DAEKCDICTD

SEQ ID NO: 157 - protein, 9 aa, Artificial sequence
  HLA restricted peptide
DALLGGYEI

SEQ ID NO: 158 - protein, 10 aa, Artificial sequence
  HLA restricted peptide
DALLGGYEIW

SEQ ID NO: 159 - protein, 9 aa, Artificial sequence
  HLA restricted peptide
DASQSSMHN

SEQ ID NO: 160 - protein, 10 aa, Artificial sequence
  HLA restricted peptide
DASQSSMHNA

SEQ ID NO: 161 - protein, 9 aa, Artificial sequence
  HLA restricted peptide
DDRESWPSV

SEQ ID NO: 162 - protein, 10 aa, Artificial sequence
  HLA restricted peptide
DDRESWPSVF

SEQ ID NO: 163 - protein, 9 aa, Artificial sequence
  HLA restricted peptide
DENFTIPYW

SEQ ID NO: 164 - protein, 10 aa, Artificial sequence
  HLA restricted peptide
DENFTIPYWD

SEQ ID NO: 165 - protein, 9 aa, Artificial sequence
  HLA restricted peptide
DEYMGGQHP

SEQ ID NO: 166 - protein, 10 aa, Artificial sequence
  HLA restricted peptide
DEYMGGQHPT

SEQ ID NO: 167 - protein, 9 aa, Artificial sequence
  HLA restricted peptide
DFAHEAPAF

SEQ ID NO: 168 - protein, 10 aa, Artificial sequence
  HLA restricted peptide
DFAHEAPAFL

SEQ ID NO: 169 - protein, 9 aa, Artificial sequence
  HLA restricted peptide
DFFISSKDL

SEQ ID NO: 170 - protein, 10 aa, Artificial sequence
  HLA restricted peptide
DGTMSQVQGS

SEQ ID NO: 171 - protein, 10 aa, Artificial sequence
  HLA restricted peptide
DIDFAHEAPA

SEQ ID NO: 172 - protein, 9 aa, Artificial sequence
  HLA restricted peptide
DINIYDLFV

SEQ ID NO: 173 - protein, 10 aa, Artificial sequence
  HLA restricted peptide
DINIYDLFVW

SEQ ID NO: 174 - protein, 9 aa, Artificial sequence
  HLA restricted peptide
DKAANFSFR

SEQ ID NO: 175 - protein, 10 aa, Artificial sequence
  HLA restricted peptide
DKAANFSFRN

SEQ ID NO: 176 - protein, 9 aa, Artificial sequence
  HLA restricted peptide
DKFFAYLTL

SEQ ID NO: 177 - protein, 10 aa, Artificial sequence
  HLA restricted peptide
DKFFAYLTLA

SEQ ID NO: 178 - protein, 10 aa, Artificial sequence
  HLA restricted peptide
DKSRTPRLPS

SEQ ID NO: 179 - protein, 9 aa, Artificial sequence
  HLA restricted peptide
DLFVWIHYY

SEQ ID NO: 180 - protein, 10 aa, Artificial sequence
  HLA restricted peptide
DLFVWIHYYV

SEQ ID NO: 181 - protein, 9 aa, Artificial sequence
  HLA restricted peptide
DLGYDYSYL

SEQ ID NO: 182 - protein, 9 aa, Artificial sequence
  HLA restricted peptide
DLSAPEKDK

SEQ ID NO: 183 - protein, 10 aa, Artificial sequence
  HLA restricted peptide
DLSAPEKDKF

SEQ ID NO: 184 - protein, 9 aa, Artificial sequence
  HLA restricted peptide
DPDSFQDYI

SEQ ID NO: 185 - protein, 10 aa, Artificial sequence
  HLA restricted peptide
DPDSFQDYIK

SEQ ID NO: 186 - protein, 9 aa, Artificial sequence
  HLA restricted peptide
DPIFLLHHA

SEQ ID NO: 187 - protein, 10 aa, Artificial sequence
  HLA restricted peptide
DPIFLLHHAF

SEQ ID NO: 188 - protein, 9 aa, Artificial sequence
  HLA restricted peptide
DRESWPSVF

SEQ ID NO: 189 - protein, 10 aa, Artificial sequence
  HLA restricted peptide
DRESWPSVFY

SEQ ID NO: 190 - protein, 9 aa, Artificial sequence
  HLA restricted peptide
DRSPCGQLS

SEQ ID NO: 191 - protein, 10 aa, Artificial sequence
  HLA restricted peptide
DRSPCGQLSG

SEQ ID NO: 192 - protein, 9 aa, Artificial sequence
  HLA restricted peptide
DSDPDSFQD

SEQ ID NO: 193 - protein, 10 aa, Artificial sequence
  HLA restricted peptide
DSDPDSFQDY

SEQ ID NO: 194 - protein, 9 aa, Artificial sequence
  HLA restricted peptide
DSFQDYIKS

SEQ ID NO: 195 - protein, 10 aa, Artificial sequence
  HLA restricted peptide
DSFQDYIKSY

SEQ ID NO: 196 - protein, 9 aa, Artificial sequence
  HLA restricted peptide
DSIFEQWLR

SEQ ID NO: 197 - protein, 10 aa, Artificial sequence
  HLA restricted peptide
DSIFEQWLRR

SEQ ID NO: 198 - protein, 9 aa, Artificial sequence
  HLA restricted peptide
DVEFCLSLT

SEQ ID NO: 199 - protein, 10 aa, Artificial sequence
  HLA restricted peptide
DVEFCLSLTQ

SEQ ID NO: 200 - protein, 10 aa, Artificial sequence
  HLA restricted peptide
DWRDAEKCDI

SEQ ID NO: 201 - protein, 9 aa, Artificial sequence
  HLA restricted peptide
DYHSLYQSH

SEQ ID NO: 202 - protein, 10 aa, Artificial sequence
  HLA restricted peptide
DYHSLYQSHL

SEQ ID NO: 203 - protein, 9 aa, Artificial sequence
  HLA restricted peptide
DYIKSYLEQ

SEQ ID NO: 204 - protein, 10 aa, Artificial sequence
  HLA restricted peptide
DYIKSYLEQA

SEQ ID NO: 205 - protein, 9 aa, Artificial sequence
  HLA restricted peptide
DYVIPIGTY

SEQ ID NO: 206 - protein, 10 aa, Artificial sequence
  HLA restricted peptide
DYVIPIGTYG

SEQ ID NO: 207 - protein, 9 aa, Artificial sequence
  HLA restricted peptide
EANAPIGHN

SEQ ID NO: 208 - protein, 10 aa, Artificial sequence
  HLA restricted peptide
EANAPIGHNR

SEQ ID NO: 209 - protein, 9 aa, Artificial sequence
  HLA restricted peptide
EAPAFLPWH

SEQ ID NO: 210 - protein, 10 aa, Artificial sequence
  HLA restricted peptide
EAPAFLPWHR

SEQ ID NO: 211 - protein, 10 aa, Artificial sequence
  HLA restricted peptide
ECCPPWSGDR

SEQ ID NO: 212 - protein, 9 aa, Artificial sequence
  HLA restricted peptide
EDYHSLYQS

SEQ ID NO: 213 - protein, 10 aa, Artificial sequence
  HLA restricted peptide
EDYHSLYQSH

SEQ ID NO: 214 - protein, 10 aa, Artificial sequence
  HLA restricted peptide
EEKQPLLMEK

SEQ ID NO: 215 - protein, 9 aa, Artificial sequence
  HLA restricted peptide
EEYNSHQSL

SEQ ID NO: 216 - protein, 10 aa, Artificial sequence
  HLA restricted peptide
EEYNSHQSLC

SEQ ID NO: 217 - protein, 9 aa, Artificial sequence
  HLA restricted peptide
EFCLSLTQY

SEQ ID NO: 218 - protein, 9 aa, Artificial sequence
  HLA restricted peptide
EGFASPLTG

SEQ ID NO: 219 - protein, 10 aa, Artificial sequence
  HLA restricted peptide
EGFASPLTGI

SEQ ID NO: 220 - protein, 9 aa, Artificial sequence
  HLA restricted peptide
EGPLRRNPG

SEQ ID NO: 221 - protein, 10 aa, Artificial sequence
  HLA restricted peptide
EGPLRRNPGN

SEQ ID NO: 222 - protein, 9 aa, Artificial sequence
  HLA restricted peptide
EIWRDIDFA

SEQ ID NO: 223 - protein, 10 aa, Artificial sequence
  HLA restricted peptide
EIWRDIDFAH

SEQ ID NO: 224 - protein, 10 aa, Artificial sequence
  HLA restricted peptide
EKCDICTDEY

SEQ ID NO: 225 - protein, 9 aa, Artificial sequence
  HLA restricted peptide
EKDKFFAYL

SEQ ID NO: 226 - protein, 10 aa, Artificial sequence
  HLA restricted peptide
EKDKFFAYLT

SEQ ID NO: 227 - protein, 9 aa, Artificial sequence
  HLA restricted peptide
EKECCPPWS

SEQ ID NO: 228 - protein, 9 aa, Artificial sequence
  HLA restricted peptide
EKEDYHSLY

SEQ ID NO: 229 - protein, 9 aa, Artificial sequence
  HLA restricted peptide
EKQPLLMEK

SEQ ID NO: 230 - protein, 10 aa, Artificial sequence
  HLA restricted peptide
ENFTIPYWDW

SEQ ID NO: 231 - protein, 9 aa, Artificial sequence
  HLA restricted peptide
EQASRIWSW

SEQ ID NO: 232 - protein, 10 aa, Artificial sequence
  HLA restricted peptide
EQASRIWSWL

SEQ ID NO: 233 - protein, 9 aa, Artificial sequence
  HLA restricted peptide
EQWLRRHRP

SEQ ID NO: 234 - protein, 10 aa, Artificial sequence
  HLA restricted peptide
EQWLRRHRPL

SEQ ID NO: 235 - protein, 9 aa, Artificial sequence
  HLA restricted peptide
ERRLLVRRN

SEQ ID NO: 236 - protein, 10 aa, Artificial sequence
  HLA restricted peptide
ERRLLVRRNI

SEQ ID NO: 237 - protein, 9 aa, Artificial sequence
  HLA restricted peptide
ESGSMDKAA

SEQ ID NO: 238 - protein, 10 aa, Artificial sequence
  HLA restricted peptide
ESWPSVFYNR

SEQ ID NO: 239 - protein, 9 aa, Artificial sequence
  HLA restricted peptide
ESYMVPFIP

SEQ ID NO: 240 - protein, 10 aa, Artificial sequence
  HLA restricted peptide
ESYMVPFIPL

SEQ ID NO: 241 - protein, 9 aa, Artificial sequence
  HLA restricted peptide
EVYPEANAP

SEQ ID NO: 242 - protein, 10 aa, Artificial sequence
  HLA restricted peptide
EVYPEANAPI

SEQ ID NO: 243 - protein, 9 aa, Artificial sequence
  HLA restricted peptide
EYMGGQHPT

SEQ ID NO: 244 - protein, 10 aa, Artificial sequence
  HLA restricted peptide
EYMGGQHPTN

SEQ ID NO: 245 - protein, 9 aa, Artificial sequence
  HLA restricted peptide
EYNSHQSLC

SEQ ID NO: 246 - protein, 10 aa, Artificial sequence
  HLA restricted peptide
EYNSHQSLCN

SEQ ID NO: 247 - protein, 9 aa, Artificial sequence
  HLA restricted peptide
FAHEAPAFL

SEQ ID NO: 248 - protein, 10 aa, Artificial sequence
  HLA restricted peptide
FAHEAPAFLP

SEQ ID NO: 249 - protein, 9 aa, Artificial sequence
  HLA restricted peptide
FASPLTGIA

SEQ ID NO: 250 - protein, 10 aa, Artificial sequence
  HLA restricted peptide
FASPLTGIAD

SEQ ID NO: 251 - protein, 9 aa, Artificial sequence
  HLA restricted peptide
FAYLTLAKH

SEQ ID NO: 252 - protein, 10 aa, Artificial sequence
  HLA restricted peptide
FAYLTLAKHT

SEQ ID NO: 253 - protein, 10 aa, Artificial sequence
  HLA restricted peptide
FCLSLTQYES

SEQ ID NO: 254 - protein, 9 aa, Artificial sequence
  HLA restricted peptide
FEQWLRRHR

SEQ ID NO: 255 - protein, 9 aa, Artificial sequence
  HLA restricted peptide
FFAYLTLAK

SEQ ID NO: 256 - protein, 10 aa, Artificial sequence
  HLA restricted peptide
FFAYLTLAKH

SEQ ID NO: 257 - protein, 10 aa, Artificial sequence
  HLA restricted peptide
FFISSKDLGY

SEQ ID NO: 258 - protein, 9 aa, Artificial sequence
  HLA restricted peptide
FFSSWQIVC

SEQ ID NO: 259 - protein, 10 aa, Artificial sequence
  HLA restricted peptide
FFSSWQIVCS

SEQ ID NO: 260 - protein, 9 aa, Artificial sequence
  HLA restricted peptide
FGFWGPNCT

SEQ ID NO: 261 - protein, 10 aa, Artificial sequence
  HLA restricted peptide
FGFWGPNCTE

SEQ ID NO: 262 - protein, 10 aa, Artificial sequence
  HLA restricted peptide
FIPLYRNGDF

SEQ ID NO: 263 - protein, 9 aa, Artificial sequence
  HLA restricted peptide
FISSKDLGY

SEQ ID NO: 264 - protein, 10 aa, Artificial sequence
  HLA restricted peptide
FLLHHAFVDS

SEQ ID NO: 265 - protein, 9 aa, Artificial sequence
  HLA restricted peptide
FLLRWEQEI

SEQ ID NO: 266 - protein, 9 aa, Artificial sequence
  HLA restricted peptide
FLPWHRLFL

SEQ ID NO: 267 - protein, 10 aa, Artificial sequence
  HLA restricted peptide
FLPWHRLFLL

SEQ ID NO: 268 - protein, 9 aa, Artificial sequence
  HLA restricted peptide
FMGFNCGNC

SEQ ID NO: 269 - protein, 10 aa, Artificial sequence
  HLA restricted peptide
FMGFNCGNCK

SEQ ID NO: 270 - protein, 10 aa, Artificial sequence
  HLA restricted peptide
FNCGNCKFGF

SEQ ID NO: 271 - protein, 9 aa, Artificial sequence
  HLA restricted peptide
FNDINIYDL

SEQ ID NO: 272 - protein, 10 aa, Artificial sequence
  HLA restricted peptide
FNDINIYDLF

SEQ ID NO: 273 - protein, 9 aa, Artificial sequence
  HLA restricted peptide
FPFTGVDDR

SEQ ID NO: 274 - protein, 10 aa, Artificial sequence
  HLA restricted peptide
FPFTGVDDRE

SEQ ID NO: 275 - protein, 9 aa, Artificial sequence
  HLA restricted peptide
FPRACVSSK

SEQ ID NO: 276 - protein, 10 aa, Artificial sequence
  HLA restricted peptide
FPRACVSSKN

SEQ ID NO: 277 - protein, 9 aa, Artificial sequence
  HLA restricted peptide
FQDYIKSYL

SEQ ID NO: 278 - protein, 10 aa, Artificial sequence
  HLA restricted peptide
FQDYIKSYLE

SEQ ID NO: 279 - protein, 9 aa, Artificial sequence
  HLA restricted peptide
FQTSAGHFP

SEQ ID NO: 280 - protein, 10 aa, Artificial sequence
  HLA restricted peptide
FQTSAGHFPR

SEQ ID NO: 281 - protein, 9 aa, Artificial sequence
  HLA restricted peptide
FRNTLEGFA

SEQ ID NO: 282 - protein, 10 aa, Artificial sequence
  HLA restricted peptide
FRNTLEGFAS

SEQ ID NO: 283 - protein, 10 aa, Artificial sequence
  HLA restricted peptide
FSFRNTLEGF

SEQ ID NO: 284 - protein, 9 aa, Artificial sequence
  HLA restricted peptide
FSSWQIVCS

SEQ ID NO: 285 - protein, 10 aa, Artificial sequence
  HLA restricted peptide
FSSWQIVCSR

SEQ ID NO: 286 - protein, 10 aa, Artificial sequence
  HLA restricted peptide
FTGVDDRESW

SEQ ID NO: 287 - protein, 9 aa, Artificial sequence
  HLA restricted peptide
FTIPYWDWR

SEQ ID NO: 288 - protein, 9 aa, Artificial sequence
  HLA restricted peptide
FVDSIFEQW

SEQ ID NO: 289 - protein, 10 aa, Artificial sequence
  HLA restricted peptide
FVDSIFEQWL

SEQ ID NO: 290 - protein, 9 aa, Artificial sequence
  HLA restricted peptide
FVWIHYYVS

SEQ ID NO: 291 - protein, 10 aa, Artificial sequence
  HLA restricted peptide
FVWIHYYVSM

SEQ ID NO: 292 - protein, 9 aa, Artificial sequence
  HLA restricted peptide
FWGPNCTER

SEQ ID NO: 293 - protein, 10 aa, Artificial sequence
  HLA restricted peptide
FWGPNCTERR

SEQ ID NO: 294 - protein, 9 aa, Artificial sequence
  HLA restricted peptide
FYNRTCQCS

SEQ ID NO: 295 - protein, 9 aa, Artificial sequence
  HLA restricted peptide
GAAMVGAVL

SEQ ID NO: 296 - protein, 10 aa, Artificial sequence
  HLA restricted peptide
GAAMVGAVLT

SEQ ID NO: 297 - protein, 9 aa, Artificial sequence
  HLA restricted peptide
GAVLTALLA

SEQ ID NO: 298 - protein, 10 aa, Artificial sequence
  HLA restricted peptide
GAVLTALLAG

SEQ ID NO: 299 - protein, 9 aa, Artificial sequence
  HLA restricted peptide
GDENFTIPY

SEQ ID NO: 300 - protein, 10 aa, Artificial sequence
  HLA restricted peptide
GDENFTIPYW

SEQ ID NO: 301 - protein, 10 aa, Artificial sequence
  HLA restricted peptide
GDFFISSKDL

SEQ ID NO: 302 - protein, 9 aa, Artificial sequence
  HLA restricted peptide
GDRSPCGQL

SEQ ID NO: 303 - protein, 10 aa, Artificial sequence
  HLA restricted peptide
GDRSPCGQLS

SEQ ID NO: 304 - protein, 9 aa, Artificial sequence
  HLA restricted peptide
GFASPLTGI

SEQ ID NO: 305 - protein, 10 aa, Artificial sequence
  HLA restricted peptide
GFASPLTGIA

SEQ ID NO: 306 - protein, 9 aa, Artificial sequence
  HLA restricted peptide
GFNCGNCKF

SEQ ID NO: 307 - protein, 10 aa, Artificial sequence
  HLA restricted peptide
GFWGPNCTER

SEQ ID NO: 308 - protein, 9 aa, Artificial sequence
  HLA restricted peptide
GGQHPTNPN

SEQ ID NO: 309 - protein, 10 aa, Artificial sequence
  HLA restricted peptide
GGQHPTNPNL

SEQ ID NO: 310 - protein, 9 aa, Artificial sequence
  HLA restricted peptide
GGYEIWRDI

SEQ ID NO: 311 - protein, 10 aa, Artificial sequence
  HLA restricted peptide
GGYEIWRDID

SEQ ID NO: 312 - protein, 9 aa, Artificial sequence
  HLA restricted peptide
GHFPRACVS

SEQ ID NO: 313 - protein, 10 aa, Artificial sequence
  HLA restricted peptide
GHFPRACVSS

SEQ ID NO: 314 - protein, 9 aa, Artificial sequence
  HLA restricted peptide
GHNRESYMV

SEQ ID NO: 315 - protein, 9 aa, Artificial sequence
  HLA restricted peptide
GIADASQSS

SEQ ID NO: 316 - protein, 10 aa, Artificial sequence
  HLA restricted peptide
GIADASQSSM

SEQ ID NO: 317 - protein, 9 aa, Artificial sequence
  HLA restricted peptide
GLVSLLCRH

SEQ ID NO: 318 - protein, 10 aa, Artificial sequence
  HLA restricted peptide
GLVSLLCRHK

SEQ ID NO: 319 - protein, 9 aa, Artificial sequence
  HLA restricted peptide
GNFMGFNCG

SEQ ID NO: 320 - protein, 10 aa, Artificial sequence
  HLA restricted peptide
GNFMGFNCGN

SEQ ID NO: 321 - protein, 10 aa, Artificial sequence
  HLA restricted peptide
GNHDKSRTPR

SEQ ID NO: 322 - protein, 9 aa, Artificial sequence
  HLA restricted peptide
GPLRRNPGN

SEQ ID NO: 323 - protein, 10 aa, Artificial sequence
  HLA restricted peptide
GPLRRNPGNH

SEQ ID NO: 324 - protein, 9 aa, Artificial sequence
  HLA restricted peptide
GPNCTERRL

SEQ ID NO: 325 - protein, 10 aa, Artificial sequence
  HLA restricted peptide
GPNCTERRLL

SEQ ID NO: 326 - protein, 9 aa, Artificial sequence
  HLA restricted peptide
GPQFPFTGV

SEQ ID NO: 327 - protein, 10 aa, Artificial sequence
  HLA restricted peptide
GPQFPFTGVD

SEQ ID NO: 328 - protein, 9 aa, Artificial sequence
  HLA restricted peptide
GQHPTNPNL

SEQ ID NO: 329 - protein, 10 aa, Artificial sequence
  HLA restricted peptide
GQHPTNPNLL

SEQ ID NO: 330 - protein, 9 aa, Artificial sequence
  HLA restricted peptide
GQLSGRGSC

SEQ ID NO: 331 - protein, 10 aa, Artificial sequence
  HLA restricted peptide
GQLSGRGSCQ

SEQ ID NO: 332 - protein, 9 aa, Artificial sequence
  HLA restricted peptide
GQMKNGSTP

SEQ ID NO: 333 - protein, 10 aa, Artificial sequence
  HLA restricted peptide
GQMKNGSTPM

SEQ ID NO: 334 - protein, 9 aa, Artificial sequence
  HLA restricted peptide
GRGSCQNIL

SEQ ID NO: 335 - protein, 10 aa, Artificial sequence
  HLA restricted peptide
GRGSCQNILL

SEQ ID NO: 336 - protein, 9 aa, Artificial sequence
  HLA restricted peptide
GSANDPIFL

SEQ ID NO: 337 - protein, 10 aa, Artificial sequence
  HLA restricted peptide
GSANDPIFLL

SEQ ID NO: 338 - protein, 9 aa, Artificial sequence
  HLA restricted peptide
GSCQNILLS

SEQ ID NO: 339 - protein, 10 aa, Artificial sequence
  HLA restricted peptide
GSCQNILLSN

SEQ ID NO: 340 - protein, 9 aa, Artificial sequence
  HLA restricted peptide
GSMDKAANF

SEQ ID NO: 341 - protein, 10 aa, Artificial sequence
  HLA restricted peptide
GSMDKAANFS

SEQ ID NO: 342 - protein, 9 aa, Artificial sequence
  HLA restricted peptide
GSTPMFNDI

SEQ ID NO: 343 - protein, 10 aa, Artificial sequence
  HLA restricted peptide
GSTPMFNDIN

SEQ ID NO: 344 - protein, 9 aa, Artificial sequence
  HLA restricted peptide
GTMSQVQGS

SEQ ID NO: 345 - protein, 10 aa, Artificial sequence
  HLA restricted peptide
GTMSQVQGSA

SEQ ID NO: 346 - protein, 9 aa, Artificial sequence
  HLA restricted peptide
GTPEGPLRR

SEQ ID NO: 347 - protein, 10 aa, Artificial sequence
  HLA restricted peptide
GTPEGPLRRN

SEQ ID NO: 348 - protein, 10 aa, Artificial sequence
  HLA restricted peptide
GTYGQMKNGS

SEQ ID NO: 349 - protein, 10 aa, Artificial sequence
  HLA restricted peptide
GVDDRESWPS

SEQ ID NO: 350 - protein, 10 aa, Artificial sequence
  HLA restricted peptide
GYDYSYLQDS

SEQ ID NO: 351 - protein, 10 aa, Artificial sequence
  HLA restricted peptide
GYEIWRDIDF

SEQ ID NO: 352 - protein, 9 aa, Artificial sequence
  HLA restricted peptide
HAFVDSIFE

SEQ ID NO: 353 - protein, 10 aa, Artificial sequence
  HLA restricted peptide
HAFVDSIFEQ

SEQ ID NO: 354 - protein, 9 aa, Artificial sequence
  HLA restricted peptide
HDKSRTPRL

SEQ ID NO: 355 - protein, 10 aa, Artificial sequence
  HLA restricted peptide
HDKSRTPRLP

SEQ ID NO: 356 - protein, 9 aa, Artificial sequence
  HLA restricted peptide
HEAPAFLPW

SEQ ID NO: 357 - protein, 10 aa, Artificial sequence
  HLA restricted peptide
HEAPAFLPWH

SEQ ID NO: 358 - protein, 9 aa, Artificial sequence
  HLA restricted peptide
HFPRACVSS

SEQ ID NO: 359 - protein, 10 aa, Artificial sequence
  HLA restricted peptide
HFPRACVSSK

SEQ ID NO: 360 - protein, 9 aa, Artificial sequence
  HLA restricted peptide
HHAFVDSIF

SEQ ID NO: 361 - protein, 9 aa, Artificial sequence
  HLA restricted peptide
HIYMDGTMS

SEQ ID NO: 362 - protein, 9 aa, Artificial sequence
  HLA restricted peptide
HIYMNGTMS

SEQ ID NO: 363 - protein, 10 aa, Artificial sequence
  HLA restricted peptide
HKRKQLPEEK

SEQ ID NO: 364 - protein, 9 aa, Artificial sequence
  HLA restricted peptide
HNALHIYMN

SEQ ID NO: 365 - protein, 10 aa, Artificial sequence
  HLA restricted peptide
HNRESYMVPF

SEQ ID NO: 366 - protein, 9 aa, Artificial sequence
  HLA restricted peptide
HPTNPNLLS

SEQ ID NO: 367 - protein, 10 aa, Artificial sequence
  HLA restricted peptide
HPTNPNLLSP

SEQ ID NO: 368 - protein, 9 aa, Artificial sequence
  HLA restricted peptide
HQSLCNGTP

SEQ ID NO: 369 - protein, 10 aa, Artificial sequence
  HLA restricted peptide
HQSLCNGTPE

SEQ ID NO: 370 - protein, 9 aa, Artificial sequence
  HLA restricted peptide
HRLFLLRWE

SEQ ID NO: 371 - protein, 10 aa, Artificial sequence
  HLA restricted peptide
HRLFLLRWEQ

SEQ ID NO: 372 - protein, 9 aa, Artificial sequence
  HLA restricted peptide
HRPLQEVYP

SEQ ID NO: 373 - protein, 10 aa, Artificial sequence
  HLA restricted peptide
HRPLQEVYPE

SEQ ID NO: 374 - protein, 9 aa, Artificial sequence
  HLA restricted peptide
HTISSDYVI

SEQ ID NO: 375 - protein, 9 aa, Artificial sequence
  HLA restricted peptide
HYYVSMDAL

SEQ ID NO: 376 - protein, 10 aa, Artificial sequence
  HLA restricted peptide
HYYVSMDALL

SEQ ID NO: 377 - protein, 9 aa, Artificial sequence
  HLA restricted peptide
IADASQSSM

SEQ ID NO: 378 - protein, 10 aa, Artificial sequence
  HLA restricted peptide
IADASQSSMH

SEQ ID NO: 379 - protein, 9 aa, Artificial sequence
  HLA restricted peptide
IDFAHEAPA

SEQ ID NO: 380 - protein, 10 aa, Artificial sequence
  HLA restricted peptide
IDFAHEAPAF

SEQ ID NO: 381 - protein, 9 aa, Artificial sequence
  HLA restricted peptide
IFDLSAPEK

SEQ ID NO: 382 - protein, 9 aa, Artificial sequence
  HLA restricted peptide
IFEQWLRRH

SEQ ID NO: 383 - protein, 10 aa, Artificial sequence
  HLA restricted peptide
IFEQWLRRHR

SEQ ID NO: 384 - protein, 9 aa, Artificial sequence
  HLA restricted peptide
IFLLHHAFV

SEQ ID NO: 385 - protein, 9 aa, Artificial sequence
  HLA restricted peptide
IGHNRESYM

SEQ ID NO: 386 - protein, 10 aa, Artificial sequence
  HLA restricted peptide
IGHNRESYMV

SEQ ID NO: 387 - protein, 9 aa, Artificial sequence
  HLA restricted peptide
IGTYGQMKN

SEQ ID NO: 388 - protein, 9 aa, Artificial sequence
  HLA restricted peptide
IHYYVSMDA

SEQ ID NO: 389 - protein, 10 aa, Artificial sequence
  HLA restricted peptide
IHYYVSMDAL

SEQ ID NO: 390 - protein, 9 aa, Artificial sequence
  HLA restricted peptide
IKSYLEQAS

SEQ ID NO: 391 - protein, 9 aa, Artificial sequence
  HLA restricted peptide
INIYDLFVW

SEQ ID NO: 392 - protein, 10 aa, Artificial sequence
  HLA restricted peptide
INIYDLFVWI

SEQ ID NO: 393 - protein, 9 aa, Artificial sequence
  HLA restricted peptide
IPIGTYGQM

SEQ ID NO: 394 - protein, 10 aa, Artificial sequence
  HLA restricted peptide
IPIGTYGQMK

SEQ ID NO: 395 - protein, 9 aa, Artificial sequence
  HLA restricted peptide
IPLYRNGDF

SEQ ID NO: 396 - protein, 10 aa, Artificial sequence
  HLA restricted peptide
IPLYRNGDFF

SEQ ID NO: 397 - protein, 9 aa, Artificial sequence
  HLA restricted peptide
IPYWDWRDA

SEQ ID NO: 398 - protein, 10 aa, Artificial sequence
  HLA restricted peptide
IPYWDWRDAE

SEQ ID NO: 399 - protein, 9 aa, Artificial sequence
  HLA restricted peptide
IQKLTGDEN

SEQ ID NO: 400 - protein, 10 aa, Artificial sequence
  HLA restricted peptide
IQKLTGDENF

SEQ ID NO: 401 - protein, 9 aa, Artificial sequence
  HLA restricted peptide
ISSDYVIPI

SEQ ID NO: 402 - protein, 10 aa, Artificial sequence
  HLA restricted peptide
ISSKDLGYDY

SEQ ID NO: 403 - protein, 9 aa, Artificial sequence
  HLA restricted peptide
IVCSRLEEY

SEQ ID NO: 404 - protein, 10 aa, Artificial sequence
  HLA restricted peptide
IVCSRLEEYN

SEQ ID NO: 405 - protein, 9 aa, Artificial sequence
  HLA restricted peptide
IWSWLLGAA

SEQ ID NO: 406 - protein, 10 aa, Artificial sequence
  HLA restricted peptide
IWSWLLGAAM

SEQ ID NO: 407 - protein, 9 aa, Artificial sequence
  HLA restricted peptide
IYDLFVWIH

SEQ ID NO: 408 - protein, 10 aa, Artificial sequence
  HLA restricted peptide
IYDLFVWIHY

SEQ ID NO: 409 - protein, 10 aa, Artificial sequence
  HLA restricted peptide
IYMDGTMSQV

SEQ ID NO: 410 - protein, 10 aa, Artificial sequence
  HLA restricted peptide
IYMNGTMSQV

SEQ ID NO: 411 - protein, 9 aa, Artificial sequence
  HLA restricted peptide
KAANFSFRN

SEQ ID NO: 412 - protein, 10 aa, Artificial sequence
  HLA restricted peptide
KAANFSFRNT

SEQ ID NO: 413 - protein, 9 aa, Artificial sequence
  HLA restricted peptide
KCDICTDEY

SEQ ID NO: 414 - protein, 10 aa, Artificial sequence
  HLA restricted peptide
KCDICTDEYM

SEQ ID NO: 415 - protein, 9 aa, Artificial sequence
  HLA restricted peptide
KDKFFAYLT

SEQ ID NO: 416 - protein, 10 aa, Artificial sequence
  HLA restricted peptide
KDKFFAYLTL

SEQ ID NO: 417 - protein, 9 aa, Artificial sequence
  HLA restricted peptide
KDLGYDYSY

SEQ ID NO: 418 - protein, 10 aa, Artificial sequence
  HLA restricted peptide
KDLGYDYSYL

SEQ ID NO: 419 - protein, 10 aa, Artificial sequence
  HLA restricted peptide
KEDYHSLYQS

SEQ ID NO: 420 - protein, 9 aa, Artificial sequence
  HLA restricted peptide
KFFAYLTLA

SEQ ID NO: 421 - protein, 10 aa, Artificial sequence
  HLA restricted peptide
KFFAYLTLAK

SEQ ID NO: 422 - protein, 9 aa, Artificial sequence
  HLA restricted peptide
KFGFWGPNC

SEQ ID NO: 423 - protein, 10 aa, Artificial sequence
  HLA restricted peptide
KFGFWGPNCT

SEQ ID NO: 424 - protein, 9 aa, Artificial sequence
  HLA restricted peptide
KHTISSDYV

SEQ ID NO: 425 - protein, 10 aa, Artificial sequence
  HLA restricted peptide
KHTISSDYVI

SEQ ID NO: 426 - protein, 9 aa, Artificial sequence
  HLA restricted peptide
KLTGDENFT

SEQ ID NO: 427 - protein, 10 aa, Artificial sequence
  HLA restricted peptide
KLTGDENFTI

SEQ ID NO: 428 - protein, 9 aa, Artificial sequence
  HLA restricted peptide
KNGSTPMFN

SEQ ID NO: 429 - protein, 9 aa, Artificial sequence
  HLA restricted peptide
KNLMEKECC

SEQ ID NO: 430 - protein, 9 aa, Artificial sequence
  HLA restricted peptide
KQLPEEKQP

SEQ ID NO: 431 - protein, 10 aa, Artificial sequence
  HLA restricted peptide
KQLPEEKQPL

SEQ ID NO: 432 - protein, 9 aa, Artificial sequence
  HLA restricted peptide
KQPLLMEKE

SEQ ID NO: 433 - protein, 10 aa, Artificial sequence
  HLA restricted peptide
KQPLLMEKED

SEQ ID NO: 434 - protein, 9 aa, Artificial sequence
  HLA restricted peptide
KRKQLPEEK

SEQ ID NO: 435 - protein, 10 aa, Artificial sequence
  HLA restricted peptide
KRKQLPEEKQ

SEQ ID NO: 436 - protein, 9 aa, Artificial sequence
  HLA restricted peptide
KSRTPRLPS

SEQ ID NO: 437 - protein, 10 aa, Artificial sequence
  HLA restricted peptide
KSRTPRLPSS

SEQ ID NO: 438 - protein, 9 aa, Artificial sequence
  HLA restricted peptide
KSYLEQASR

SEQ ID NO: 439 - protein, 10 aa, Artificial sequence
  HLA restricted peptide
KSYLEQASRI

SEQ ID NO: 440 - protein, 9 aa, Artificial sequence
  HLA restricted peptide
LAGLVSLLC

SEQ ID NO: 441 - protein, 10 aa, Artificial sequence
  HLA restricted peptide
LAGLVSLLCR

SEQ ID NO: 442 - protein, 10 aa, Artificial sequence
  HLA restricted peptide
LAKHTISSDY

SEQ ID NO: 443 - protein, 9 aa, Artificial sequence
  HLA restricted peptide
LAVLYCLLW

SEQ ID NO: 444 - protein, 10 aa, Artificial sequence
  HLA restricted peptide
LAVLYCLLWS

SEQ ID NO: 445 - protein, 10 aa, Artificial sequence
  HLA restricted peptide
LCNGTPEGPL

SEQ ID NO: 446 - protein, 9 aa, Artificial sequence
  HLA restricted peptide
LCRHKRKQL

SEQ ID NO: 447 - protein, 10 aa, Artificial sequence
  HLA restricted peptide
LCRHKRKQLP

SEQ ID NO: 448 - protein, 9 aa, Artificial sequence
  HLA restricted peptide
LEEYNSHQS

SEQ ID NO: 449 - protein, 10 aa, Artificial sequence
  HLA restricted peptide
LEEYNSHQSL

SEQ ID NO: 450 - protein, 9 aa, Artificial sequence
  HLA restricted peptide
LEGFASPLT

SEQ ID NO: 451 - protein, 9 aa, Artificial sequence
  HLA restricted peptide
LEQASRIWS

SEQ ID NO: 452 - protein, 10 aa, Artificial sequence
  HLA restricted peptide
LEQASRIWSW

SEQ ID NO: 453 - protein, 10 aa, Artificial sequence
  HLA restricted peptide
LFLLRWEQEI

SEQ ID NO: 454 - protein, 9 aa, Artificial sequence
  HLA restricted peptide
LFVWIHYYV

SEQ ID NO: 455 - protein, 10 aa, Artificial sequence
  HLA restricted peptide
LFVWIHYYVS

SEQ ID NO: 456 - protein, 9 aa, Artificial sequence
  HLA restricted peptide
LGAAMVGAV

SEQ ID NO: 457 - protein, 10 aa, Artificial sequence
  HLA restricted peptide
LGAAMVGAVL

SEQ ID NO: 458 - protein, 10 aa, Artificial sequence
  HLA restricted peptide
LGGYEIWRDI

SEQ ID NO: 459 - protein, 10 aa, Artificial sequence
  HLA restricted peptide
LGPQFPFTGV

SEQ ID NO: 460 - protein, 9 aa, Artificial sequence
  HLA restricted peptide
LGYDYSYLQ

SEQ ID NO: 461 - protein, 10 aa, Artificial sequence
  HLA restricted peptide
LGYDYSYLQD

SEQ ID NO: 462 - protein, 9 aa, Artificial sequence
  HLA restricted peptide
LHHAFVDSI

SEQ ID NO: 463 - protein, 10 aa, Artificial sequence
  HLA restricted peptide
LHHAFVDSIF

SEQ ID NO: 464 - protein, 9 aa, Artificial sequence
  HLA restricted peptide
LHIYMDGTM

SEQ ID NO: 465 - protein, 10 aa, Artificial sequence
  HLA restricted peptide
LHIYMDGTMS

SEQ ID NO: 466 - protein, 9 aa, Artificial sequence
  HLA restricted peptide
LHIYMNGTM

SEQ ID NO: 467 - protein, 10 aa, Artificial sequence
  HLA restricted peptide
LHIYMNGTMS

SEQ ID NO: 468 - protein, 9 aa, Artificial sequence
  HLA restricted peptide
LLAGLVSLL

SEQ ID NO: 469 - protein, 10 aa, Artificial sequence
  HLA restricted peptide
LLAGLVSLLC

SEQ ID NO: 470 - protein, 9 aa, Artificial sequence
  HLA restricted peptide
LLAVLYCLL

SEQ ID NO: 471 - protein, 10 aa, Artificial sequence
  HLA restricted peptide
LLAVLYCLLW

SEQ ID NO: 472 - protein, 10 aa, Artificial sequence
  HLA restricted peptide
LLCRHKRKQL

SEQ ID NO: 473 - protein, 9 aa, Artificial sequence
  HLA restricted peptide
LLGAAMVGA

SEQ ID NO: 474 - protein, 10 aa, Artificial sequence
  HLA restricted peptide
LLGAAMVGAV

SEQ ID NO: 475 - protein, 9 aa, Artificial sequence
  HLA restricted peptide
LLGGYEIWR

SEQ ID NO: 476 - protein, 9 aa, Artificial sequence
  HLA restricted peptide
LLHHAFVDS

SEQ ID NO: 477 - protein, 10 aa, Artificial sequence
  HLA restricted peptide
LLHHAFVDSI

SEQ ID NO: 478 - protein, 9 aa, Artificial sequence
  HLA restricted peptide
LLMEKEDYH

SEQ ID NO: 479 - protein, 10 aa, Artificial sequence
  HLA restricted peptide
LLMEKEDYHS

SEQ ID NO: 480 - protein, 10 aa, Artificial sequence
  HLA restricted peptide
LLRWEQEIQK

SEQ ID NO: 481 - protein, 9 aa, Artificial sequence
  HLA restricted peptide
LLSPASFFS

SEQ ID NO: 482 - protein, 10 aa, Artificial sequence
  HLA restricted peptide
LLSPASFFSS

SEQ ID NO: 483 - protein, 10 aa, Artificial sequence
  HLA restricted peptide
LLVRRNIFDL

SEQ ID NO: 484 - protein, 9 aa, Artificial sequence
  HLA restricted peptide
LLWSFQTSA

SEQ ID NO: 485 - protein, 10 aa, Artificial sequence
  HLA restricted peptide
LLWSFQTSAG

SEQ ID NO: 486 - protein, 10 aa, Artificial sequence
  HLA restricted peptide
LMEKECCPPW

SEQ ID NO: 487 - protein, 9 aa, Artificial sequence
  HLA restricted peptide
LMEKEDYHS

SEQ ID NO: 488 - protein, 10 aa, Artificial sequence
  HLA restricted peptide
LMEKEDYHSL

SEQ ID NO: 489 - protein, 9 aa, Artificial sequence
  HLA restricted peptide
LPEEKQPLL

SEQ ID NO: 490 - protein, 10 aa, Artificial sequence
  HLA restricted peptide
LPEEKQPLLM

SEQ ID NO: 491 - protein, 9 aa, Artificial sequence
  HLA restricted peptide
LPSSADVEF

SEQ ID NO: 492 - protein, 10 aa, Artificial sequence
  HLA restricted peptide
LPSSADVEFC

SEQ ID NO: 493 - protein, 9 aa, Artificial sequence
  HLA restricted peptide
LPWHRLFLL

SEQ ID NO: 494 - protein, 10 aa, Artificial sequence
  HLA restricted peptide
LPWHRLFLLR

SEQ ID NO: 495 - protein, 9 aa, Artificial sequence
  HLA restricted peptide
LQDSDPDSF

SEQ ID NO: 496 - protein, 10 aa, Artificial sequence
  HLA restricted peptide
LQDSDPDSFQ

SEQ ID NO: 497 - protein, 9 aa, Artificial sequence
  HLA restricted peptide
LQEVYPEAN

SEQ ID NO: 498 - protein, 10 aa, Artificial sequence
  HLA restricted peptide
LQEVYPEANA

SEQ ID NO: 499 - protein, 9 aa, Artificial sequence
  HLA restricted peptide
LRRHRPLQE

SEQ ID NO: 500 - protein, 10 aa, Artificial sequence
  HLA restricted peptide
LRRHRPLQEV

SEQ ID NO: 501 - protein, 9 aa, Artificial sequence
  HLA restricted peptide
LRRNPGNHD

SEQ ID NO: 502 - protein, 10 aa, Artificial sequence
  HLA restricted peptide
LRRNPGNHDK

SEQ ID NO: 503 - protein, 9 aa, Artificial sequence
  HLA restricted peptide
LRWEQEIQK

SEQ ID NO: 504 - protein, 10 aa, Artificial sequence
  HLA restricted peptide
LRWEQEIQKL

SEQ ID NO: 505 - protein, 9 aa, Artificial sequence
  HLA restricted peptide
LSAPEKDKF

SEQ ID NO: 506 - protein, 10 aa, Artificial sequence
  HLA restricted peptide
LSAPEKDKFF

SEQ ID NO: 507 - protein, 9 aa, Artificial sequence
  HLA restricted peptide
LSGRGSCQN

SEQ ID NO: 508 - protein, 10 aa, Artificial sequence
  HLA restricted peptide
LSGRGSCQNI

SEQ ID NO: 509 - protein, 10 aa, Artificial sequence
  HLA restricted peptide
LSLTQYESGS

SEQ ID NO: 510 - protein, 10 aa, Artificial sequence
  HLA restricted peptide
LSNAPLGPQF

SEQ ID NO: 511 - protein, 9 aa, Artificial sequence
  HLA restricted peptide
LSPASFFSS

SEQ ID NO: 512 - protein, 10 aa, Artificial sequence
  HLA restricted peptide
LSPASFFSSW

SEQ ID NO: 513 - protein, 9 aa, Artificial sequence
  HLA restricted peptide
LTALLAGLV

SEQ ID NO: 514 - protein, 10 aa, Artificial sequence
  HLA restricted peptide
LTALLAGLVS

SEQ ID NO: 515 - protein, 9 aa, Artificial sequence
  HLA restricted peptide
LTGDENFTI

SEQ ID NO: 516 - protein, 10 aa, Artificial sequence
  HLA restricted peptide
LTGIADASQS

SEQ ID NO: 517 - protein, 9 aa, Artificial sequence
  HLA restricted peptide
LTLAKHTIS

SEQ ID NO: 518 - protein, 10 aa, Artificial sequence
  HLA restricted peptide
LTLAKHTISS

SEQ ID NO: 519 - protein, 9 aa, Artificial sequence
  HLA restricted peptide
LTQYESGSM

SEQ ID NO: 520 - protein, 9 aa, Artificial sequence
  HLA restricted peptide
LVRRNIFDL

SEQ ID NO: 521 - protein, 10 aa, Artificial sequence
  HLA restricted peptide
LVRRNIFDLS

SEQ ID NO: 522 - protein, 9 aa, Artificial sequence
  HLA restricted peptide
LVSLLCRHK

SEQ ID NO: 523 - protein, 10 aa, Artificial sequence
  HLA restricted peptide
LVSLLCRHKR

SEQ ID NO: 524 - protein, 10 aa, Artificial sequence
  HLA restricted peptide
LYCLLWSFQT

SEQ ID NO: 525 - protein, 9 aa, Artificial sequence
  HLA restricted peptide
LYRNGDFFI

SEQ ID NO: 526 - protein, 10 aa, Artificial sequence
  HLA restricted peptide
LYRNGDFFIS

SEQ ID NO: 527 - protein, 10 aa, Artificial sequence
  HLA restricted peptide
MDALLGGYEI

SEQ ID NO: 528 - protein, 9 aa, Artificial sequence
  HLA restricted peptide
MDKAANFSF

SEQ ID NO: 529 - protein, 10 aa, Artificial sequence
  HLA restricted peptide
MDKAANFSFR

SEQ ID NO: 530 - protein, 9 aa, Artificial sequence
  HLA restricted peptide
MEKECCPPW

SEQ ID NO: 531 - protein, 10 aa, Artificial sequence
  HLA restricted peptide
MEKECCPPWS

SEQ ID NO: 532 - protein, 9 aa, Artificial sequence
  HLA restricted peptide
MEKEDYHSL

SEQ ID NO: 533 - protein, 10 aa, Artificial sequence
  HLA restricted peptide
MEKEDYHSLY

SEQ ID NO: 534 - protein, 10 aa, Artificial sequence
  HLA restricted peptide
MFNDINIYDL

SEQ ID NO: 535 - protein, 9 aa, Artificial sequence
  HLA restricted peptide
MGFNCGNCK

SEQ ID NO: 536 - protein, 10 aa, Artificial sequence
  HLA restricted peptide
MGFNCGNCKF

SEQ ID NO: 537 - protein, 10 aa, Artificial sequence
  HLA restricted peptide
MGGQHPTNPN

SEQ ID NO: 538 - protein, 9 aa, Artificial sequence
  HLA restricted peptide
MHNALHIYM

SEQ ID NO: 539 - protein, 10 aa, Artificial sequence
  HLA restricted peptide
MHNALHIYMN

SEQ ID NO: 540 - protein, 9 aa, Artificial sequence
  HLA restricted peptide
MKNGSTPMF

SEQ ID NO: 541 - protein, 10 aa, Artificial sequence
  HLA restricted peptide
MKNGSTPMFN

SEQ ID NO: 542 - protein, 9 aa, Artificial sequence
  HLA restricted peptide
MLLAVLYCL

SEQ ID NO: 543 - protein, 10 aa, Artificial sequence
  HLA restricted peptide
MLLAVLYCLL

SEQ ID NO: 544 - protein, 9 aa, Artificial sequence
  HLA restricted peptide
MSQVQGSAN

SEQ ID NO: 545 - protein, 9 aa, Artificial sequence
  HLA restricted peptide
MVGAVLTAL

SEQ ID NO: 546 - protein, 10 aa, Artificial sequence
  HLA restricted peptide
MVGAVLTALL

SEQ ID NO: 547 - protein, 9 aa, Artificial sequence
  HLA restricted peptide
MVPFIPLYR

SEQ ID NO: 548 - protein, 10 aa, Artificial sequence
  HLA restricted peptide
MVPFIPLYRN

SEQ ID NO: 549 - protein, 9 aa, Artificial sequence
  HLA restricted peptide
NALHIYMDG

SEQ ID NO: 550 - protein, 10 aa, Artificial sequence
  HLA restricted peptide
NALHIYMDGT

SEQ ID NO: 551 - protein, 9 aa, Artificial sequence
  HLA restricted peptide
NALHIYMNG

SEQ ID NO: 552 - protein, 10 aa, Artificial sequence
  HLA restricted peptide
NALHIYMNGT

SEQ ID NO: 553 - protein, 9 aa, Artificial sequence
  HLA restricted peptide
NAPIGHNRE

SEQ ID NO: 554 - protein, 10 aa, Artificial sequence
  HLA restricted peptide
NAPIGHNRES

SEQ ID NO: 555 - protein, 10 aa, Artificial sequence
  HLA restricted peptide
NAPLGPQFPF

SEQ ID NO: 556 - protein, 9 aa, Artificial sequence
  HLA restricted peptide
NCGNCKFGF

SEQ ID NO: 557 - protein, 10 aa, Artificial sequence
  HLA restricted peptide
NCGNCKFGFW

SEQ ID NO: 558 - protein, 10 aa, Artificial sequence
  HLA restricted peptide
NCKFGFWGPN

SEQ ID NO: 559 - protein, 9 aa, Artificial sequence
  HLA restricted peptide
NCTERRLLV

SEQ ID NO: 560 - protein, 10 aa, Artificial sequence
  HLA restricted peptide
NCTERRLLVR

SEQ ID NO: 561 - protein, 9 aa, Artificial sequence
  HLA restricted peptide
NDINIYDLF

SEQ ID NO: 562 - protein, 10 aa, Artificial sequence
  HLA restricted peptide
NDINIYDLFV

SEQ ID NO: 563 - protein, 10 aa, Artificial sequence
  HLA restricted peptide
NDPIFLLHHA

SEQ ID NO: 564 - protein, 9 aa, Artificial sequence
  HLA restricted peptide
NFMGFNCGN

SEQ ID NO: 565 - protein, 10 aa, Artificial sequence
  HLA restricted peptide
NFMGFNCGNC

SEQ ID NO: 566 - protein, 9 aa, Artificial sequence
  HLA restricted peptide
NFTIPYWDW

SEQ ID NO: 567 - protein, 10 aa, Artificial sequence
  HLA restricted peptide
NFTIPYWDWR

SEQ ID NO: 568 - protein, 9 aa, Artificial sequence
  HLA restricted peptide
NGDFFISSK

SEQ ID NO: 569 - protein, 10 aa, Artificial sequence
  HLA restricted peptide
NGSTPMFNDI

SEQ ID NO: 570 - protein, 10 aa, Artificial sequence
  HLA restricted peptide
NGTMSQVQGS

SEQ ID NO: 571 - protein, 9 aa, Artificial sequence
  HLA restricted peptide
NGTPEGPLR

SEQ ID NO: 572 - protein, 10 aa, Artificial sequence
  HLA restricted peptide
NGTPEGPLRR

SEQ ID NO: 573 - protein, 9 aa, Artificial sequence
  HLA restricted peptide
NHDKSRTPR

SEQ ID NO: 574 - protein, 10 aa, Artificial sequence
  HLA restricted peptide
NHDKSRTPRL

SEQ ID NO: 575 - protein, 10 aa, Artificial sequence
  HLA restricted peptide
NIFDLSAPEK

SEQ ID NO: 576 - protein, 9 aa, Artificial sequence
  HLA restricted peptide
NILLSNAPL

SEQ ID NO: 577 - protein, 9 aa, Artificial sequence
  HLA restricted peptide
NIYDLFVWI

SEQ ID NO: 578 - protein, 10 aa, Artificial sequence
  HLA restricted peptide
NIYDLFVWIH

SEQ ID NO: 579 - protein, 9 aa, Artificial sequence
  HLA restricted peptide
NLLSPASFF

SEQ ID NO: 580 - protein, 10 aa, Artificial sequence
  HLA restricted peptide
NLLSPASFFS

SEQ ID NO: 581 - protein, 9 aa, Artificial sequence
  HLA restricted peptide
NPGNHDKSR

SEQ ID NO: 582 - protein, 10 aa, Artificial sequence
  HLA restricted peptide
NPGNHDKSRT

SEQ ID NO: 583 - protein, 9 aa, Artificial sequence
  HLA restricted peptide
NPNLLSPAS

SEQ ID NO: 584 - protein, 10 aa, Artificial sequence
  HLA restricted peptide
NPNLLSPASF

SEQ ID NO: 585 - protein, 9 aa, Artificial sequence
  HLA restricted peptide
NRESYMVPF

SEQ ID NO: 586 - protein, 10 aa, Artificial sequence
  HLA restricted peptide
NRESYMVPFI

SEQ ID NO: 587 - protein, 9 aa, Artificial sequence
  HLA restricted peptide
NRTCQCSGN

SEQ ID NO: 588 - protein, 10 aa, Artificial sequence
  HLA restricted peptide
NRTCQCSGNF

SEQ ID NO: 589 - protein, 10 aa, Artificial sequence
  HLA restricted peptide
NSHQSLCNGT

SEQ ID NO: 590 - protein, 10 aa, Artificial sequence
  HLA restricted peptide
NTLEGFASPL

SEQ ID NO: 591 - protein, 9 aa, Artificial sequence
  HLA restricted peptide
PAFLPWHRL

SEQ ID NO: 592 - protein, 10 aa, Artificial sequence
  HLA restricted peptide
PAFLPWHRLF

SEQ ID NO: 593 - protein, 10 aa, Artificial sequence
  HLA restricted peptide
PASFFSSWQI

SEQ ID NO: 594 - protein, 9 aa, Artificial sequence
  HLA restricted peptide
PEEKQPLLM

SEQ ID NO: 595 - protein, 9 aa, Artificial sequence
  HLA restricted peptide
PEKDKFFAY

SEQ ID NO: 596 - protein, 10 aa, Artificial sequence
  HLA restricted peptide
PEKDKFFAYL

SEQ ID NO: 597 - protein, 9 aa, Artificial sequence
  HLA restricted peptide
PIFLLHHAF

SEQ ID NO: 598 - protein, 10 aa, Artificial sequence
  HLA restricted peptide
PIFLLHHAFV

SEQ ID NO: 599 - protein, 9 aa, Artificial sequence
  HLA restricted peptide
PIGHNRESY

SEQ ID NO: 600 - protein, 9 aa, Artificial sequence
  HLA restricted peptide
PIGTYGQMK

SEQ ID NO: 601 - protein, 9 aa, Artificial sequence
  HLA restricted peptide
PLGPQFPFT

SEQ ID NO: 602 - protein, 9 aa, Artificial sequence
  HLA restricted peptide
PLLMEKEDY

SEQ ID NO: 603 - protein, 9 aa, Artificial sequence
  HLA restricted peptide
PLYRNGDFF

SEQ ID NO: 604 - protein, 10 aa, Artificial sequence
  HLA restricted peptide
PLYRNGDFFI

SEQ ID NO: 605 - protein, 9 aa, Artificial sequence
  HLA restricted peptide
PMFNDINIY

SEQ ID NO: 606 - protein, 10 aa, Artificial sequence
  HLA restricted peptide
PNCTERRLLV

SEQ ID NO: 607 - protein, 9 aa, Artificial sequence
  HLA restricted peptide
PNLLSPASF

SEQ ID NO: 608 - protein, 10 aa, Artificial sequence
  HLA restricted peptide
PNLLSPASFF

SEQ ID NO: 609 - protein, 10 aa, Artificial sequence
  HLA restricted peptide
PPWSGDRSPC

SEQ ID NO: 610 - protein, 9 aa, Artificial sequence
  HLA restricted peptide
PQFPFTGVD

SEQ ID NO: 611 - protein, 10 aa, Artificial sequence
  HLA restricted peptide
PQFPFTGVDD

SEQ ID NO: 612 - protein, 9 aa, Artificial sequence
  HLA restricted peptide
PRACVSSKN

SEQ ID NO: 613 - protein, 10 aa, Artificial sequence
  HLA restricted peptide
PRACVSSKNL

SEQ ID NO: 614 - protein, 9 aa, Artificial sequence
  HLA restricted peptide
PRLPSSADV

SEQ ID NO: 615 - protein, 10 aa, Artificial sequence
  HLA restricted peptide
PRLPSSADVE

SEQ ID NO: 616 - protein, 10 aa, Artificial sequence
  HLA restricted peptide
PSSADVEFCL

SEQ ID NO: 617 - protein, 9 aa, Artificial sequence
  HLA restricted peptide
QASRIWSWL

SEQ ID NO: 618 - protein, 10 aa, Artificial sequence
  HLA restricted peptide
QASRIWSWLL

SEQ ID NO: 619 - protein, 9 aa, Artificial sequence
  HLA restricted peptide
QCSGNFMGF

SEQ ID NO: 620 - protein, 10 aa, Artificial sequence
  HLA restricted peptide
QCSGNFMGFN

SEQ ID NO: 621 - protein, 9 aa, Artificial sequence
  HLA restricted peptide
QDSDPDSFQ

SEQ ID NO: 622 - protein, 9 aa, Artificial sequence
  HLA restricted peptide
QDYIKSYLE

SEQ ID NO: 623 - protein, 9 aa, Artificial sequence
  HLA restricted peptide
QEIQKLTGD

SEQ ID NO: 624 - protein, 10 aa, Artificial sequence
  HLA restricted peptide
QEIQKLTGDE

SEQ ID NO: 625 - protein, 9 aa, Artificial sequence
  HLA restricted peptide
QEVYPEANA

SEQ ID NO: 626 - protein, 10 aa, Artificial sequence
  HLA restricted peptide
QEVYPEANAP

SEQ ID NO: 627 - protein, 10 aa, Artificial sequence
  HLA restricted peptide
QFPFTGVDDR

SEQ ID NO: 628 - protein, 9 aa, Artificial sequence
  HLA restricted peptide
QGSANDPIF

SEQ ID NO: 629 - protein, 10 aa, Artificial sequence
  HLA restricted peptide
QGSANDPIFL

SEQ ID NO: 630 - protein, 9 aa, Artificial sequence
  HLA restricted peptide
QHPTNPNLL

SEQ ID NO: 631 - protein, 10 aa, Artificial sequence
  HLA restricted peptide
QHPTNPNLLS

SEQ ID NO: 632 - protein, 10 aa, Artificial sequence
  HLA restricted peptide
QIVCSRLEEY

SEQ ID NO: 633 - protein, 9 aa, Artificial sequence
  HLA restricted peptide
QKLTGDENF

SEQ ID NO: 634 - protein, 10 aa, Artificial sequence
  HLA restricted peptide
QKLTGDENFT

SEQ ID NO: 635 - protein, 9 aa, Artificial sequence
  HLA restricted peptide
QLPEEKQPL

SEQ ID NO: 636 - protein, 10 aa, Artificial sequence
  HLA restricted peptide
QLPEEKQPLL

SEQ ID NO: 637 - protein, 10 aa, Artificial sequence
  HLA restricted peptide
QLSGRGSCQN

SEQ ID NO: 638 - protein, 9 aa, Artificial sequence
  HLA restricted peptide
QMKNGSTPM

SEQ ID NO: 639 - protein, 10 aa, Artificial sequence
  HLA restricted peptide
QMKNGSTPMF

SEQ ID NO: 640 - protein, 10 aa, Artificial sequence
  HLA restricted peptide
QNILLSNAPL

SEQ ID NO: 641 - protein, 9 aa, Artificial sequence
  HLA restricted peptide
QPLLMEKED

SEQ ID NO: 642 - protein, 10 aa, Artificial sequence
  HLA restricted peptide
QPLLMEKEDY

SEQ ID NO: 643 - protein, 9 aa, Artificial sequence
  HLA restricted peptide
QSSMHNALH

SEQ ID NO: 644 - protein, 10 aa, Artificial sequence
  HLA restricted peptide
QSSMHNALHI

SEQ ID NO: 645 - protein, 9 aa, Artificial sequence
  HLA restricted peptide
QTSAGHFPR

SEQ ID NO: 646 - protein, 10 aa, Artificial sequence
  HLA restricted peptide
QTSAGHFPRA

SEQ ID NO: 647 - protein, 10 aa, Artificial sequence
  HLA restricted peptide
QVQGSANDPI

SEQ ID NO: 648 - protein, 9 aa, Artificial sequence
  HLA restricted peptide
QWLRRHRPL

SEQ ID NO: 649 - protein, 9 aa, Artificial sequence
  HLA restricted peptide
QYESGSMDK

SEQ ID NO: 650 - protein, 10 aa, Artificial sequence
  HLA restricted peptide
QYESGSMDKA

SEQ ID NO: 651 - protein, 9 aa, Artificial sequence
  HLA restricted peptide
RACVSSKNL

SEQ ID NO: 652 - protein, 10 aa, Artificial sequence
  HLA restricted peptide
RACVSSKNLM

SEQ ID NO: 653 - protein, 9 aa, Artificial sequence
  HLA restricted peptide
RDAEKCDIC

SEQ ID NO: 654 - protein, 10 aa, Artificial sequence
  HLA restricted peptide
RDAEKCDICT

SEQ ID NO: 655 - protein, 9 aa, Artificial sequence
  HLA restricted peptide
RDIDFAHEA

SEQ ID NO: 656 - protein, 9 aa, Artificial sequence
  HLA restricted peptide
RESWPSVFY

SEQ ID NO: 657 - protein, 10 aa, Artificial sequence
  HLA restricted peptide
RESWPSVFYN

SEQ ID NO: 658 - protein, 9 aa, Artificial sequence
  HLA restricted peptide
RESYMVPFI

SEQ ID NO: 659 - protein, 9 aa, Artificial sequence
  HLA restricted peptide
RGSCQNILL

SEQ ID NO: 660 - protein, 10 aa, Artificial sequence
  HLA restricted peptide
RGSCQNILLS

SEQ ID NO: 661 - protein, 9 aa, Artificial sequence
  HLA restricted peptide
RHRPLQEVY

SEQ ID NO: 662 - protein, 9 aa, Artificial sequence
  HLA restricted peptide
RIWSWLLGA

SEQ ID NO: 663 - protein, 10 aa, Artificial sequence
  HLA restricted peptide
RIWSWLLGAA

SEQ ID NO: 664 - protein, 10 aa, Artificial sequence
  HLA restricted peptide
RLEEYNSHQS

SEQ ID NO: 665 - protein, 9 aa, Artificial sequence
  HLA restricted peptide
RLFLLRWEQ

SEQ ID NO: 666 - protein, 10 aa, Artificial sequence
  HLA restricted peptide
RLFLLRWEQE

SEQ ID NO: 667 - protein, 9 aa, Artificial sequence
  HLA restricted peptide
RLLVRRNIF

SEQ ID NO: 668 - protein, 10 aa, Artificial sequence
  HLA restricted peptide
RLPSSADVEF

SEQ ID NO: 669 - protein, 9 aa, Artificial sequence
  HLA restricted peptide
RNGDFFISS

SEQ ID NO: 670 - protein, 10 aa, Artificial sequence
  HLA restricted peptide
RNGDFFISSK

SEQ ID NO: 671 - protein, 9 aa, Artificial sequence
  HLA restricted peptide
RNPGNHDKS

SEQ ID NO: 672 - protein, 10 aa, Artificial sequence
  HLA restricted peptide
RNPGNHDKSR

SEQ ID NO: 673 - protein, 9 aa, Artificial sequence
  HLA restricted peptide
RNTLEGFAS

SEQ ID NO: 674 - protein, 9 aa, Artificial sequence
  HLA restricted peptide
RPLQEVYPE

SEQ ID NO: 675 - protein, 10 aa, Artificial sequence
  HLA restricted peptide
RPLQEVYPEA

SEQ ID NO: 676 - protein, 9 aa, Artificial sequence
  HLA restricted peptide
RRHRPLQEV

SEQ ID NO: 677 - protein, 10 aa, Artificial sequence
  HLA restricted peptide
RRHRPLQEVY

SEQ ID NO: 678 - protein, 9 aa, Artificial sequence
  HLA restricted peptide
RRLLVRRNI

SEQ ID NO: 679 - protein, 10 aa, Artificial sequence
  HLA restricted peptide
RRLLVRRNIF

SEQ ID NO: 680 - protein, 9 aa, Artificial sequence
  HLA restricted peptide
RRNIFDLSA

SEQ ID NO: 681 - protein, 10 aa, Artificial sequence
  HLA restricted peptide
RRNIFDLSAP

SEQ ID NO: 682 - protein, 9 aa, Artificial sequence
  HLA restricted peptide
RRNPGNHDK

SEQ ID NO: 683 - protein, 10 aa, Artificial sequence
  HLA restricted peptide
RRNPGNHDKS

SEQ ID NO: 684 - protein, 10 aa, Artificial sequence
  HLA restricted peptide
RSPCGQLSGR

SEQ ID NO: 685 - protein, 9 aa, Artificial sequence
  HLA restricted peptide
RTCQCSGNF

SEQ ID NO: 686 - protein, 10 aa, Artificial sequence
  HLA restricted peptide
RTCQCSGNFM

SEQ ID NO: 687 - protein, 9 aa, Artificial sequence
  HLA restricted peptide
RTPRLPSSA

SEQ ID NO: 688 - protein, 9 aa, Artificial sequence
  HLA restricted peptide
RWEQEIQKL

SEQ ID NO: 689 - protein, 10 aa, Artificial sequence
  HLA restricted peptide
RWEQEIQKLT

SEQ ID NO: 690 - protein, 9 aa, Artificial sequence
  HLA restricted peptide
SADVEFCLS

SEQ ID NO: 691 - protein, 10 aa, Artificial sequence
  HLA restricted peptide
SADVEFCLSL

SEQ ID NO: 692 - protein, 9 aa, Artificial sequence
  HLA restricted peptide
SAGHFPRAC

SEQ ID NO: 693 - protein, 10 aa, Artificial sequence
  HLA restricted peptide
SAGHFPRACV

SEQ ID NO: 694 - protein, 9 aa, Artificial sequence
  HLA restricted peptide
SANDPIFLL

SEQ ID NO: 695 - protein, 10 aa, Artificial sequence
  HLA restricted peptide
SANDPIFLLH

SEQ ID NO: 696 - protein, 9 aa, Artificial sequence
  HLA restricted peptide
SAPEKDKFF

SEQ ID NO: 697 - protein, 10 aa, Artificial sequence
  HLA restricted peptide
SAPEKDKFFA

SEQ ID NO: 698 - protein, 9 aa, Artificial sequence
  HLA restricted peptide
SCQNILLSN

SEQ ID NO: 699 - protein, 10 aa, Artificial sequence
  HLA restricted peptide
SCQNILLSNA

SEQ ID NO: 700 - protein, 9 aa, Artificial sequence
  HLA restricted peptide
SDPDSFQDY

SEQ ID NO: 701 - protein, 10 aa, Artificial sequence
  HLA restricted peptide
SDPDSFQDYI

SEQ ID NO: 702 - protein, 9 aa, Artificial sequence
  HLA restricted peptide
SDYVIPIGT

SEQ ID NO: 703 - protein, 10 aa, Artificial sequence
  HLA restricted peptide
SDYVIPIGTY

SEQ ID NO: 704 - protein, 9 aa, Artificial sequence
  HLA restricted peptide
SFFSSWQIV

SEQ ID NO: 705 - protein, 10 aa, Artificial sequence
  HLA restricted peptide
SFFSSWQIVC

SEQ ID NO: 706 - protein, 9 aa, Artificial sequence
  HLA restricted peptide
SFQDYIKSY

SEQ ID NO: 707 - protein, 10 aa, Artificial sequence
  HLA restricted peptide
SFQDYIKSYL

SEQ ID NO: 708 - protein, 9 aa, Artificial sequence
  HLA restricted peptide
SFQTSAGHF

SEQ ID NO: 709 - protein, 9 aa, Artificial sequence
  HLA restricted peptide
SFRNTLEGF

SEQ ID NO: 710 - protein, 10 aa, Artificial sequence
  HLA restricted peptide
SFRNTLEGFA

SEQ ID NO: 711 - protein, 10 aa, Artificial sequence
  HLA restricted peptide
SGDRSPCGQL

SEQ ID NO: 712 - protein, 9 aa, Artificial sequence
  HLA restricted peptide
SGNFMGFNC

SEQ ID NO: 713 - protein, 9 aa, Artificial sequence
  HLA restricted peptide
SGRGSCQNI

SEQ ID NO: 714 - protein, 10 aa, Artificial sequence
  HLA restricted peptide
SGRGSCQNIL

SEQ ID NO: 715 - protein, 9 aa, Artificial sequence
  HLA restricted peptide
SGSMDKAAN

SEQ ID NO: 716 - protein, 10 aa, Artificial sequence
  HLA restricted peptide
SGSMDKAANF

SEQ ID NO: 717 - protein, 9 aa, Artificial sequence
  HLA restricted peptide
SHQSLCNGT

SEQ ID NO: 718 - protein, 9 aa, Artificial sequence
  HLA restricted peptide
SIFEQWLRR

SEQ ID NO: 719 - protein, 10 aa, Artificial sequence
  HLA restricted peptide
SIFEQWLRRH

SEQ ID NO: 720 - protein, 9 aa, Artificial sequence
  HLA restricted peptide
SKDLGYDYS

SEQ ID NO: 721 - protein, 10 aa, Artificial sequence
  HLA restricted peptide
SKDLGYDYSY

SEQ ID NO: 722 - protein, 9 aa, Artificial sequence
  HLA restricted peptide
SKNLMEKEC

SEQ ID NO: 723 - protein, 10 aa, Artificial sequence
  HLA restricted peptide
SKNLMEKECC

SEQ ID NO: 724 - protein, 9 aa, Artificial sequence
  HLA restricted peptide
SLLCRHKRK

SEQ ID NO: 725 - protein, 9 aa, Artificial sequence
  HLA restricted peptide
SLTQYESGS

SEQ ID NO: 726 - protein, 10 aa, Artificial sequence
  HLA restricted peptide
SLTQYESGSM

SEQ ID NO: 727 - protein, 9 aa, Artificial sequence
  HLA restricted peptide
SMDALLGGY

SEQ ID NO: 728 - protein, 9 aa, Artificial sequence
  HLA restricted peptide
SMDKAANFS

SEQ ID NO: 729 - protein, 10 aa, Artificial sequence
  HLA restricted peptide
SMDKAANFSF

SEQ ID NO: 730 - protein, 9 aa, Artificial sequence
  HLA restricted peptide
SMHNALHIY

SEQ ID NO: 731 - protein, 10 aa, Artificial sequence
  HLA restricted peptide
SMHNALHIYM

SEQ ID NO: 732 - protein, 9 aa, Artificial sequence
  HLA restricted peptide
SNAPLGPQF

SEQ ID NO: 733 - protein, 9 aa, Artificial sequence
  HLA restricted peptide
SPASFFSSW

SEQ ID NO: 734 - protein, 10 aa, Artificial sequence
  HLA restricted peptide
SPASFFSSWQ

SEQ ID NO: 735 - protein, 9 aa, Artificial sequence
  HLA restricted peptide
SPCGQLSGR

SEQ ID NO: 736 - protein, 10 aa, Artificial sequence
  HLA restricted peptide
SPCGQLSGRG

SEQ ID NO: 737 - protein, 9 aa, Artificial sequence
  HLA restricted peptide
SPLTGIADA

SEQ ID NO: 738 - protein, 10 aa, Artificial sequence
  HLA restricted peptide
SPLTGIADAS

SEQ ID NO: 739 - protein, 9 aa, Artificial sequence
  HLA restricted peptide
SQSSMHNAL

SEQ ID NO: 740 - protein, 10 aa, Artificial sequence
  HLA restricted peptide
SQSSMHNALH

SEQ ID NO: 741 - protein, 9 aa, Artificial sequence
  HLA restricted peptide
SQVQGSAND

SEQ ID NO: 742 - protein, 10 aa, Artificial sequence
  HLA restricted peptide
SQVQGSANDP

SEQ ID NO: 743 - protein, 9 aa, Artificial sequence
  HLA restricted peptide
SRIWSWLLG

SEQ ID NO: 744 - protein, 10 aa, Artificial sequence
  HLA restricted peptide
SRIWSWLLGA

SEQ ID NO: 745 - protein, 9 aa, Artificial sequence
  HLA restricted peptide
SRLEEYNSH

SEQ ID NO: 746 - protein, 10 aa, Artificial sequence
  HLA restricted peptide
SRLEEYNSHQ

SEQ ID NO: 747 - protein, 9 aa, Artificial sequence
  HLA restricted peptide
SRTPRLPSS

SEQ ID NO: 748 - protein, 10 aa, Artificial sequence
  HLA restricted peptide
SRTPRLPSSA

SEQ ID NO: 749 - protein, 9 aa, Artificial sequence
  HLA restricted peptide
SSADVEFCL

SEQ ID NO: 750 - protein, 10 aa, Artificial sequence
  HLA restricted peptide
SSADVEFCLS

SEQ ID NO: 751 - protein, 9 aa, Artificial sequence
  HLA restricted peptide
SSDYVIPIG

SEQ ID NO: 752 - protein, 10 aa, Artificial sequence
  HLA restricted peptide
SSDYVIPIGT

SEQ ID NO: 753 - protein, 9 aa, Artificial sequence
  HLA restricted peptide
SSKDLGYDY

SEQ ID NO: 754 - protein, 10 aa, Artificial sequence
  HLA restricted peptide
SSKDLGYDYS

SEQ ID NO: 755 - protein, 10 aa, Artificial sequence
  HLA restricted peptide
SSKNLMEKEC

SEQ ID NO: 756 - protein, 9 aa, Artificial sequence
  HLA restricted peptide
SSMHNALHI

SEQ ID NO: 757 - protein, 10 aa, Artificial sequence
  HLA restricted peptide
SSMHNALHIY

SEQ ID NO: 758 - protein, 9 aa, Artificial sequence
  HLA restricted peptide
SSWQIVCSR

SEQ ID NO: 759 - protein, 10 aa, Artificial sequence
  HLA restricted peptide
SSWQIVCSRL

SEQ ID NO: 760 - protein, 9 aa, Artificial sequence
  HLA restricted peptide
STPMFNDIN

SEQ ID NO: 761 - protein, 10 aa, Artificial sequence
  HLA restricted peptide
STPMFNDINI

SEQ ID NO: 762 - protein, 10 aa, Artificial sequence
  HLA restricted peptide
SVFYNRTCQC

SEQ ID NO: 763 - protein, 9 aa, Artificial sequence
  HLA restricted peptide
SWLLGAAMV

SEQ ID NO: 764 - protein, 9 aa, Artificial sequence
  HLA restricted peptide
SWPSVFYNR

SEQ ID NO: 765 - protein, 10 aa, Artificial sequence
  HLA restricted peptide
SWPSVFYNRT

SEQ ID NO: 766 - protein, 9 aa, Artificial sequence
  HLA restricted peptide
SWQIVCSRL

SEQ ID NO: 767 - protein, 9 aa, Artificial sequence
  HLA restricted peptide
SYLEQASRI

SEQ ID NO: 768 - protein, 10 aa, Artificial sequence
  HLA restricted peptide
SYLEQASRIW

SEQ ID NO: 769 - protein, 10 aa, Artificial sequence
  HLA restricted peptide
SYLQDSDPDS

SEQ ID NO: 770 - protein, 9 aa, Artificial sequence
  HLA restricted peptide
SYMVPFIPL

SEQ ID NO: 771 - protein, 10 aa, Artificial sequence
  HLA restricted peptide
SYMVPFIPLY

SEQ ID NO: 772 - protein, 9 aa, Artificial sequence
  HLA restricted peptide
TALLAGLVS

SEQ ID NO: 773 - protein, 10 aa, Artificial sequence
  HLA restricted peptide
TALLAGLVSL

SEQ ID NO: 774 - protein, 9 aa, Artificial sequence
  HLA restricted peptide
TCQCSGNFM

SEQ ID NO: 775 - protein, 9 aa, Artificial sequence
  HLA restricted peptide
TERRLLVRR

SEQ ID NO: 776 - protein, 10 aa, Artificial sequence
  HLA restricted peptide
TERRLLVRRN

SEQ ID NO: 777 - protein, 9 aa, Artificial sequence
  HLA restricted peptide
TGDENFTIP

SEQ ID NO: 778 - protein, 10 aa, Artificial sequence
  HLA restricted peptide
TGDENFTIPY

SEQ ID NO: 779 - protein, 9 aa, Artificial sequence
  HLA restricted peptide
TGIADASQS

SEQ ID NO: 780 - protein, 10 aa, Artificial sequence
  HLA restricted peptide
TGIADASQSS

SEQ ID NO: 781 - protein, 9 aa, Artificial sequence
  HLA restricted peptide
TGVDDRESW

SEQ ID NO: 782 - protein, 10 aa, Artificial sequence
  HLA restricted peptide
TISSDYVIPI

SEQ ID NO: 783 - protein, 9 aa, Artificial sequence
  HLA restricted peptide
TLAKHTISS

SEQ ID NO: 784 - protein, 9 aa, Artificial sequence
  HLA restricted peptide
TLEGFASPL

SEQ ID NO: 785 - protein, 10 aa, Artificial sequence
  HLA restricted peptide
TLEGFASPLT

SEQ ID NO: 786 - protein, 9 aa, Artificial sequence
  HLA restricted peptide
TMSQVQGSA

SEQ ID NO: 787 - protein, 10 aa, Artificial sequence
  HLA restricted peptide
TMSQVQGSAN

SEQ ID NO: 788 - protein, 9 aa, Artificial sequence
  HLA restricted peptide
TNPNLLSPA

SEQ ID NO: 789 - protein, 10 aa, Artificial sequence
  HLA restricted peptide
TNPNLLSPAS

SEQ ID NO: 790 - protein, 9 aa, Artificial sequence
  HLA restricted peptide
TPEGPLRRN

SEQ ID NO: 791 - protein, 10 aa, Artificial sequence
  HLA restricted peptide
TPEGPLRRNP

SEQ ID NO: 792 - protein, 9 aa, Artificial sequence
  HLA restricted peptide
TPMFNDINI

SEQ ID NO: 793 - protein, 10 aa, Artificial sequence
  HLA restricted peptide
TPMFNDINIY

SEQ ID NO: 794 - protein, 9 aa, Artificial sequence
  HLA restricted peptide
TPRLPSSAD

SEQ ID NO: 795 - protein, 10 aa, Artificial sequence
  HLA restricted peptide
TPRLPSSADV

SEQ ID NO: 796 - protein, 9 aa, Artificial sequence
  HLA restricted peptide
TQYESGSMD

SEQ ID NO: 797 - protein, 10 aa, Artificial sequence
  HLA restricted peptide
TQYESGSMDK

SEQ ID NO: 798 - protein, 9 aa, Artificial sequence
  HLA restricted peptide
TSAGHFPRA

SEQ ID NO: 799 - protein, 10 aa, Artificial sequence
  HLA restricted peptide
TSAGHFPRAC

SEQ ID NO: 800 - protein, 9 aa, Artificial sequence
  HLA restricted peptide
TYGQMKNGS

SEQ ID NO: 801 - protein, 10 aa, Artificial sequence
  HLA restricted peptide
TYGQMKNGST

SEQ ID NO: 802 - protein, 9 aa, Artificial sequence
  HLA restricted peptide
VCSRLEEYN

SEQ ID NO: 803 - protein, 10 aa, Artificial sequence
  HLA restricted peptide
VCSRLEEYNS

SEQ ID NO: 804 - protein, 9 aa, Artificial sequence
  HLA restricted peptide
VDDRESWPS

SEQ ID NO: 805 - protein, 10 aa, Artificial sequence
  HLA restricted peptide
VDDRESWPSV

SEQ ID NO: 806 - protein, 9 aa, Artificial sequence
  HLA restricted peptide
VDSIFEQWL

SEQ ID NO: 807 - protein, 10 aa, Artificial sequence
  HLA restricted peptide
VDSIFEQWLR

SEQ ID NO: 808 - protein, 9 aa, Artificial sequence
  HLA restricted peptide
VEFCLSLTQ

SEQ ID NO: 809 - protein, 10 aa, Artificial sequence
  HLA restricted peptide
VEFCLSLTQY

SEQ ID NO: 810 - protein, 9 aa, Artificial sequence
  HLA restricted peptide
VFYNRTCQC

SEQ ID NO: 811 - protein, 10 aa, Artificial sequence
  HLA restricted peptide
VFYNRTCQCS

SEQ ID NO: 812 - protein, 9 aa, Artificial sequence
  HLA restricted peptide
VGAVLTALL

SEQ ID NO: 813 - protein, 10 aa, Artificial sequence
  HLA restricted peptide
VGAVLTALLA

SEQ ID NO: 814 - protein, 10 aa, Artificial sequence
  HLA restricted peptide
VIPIGTYGQM

SEQ ID NO: 815 - protein, 9 aa, Artificial sequence
  HLA restricted peptide
VLTALLAGL

SEQ ID NO: 816 - protein, 10 aa, Artificial sequence
  HLA restricted peptide
VLTALLAGLV

SEQ ID NO: 817 - protein, 9 aa, Artificial sequence
  HLA restricted peptide
VLYCLLWSF

SEQ ID NO: 818 - protein, 10 aa, Artificial sequence
  HLA restricted peptide
VLYCLLWSFQ

SEQ ID NO: 819 - protein, 9 aa, Artificial sequence
  HLA restricted peptide
VPFIPLYRN

SEQ ID NO: 820 - protein, 10 aa, Artificial sequence
  HLA restricted peptide
VPFIPLYRNG

SEQ ID NO: 821 - protein, 9 aa, Artificial sequence
  HLA restricted peptide
VQGSANDPI

SEQ ID NO: 822 - protein, 10 aa, Artificial sequence
  HLA restricted peptide
VQGSANDPIF

SEQ ID NO: 823 - protein, 9 aa, Artificial sequence
  HLA restricted peptide
VRRNIFDLS

SEQ ID NO: 824 - protein, 10 aa, Artificial sequence
  HLA restricted peptide
VRRNIFDLSA

SEQ ID NO: 825 - protein, 9 aa, Artificial sequence
  HLA restricted peptide
VSLLCRHKR

SEQ ID NO: 826 - protein, 10 aa, Artificial sequence
  HLA restricted peptide
VSLLCRHKRK

SEQ ID NO: 827 - protein, 10 aa, Artificial sequence
  HLA restricted peptide
VSMDALLGGY

SEQ ID NO: 828 - protein, 9 aa, Artificial sequence
  HLA restricted peptide
VSSKNLMEK

SEQ ID NO: 829 - protein, 9 aa, Artificial sequence
  HLA restricted peptide
VWIHYYVSM

SEQ ID NO: 830 - protein, 9 aa, Artificial sequence
  HLA restricted peptide
VYPEANAPI

SEQ ID NO: 831 - protein, 9 aa, Artificial sequence
  HLA restricted peptide
WDWRDAEKC

SEQ ID NO: 832 - protein, 9 aa, Artificial sequence
  HLA restricted peptide
WEQEIQKLT

SEQ ID NO: 833 - protein, 9 aa, Artificial sequence
  HLA restricted peptide
WGPNCTERR

SEQ ID NO: 834 - protein, 10 aa, Artificial sequence
  HLA restricted peptide
WGPNCTERRL

SEQ ID NO: 835 - protein, 9 aa, Artificial sequence
  HLA restricted peptide
WHRLFLLRW

SEQ ID NO: 836 - protein, 10 aa, Artificial sequence
  HLA restricted peptide
WIHYYVSMDA

SEQ ID NO: 837 - protein, 10 aa, Artificial sequence
  HLA restricted peptide
WLLGAAMVGA

SEQ ID NO: 838 - protein, 9 aa, Artificial sequence
  HLA restricted peptide
WPSVFYNRT

SEQ ID NO: 839 - protein, 10 aa, Artificial sequence
  HLA restricted peptide
WPSVFYNRTC

SEQ ID NO: 840 - protein, 10 aa, Artificial sequence
  HLA restricted peptide
WQIVCSRLEE

SEQ ID NO: 841 - protein, 9 aa, Artificial sequence
  HLA restricted peptide
WRDAEKCDI

SEQ ID NO: 842 - protein, 10 aa, Artificial sequence
  HLA restricted peptide
WRDAEKCDIC

SEQ ID NO: 843 - protein, 9 aa, Artificial sequence
  HLA restricted peptide
WRDIDFAHE

SEQ ID NO: 844 - protein, 10 aa, Artificial sequence
  HLA restricted peptide
WRDIDFAHEA

SEQ ID NO: 845 - protein, 9 aa, Artificial sequence
  HLA restricted peptide
WSFQTSAGH

SEQ ID NO: 846 - protein, 10 aa, Artificial sequence
  HLA restricted peptide
WSFQTSAGHF

SEQ ID NO: 847 - protein, 9 aa, Artificial sequence
  HLA restricted peptide
WSWLLGAAM

SEQ ID NO: 848 - protein, 10 aa, Artificial sequence
  HLA restricted peptide
WSWLLGAAMV

SEQ ID NO: 849 - protein, 9 aa, Artificial sequence
  HLA restricted peptide
YCLLWSFQT

SEQ ID NO: 850 - protein, 10 aa, Artificial sequence
  HLA restricted peptide
YCLLWSFQTS

SEQ ID NO: 851 - protein, 9 aa, Artificial sequence
  HLA restricted peptide
YDLFVWIHY

SEQ ID NO: 852 - protein, 10 aa, Artificial sequence
  HLA restricted peptide
YDLFVWIHYY

SEQ ID NO: 853 - protein, 9 aa, Artificial sequence
  HLA restricted peptide
YDYSYLQDS

SEQ ID NO: 854 - protein, 9 aa, Artificial sequence
  HLA restricted peptide
YEIWRDIDF

SEQ ID NO: 855 - protein, 10 aa, Artificial sequence
  HLA restricted peptide
YEIWRDIDFA

SEQ ID NO: 856 - protein, 9 aa, Artificial sequence
  HLA restricted peptide
YESGSMDKA

SEQ ID NO: 857 - protein, 10 aa, Artificial sequence
  HLA restricted peptide
YESGSMDKAA

SEQ ID NO: 858 - protein, 9 aa, Artificial sequence
  HLA restricted peptide
YGQMKNGST

SEQ ID NO: 859 - protein, 9 aa, Artificial sequence
  HLA restricted peptide
YHSLYQSHL

SEQ ID NO: 860 - protein, 9 aa, Artificial sequence
  HLA restricted peptide
YIKSYLEQA

SEQ ID NO: 861 - protein, 10 aa, Artificial sequence
  HLA restricted peptide
YIKSYLEQAS

SEQ ID NO: 862 - protein, 9 aa, Artificial sequence
  HLA restricted peptide
YLEQASRIW

SEQ ID NO: 863 - protein, 10 aa, Artificial sequence
  HLA restricted peptide
YLEQASRIWS

SEQ ID NO: 864 - protein, 9 aa, Artificial sequence
  HLA restricted peptide
YLQDSDPDS

SEQ ID NO: 865 - protein, 10 aa, Artificial sequence
  HLA restricted peptide
YLQDSDPDSF

SEQ ID NO: 866 - protein, 9 aa, Artificial sequence
  HLA restricted peptide
YLTLAKHTI

SEQ ID NO: 867 - protein, 10 aa, Artificial sequence
  HLA restricted peptide
YLTLAKHTIS

SEQ ID NO: 868 - protein, 9 aa, Artificial sequence
  HLA restricted peptide
YMDGTMSQV

SEQ ID NO: 869 - protein, 9 aa, Artificial sequence
  HLA restricted peptide
YMGGQHPTN

SEQ ID NO: 870 - protein, 9 aa, Artificial sequence
  HLA restricted peptide
YMNGTMSQV

SEQ ID NO: 871 - protein, 9 aa, Artificial sequence
  HLA restricted peptide
YMVPFIPLY

SEQ ID NO: 872 - protein, 10 aa, Artificial sequence
  HLA restricted peptide
YMVPFIPLYR

SEQ ID NO: 873 - protein, 10 aa, Artificial sequence
  HLA restricted peptide
YNRTCQCSGN

SEQ ID NO: 874 - protein, 9 aa, Artificial sequence
  HLA restricted peptide
YNSHQSLCN

SEQ ID NO: 875 - protein, 9 aa, Artificial sequence
  HLA restricted peptide
YPEANAPIG

SEQ ID NO: 876 - protein, 10 aa, Artificial sequence
  HLA restricted peptide
YPEANAPIGH

SEQ ID NO: 877 - protein, 9 aa, Artificial sequence
  HLA restricted peptide
YRNGDFFIS

SEQ ID NO: 878 - protein, 10 aa, Artificial sequence
  HLA restricted peptide
YRNGDFFISS

SEQ ID NO: 879 - protein, 9 aa, Artificial sequence
  HLA restricted peptide
YVIPIGTYG

SEQ ID NO: 880 - protein, 10 aa, Artificial sequence
  HLA restricted peptide
YVIPIGTYGQ

SEQ ID NO: 881 - protein, 9 aa, Artificial sequence
  HLA restricted peptide
YWDWRDAEK

SEQ ID NO: 882 - protein, 10 aa, Artificial sequence
  HLA restricted peptide
YWDWRDAEKC

SEQ ID NO: 883 - protein, 9 aa, Artificial sequence
  HLA restricted peptide
YYVSMDALL

SEQ ID NO: 884 - protein, 219 aa, Artificial sequence
  MC1 Ab, VL-CL polypeptide sequence
DVVMTQSPLSLPVTPGEPASISCRSSQSLLHSIGYNYLHWFLQKPGQSPQLLIYLGSNRASGVPDRFSGSGSGTDFTLKISRVEAEDVGVYYCMQALQTPPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 885 - protein, 225 aa, Artificial sequence
  MC1 Ab, VH-CH polypeptide sequence
QLQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCVGSPYGDYVLDYWGQGTLVTVSSASTKGPSVFPLAPSSKSTSGGTAALSNTKVDKGCLVKDYFPEPVTVSWNSGALTSGVHTFPAVLQSSGLYSLSSVVTVPSSSLGTQTYICNVNHKPKVEPKSCAAA

SEQ ID NO: 886 - protein, 16 aa, Artificial sequence
  B2 VL-CL CDR1
RSSQSLLHSIGYNYLD

SEQ ID NO: 887 - protein, 7 aa, Artificial sequence
  B2 VL-CL CDR2
LGSNRAS

SEQ ID NO: 888 - protein, 9 aa, Artificial sequence
  B2 VL-CL CDR3
MQALQTPLT

SEQ ID NO: 889 - protein, 5 aa, Artificial sequence
  B2 VH-CH CDR1
SYGMH

SEQ ID NO: 890 - protein, 17 aa, Artificial sequence
  B2 VH-CH CDR2
VISYDGSNKYYTDSVNG

SEQ ID NO: 891 - protein, 12 aa, Artificial sequence
  B2 VH-CH CDR3
GGGYYETSGPDY

SEQ ID NO: 892 - protein, 219 aa, Artificial sequence
  B2 Ab, VL-CL polypeptide sequence
EIVLTQSPLSLPVTPGEPASISCRSSQSLLHSIGYNYLDWYLQKPGQSPQLLIYLGSNRASGVPDRFSGSGSGTDFTLKISRVEAEDVGVYYCMQALQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 893 - protein, 227 aa, Artificial sequence
  B2 Ab, VH-CH polypeptide sequence
EVQLVESGGGVVQPGRSLRLSCAASGFTFRSYGMHWVRQAPGKGLEWVAVISYDGSNKYYTDSVNGRFTISRDNSKNTLYLQMNSLRAEDTAVYYCARGGGYYETSGPDYWGQGTLVTVSSASTKGPSVFPLAPSSKSTSGGTAALSNTKVDKGCLVKDYFPEPVTVSWNSGALTSGVHTFPAVLQSSGLYSLSSVVTVPSSSLGTQTYICNVNHKPKVEPKSCAAA

SEQ ID NO: 894 - protein, 16 aa, Artificial sequence
  MC1 VL-CL CDR1
RSSQSLLHSIGYNYLH

SEQ ID NO: 895 - protein, 7 aa, Artificial sequence
  MC1 VL-CL CDR2
LGSNRAS

SEQ ID NO: 896 - protein, 9 aa, Artificial sequence
  MC1 VL-CL CDR3
MQALQTPPT

SEQ ID NO: 897 - protein, 6 aa, Artificial sequence
  MC1 VH-CH CDR1
SSNWWS

SEQ ID NO: 898 - protein, 16 aa, Artificial sequence
  MC1 VH-CH CDR2
EIYHSGSTNYNPSLKS

SEQ ID NO: 899 - protein, 10 aa, Artificial sequence
  MC1 VH-CH CDR3
SPYGDYVLDY

SEQ ID NO: 900 - protein, 15 aa, Artificial sequence
  A fusion peptide for site specific biotinylation
LHHILDAQKMVWNHR

SEQ ID NO: 901 - DNA, 23 bp, Artificial sequence
  Single strand DNA oligonucleotide
agcggataacaatttcacacagg

SEQ ID NO: 902 - DNA, 24 bp, Artificial sequence
  Single strand DNA oligonucleotide
tttgtcgtctttccagacgttagt

SEQ ID NO: 903 - DNA, 336 bp, Artificial sequence
  MC1 Ab, VL coding sequence
gatgttgtgatgactcagtctccactctccctgcccgtcacccctggagagccggcctccatctcctgcaggtcaagtcagagcctcctgcatagtattggatacaactatttgcattggttcctgcagaagccagggcagtctccacaactcctgatctacctgggttctaatcgggcctccggggtccctgacaggttcagtggcagtggatcaggcacagattttacactgaaaatcagcagagtggaggctgaggatgttggggtttattactgcatgcaagctctgcaaactcctcccaccttcggccaagggacacgactggagattaaa

SEQ ID NO: 904 - DNA, 321 bp, Artificial sequence
  MC1 Ab, CL coding sequence
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 905 - DNA, 357 bp, Artificial sequence
  MC1 Ab, VH coding sequence
cagctgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgttgggtctccctacggtgactacgtccttgactactggggccagggcaccctggtcaccgtctcaagc

SEQ ID NO: 906 - DNA, 390 bp, Artificial sequence
  MC1 Ab, CH coding sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgcataa

SEQ ID NO: 907 - DNA, 336 bp, Artificial sequence
  B2 Ab, VL coding sequence
gaaattgtgctgactcagtctccactctccctgcccgtcacccctggagagccggcctccatctcctgcaggtctagtcagagcctcctgcatagtattggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 908 - DNA, 321 bp, Artificial sequence; identical to SEQ ID NO: 904
  B2 Ab, CL coding sequence

SEQ ID NO: 909 - DNA, 348 bp, Artificial sequence
  B2 Ab, VH coding sequence
gaggtgcagctcgtggagtctgggggaggcgtggtccagcctgggaggtccctgagactctcctgtgcagcgtctggattcaccttcaggagctatggcatgcactgggtccggcaggctccaggcaaggggctggagtgggtggcagttatatcatatgatggaagtaataaatactatacagactccgtaaacggccggttcaccatctccagagacaattccaagaacacgctgtatctgcaaatgaacagcctgagagctgaggacacggctgtgtattactgtgcgagaggagggggttactatgaaactagtggccctgactactggggccagggaaccctg

SEQ ID NO: 910 - DNA, 390 bp, Artificial sequence; identical to SEQ ID NO: 906
  B2 Ab, CH coding sequence